(19) United States
(12) Patent Application Publication
(10) Pub. No.: US 2025/0101099 A1
(43) Pub. Date: Mar. 27, 2025

(54) CHIMERIC ANTIGEN RECEPTOR T CELLS (CAR-T) FOR THE TREATMENT OF CANCER

(71) Applicant: Washington University, St. Louis, MO (US)

(72) Inventors: John DiPersio, St. Louis, MO (US); Matthew Cooper, St. Louis, MO (US); Julie O'Neal, St. Louis, MO (US)

(21) Appl. No.: 18/759,025

(22) Filed: Jun. 28, 2024

Related U.S. Application Data
(62) Division of application No. 16/428,624, filed on May 31, 2019, now abandoned.
(60) Provisional application No. 62/799,513, filed on Jan. 31, 2019.
(60) Provisional application No. 62/678,878, filed on May 31, 2018.

(51) Int. Cl. C07K 16/28; A61K 38/00; A61K 39/00; A61P 35/00; C07K 14/705; C07K 14/725

(52) U.S. Cl. CPC C07K 16/2803; A61K 40/11; A61K 40/31; A61K 40/421; A61K 40/4211; A61K 40/4215; A61P 35/00; C07K 14/7051; C07K 14/70521; C07K 14/70578; C07K 16/2806; A61K 38/00; A61K 2039/505; A61K 2239/29; A61K 2239/48; C07K 2317/622; C07K 2317/76; C07K 2319/02; C07K 2319/30; C07K 2319/33

(57) ABSTRACT

Disclosed herein are genome-edited chimeric antigen receptor T cells (CAR-T), which can be derived from a cytotoxic T cells, a viral-specific cytotoxic T cell, memory T cells, or gamma delta (γδ) T cells, and comprise one or more chimeric antigen receptors (CARs) targeting one or more antigens, wherein the CAR-T cell is deficient in one or more antigens to which the one or more CARs specifically binds. In particular, the present disclosure relates to engineered mono, dual, and tandem chimeric antigen receptor (CAR)-bearing T cells (CAR-T) and methods of immunotherapy for the treatment of cancer.

This application is a divisional application of U.S. application Ser. No. 16/428,624, filed May 31, 2019, which claims the benefit of priority of U.S. Provisional Patent Application No. 62/799,513, filed on Jan. 31, 2019, and U.S. Provisional Patent Application No. 62/678,878 filed on May 31, 2018, the disclosures of which are hereby incorporated by reference as if written herein in their entireties.

### SEQUENCE LISTING

The instant application contains a Sequence Listing which has been submitted electronically in Extensible Markup Language (.xml) format and is hereby incorporated by reference in its entirety. Said XML copy, created on Jun. 26, 2024, is named 047563_810161.xml and is 762,142 bytes in size.

### FIELD OF INVENTION

Disclosed herein are genome-edited chimeric antigen receptor T cells (CAR-T) and methods of using them for immunotherapy. In particular, the disclosure relates to T cells that can be genetically modified to express one or more chimeric antigen receptors (CARs) and methods of using the same for the treatment of cancer.

### BACKGROUND

T cells, a type of lymphocyte, play a central role in cell-mediated immunity. They are distinguished from other lymphocytes, such as B cells and natural killer cells (NK cells), by the presence of a T-cell receptor (TCR) on the cell surface. T helper cells (TH), also called CD4⁺ T or CD4 T cells, express CD4 glycoprotein on their surface. Helper T cells are activated when exposed to peptide antigens presented by MHC (major histocompatibility complex) class II molecules. Once activated, these cells proliferate rapidly and secrete cytokines that regulate immune response. Cytotoxic T cells (Tc), also known as CD8⁺ T cells or CD8 T cells, express CD8 glycoprotein on the cell surface. The CD8⁺ T cells are activated when exposed to peptide antigens presented by MHC class I molecules. Memory T cells, a subset of T cells, persist long term and respond to their cognate antigen, thus providing the immune system with “memory” against past infections and/or tumor cells. Gamma delta (γδ) T cells are the prototype of ‘unconventional’ T cells and represent a relatively small subset of T cells in peripheral blood. They are defined by expression of heterodimeric T-cell receptors (TCRs) composed of γ and δ chains. This sets them apart from the CD4⁺ helper T cells and CD8⁺ cytotoxic T cells. Viral-specific cytotoxic T lymphocytes are T cells with reactivity against viral antigens, notably Epstein-Barr virus (EBV) and cytomegalovirus (CMV).

The T cells described herein can be genetically modified to express chimeric antigen receptors (CARs), which are fusion proteins comprised of an antigen recognition moiety and T cell activation domains. T cells expressing CARs can recognize a specific protein, i.e., antigen on tumor cells. These T cells expressing CARs can be expanded in the laboratory prior to infusion into a patient.

Clinical trials have shown high response rates after anti-CD19 CAR infusion in patients with B cell malignancies, including diffuse large B cell lymphoma (DLBCL) and B cell-precursor acute lymphoblastic leukemia (ALL), resulting in two FDA approved therapies Yescarta™ (axicabtagene ciiloleucel, Kite Pharma/Gilead) and Kymriah™ (tisagenlecleucel, Novartis). Despite these successes, the development of CAR-T cell therapy against T cell malignancies has proven problematic, in part due to the shared expression of target antigens between malignant T cells and effector T cells. Among the most general challenges are: (1) the antigen target(s) for the chimeric antigen receptor(s); (2) CAR design, i.e., mono CAR, dual CAR, tandem CAR; and (3) tumor heterogeneity, particularly the variance in the surface expression of tumor antigens. Therefore, there remains a need for improved chimeric antigen receptor (CAR)-based immunotherapies, which utilize genome-editing and construction of mono, dual, and tandem CARs, for more effective, safe, and efficient targeting of cancers, including T-cell associated malignancies.

### BRIEF DESCRIPTION OF THE DRAWINGS

FIG. 1 shows a schematic of a dual CAR-T cell (dCAR-T cell).

FIG. 2 shows a schematic of a tandem CAR-T cell (tCAR-T cell).

FIG. 3 shows a schematic of dual and tandem CAR constructs.

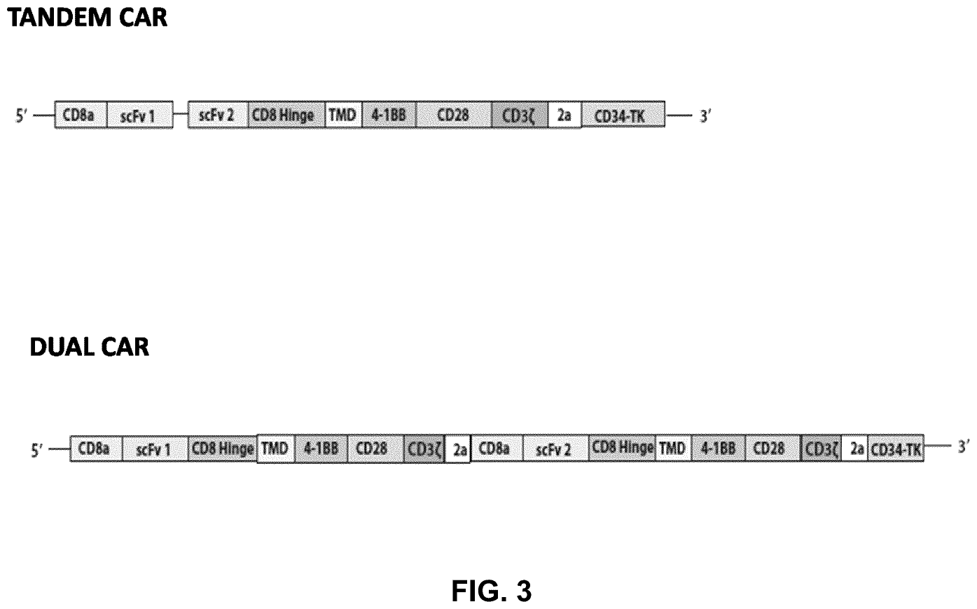

FIG. 4 shows a schematic of tandem targeting CAR constructs.

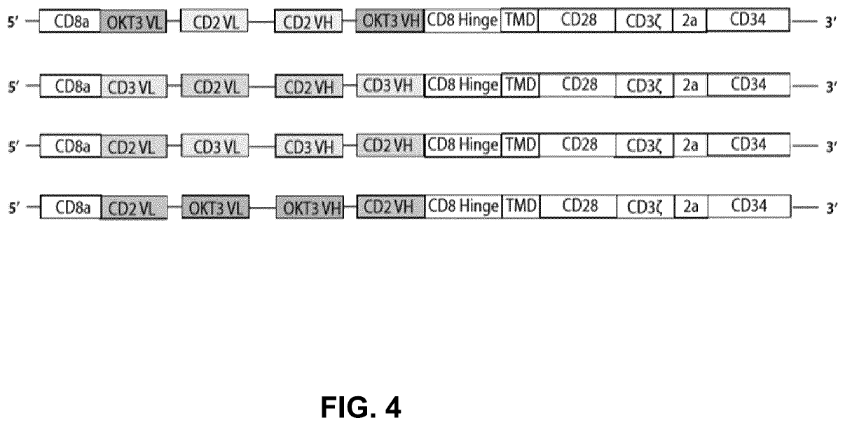

FIG. 5 shows the purity of CAR-T product without mechanical depletion of CD3+ or CD2+ CAR-T cells. As shown through FACS analysis, there is a high purity of CD3⁻ and CD2⁻ CAR-T cells without a requirement for magnetic depletion of CD3+ cells. Representative FACS plots show FITC-staining for CD3 (y-axis) and CD2 (x-axis). Clones 5 (top) and 6 (bottom) shown.

FIG. 6 shows the purity of CAR-T product without mechanical depletion of CD3+ or CD2+ CAR-T cells. As shown through FACS analysis, there is a high purity of CD3− and CD2− CAR-T cells without a requirement for magnetic depletion selection of CD3+ cells. Representative FACS plots show FITC-staining for CD3 (y-axis) and CD2 (x-axis). Clones 7 (top) and 8 (bottom) shown.

FIG. 7 shows the purity of CAR-T product without mechanical depletion of CD3+ or CD2+ CAR-T cells. As shown through FACS analysis, there is a high purity of CD3− and CD2− CAR-T cells without a requirement for magnetic depletion selection of CD3+ cells. Representative FACS plots show FITC-staining for CD3 (y-axis) and CD2 (x-axis). Clones 13 (top) and 14 (bottom) shown.

FIG. 8 shows the purity of CAR-T product without mechanical depletion of CD3+ or CD2+ CAR-T cells. As shown through FACS analysis, there is a high purity of CD3− and CD2− CAR-T cells without a requirement for magnetic depletion selection of CD3+ cells. Representative FACS plots show FITC-staining for CD3 (y-axis) and CD2 (x-axis). Clones 15 (top) and 16 (bottom) shown.

FIG. 9A shows tumor cell killing of tandem CD2-CD3 CAR-T Clones 5 (top) and 6 (bottom); the legend shows ratio of effector to target cells (E:T ratio).

FIG. 9B shows tumor cell killing of tandem CD2-CD3 CAR-T Clones 7 (top) and 8 (bottom); the legend shows ratio of effector to target cells (E:T ratio).

FIG. 9C shows tumor cell killing of tandem CD2-CD3 CAR-T Clones 13 (top) and 14 (bottom); the legend shows ratio of effector to target cells (E:T ratio).

FIG. 9D shows tumor cell killing of tandem CD2-CD3 CAR-T Clones 15 (top) and 16 (bottom); the legend shows ratio of effector to target cells (E:T ratio).

FIG. 10A shows a schematic of a BCMA CAR construct to be transduced into T cells which will target BCMA.

FIG. 10B shows a tumor cell killing of BCMA-CAR-T cells in a ⁵¹Cr-release assay. Efficient killing of BCMA-CAR-T cells were observed at multiple Effector to Target (E:T) ratios. Non-transduced activated T cells and CD19-CAR-T cells were used as negative controls and did not induce killing of MM.1 S-CG cells.

FIG. 10C shows in vivo efficacy of BCMA CAR-T cells. All seven mice treated with BCMA CAR-Ts lived to almost 150 days or more compared to controls which died around Day 50.

FIG. 10D shows serial bioluminescent imaging (BLI) measured in photo flux revealed showed a robust reduction of signal to background levels that never increased throughout the duration of the experiment in mice which received treatment with BCMA CAR-T cells.

FIG. 11A. shows a schematic of a CS1-CAR construct to be transduced into T cells which will target CS1.

FIG. 11B shows in vivo efficacy of CS1-CAR-T cells. Mice were engrafted with MM.1 S-CG cells and MM.1 S-CG cells lacking CS1 (using CAS9/CRISPR technology; MM.1 S-CGACS1) as a method to test the specificity of CS1-CAR-T cells. All mice treated with CS1-CAR-T cells (n=10) lived >90 days while median survival of CD19-control mice (n=8) was 43 days.

FIG. 11C shows serial bioluminescent imaging (BLI) showed mice treated with CS1-CAR-T cells had a three-log decrease in photon flux and clearance of marrow tumor (Experiment 1 through Experiment 3).

FIG. 12A shows schematics of mono (CD19, CS1) and tandem (BCMA-CS1) constructs.

FIG. 12B shows FACS analysis of Jurkat cells expressing CD19 CAR did not bind to either BCMA or CS1 protein (lower left quadrant of each plot). Jurkat cells expressing BCMA CAR protein bound BCMA protein (upper left quadrant of each plot). Jurkat cells expressing CS1 CAR protein bound CS1 protein (lower right quadrant of each plot). Jurkat cells expressing the tandem BCMA-CS1 CAR protein bound to both recombinant proteins (upper right quadrant of each plot), suggesting expression of both scFvs.

FIG. 12C shows in vitro efficacy of single and tandem CAR-T cells using standard four-hour chromium release (⁵¹Cr) assays. BCMA-CS1 tCAR-T cells killed MM.1S-CG cells with similar efficacy of both single-antigen targeted BCMA and CS1 CAR-T cells.

FIG. 13 shows testing efficacy of CD2*CD3Δ-dCARTΔCD2ΔCD3 in a xenogeneic model of T-ALL.

### DETAILED DESCRIPTION

The following disclosure will detail embodiments, alternatives, and uses engineered cells and the use sch cells in, for example, immunotherapy and adoptive cell transfer for the treatment of diseases. Accordingly, provided herein are the following embodiments.

Embodiment 1. A CAR-T cell, which comprises one or more chimeric antigen receptors (CARs) targeting one or more antigens, wherein the CAR-T cell is deficient in a subunit of the T cell receptor complex and/or is deficient in at least one or more antigens to which the one or more CARs specifically binds.

Embodiment 2. A CAR-T cell, which comprises one or more chimeric antigen receptors (CARs) targeting one or more antigens, wherein the CAR-T cell is deficient in one or more antigens to which the one or more CARs specifically binds.

Embodiment 3. The CAR-T cell as recited in Embodiment 1, wherein the subunit of the T cell receptor complex is chosen from TCRα, TCRβ, TCRδ, TCRγ, CD3ε, CD3γ, CD3δ, and CD3ζ.

Embodiment 4. The CAR-T cell as recited in any of Embodiments 1-2, wherein the chimeric antigen receptor (CAR) specifically binds one or more antigens expressed on a malignant T cell or myeloma cell.

Embodiment 5. The CAR-T cell as recited in any of Embodiments 1-4, wherein the chimeric antigen receptor (CAR) displays at least 95% sequence identity to an amino acid sequence chosen from SEQ ID NO:32, SEQ ID NO:33, SEQ ID NO:34, SEQ ID NO:35, SEQ ID NO:36, SEQ ID NO:37, SEQ ID NO:38 or SEQ ID NO:39.

Embodiment 6. The CAR-T cell as recited in any of Embodiments 1-4, wherein the chimeric antigen receptor (CAR) displays at least 98% sequence identity to an amino acid sequence chosen from SEQ ID NO:32, SEQ ID NO:33, SEQ ID NO:34, SEQ ID NO:35, SEQ ID NO:36, SEQ ID NO:37, SEQ ID NO:38 or SEQ ID NO:39.

Embodiment 7. The CAR-T cell as recited in any of Embodiments 1-4, wherein the chimeric antigen receptor (CAR) is an amino acid sequence chosen from SEQ ID NO:32, SEQ ID NO:33, SEQ ID NO:34, SEQ ID NO:35, SEQ ID NO:36, SEQ ID NO:37, SEQ ID NO:38 or SEQ ID NO:39.

Embodiment 8. The CAR-T cell as recited in any of Embodiments 1-4, wherein the chimeric antigen receptor(s) specifically binds one or more antigen(s) chosen from BCMA, CS1, CD38, CD138, CD19, CD33, CD123, CD371, CD117, CD135, Tim-3, CD5, CD7, CD2, CD4, CD3, CD79A, CD79B, APRIL, CD56, and CD1a.

Embodiment 9. The CAR-T cell as recited in any of Embodiments 1-5, wherein the chimeric antigen receptor(s) specifically binds at least one antigen expressed on a malignant T cell.

Embodiment 10. The CAR-T cell as recited in Embodiment 9, wherein the antigen expressed on a malignant T cell is chosen from CD2, CD3, CD4, CD5, CD7, TCRA, and TCRβ.

Embodiment 11. The CAR-T cell as recited in any of Embodiments 1-5, wherein the chimeric antigen receptor specifically binds at least one antigen expressed on a malignant plasma cell.

Embodiment 12. The CAR-T cell as recited in Embodiment 11, wherein the antigen expressed on a malignant plasma cell is chosen from BCMA, CS1, CD38, CD79A, CD79B, CD138, and CD19.

Embodiment 13. The CAR-T cell as recited in any of Embodiments 1-5, wherein the chimeric antigen receptor(s) specifically binds at least one antigen expressed on a malignant B cell.

Embodiment 14. The CAR-T cell as recited in Embodiment 13, wherein the antigen expressed on a malignant B cell is chosen from CD19, CD20, CD21, CD22, CD23, CD24, CD25, CD27, CD38, and CD45.

Embodiment 15. The CAR-T cell as recited in Embodiment 14, wherein the antigen expressed on a malignant B cell is chosen from CD19 and CD20.

Embodiment 16. The CAR-T cell as recited in any of Embodiments 1-15, wherein the CAR-T cell further comprises a suicide gene.

Embodiment 17. The CAR-T cell as recited in any of Embodiments 1-16, wherein endogenous T cell receptor mediated signaling is blocked in the CAR-T cell.

Embodiment 18. The CAR-T cell as recited in any of Embodiments 1-17, wherein the CAR-T cells do not induce alloreactivity or graft-versus-host disease.

Embodiment 19. The CAR-T cell as recited in any of Embodiments 1-18, wherein the CAR-T cells do not induce fratricide.

Embodiment 20. A dual or tandem CAR-T cell as recited in any of Embodiments 1-19.

Embodiment 21. The CAR-T cell as recited in Embodiment 20, wherein the wherein the CAR(s) specifically bind(s) two different targets chosen from: CD2xCD3ε, CD2xCD4, CD2xCD5, CD2xCD7, CD3εxCD4, CD3εxCD5, CD3εxCD7, CD4xCD5, CD4xCD7, CD5xCD7, TRACxCD2, TRACxCD3ε, TRACxCD4, TRACxCD5, TRACxCD7, TCRβxCD2, TCRβxCD3ε, TCRβxCD4, TCRβxCD7, CD2xCD3ε, CD2xCD4, CD2xCD5, CD2xCD7, CD3εxCD4, CD3εxCD5, CD3εxCD7, CD4xCD5, CD4xCD7, CD5xCD7, TRACxCD2, TRACxCD3ε, TRACxCD4, TRACxCD5, TRACxCD7, TCRβxCD2, TCRβxCD3ε, TCRβxCD4, TCRβxCD5, TCRβxCD7, BCMAxCS1, BCMAxCD19, BCMAxCD38, CS1xCD19, CD19xCD38, APRILxCS1, APRILxBCMA, APRILxCD19, APRILxCD38, CS1xCD38, CD79AxBCMA, CD79AxCS1, CD79AxCD19, CD79AxCD38, CD79AxCD38, CD79AxAPRIL, CD79AxCD79B, CD79BxBCMA, CD79BxCS1, CD79BxCD19, CD79BxCD38, CD79BxAPRIL, CD79BxCD79A, CD138xBCMA, CD138xCS1, CD138xCD19, CD138xCD38, CD138xAPRIL, CD138xCD79A, CD138xCD79B, CD138xBCMA, and CD138xCS1.

Embodiment 22. The CAR-T cell as recited in Embodiment 21, wherein the CAR(s) specifically bind(s) two different targets chosen from: CD2xCD3ε, CD2xCD4, CD2xCD5, CD2xCD7, CD3εxCD4, CD3εxCD5, CD3εxCD7, CD4xCD5, CD4xCD7, CD5xCD7, TRACxCD2, TRACxCD3ε, TRACxCD4, TRACxCD5, TRACxCD7, TCRβxCD2, TCRβxCD3ε, TCRβxCD4, TCRβxCD7, CD2xCD3ε, CD2xCD4, CD2xCD5, CD2xCD7, CD3εxCD4, CD3εxCD5, CD3εxCD7, CD4xCD5, CD4xCD7, CD5xCD7, TRACxCD2, TRACxCD3ε, TRACxCD4, TRACxCD5, TRACxCD7, TCRβxCD2, TCRβxCD3ε, TCRβxCD4, TCRβxCD5, and TCRβxCD7.

Embodiment 23. The CAR-T cell as recited in Embodiment 21, wherein the CAR(s) specifically bind(s) two different targets chosen from: BCMAxCS1, BCMAxCD19, BCMAxCD38, CS1xCD19, CD19xCD38, APRILxCS1, APRILxBCMA, APRILxCD19, APRILxCD38, CS1xCD38, CD79AxBCMA, CD79AxCS1, CD79AxCD19, CD79AxCD38, CD79AxCD38, CD79AxAPRIL, CD79AxCD79B, CD79BxBCMA, CD79BxCS1, CD79BxCD19, CD79BxCD38, CD79BxAPRIL, CD79BxCD79A, CD138xBCMA, CD138xCS1, CD138xCD19, CD138xCD38, CD138xAPRIL, CD138xCD79A, CD138xCD79B, CD138xBCMA, and CD138xCS1.

Embodiment 24. The CAR-T cell as recited in Embodiment 21, wherein the CAR(s) specifically bind(s) two different targets chosen from: CD123xCD371, CD123xCLEC12A, CD123xCD117, CD123xFLT3, CD123xCD7, CD123xTim3, CD371xCLEC12A, CD371xCD117, CD371xFLT3, CD371xCD7, CD371xTim3, CLEC12AxCD117, CLEC12AxFLT3, CLEC12AxCD7, CLEC12AxTim3, CD117xFLT3, CD117xCD7, CD117xTim3, FLT3xCD7, FLT3xTim3, and CD7xTim3.

Embodiment 25. A dual CAR-T cell as recited in any of Embodiments 21-24.

Embodiment 26. A tandem CAR-T cell as recited in any of Embodiments 21-34.

Embodiment 27. The CAR-T cell as recited in any of Embodiments 1-26, wherein the CAR-T cell further comprises a suicide gene.

Embodiment 28. The CAR-T cell as recited in any of Embodiments 1-26, wherein endogenous T cell receptor mediated signaling is blocked in the CAR-T cell.

Embodiment 29. The CAR-T cell as recited in any of Embodiments 1-26, wherein the CAR-T cells do not induce alloreactivity or graft-versus-host disease.

Embodiment 30. The CAR-T cell as recited in any of Embodiments 1-26, wherein the CAR-T cells do not induce fratricide.

Embodiment 31. A dual or tandem chimeric antigen receptor (dCAR or tCAR) targeting two or more plasma cell antigens.

Embodiment 32. The CAR as recited in Embodiment 31, wherein the plasma cell antigen(s) is/are chosen from BCMA, CS1, CD38, CD79A, CD79B, CD138, and CD19.

Embodiment 33. The CAR as recited in Embodiment 32, wherein the CAR(s) specifically bind(s) two different targets chosen from: BCMAxCS1, BCMAxCD19, BCMAxCD38, CS1xCD19, CD19xCD38, APRILxCS1, APRILxBCMA, APRILxCD19, APRILxCD38, CS1xCD38, CD79AxBCMA, CD79AxCS1, CD79AxCD19, CD79AxCD38, CD79AxCD38, CD79AxAPRIL, CD79AxCD79B, CD79BxBCMA, CD79BxCS1, CD79BxCD19, CD79BxCD38, CD79BxAPRIL, CD79BxCD79A, CD138xBCMA, CD138xCS1, CD138xCD19, CD138xCD38, CD138xAPRIL, CD138xCD79A, CD138xCD79B, CD138xBCMA, and CD138xCS1.

Embodiment 34. The CAR as recited in any of Embodiments 31-33, wherein the CAR is a dCAR.

Embodiment 35. The CAR as recited in any of Embodiments 31-33, wherein the CAR is a tCAR.

Embodiment 36. A dual or tandem chimeric antigen receptor (dCAR or tCAR) targeting two or more leukemia cell antigens.

Embodiment 37. The CAR as recited in Embodiment 36, wherein the plasma cell antigen(s) is/are chosen from CD123, CLEC12A, CD117, FLT3, CD7 and Tim3.

Embodiment 38. The CAR as recited in Embodiment 37, wherein the CAR(s) specifically bind(s) two different targets chosen from: CD123xCD371, CD123xCLEC12A, CD123xCD117, CD123xFLT3, CD123xCD7, CD123xTim3, CD371xCLEC12A, CD371xCD117, CD371xFLT3, CD371xCD7, CD371xTim3, CLEC12AxCD117, CLEC12AxFLT3, CLEC12AxCD7, CLEC12AxTim3, CD117xFLT3, CD117xCD7, CD117xTim3, FLT3xCD7, FLT3xTim3, and CD7xTim3.

Embodiment 39. The CAR as recited in any of Embodiments 36-38, wherein the CAR is a dCAR.

Embodiment 40. The CAR as recited in any of Embodiments 36-38, wherein the CAR is a tCAR.

Embodiment 41. A tandem chimeric antigen receptor (tCAR) targeting two or more T-cell antigens.

Embodiment 42. The tCAR as recited in Embodiment 41, wherein the T-cell antigens chosen from CD5, CD7, CD2, CD4, and CD3.

Embodiment 43. The tCAR as recited in Embodiment 42, targeting a pair of (i.e., two) antigens.

Embodiment 44. The tCAR as recited in Embodiment 43, wherein the antigen pair is chosen from CD2xCD3ε, CD2xCD4, CD2xCD5, CD2xCD7, CD3εxCD4, CD3εxCD5, CD3εxCD7, CD4xCD5, CD4xCD7, CD5xCD7, TRACxCD2, TRACxCD3ε, TRACxCD4, TRACxCD5, TRACxCD7, TCRβxCD2, TCRβxCD4, TCRβxCD7, CD2xCD3ε, CD2xCD4, CD2xCD5, CD2xCD7, CD3εxCD4, CD3εxCD5, CD4xCD5, CD4xCD7, CD5xCD7, TRACxCD2, TRACxCD3ε, TRACxCD4, TRACxCD5, TRACxCD7, TCRβxCD2, TCRβxCD3ε, TCRβxCD4, TCRβxCD5, and TCRβxCD7.

Embodiment 45. The tCAR as recited in Embodiment 43, wherein the antigen pair is chosen from CD2xCD3ε, CD2xCD4, CD2xCD5, CD2xCD7, CD3εxCD4, CD3εxCD5, CD3εxCD7, CD4xCD5, CD4xCD7, and CD5xCD7.

Embodiment 46. The tCAR as recited in any of Embodiments 35 and 40-45, wherein the CAR construct is a linear tCAR construct.

Embodiment 47. The tCAR as recited in Embodiment 46, wherein the linear tCAR construct comprises a first heavy (V_{H}) chain variable fragment and a first light (V_{L}) chain variable fragment, designated V_{H}1 and V_{L}1, joined by a (GGGGS)₂₋₆ (SEQ ID NO:447) linker to a second light (V_{L}) chain variable fragment and a first heavy (V_{H}) chain variable fragment, designated V_{L}2 and V_{H}2.

Embodiment 48. The tCAR as recited in Embodiment 46, wherein the linear tCAR construct comprises a first heavy (V_{H}) chain variable fragment and a first light (V_{L}) chain variable fragment, designated V_{H}2 and V_{L}2, joined by a (GGGGS)₂₋₆ (SEQ ID NO:447) linker to a second light (V_{L}) chain variable fragment and a first heavy (V_{H}) chain variable fragment, designated V_{H}1 and V_{L}1.

Embodiment 49. The tCAR as recited in Embodiment 46, wherein the linear tCAR construct comprises a first light (V_{L}) chain variable fragment and a first heavy (V_{H}) chain variable fragment, designated V_{L}1 and V_{H}1, joined by a (GGGGS)₂₋₆ (SEQ ID NO:447) linker to a second heavy (V_{H}) chain variable fragment and a first light (V_{L}) chain variable fragment, designated V_{H}2 and V_{L}2.

Embodiment 50. The tCAR as recited in Embodiment 46, wherein the linear tCAR construct comprises a first light (V_{L}) chain variable fragment and a first heavy (V_{H}) chain variable fragment, designated V_{L}2 and V_{H}2, joined by a (GGGGS)₂₋₆ (SEQ ID NO:447) linker to a second heavy (V_{H}) chain variable fragment and a first light (V_{L}) chain variable fragment, designated V_{H}1 and V_{L}1.

Embodiment 51. The tCAR as recited in Embodiment 46, wherein the linear tCAR construct comprises a structure chosen from 7-I to 7-XXXII.

Embodiment 52. The tCAR as recited in any of Embodiments 35 and 40-45, wherein the CAR construct is a hairpin tCAR construct.

Embodiment 53. The tCAR as recited in Embodiment 52, wherein the hairpin tCAR construct comprises a first heavy (V_{H}) chain variable fragment derived from a first scFv, and a second heavy (V_{H}) chain variable fragment derived from a second scFv, designated V_{H}1 and V_{H}2, joined by a (GGGGS)₂₋₆ (SEQ ID NO:447) linker to a first light (V_{L}) chain variable fragment derived from the second scFv, and a second light (V_{L}) chain variable fragment derived from the first scFv, designated V_{L}2 and V₁2.

Embodiment 54. The tCAR as recited in Embodiment 52, wherein the hairpin tCAR construct comprises a second heavy (V_{H}) chain variable fragment derived from a second scFv, and a first heavy (V_{H}) chain variable fragment derived from a first scFv, designated V_{H}2 and V_{H}1, joined by a (GGGGS)₂₋₆ (SEQ ID NO:447) linker to a first light (V_{L}) chain variable fragment derived from the first scFv, and a second light (V_{L}) chain variable fragment derived from the second scFv, designated V_{L}1 and V_{L}2.

Embodiment 55. The tCAR as recited in Embodiment 52, wherein the hairpin tCAR construct comprises a first light (V_{L}) chain variable fragment derived from a first scFv, and a second light (V_{L}) chain variable fragment derived from a second scFv, designated V_{L}1 and V_{L}2, joined by a (GGGGS)₂₋₆ (SEQ ID NO:447) linker to a first heavy (V_{H}) chain variable fragment derived from the first scFv, and a second heavy (V_{L}) chain variable fragment derived from the second scFv, designated V_{H}2 and V_{H}1.

Embodiment 56. The tCAR as recited in Embodiment 52, wherein the hairpin tCAR construct comprises a second light (V_{L}) chain variable fragment derived from a second scFv, and a first light (V_{L}) chain variable fragment derived from a first scFv, designated V_{L}2 and V_{L}1, joined by a (GGGGS)₂₋₆ (SEQ ID NO:447) linker to a first heavy (V_{H}) chain variable fragment derived from the first scFv, and a second light heavy (V_{H}) variable fragment derived from the second scFv, designated V_{H}1 and V_{H}2.

Embodiment 57. The tCAR as recited in Embodiment 52, wherein the hairpin tCAR construct comprises a structure chosen from 9-I to 9-XXXII.

Embodiment 58. The tCAR as recited in any of Embodiments 35 and 40-45, wherein the CAR construct is a hairpin DSB tCAR construct with a (Cys=Cys) Double-Stranded Bond (DSB) in the linker.

Embodiment 59. The tCAR as recited in Embodiment 58, wherein the hairpin tCAR construct comprises a first heavy (V_{H}) chain variable fragment derived from a first scFv, and a second heavy (V_{H}) chain variable fragment derived from a second scFv, designated V_{H}1 and V_{H}2, joined by a (GGGGS)₀₋₁-(GGGGC)₁-(GGGGS)₁₋₂-(GGGGP)₁-(GGGGS)₂₋₃-(GGGGC)₁-(GGGGS)₀₋₁ (SEQ ID NO:448) linker to a first light (V_{L}) chain variable fragment derived from the second scFv, and a second light (V_{L}) chain variable fragment derived from the first scFv, designated V_{L}2 and V₁2.

Embodiment 60. The tCAR as recited in Embodiment 58, wherein the hairpin tCAR construct comprises a second heavy (V_{H}) chain variable fragment derived from a second scFv, and a first heavy (V_{H}) chain variable fragment derived from a first scFv, designated V_{H}2 and V_{H}1, joined by a (GGGGS)₀₋₁-(GGGGC)₁-(GGGGS)₁₋₂-(GGGGP)₁-(GGGGS)₂₋₃-(GGGGC)₁-(GGGGS)₀₋₁ (SEQ ID NO:448) linker to a first light (V_{L}) chain variable fragment derived from the first scFv, and a second light (V_{L}) chain variable fragment derived from the second scFv, designated V_{L}1 and V_{L}2.

Embodiment 61. The tCAR as recited in Embodiment 58, wherein the hairpin tCAR construct comprises a first light (V_{L}) chain variable fragment derived from a first scFv, and a second light (V_{L}) chain variable fragment derived from a second scFv, designated V_{L}1 and V_{L}2, joined by a (GGGGS)₀₋₁-(GGGGC)₁-(GGGGS)₁₋₂-(GGGGP)₁-(GGGGS)₂₋₃-(GGGGC)₁-(GGGGS)₀₋₁ (SEQ ID NO:448) linker to a first heavy (V_{H}) chain variable fragment derived from the first scFv, and a second heavy (V_{L}) chain variable fragment derived from the second scFv, designated V_{H}2 and V_{H}1.

Embodiment 62. The tCAR as recited in Embodiment 58, wherein the hairpin tCAR construct comprises a second light (V_{L}) chain variable fragment derived from a second scFv, and a first light (V_{L}) chain variable fragment derived from a first scFv, designated V_{L}2 and V_{L}1, joined by a (GGGGS)₀₋₁-(GGGGC)₁-(GGGGS)₁₋₂-(GGGGP)₁-(GGGGS)₂₋₃-(GGGGC)₁-(GGGGS)₀₋₁ (SEQ ID NO:448) linker to a first heavy (V_{H}) chain variable fragment derived from the first scFv, and a second light heavy (V_{H}) variable fragment derived from the second scFv, designated V_{H}1 and V_{H}2.

Embodiment 63. The tCAR as recited in Embodiment 58, wherein the hairpin DSB tCAR construct comprises a structure chosen from 11-I to 11-XXXII.

Embodiment 64. The tCAR as recited in any of Embodiments 41-63, wherein each of the V_{H} and V_{L} chains is derived from an scFv that recognizes a different antigen chosen from CD5, CD7, CD2, CD4, and CD3.

Embodiment 65. The tCAR as recited in Embodiment 64, wherein each of the V_{H} and V_{L} chains is different and displays at least 95% sequence identity to an amino acid sequence chosen from SEQ ID NO:12 to SEQ ID NO:31.

Embodiment 66. The tCAR as recited in Embodiment 64, wherein each of the V_{H} and V_{L} chains is different and displays at least 98% sequence identity to an amino acid sequence chosen from SEQ ID NO:12 to SEQ ID NO:31.

Embodiment 67. The tCAR as recited in Embodiment 64, wherein each of the V_{H} and V_{L} chains is different and is a sequence chosen from SEQ ID NO:12 to SEQ ID NO:31.

Embodiment 68. The tCAR as recited in any of Embodiments 35, 39, and 41-67, comprising at least one costimulatory domain chosen from CD28 and 4-1BB.

Embodiment 69. The tCAR as recited in Embodiment 68, wherein the costimulatory domain is CD28.

Embodiment 70. The tCAR as recited in any of Embodiments 35 and 40-69, comprising a CD3c signaling domain.

Embodiment 71. The tCAR as recited in any of Embodiments 41-63 and 68-70, wherein the each of the V_{H} and V_{L} chains is derived from an scFv recognizing CD2 or an scFv recognizing CD3.

Embodiment 72. The tCAR as recited in Embodiment 64, wherein the tCAR construct is chosen from Clone 5, Clone 6, Clone 7, Clone 8, Clone 13, Clone 14, Clone 15, and Clone 16.

Embodiment 73. The tCAR as recited in Embodiment 64, wherein the tCAR construct displays at least 95% sequence identity to an amino acid sequence chosen from SEQ ID NO:41 to SEQ ID NO:46.

Embodiment 74. A tandem chimeric antigen receptor (CAR) T cell (tCAR-T cell), which comprises a tCAR targeting two or more T-cell antigens, as recited in any of Embodiments 35 and 40-73.

Embodiment 75. The tCAR-T cell as recited in Embodiment 74, wherein the cell is deficient in one or more antigens to which the one or more CARs specifically binds.

Embodiment 76. The tCAR-T cell as recited in either of Embodiments 74 and 75, wherein the tCAR-T cell is deficient in a subunit of the T cell receptor complex.

Embodiment 77. The tCAR-T cell as recited in Embodiment 76, wherein the subunit of the T cell receptor complex is chosen from TCRα(TRAC), TCRβ, TCRδ, TCRγ, CD3ε, CD3γ, CD3δ, and CD3ζ.

Embodiment 78. The tCAR-T cell as recited in Embodiment 77, wherein the subunit of the T cell receptor complex is chosen from TCRα(TRAC) and CD3ε.

Embodiment 79. The tCAR-T cell as recited in Embodiment 78, wherein the subunit of the T cell receptor complex is TRAC.

Embodiment 80. The tCAR-T cell as recited in any of Embodiments 35 and 40-79, wherein the CAR-T cell further comprises a suicide gene.

Embodiment 81. The tCAR-T cell as recited in any of Embodiments 35 and 40-80, wherein endogenous T cell receptor mediated signaling is blocked in the CAR-T cell.

Embodiment 82. The tCAR-T cell as recited in any of Embodiments 35 and 40-81, wherein the CAR-T cells do not induce alloreactivity or graft-versus-host disease.

Embodiment 83. The tCAR-T cell as recited in any of Embodiments 35 and 40-82, wherein the CAR-T cells do not induce fratricide.

Embodiment 84. A tandem CAR-T cell having a CAR targeting CD2 and CD3, wherein the CAR-T cell is deficient in a subunit of the T cell receptor complex and is deficient in CD2.

Embodiment 85. The CAR-T cell as recited in Embodiment 85, wherein the CAR displays at least 95% sequence identity to an amino acid sequence chosen from SEQ ID NO:41 to SEQ ID NO:44.

Embodiment 86. The CAR-T cell as recited in Embodiment 85, wherein the CAR displays at least 98% sequence identity to an amino acid sequence chosen from SEQ ID NO:41 to SEQ ID NO:44.

Embodiment 87. The CAR-T cell as recited in Embodiment 85, wherein the CAR is an amino acid sequence chosen from SEQ ID NO:41 to SEQ ID NO:44.

Embodiment 88. A tandem CAR-T cell having a CAR targeting CD2 and CD7, wherein the CAR-T cell is deficient in a subunit of the T cell receptor complex and is deficient in CD2 and CD7.

Embodiment 89. The CAR-T cell as recited in Embodiment 88, wherein the CAR displays at least 95% sequence identity to an amino acid sequence chosen from SEQ ID NO:45 to SEQ ID NO:46.

Embodiment 90. The CAR-T cell as recited in Embodiment 88, wherein the CAR displays at least 98% sequence identity to an amino acid sequence chosen from SEQ ID NO:45 to SEQ ID NO:46.

Embodiment 91. The CAR-T cell as recited in Embodiment 88, wherein the CAR is an amino acid sequence chosen from SEQ ID NO:45 to SEQ ID NO:46.

Embodiment 92. A CAR-T cell, which comprises a chimeric antigen receptor (CAR) targeting CD7, wherein the CAR-T cell is deficient in TRAC and deficient in CD7, and comprises a CD28 costimulatory domain and a CD3ζ signaling domain.

Embodiment 93. The CAR-T cell as recited in Embodiment 92, wherein the CAR displays at least 95% sequence identity to an amino acid sequence chosen from SEQ ID NO:32 to SEQ ID NO:39.

Embodiment 94. The CAR-T cell as recited in Embodiment 92, wherein the CAR displays at least 98% sequence identity to an amino acid sequence chosen from SEQ ID NO:32 to SEQ ID NO:39.

Embodiment 95. The CAR-T cell as recited in Embodiment 92, wherein the CAR is an amino acid sequence chosen from SEQ ID NO:32 to SEQ ID NO:39.

A therapeutic composition comprising a population of CAR-T cells as recited in any of any of Embodiments 1-30 and 74-95, or comprising a population of CAR-T cells comprising CAR(s) as recited in any of Embodiments 31-73, and at least one therapeutically acceptable carrier and/or adjuvant.

Embodiment 96. A method of treatment of cancer in a patient comprising administering genome-edited CAR-T cell, population of genome-edited CAR-T cells, dual CAR-T cells, or tandem CAR-T as recited in any of any of Embodiments 1-30 and 74-95, or comprising a population of CAR-T cells comprising CAR(s) as recited in any of Embodiments 31-73, to a patient in need thereof.

Embodiment 97. The method as recited in Embodiment 97, wherein the cancer is a hematologic malignancy.

Embodiment 98. The method as recited in Embodiment 98, wherein the hematologic malignancy is a T-cell malignancy.

Embodiment 99. The method as recited in Embodiment 99, wherein the T cell malignancy is T-cell acute lymphoblastic leukemia (T-ALL).

Embodiment 100. The method as recited in Embodiment 99, wherein the T cell malignancy is non-Hodgkin's lymphoma.

Embodiment 101. The method as recited in Embodiment 99, wherein the T cell malignancy is T-cell chronic lymphocytic leukemia (T-CLL).

Embodiment 102. The method as recited in Embodiment 98, wherein the hematologic malignancy is multiple myeloma.

Embodiment 103. The method as recited in Embodiment 98, wherein the hematologic malignancy is acute myeloid leukemia (AML).

Embodiment 104. A method of making a CAR-T cell as recited in any embodiment above or herein, using Cas9-CRISPR and a gRNA chosen from those disclosed herein.

Embodiment 105. A method of making a CAR-T cell as recited in any embodiment above or herein, using Cas9-CRISPR and a gRNA chosen from those disclosed Table 12 and Tables 15-47.

Embodiment 106. A method of making a CAR-T cell as recited in any embodiment above or herein, using Cas9-CRISPR and a gRNA chosen from those disclosed in Table 12 and those in boldface in Tables 15-47.

Embodiment 107. A method of making a CAR-T cell as recited in any embodiment above or herein, using Cas9-CRISPR and a gRNA chosen from those disclosed in Tables 12.

Disclosed herein is a genome-edited CAR-T cell, derived from a helper T cell, a cytotoxic T cell, a viral-specific cytotoxic T cell, a memory T cell, or a gamma delta (γδ) T cell, which comprise one or more chimeric antigen receptors (CARs) targeting one or more antigens, wherein the CAR-T cell is deficient in one or more antigens to which the one or more CARs specifically binds.

Also provided is a genome-edited CAR-T cell, derived from a helper T cell, a cytotoxic T cell, a viral-specific cytotoxic T cell, a memory T cell, or a gamma delta (γδ) T cell, which comprise one or more chimeric antigen receptors (CARs) targeting one or more antigens, wherein CAR-T cell is deficient in a subunit of the T cell receptor complex and one or more antigens to which the one or more CARs specifically binds.

Also provided is a CAR-T cell, derived from a helper T cell, a cytotoxic T cell, a viral-specific cytotoxic T cell, a memory T cell, or a gamma delta (γδ) T cell, in which the deficient subunit of the T cell receptor complex is selected from TCRα, TCRβ, TCRδ, TCRγ, CD3ε, CD3γ, CD3δ, and CD3ζ.

In certain embodiments, the chimeric antigen receptor specifically binds at least one antigen expressed on a malignant T cell.

In certain embodiments, one or more antigens is selected from BCMA, CS1, CD38, CD138, CD19, CD33, CD123, CD371, CD117, CD135, Tim-3, CD5, CD7, CD2, CD4, CD3, CD79A, CD79B, APRIL, CD56, and CD1a.

In certain embodiments, CAR-T cell further comprises a suicide gene therapy system.

In certain embodiments, the endogenous T cell receptor-mediated signaling is blocked in the CAR-T cell.

In certain embodiments, the CAR-T cell does not induce alloreactivity or graft-versus-host disease.

In certain embodiments, the CAR-T cells do not induce fratricide.

Also provided is a dual or tandem CAR-T cell.

Also provided is a pharmaceutical composition comprising a population of CAR-T cells as disclosed herein, and at least one therapeutically acceptable carrier and/or adjuvant.

Also provided are methods for treating hematologic malignancies comprising administering a genome-edited CAR-T cell, a population of genome-edited CAR-T cells, wherein the population of genome-edited CAR-T cells are mono CAR-T cells, dual CAR-T cells, or tandem CAR-T cells as disclosed herein, or pharmaceutical compositions comprising them as disclosed herein to a patient in need thereof.

In certain embodiments, the hematologic malignancy is a T-cell malignancy.

In certain embodiments, the T cell malignancy is T-cell acute lymphoblastic leukemia (T-ALL).

In certain embodiments, the T cell malignancy is non-Hodgkin's lymphoma.

In certain embodiments, the T cell malignancy is T-cell chronic lymphocytic leukemia (T-CLL).

In certain embodiments, the hematologic malignancy is multiple myeloma.

In certain embodiments, the hematologic malignancy is acute myeloid leukemia (AML).

### CAR-T Cells

The present disclosure provides chimeric antigen receptor-bearing T cells (CAR-T cells), pharmaceutical compositions comprising them, and methods of immunotherapy for the treatment of cancer, specifically hematologic malignancies.

A CAR-T cell is a T cell which expresses a chimeric antigen receptor. The T cell expressing a CAR molecule may be a helper T cell, a cytotoxic T cell, a viral-specific cytotoxic T cell, a memory T cell, or a gamma delta (γδ) T cell.

A chimeric antigen receptor (CAR), is a recombinant fusion protein comprising: 1) an extracellular ligand-binding domain, i.e., an antigen-recognition domain, 2) a transmembrane domain, and 3) a signaling transducing domain.

The extracellular ligand-binding domain is an oligo- or polypeptide that is capable of binding a ligand. Preferably, the extracellular ligand-binding domain will be capable of interacting with a cell surface molecule which may be an antigen, a receptor, a peptide ligand, a protein ligand of the target, or a polypeptide of the target. The extracellular ligand-binding domain can specifically bind to an antigen with an affinity constant or affinity of interaction (K_{D}) between about 0.1 pM to about 10 pM, to about 0.1 pM to about 1 pM, or more preferably to about 0.1 pM to about 100 nM. Methods for determining the affinity constant or affinity of interaction (K_{D}) are well-known in the art. In some instances, the extracellular ligand-binding domain is chosen to recognize a ligand that acts as a cell surface marker on target cells associated with particular disease states.

In one embodiment, the extracellular ligand-binding domain comprises a single chain antibody fragment (scFv) comprising the light (V_{L}) and the heavy (V_{H}) variable fragment joined by a linker (e.g., GGGGS₍₂₋₆₎) (SEQ ID NO:447) and confers specificity for either a T cell antigen or an antigen that is not specific to a T cell. In one embodiment, the chimeric antigen receptor of a CAR-T cell may bind to an T cell-specific antigen expressed or overexpressed on a malignant T cell for which a CAR-T cell is deficient in the antigen (e.g., a genome-edited CAR-T cell).

Non-limiting examples of CAR-targeted antigens expressed on malignant T cells include CD5, CD7, CD2, CD4, and CD3. In one embodiment, a CAR-T cell of the present disclosure comprises a chimeric antigen receptor with an extracellular ligand-binding domain that specifically binds to CD5.

In another embodiment, a CAR-T cell of the present disclosure comprises a chimeric antigen receptor with an extracellular ligand-binding domain that specifically binds to CD7. In another words, the CAR which specifically binds CD7, comprises an extracellular ligand-binding domain comprising a polypeptide sequence displaying at least 80%, 90%, 95%, 97%, or 99% identity with an amino acid sequence selected from SEQ ID NO:20 and SEQ ID NO:21, and linked together by a flexible linker comprising the sequence (GGGGS)₃₋₄ (SEQ ID NO:449).

In another embodiment, a CAR-T cell of the present disclosure comprises a chimeric antigen receptor with an extracellular ligand-binding domain that specifically binds to CD2. In another words, the CAR which specifically binds CD2, comprises an extracellular ligand-binding domain comprising a polypeptide sequence displaying at least 80%, 90%, 95%, 97%, or 99% identity with an amino acid sequence selected from SEQ ID NO:12; and SEQ ID NO:13 or SEQ ID:14 and SEQ ID NO:15, and linked together by a flexible linker comprising the sequence (GGGGS)₃₋₄ (SEQ ID NO:449).

In yet another embodiment, a CAR-T cell of the present disclosure comprises a chimeric antigen receptor with an extracellular ligand-binding domain that specifically binds to CD4.

In still another embodiment, a CAR-T cell of the present disclosure comprises an extracellular ligand-binding domain of a chimeric antigen receptor that specifically binds to CD3. In another words, the CAR which specifically binds CD3, comprises an extracellular ligand-binding domain comprising a polypeptide sequence displaying at least 80%, 90%, 95%, 97%, or 99% identity with an amino acid sequence selected from SEQ ID NO:16; and SEQ ID NO:17 or SEQ ID:18 and SEQ ID NO:19, and linked together by a flexible linker comprising the sequence (GGGGS)₃₋₄ (SEQ ID NO:449).

Non-limiting examples of CAR-targeted antigens expressed on the surface of leukemia cells (e.g., abnormal myeloblasts, red blood cells, or platelets) include CD123 (IL3RA), CD371 (CLL-1; CLEC12A), CD117 (c-kit), and CD135 (FLT3), CD7, and Tim3. A CAR may be constructed with an extracellular ligand-binding domain to target these antigens for treatment of leukemia, i.e., acute myeloid leukemia (AML).

Non-limiting examples of CAR-targeted antigens expressed on the surface of a multiple myeloma cell (e.g., a malignant plasma cell) include BCMA, CS1, CD38, CD79A, CD79B, CD138, and CD19. A CAR may be constructed with an extracellular ligand-binding domain to target these antigens for treatment of multiple myeloma. In another embodiment, the CAR may be constructed with a portion of the APRIL protein, targeting the ligand for the B-Cell Maturation Antigen (BCMA) and Transmembrane Activator and CAML Interactor (TACI), effectively co-targeting both BCMA and TACI for the treatment of multiple myeloma. A signal peptide directs the transport of a secreted or transmembrane protein to the cell membrane and/or cell surface to allow for correct localization of the polypeptide. Particularly, the signal peptide of the present disclosure directs the appended polypeptide, i.e., the CAR receptor, to the cell membrane wherein the extracellular ligand-binding domain of the appended polypeptide is displayed on the cell surface, the transmembrane domain of the appended polypeptide spans the cell membrane, and the signaling transducing domain of the appended polypeptide is in the cytoplasmic portion of the cell. In one embodiment, the signal peptide is the signal peptide from human CD8α (SEQ ID NO:1). In one embodiment, the signal peptide is a functional fragment of the CD8α signal peptide. A functional fragment is defined as a fragment of at least 10 amino acids of the CD8α signal peptide that directs the appended polypeptide to the cell membrane and/or cell surface. Examples of functional fragments of the human CD8α signal peptide include the amino acid sequences MALPVTALLLPLALLLHAA, MALPVTALLLP, PVTALLLPLALL, and LLLPLALLLHAARP.

Typically, the extracellular ligand-binding domain is linked to the signaling transducing domain of the chimeric antigen receptor (CAR) by a transmembrane domain (Tm). The transmembrane domain traverses the cell membrane, anchors the CAR to the T cell surface, and connects the extracellular ligand-binding domain to the signaling transducing domain, impacting the expression of the CAR on the T cell surface.

The distinguishing feature of the transmembrane domain in the present disclosure is the ability to be expressed at the surface of an immune cell to direct an immune cell response against a pre-defined target cell. The transmembrane domain can be derived from natural or synthetic sources. Alternatively, the transmembrane domain of the present disclosure may be derived from any membrane-bound or transmembrane protein.

Non-limiting examples of transmembrane polypeptides of the present disclosure alpha, beta or zeta chain of the T-cell receptor, CD28, CD3 epsilon, CD45, CD4, CDS, CDS, CD9, CD16, CD22, CD33, CD37, CD64, CDS0, CD86, CD134, CD137 and CD154. Alternatively, the transmembrane domain can be synthetic and comprise predominantly hydrophobic amino acid residues (e.g., leucine and valine). In one embodiment, the transmembrane domain is derived from the T-cell surface glycoprotein CD8 alpha chain isoform 1 precursor (NP_001139345.1) (SEQ ID NO:4), and more preferably CD28 (SEQ ID NO:3). The transmembrane domain can further comprise a hinge region between extracellular ligand-binding domain and said transmembrane domain. The term “hinge region” generally means any oligo- or polypeptide that functions to link the transmembrane domain to the extracellular ligand-binding domain. In particular, hinge region is used to provide more flexibility and accessibility for the extracellular ligand-binding domain. A hinge region may comprise up to 300 amino acids, preferably 10 to 100 amino acids and most preferably 25 to 50 amino acids. Hinge region may be derived from all or parts of naturally-occurring molecules such as CD28, 4-1BB (CD137), OX-40 (CD134), CD3ζ, the T cell receptor a or β chain, CD45, CD4, CD5, CD8, CD8, CD9, CD16, CD22, CD33, CD37, CD64, CD80, CD86, ICOS, CD154 or from all or parts of an antibody constant region. Alternatively, the hinge region may be a synthetic sequence that corresponds to a naturally-occurring hinge sequence or the hinge region may be an entirely synthetic hinge sequence. In one embodiment, the hinge domain comprises a part of human CD8a (SEQ ID NO:2), FcγRIIIα receptor, or IgG1, and have at least 80%, 90%, 95%, 97%, or 99% sequence identity thereto.

A chimeric antigen receptor (CAR) of the present disclosure comprises a signal transducing domain or intracellular signaling domain of a CAR which is responsible for intracellular signaling following the binding of the extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper T cell activity, including the secretion of cytokines. Thus, the term “signal transducing domain” refers to the portion of a protein which transduces the effector signal function signal and directs the cell to perform a specialized function.

Examples of signal transducing domains for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Non-limiting examples of ITAM that can be used in the present disclosure can include those derived from TCRζ, FcRγ, FcRβ, FcRε, CD3γ, CD3δ, CD3ε, CDS, CD22, CD79a, CD79b and CD66d. In one embodiment, the signaling transducing domain of the CAR can comprise the CD3ζ signaling domain with an amino acid sequence of at least 80%, 90%, 95%, 97%, or 99% sequence identity thereto.

In addition, the CAR-T cells of the present disclosure may further comprise one or more suicide gene therapy systems. Suitable suicide gene therapy systems known in the art include, but are not limited to, several herpes simplex virus thymidine kinase (HSVtk)/ganciclovir (GCV) or inducible caspase 9 proteins. In one embodiment, the suicide gene is a chimeric CD34/thymidine kinase.

T cells disclosed herein may be deficient in an antigen to which the chimeric antigen receptor specifically binds and are therefore fratricide-resistant. In some embodiments, the antigen of the T cell is modified such that the chimeric antigen receptor no longer specifically binds the modified antigen. For example, the epitope of the antigen recognized by the chimeric antigen receptor may be modified by one or more amino acid changes (e.g., substitutions or deletions) or the epitope may be deleted from the antigen. In other embodiments, expression of the antigen is reduced in the T cell by at least 50%, at least 60%, at least 70%, at least 80%, at least 90% or more. Methods for decreasing the expression of a protein are known in the art and include, but are not limited to, modifying or replacing the promoter operably linked to the nucleic acid sequence encoding the protein. In still other embodiments, the T cell is modified such that the antigen is not expressed, e.g., by deletion or disruption of the gene encoding the antigen. In each of the above embodiments, the T cell may be deficient in one or preferably all the antigens to which the chimeric antigen receptor specifically binds. Methods for genetically modifying a T cell to be deficient in an antigen are well known in art, and non-limiting examples are provided above. In an exemplary embodiment, CRISPR/cas9 gene editing can be used to modify a T cell to be deficient in an antigen, for example as described below. Alternatively, TALENs may be used to edit genes.

In an variation of the method above, a construct encoding one or more protein expression blocker (PEBL) may be transduced into the cell, either as the editing step or part of the editing step, or as part of CAR transduction. For example, an construct encoding an antibody-derived single-chain variable fragment specific for CD3ε may be transduced, e.g. by a lentiviral vector. Once expressed, the PEBL colocalizes intracellularly with CD3ε, blocking surface CD3 and TCRαβ expression. Accordingly, PEBL blockade of surface CD3/TCRαβ expression is an alternative method of preparing allogeneic CAR-T cells. Furthermore, PEBL and CAR expression can be combined in a single construct. Either of these methods may be achieved using the methods disclosed herein, and PEBLs may be produced for blockade of any of the targets of gene suppression disclosed herein.

The methods described above may be adapted to insert a CAR into a locus for a gene encoding an antigen, cell surface protein, or secretable protein, such as a cytokine. In this way, editing of the genome is effected by transfection of CAR. Thereafter, cells may be activated as described herein, removing separate genome editing step in certain embodiments. Ideally, such a step should be performed while cells are actively dividing. Such methods are also expected to result in robust expansion of engineered cells.

In certain circumstances, an T cell may be selected for deficiency in the antigen to which the chimeric antigen receptor specifically binds. Certain T cells will produce and display less of a given surface protein; instead if deleting or non-functionalizing the antigen that will be the target of the T-CAR, the T cell can be selected for deficiency in the antigen, and the population of antigen-deficient cells expanded for transduction of the CAR. Such a cell would also be fratricide-resistant.

TABLE 1
Amino acid sequences of different CAR components.

| | SEQ ID | |
|---|---|---|
| Functional domains | NO: | Amino acid sequence |
| CD8α signal peptide | SEQ ID | MALPVTALLLPLALLLHAARP |
| | NO: 1 | |
| CD8α hinge | SEQ ID | TTTPAPRPPTPAPTIASQPLSLRPEACRPAAGGA |
| | NO: 2 | VHTRGLDFACD |
| CD28 Transmembrane | SEQ ID | FWVLVVVGGVLACYSLLVTVAFIIFWV |
| (Tₘ) domain | NO: 3 | |
| Surface glycoprotein CD8 | SEQ ID | MALPVTALLLPLALLLHAARPSQFRVSPLDRT |
| alpha chain isoform 1 | NO: 4 | WNLGETVELKCQVLLSNPTSGCSWLFQPRGAA |
| precursor | | ASPTFLLYLSQNKPKAAEGLDTQRFSGKRLGDT |
| (NP_001139345.1) | | FVLTLSDFRRENEGYYFCSALSNSIMYFSHFVP |
| | | VFLPAKPTTTPAPRPPTPAPTIASQPLSLRPEACR |
| | | PAAGGAVHTRGLDFACDIYIWAPLAGTCGVLL |
| | | LSLVITLYCNHRNRRRVCKCPRPVVKSGDKPSL |
| | | SARYV |
| 4-1BB costimulatory | SEQ ID | KRGRKKLLYIFKQPFMRPVQTTQEEDGCSCRFP |
| domain | NO: 5 | EEEEGGCEL |
| CD28 costimulatory | SEQ ID | RSKRSRLLHSDYMNMTPRRPGPTRKHYQPYAP |
| domain | NO: 6 | PRDFAAYRS |
| CD3 zeta (ζ) | SEQ ID | RVKFSRSADAPAYKQGQNQLYNELNLGRREEY |
| | NO: 7 | DVLDKRRGRDPEMGGKPRRKNPQEGLYNELQ |
| | | KDKMAEAYSEIGMKGERRRGKGHDGLYQGLS |
| | | TATKDTYDALHMQALPPR |
| P2A peptide | SEQ ID | GSGATNFSLLKQAGDVEENPGP |
| | NO: 8 | |
| (GGGGS)₄ linker | SEQ ID | GGGGSGGGGSGGGGSGGGGS |
| | NO: 9 | |
| hCD34 | SEQ ID | MPRGWTALCLLSLLPSGFMSLDNNGTATPELP |
| | NO: 10 | TQGTFSNVSTNVSYQETTTPSTLGSTSLHPVSQ |
| | | HGNEATTNITETTVKFTSTSVITSVYGNTNSSVQ |
| | | SQTSVISTVFTTPANVSTPETTLKPSLSPGNVSD |
| | | LSTTSTSLATSPTKPYTSSSPILSDIKAEIKCSGIR |
| | | EVKLTQGICLEQNKTSSCAEFKKDRGEGLARV |
| | | LCGEEQADADAGAQVCSLLLAQSEVRPQCLLL |
| | | VLANRTEISSKLQLMKKHQSDLKKLGILDFTEQ |
| | | DVASHQSYSQKTLIALVTSGALLAVLGITGYFL |
| | | MNRRSWSPI |
| Human-Herpes Simplex | SEQ ID | MPRGWTALCLLSLLPSGFMSLDNNGTATPELP |
| Virus-1 (HSV)-thymidine | NO: 11 | TQGTFSNVSTNVSYQETTTPSTLGSTSLHPVSQ |
| kinase (TK) | | HGNEATTNITETTVKFTSTSVITSVYGNTNSSVQ |
| | | SQTSVISTVFTTPANVSTPETTLKPSLSPGNVSD |
| | | LSTTSTSLATSPTKPYTSSSPILSDIKAEIKCSGIR |
| | | EVKLTQGICLEQNKTSSCAEFKKDRGEGLARV |
| | | LCGEEQADADAGAQVCSLLLAQSEVRPQCLLL |
| | | VLANRTEISSKLQLMKKHQSDLKKLGILDFTEQ |
| | | DVASHQSYSQKTLIALVTSGALLAVLGITGYFL |
| | | MNRRSWSPTGEGGGGGDLGGVKLPHLFGKRL |
| | | VEARMASYPCHQHASAFDQAARSRGHSNRRT |
| | | ALRPRRQQEATEVRLEQKMPTLLRVYIDGPHG |
| | | MGKTTTTQLLVALGSRDDIVYVPEPMTYWQV |
| | | LGASETIANIYTTQHRLDQGEISAGDAAVVMTS |
| | | AQITMGMPYAVTDAVLAPHVGGEAGSSHAPPP |
| | | ALTLLLDRHPIAVMLCYPAARYLMGSMTPQAV |
| | | LAFVALIPPTLPGTNIVLGALPEDRHIDRLAKRQ |
| | | RPGERLDLAMLAAIRRVYGLLANTVRYLQGGG |
| | | SWWEDWGQLSGTAVPPQGAEPQSNAGPRPHIG |
| | | DTLFTLFRAPELLAPNGDLYNVFAWALDVLAK |
| | | RLRPMHVFILDYDQSPAGCRDALLQLTSGMVQ |
| | | THVTTPGSIPTICDLARTFAREMGEAN |

TABLE 2
Amino acid sequences of the variable heavy (V_{H}) and variable light
(V_{L}) chains of the scFvs.

| | SEQ ID | |
|---|---|---|
| ScFv sequences | NO: | Amino acid sequence |
| CD2 heavy chain variable | SEQ ID | EVKLEESGAELVKPGASVKLSCRTSGFN1KDTI |
| region (35.1 A TCC®HB- | NO: 12 | HWVKQRPEQGLKWIGRIDPANGNTKYDPKFQ |
| 222™) | | DKATVTADTSSNTAYLQLSLTSEDTAVYYCV |
| | | TYAYDGNWYFDVWGAGTAVTVSS |
| CD2 light chain variable | SEQ ID | DIKNITQSPSSMYVSLGERVTITCKASQDINSFL |
| region (35.1 ATCC®HB- | NO: 13 | SWFQQKPGKSPKTLIYRANRLVDGVPSRFSGS |
| 222^{™}) | | GSGQDYSLTISSLEYEDMEIYYCLQYDEFPYTF |
| | | GGGTKLEMKR |
| CD2 heavy chain variable | SEQ ID | EVQLEESGAELVRPGTSVKLSCKASGYTFTSY |
| region (OKT 11 | NO: 14 | WMHWIKQRPEQGLEWIGRIDPYDSETHYNEK |
| ATCC®CRL-8027^{™}) | | FKDKAILSVDKSSSTAYIQLSSLTSDDSAVYYC |
| | | SRRDAKYDGYALDYWGQGTSVTVSS |
| CD2 light chain variable | SEQ ID | DIMVMTQAAPSVPVTPGESVSISCRSSKTLL |
| region (OKT 11 | NO: 15 | HSNGNTYLYWFLQRPGQSPQVLIYRMSNLAS |
| ATCC®CRL-8027^{™}) | | GVPNRFSGSGSETTFTLRISRVEAEDVGIYYCM |
| | | QHLEYPYTFGGGTKLEIER |
| CD3 heavy chain variable | SEQ ID | GSQVQLQQSGAELARPGASVKMSCKASGYTF |
| region (OKT 3) | NO: 16 | TRYTMHWVKQRPGQGLEWIGYINPSRGYTNY |
| | | NQKFKDKATLTTDKSSSTAYMQLSSLTSEDSA |
| | | VYYCARYYDDHYCLDYWGQGTTLTVSS |
| CD3 light chain variable | SEQ ID | QIVLTQSPAIMSASPGEKVTMTCSASSSVSYM |
| region (OKT 3) | NO: 17 | NWYQQKSGTSPKRWIYDTSKLASGVPAHFRG |
| | | SGSGTSYSLTISGMEAEDAATYYCQQWSSNPF |
| | | TFGSGTKLEINR |
| CD3 heavy chain variable | SEQ ID | EVQLVESGGGLVQPGGSLRLSCAASGYSFTGY |
| region (UCHT1) | NO: 18 | TMNWVRQAPGKCLEWVALINPYKGVSTYNQ |
| | | KFKDRFTISVDKSKNTAYLQMNSLRAEDTAV |
| | | YYCARSGYYGDSDWYFDVWGQGTLVTVSS |
| CD3 heavy chain variable | SEQ ID | DIQMTQSPSSLSASVGDRVTITCRASQDIRNYL |
| region (UCHT1) | NO: 19 | NWYQQKPGKAPKLLIYYTSRLESGVPSRFSGS |
| | | GSGTDYTLTISSLQPEDFATYYCQQGNTLPWT |
| | | FGCGTKVEIK |
| CD7 heavy chain variable | SEQ ID | EVQLVESGGGLVKPGGSLKLSCAASGLTFSSY |
| region | NO: 20 | AMSWVRQTPEKRLEWVASISSGGFTYYPDSV |
| | | KGRFTISRDNARNILYLQMSSLRSEDTAMYYC |
| | | ARDEVRGYLDVWGAGTTVTVS |
| CD7 light chain variable | SEQ ID | DIQMTQTTSSLSASLGDRVTISCSASQGISNYL |
| region | NO: 21 | NWYQQKPDGTVKLLIYYTSSLHSGVPSRFSGS |
| | | GSGTDYSLTISNLEPEDIATYYCQQYSKLPYTF |
| | | GGGTKLEIKR |
| FTL3 heavy chain | SEQ ID | EVQLVQSGAEVKKPGASVKVSCKASGYTFTS |
| variable region (EB10) | NO: 22 | YYMHWVRQAPGQGLEWMGIINPSGGSTSYAQ |
| | | KFQGRVTMTRDTSTSTVYMELSSLRSEDTAVY |
| | | YCARGVGAHDAFDIWGQGTTVTVSS |
| FTL3 light chain variable | SEQ ID | DVVMTQSPLSLPVTPGEPASISCRSSQSLLHSN |
| region (EB10) | NO: 23 | GNNYLDWYLQKPGQSPQLLIYLGSNRASGVP |
| | | DRFSGSGSDTDFTLQISRVEAEDVGVYYCMQG |
| | | THPAISFGQGTRLEIK |
| FTL3 heavy chain | SEQ ID | EVQLVQSGAEVKKPGSSVKVSCKASGGTFSSY |
| variable region (NC7) | NO: 24 | AISWVRQAPGQGLEWMGGIIPIFGTANYAQKF |
| | | QGRVTITADKSTSTAYMELSSLRSEDTAVYYC |
| | | ATFALFGFREQAFDIWGQGTTVTVSS |
| FTL3 light chain variable | SEQ ID | DIQMTQSPSSLSASVGDRVTITCRASQSISSYLN |
| region (NC7) | NO: 25 | WYQQKPGKAPKLLIYAASSLQSGVPSRFSGSG |
| | | SGTDFTLTISSLQPEDLATYYCQQSYSTPFTFGP |
| | | GTKVDIK |
| FTL3 heavy chain | SEQ ID | EVQLVQSGAEVKKPGASVKVSCKASGYTFTS |
| variable region (D3-D4) | NO: 26 | YYMHWARQAPGQGLEWMGIINPSGGSTSYAQ |
| | | KFQGRVTMTRDTSTSTVYMELSSLRSEDTAVY |
| | | YCARVVAAAVADYWGQGTLVTVSS |
| FTL3 light chain variable | SEQ ID | DVVMTQSPLSLPVTPGEPASISCRSSQSLLHSN |
| region (D3-D4) | NO: 27 | GYNYLDWYLQKPGQSPQLLIYLGSNRASGVP |
| | | DRFSGSGSGTDFTLKISRVEAEDVGVYYCMQS |
| | | LQTPFTFGPGTKVDIK |
| CS1 heavy chain variable | SEQ ID | QVQLQQPGAELVRPGASVKLSCKASGYSFTTY |
| region | NO: 28 | WMNWVKQRPGQGLEWIGMIHPSDSETRL |
| | | NQKFKDKATLTVDKSSSTAYMQLSSPTSEDSA |
| | | VYYCARSTMIATRAMDYWGQGTSVTVSS |
| CS1 light chain variable | SEQ ID | DIVMTQSQKSMSTSVGDRVSITCKASQDVITG |
| region | NO: 29 | VAWYQQKPGQSPKLLIYSASYRYTGVPD |
| | | RFTGSGSGTDFTFTISNVQAEDLAVYYCQQHY |
| | | STPLTFGAGTKLELK |
| CD33 heavy chain | SEQ ID | QVQLQQPGAEVVKPGASVKMSCKASGYTFTS |
| variable region | NO: 30 | YYIHWIKQTPGQGLEWVGVIYPGNDDISYNQK |
| | | FQGKATLTADKSSTTAYMQLSSLTSEDSAVYY |
| | | CAREVRLRYFDVWGQGTTVTVSSSG |
| CD33 light chain variable | SEQ ID | GSEIVLTQSPGSLAVSPGERVTMSCKSSQSVFF |
| region | NO: 31 | SSSQKNYLAWYQQIPGQSPRLLIYWASTRESG |
| | | VPDRFTGSGSGTDFTLTISSVQPEDLAIYYCHQ |
| | | YLSSRTFGQGTKLEIKR |

Mono CAR-T Cells (mCAR-T)

The CAR-T cells encompassed by the present disclosure are deficient in one or more antigens to which the chimeric antigen receptor specifically binds and are therefore fratricide-resistant. In some embodiments, the one or more antigens of the T cell is modified such the chimeric antigen receptor no longer specifically binds the one or more modified antigens. For example, the epitope of the one or more antigens recognized by the chimeric antigen receptor may be modified by one or more amino acid changes (e.g., substitutions or deletions) or the epitope may be deleted from the antigen. In other embodiments, expression of the one or more antigens is reduced in the T cell by at least 50%, at least 60%, at least 70%, at least 80%, at least 90% or more. Methods for decreasing the expression of a protein are known in the art and include, but are not limited to, modifying or replacing the promoter operably linked to the nucleic acid sequence encoding the protein. In still other embodiments, the T cell is modified such that the one or more antigens is not expressed, e.g., by deletion or disruption of the gene encoding the one or more antigens. In each of the above embodiments, the CAR-T cell may be deficient in one or preferably all the antigens to which the chimeric antigen receptor specifically binds. The methods to genetically modify a T cell to be deficient in one or more antigens are well known in art and non-limiting examples are provided herein. In embodiments described in Examples 1-6, the CRISPR-Cas9 system is used to modify a T cell to be deficient in one or more antigens.

CAR-T cells encompassed by the present disclosure may further be deficient in endogenous T cell receptor (TCR) signaling as a result of deleting a part of the T Cell Receptor (TCR)-CD3 complex. In various embodiments it may be desirable to eliminate or suppress endogenous TCR signaling in CAR-T cells disclosed herein. For example, decreasing or eliminating endogenous TCR signaling in CAR-T cells may prevent or reduce graft versus host disease (GvHD) when allogenic T cells are used to produce the CAR-T cells. Methods for eliminating or suppressing endogenous TCR signaling are known in the art and include, but are not limited to, deleting a part of the TCR-CD3 receptor complex, e.g., the TCR receptor alpha chain (TRAC), the TCR receptor beta chain (TCRβ), TCRδ, TCRγ, CD3ε, CD3γ, and/or CD3δ. Deleting a part of the TCR receptor complex may block TCR mediated signaling and may thus permit the safe use of allogeneic T cells as the source of CAR-T cells without inducing life-threatening GvHD.

In addition, the CAR-T cells encompassed by the present disclosure may further comprise one or more suicide genes as described herein.

In an embodiment, the disclosure provides a T cell comprising a chimeric antigen receptor that specifically binds CD5, wherein the T cell is deficient in CD5, e.g., CD5ΔCART5 cell. In non-limiting examples the deficiency in CD5 resulted from (a) modification of CD5 expressed by the T cell such that the chimeric antigen receptor no longer specifically binds the modified CD5, (b) modification of the T cell such that expression of the antigen is reduced in the T cell by at least 50%, at least 60%, at least 70%, at least 80%, at least 90% or more, or (c) modification of the T cell such that CD5 is not expressed (e.g., by deletion or disruption of the gene encoding CD5). In further embodiments, the T cell comprises a suicide gene and/or a modification such that endogenous T cell receptor (TCR) mediated signaling is blocked in the T cell. In non-limiting examples, a protein coding sequence of a modified Human-Herpes Simplex Virus-1-thymidine kinase (TK) gene fused in-frame to the extracellular and transmembrane domains of the human CD34 cDNA is expressed in CD5ΔCART5 cells.

In another embodiment, the disclosure provides a T cell comprising a chimeric antigen receptor that specifically binds CD7, wherein the T cell is deficient in CD7, e.g., CD7ΔCART7 cell. In non-limiting examples the deficiency in CD7 resulted from (a) modification of CD7 expressed by the T cell such that the chimeric antigen receptor no longer specifically binds the modified CD7, (b) modification of the T cell such that expression of the antigen is reduced in the T cell by at least 50%, at least 60%, at least 70%, at least 80%, at least 90% or more, or (c) modification of the T cell such that CD7 is not expressed (e.g., by deletion or disruption of the gene encoding CD7). In further embodiments, the T cell comprises a suicide gene and/or a modification such that endogenous T cell receptor (TCR) mediated signaling is blocked in the T cell. In non-limiting examples, a protein coding sequence of a modified Human-Herpes Simplex Virus-1-thymidine kinase (TK) gene fused in-frame to the extracellular and transmembrane domains of the human CD34 cDNA is expressed in CD7ΔCART7 cells.

In another embodiment, the disclosure provides a T cell comprising a chimeric antigen receptor that specifically binds CD2, wherein the T cell is deficient in CD2, e.g., CD2ΔCART2 cell. In non-limiting examples the deficiency in CD2 resulted from (a) modification of CD2 expressed by the T cell such that the chimeric antigen receptor no longer specifically binds the modified CD2, (b) modification of the T cell such that expression of the antigen is reduced in the T cell by at least 50%, at least 60%, at least 70%, at least 80%, at least 90% or more, or (c) modification of the T cell such that CD2 is not expressed (e.g., by deletion or disruption of the gene encoding CD2). In further embodiments, the T cell comprises a suicide gene and/or a modification such that endogenous T cell receptor (TCR) mediated signaling is blocked in the T cell. In non-limiting examples, a protein coding sequence of a modified Human-Herpes Simplex Virus-1-thymidine kinase (TK) gene fused in-frame to the extracellular and transmembrane domains of the human CD34 cDNA is expressed in CD2ΔCART2 cells.

In another embodiment, the disclosure provides a T cell comprising a chimeric antigen receptor that specifically binds CD4, wherein the T cell is deficient in CD4, e.g., CD4ΔCART4 cell. In non-limiting examples the deficiency in CD4 resulted from (a) modification of CD4 expressed by the T cell such that the chimeric antigen receptor no longer specifically binds the modified CD4, (b) modification of the T cell such that expression of the antigen is reduced in the T cell by at least 50%, at least 60%, at least 70%, at least 80%, at least 90% or more, or (c) modification of the T cell such that CD4 is not expressed (e.g., by deletion or disruption of the gene encoding CD4). In further embodiments, the T cell comprises a suicide gene and/or a modification such that endogenous T cell receptor (TCR) mediated signaling is blocked in the T cell. In non-limiting examples, a protein coding sequence of a modified Human-Herpes Simplex Virus-1-thymidine kinase (TK) gene fused in-frame to the extracellular and transmembrane domains of the human CD34 cDNA is expressed in the CD4ΔCART4 cells.

In another embodiment, the disclosure provides a T cell comprising a chimeric antigen receptor that specifically binds CD3, wherein the T cell is deficient in CD3ε, e.g., CD3ΔCART3e cell. In non-limiting examples the deficiency in CD3 resulted from (a) modification of CD3 expressed by the T cell such that the chimeric antigen receptor no longer specifically binds the modified CD3, (b) modification of the T cell such that expression of the antigen is reduced in the T cell by at least 50%, at least 60%, at least 70%, at least 80%, at least 90% or more, or (c) modification of the T cell such that CD3 is not expressed (e.g., by deletion or disruption of the gene encoding CD3ε). In further embodiments, the T cell comprises a suicide gene and/or a modification such that endogenous T cell receptor (TCR) mediated signaling is blocked in the T cell. In non-limiting examples, a protein coding sequence of a modified Human-Herpes Simplex Virus-1-thymidine kinase (TK) gene fused in-frame to the extracellular and transmembrane domains of the human CD34 cDNA is expressed in the CD3ΔCART38 cells.

Disclosed are embodiments of CAR amino acid sequences that can be expressed on the surface of a genome-edited CAR-T cell derived from a cytotoxic T cell, a memory T cell, or a gamma delta (γδ) T cell.

TABLE 3
Amino Acid Sequences of Mono Chimeric Antigen Receptors (CARs).

| Mono CAR | SEQ ID | |
|---|---|---|
| Constructs | NO: | Amino acid sequence |
| CD7-CAR-4- | SEQ ID | MALPVTALLLPLALLLHAARPDIQMTQTTSSLSASLG |
| 1BB_CD34 | NO: 32 | DRVTISCSASQGISNYLNWYQQKPDGTVKLLIYYTSS |
| | | LHSGVPSRFSGSGSGTDYSLTISNLEPEDIATYYCQQY |
| | | SKLPYTFGGGTKLEIKRGGGGSGGGGSGGGGSGGGG |
| | | SEVQLVESGGGLVKPGGSLKLSCAASGLTFSSYAMS |
| | | WVRQTPEKRLEWVASISSGGFTYYPDSVKGRFTISRD |
| | | NARNILYLQMSSLRSEDTAMYYCARDEVRGYLDVW |
| | | GAGTTVTVSPRASTTTPAPRPPTPAPTIASQPLSLRPEA |
| | | CRPAAGGAVHTRGLDFACDFWVLVVVGGVLACYSL |
| | | LVTVAFIIFWVKRGRKKLLYIFKQPFMRPVQTTQEED |
| | | GCSCRFPEEEEGGCELRVKFSRSADAPAYKQGQNQL |
| | | YNELNLGRREEYDVLDKRRGRDPEMGGKPRRKNPQ |
| | | EGLYNELQKDKMAEAYSEIGMKGERRRGKGHDGLY |
| | | QGLSTATKDTYDALHMQALPPRRTDGSGATNFSLLK |
| | | QAGDVEENPGPVSEAMPRGWTALCLLSLLPSGFMSL |
| | | DNNGTATPELPTQGTFSNVSTNVSYQETTTPSTLGSTS |
| | | LHPVSQHGNEATTNITETTVKFTSTSVITSVYGNTNSS |
| | | VQSQTSVISTVFTTPANVSTPETTLKPSLSPGNVSDLS |
| | | TTSTSLATSPTKPYTSSSPILSDIKAEIKCSGIREVKLTQ |
| | | GICLEQNKTSSCAEFKKDRGEGLARVLCGEEQADAD |
| | | AGAQVCSLLLAQSEVRPQCLLLVLANRTEISSKLQLM |
| | | KKHQSDLKKLGILDFTEQDVASHQSYSQKTLIALVTS |
| | | GALLAVLGITGYFLMNRRSWSPI |
| CD7-CAR-4- | SEQ ID | MALPVTALLLPLALLLHAARPDIQMTQTTSSLSASLG |
| 1BB_CD34_TK | NO: 33 | DRVTISCSASQGISNYLNWYQQKPDGTVKLLIYYTSS |
| | | LHSGVPSRFSGSGSGTDYSLTISNLEPEDIATYYCQQY |
| | | SKLPYTFGGGTKLEIKRGGGGSGGGGSGGGGSGGGG |
| | | SEVQLVESGGGLVKPGGSLKLSCAASGLTFSSYAMS |
| | | WVRQTPEKRLEWVASISSGGFTYYPDSVKGRFTISRD |
| | | NARNILYLQMSSLRSEDTAMYYCARDEVRGYLDVW |
| | | GAGTTVTVSPRASTTTPAPRPPTPAPTIASQPLSLRPEA |
| | | CRPAAGGAVHTRGLDFACDFWVLVVVGGVLACYSL |
| | | LVTVAFIIFWVKRGRKKLLYIFKQPFMRPVQTTQEED |
| | | GCSCRFPEEEEGGCELRVKFSRSADAPAYKQGQNQL |
| | | YNELNLGRREEYDVLDKRRGRDPEMGGKPRRKNPQ |
| | | EGLYNELQKDKMAEAYSEIGMKGERRRGKGHDGLY |
| | | QGLSTATKDTYDALHMQALPPRRTDGSGATNFSLLK |
| | | QAGDVEENPGPVSEAMPRGWTALCLLSLLPSGFMSL |
| | | DNNGTATPELPTQGTFSNVSTNVSYQETTTPSTLGSTS |
| | | LHPVSQHGNEATTNITETTVKFTSTSVITSVYGNTNSS |
| | | VQSQTSVISTVFTTPANVSTPETTLKPSLSPGNVSDLS |
| | | TTSTSLATSPTKPYTSSSPILSDIKAEIKCSGIREVKLTQ |
| | | GICLEQNKTSSCAEFKKDRGEGLARVLCGEEQADAD |
| | | AGAQVCSLLLAQSEVRPQCLLLVLANRTEISSKLQLM |
| | | KKHQSDLKKLGILDFTEQDVASHQSYSQKTLIALVTS |
| | | GALLAVLGITGYFLMNRRSWSPTGEGGGGGDLGGV |
| | | KLPHLFGKRLVEARMASYPCHQHASAFDQAARSRG |
| | | HSNRRTALRPRRQQEATEVRLEQKMPTLLRVYIDGP |
| | | HGMGKTTTTQLLVALGSRDDIVYVPEPMTYWQVLG |
| | | ASETIANIYTTQHRLDQGEISAGDAAVVMTSAQITMG |
| | | MPYAVTDAVLAPHVGGEAGSSHAPPPALTLLLDRHPI |
| | | AVMLCYPAARYLMGSMTPQAVLAFVALIPPTLPGTN |
| | | IVLGALPEDRHIDRLAKRQRPGERLDLAMLAAIRRVY |
| | | GLLANTVRYLQGGGSWWEDWGQLSGTAVPPQGAEP |
| | | QSNAGPRPHIGDTLFTLFRAPELLAPNGDLYNVFAWA |
| | | LDVLAKRLRPMHVFILDYDQSPAGCRDALLQLTSGM |
| | | VQTHVTTPGSIPTICDLARTFAREMGEAN |
| CD7-CAR- | SEQ ID | MALPVTALLLPLALLLHAARPDIQMTQTTSSLSASLG |
| CD28_CD34 | NO: 34 | DRVTISCSASQGISNYLNWYQQKPDGTVKLLIYYTSS |
| | | LHSGVPSRFSGSGSGTDYSLTISNLEPEDIATYYCQQY |
| | | SKLPYTFGGGTKLEIKRGGGGSGGGGSGGGGSGGGG |
| | | SEVQLVESGGGLVKPGGSLKLSCAASGLTFSSYAMS |
| | | WVRQTPEKRLEWVASISSGGFTYYPDSVKGRFTISRD |
| | | NARNILYLQMSSLRSEDTAMYYCARDEVRGYLDVW |
| | | GAGTTVTVSPRASTTTPAPRPPTPAPTIASQPLSLRPEA |
| | | CRPAAGGAVHTRGLDFACDFWVLVVVGGVLACYSL |
| | | LVTVAFIIFWVRSKRSRLLHSDYMNMTPRRPGPTRKH |
| | | YQPYAPPRDFAAYRSRVKFSRSADAPAYKQGQNQLY |
| | | NELNLGRREEYDVLDKRRGRDPEMGGKPRRKNPQE |
| | | GLYNELQKDKMAEAYSEIGMKGERRRGKGHDGLYQ |
| | | GLSTATKDTYDALHMQALPPRRTDGSGATNFSLLKQ |
| | | AGDVEENPGPVSEAMPRGWTALCLLSLLPSGFMSLD |
| | | NNGTATPELPTQGTFSNVSTNVSYQETTTPSTLGSTSL |
| | | HPVSQHGNEATTNITETTVKFTSTSVITSVYGNTNSSV |
| | | QSQTSVISTVFTTPANVSTPETTLKPSLSPGNVSDLSTT |
| | | STSLATSPTKPYTSSSPILSDIKAEIKCSGIREVKLTQGI |
| | | CLEQNKTSSCAEFKKDRGEGLARVLCGEEQADADAG |
| | | AQVCSLLLAQSEVRPQCLLLVLANRTEISSKLQLMKK |
| | | HQSDLKKLGILDFTEQDVASHQSYSQKTLIALVTSGA |
| | | LLAVLGITGYFLMNRRSWSPI |
| CD7-CAR- | SEQ ID | MALPVTALLLPLALLLHAARPDIQMTQTTSSLSASLG |
| CD28_CD34_TK | NO: 35 | DRVTISCSASQGISNYLNWYQQKPDGTVKLLIYYTSS |
| | | LHSGVPSRFSGSGSGTDYSLTISNLEPEDIATYYCQQY |
| | | SKLPYTFGGGTKLEIKRGGGGSGGGGSGGGGSGGGG |
| | | SEVQLVESGGGLVKPGGSLKLSCAASGLTFSSYAMS |
| | | WVRQTPEKRLEWVASISSGGFTYYPDSVKGRFTISRD |
| | | NARNILYLQMSSLRSEDTAMYYCARDEVRGYLDVW |
| | | GAGTTVTVSPRASTTTPAPRPPTPAPTIASQPLSLRPEA |
| | | CRPAAGGAVHTRGLDFACDFWVLVVVGGVLACYSL |
| | | LVTVAFIIFWVRSKRSRLLHSDYMNMTPRRPGPTRKH |
| | | YQPYAPPRDFAAYRSRVKFSRSADAPAYKQGQNQLY |
| | | NELNLGRREEYDVLDKRRGRDPEMGGKPRRKNPQE |
| | | GLYNELQKDKMAEAYSEIGMKGERRRGKGHDGLYQ |
| | | GLSTATKDTYDALHMQALPPRRTDGSGATNFSLLKQ |
| | | AGDVEENPGPVSEAMPRGWTALCLLSLLPSGFMSLD |
| | | NNGTATPELPTQGTFSNVSTNVSYQETTTPSTLGSTSL |
| | | HPVSQHGNEATTNITETTVKFTSTSVITSVYGNTNSSV |
| | | QSQTSVISTVFTTPANVSTPETTLKPSLSPGNVSDLSTT |
| | | STSLATSPTKPYTSSSPILSDIKAEIKCSGIREVKLTQGI |
| | | CLEQNKTSSCAEFKKDRGEGLARVLCGEEQADADAG |
| | | AQVCSLLLAQSEVRPQCLLLVLANRTEISSKLQLMKK |
| | | HQSDLKKLGILDFTEQDVASHQSYSQKTLIALVTSGA |
| | | LLAVLGITGYFLMNRRSWSPTGEGGGGGDLGGVKLP |
| | | HLFGKRLVEARMASYPCHQHASAFDQAARSRGHSN |
| | | RRTALRPRRQQEATEVRLEQKMPTLLRVYIDGPHGM |
| | | GKTTTTQLLVALGSRDDIVYVPEPMTYWQVLGASET |
| | | IANIYTTQHRLDQGEISAGDAAVVMTSAQITMGMPY |
| | | AVTDAVLAPHVGGEAGSSHAPPPALTLLLDRHPIAV |
| | | MLCYPAARYLMGSMTPQAVLAFVALIPPTLPGTNIVL |
| | | GALPEDRHIDRLAKRQRPGERLDLAMLAAIRRVYGL |
| | | LANTVRYLQGGGSWWEDWGQLSGTAVPPQGAEPQS |
| | | NAGPRPHIGDTLFTLFRAPELLAPNGDLYNVFAWAL |
| | | DVLAKRLRPMHVFILDYDQSPAGCRDALLQLTSGMV |
| | | QTHVTTPGSIPTICDLARTFAREMGEAN |
| CD79B-CAR- | SEQ ID | MALPVTALLLPLALLLHAARPGSDIQLTQSPSSLSASV |
| CD28_CD34 | NO: 36 | GDRVTITCKASQSVDYEGDSFLNWYQQKPGKAPKLL |
| | | IYAASNLESGVPSRFSGSGSGTDFTLTISSLQPEDFATY |
| | | YCQQSNEDPLTFGQGTKVEIKRGGGGSGGGGSGGGG |
| | | SGGGGSGGGGSEVQLVESGGGLVQPGGSLRLSCAAS |
| | | GYTFSSYWIEWVRQAPGKGLEWIGEILPGGGDTNYN |
| | | EIFKGRATFSADTSKNTAYLQMNSLRAEDTAVYYCT |
| | | RRVPIRLDYWGQGTLVTVSSPRASTTTPAPRPPTPAPT |
| | | IASQPLSLRPEACRPAAGGAVHTRGLDFACDFWVLV |
| | | VVGGVLACYSLLVTVAFIIFWVRSKRSRLLHSDYMN |
| | | MTPRRPGPTRKHYQPYAPPRDFAAYRSRVKFSRSAD |
| | | APAYKQGQNQLYNELNLGRREEYDVLDKRRGRDPE |
| | | MGGKPRRKNPQEGLYNELQKDKMAEAYSEIGMKGE |
| | | RRRGKGHDGLYQGLSTATKDTYDALHMQALPPRRT |
| | | DGSGATNFSLLKQAGDVEENPGPVSEAMPRGWTALC |
| | | LLSLLPSGFMSLDNNGTATPELPTQGTFSNVSTNVSY |
| | | QETTTPSTLGSTSLHPVSQHGNEATTNITETTVKFTST |
| | | SVITSVYGNTNSSVQSQTSVISTVFTTPANVSTPETTL |
| | | KPSLSPGNVSDLSTTSTSLATSPTKPYTSSSPILSDIKAE |
| | | IKCSGIREVKLTQGICLEQNKTSSCAEFKKDRGEGLA |
| | | RVLCGEEQADADAGAQVCSLLLAQSEVRPQCLLLVL |
| | | ANRTEISSKLQLMKKHQSDLKKLGILDFTEQDVASHQ |
| | | SYSQKTLIALVTSGALLAVLGITGYFLMNRRSWSPTG |
| | | EGGGGGFKRDLGGVKLPHLFGKRLVEARMASYPCH |
| | | QHASAFDQAARSRGHSNRRTALRPRRQQEATEVRLE |
| | | QKMPTLLRVYIDGPHGMGKTTTTQLLVALGSRDDIV |
| | | YVPEPMTYWQVLGASETIANIYTTQHRLDQGEISAGD |
| | | AAVVMTSAQITMGMPYAVTDAVLAPHVGGEAGSSH |
| | | APPPALTLLLDRHPIAVMLCYPAARYLMGSMTPQAV |
| | | LAFVALIPPTLPGTNIVLGALPEDRHIDRLAKRQRPGE |
| | | RLDLAMLAAIRRVYGLLANTVRYLQGGGSWWEDW |
| | | GQLSGTAVPPQGAEPQSNAGPRPHIGDTLFTLFRAPE |
| | | LLAPNGDLYNVFAWALDVLAKRLRPMHVFILDYDQ |
| | | SPAGCRDALLQLTSGMVQTHVTTPGSIPTICDLARTF |
| | | AREMGEAN |
| CD2-CAR- | SEQ ID | MALPVTALLLPLALLLHAARPDIVMTQAAPSVPVTPG |
| CD28_CD34 | NO: 37 | ESVSISCRSSKTLLHSNGNTYLYWFLQRPGQSPQVLIY |
| | | RMSNLASGVPNRFSGSGSETTFTLRISRVEAEDVGIYY |
| | | CMQHLEYPYTFGGGTKLEIERGGGGSGGGGSGGGGS |
| | | GGGGSEVQLEESGAELVRPGTSVKLSCKASGYTFTSY |
| | | WMHWIKQRPEQGLEWIGRIDPYDSETHYNEKFKDKA |
| | | ILSVDKSSSTAYIQLSSLTSDDSAVYYCSRRDAKYDG |
| | | YALDYWGQGTSVTVSSPRASTTTPAPRPPTPAPTIAS |
| | | QPLSLRPEACRPAAGGAVHTRGLDFACDFWVLVVV |
| | | GGVLACYSLLVTVAFIIFWVRSKRSRLLHSDYMNMT |
| | | PRRPGPTRKHYQPYAPPRDFAAYRSRVKFSRSADAPA |
| | | YKQGQNQLYNELNLGRREEYDVLDKRRGRDPEMGG |
| | | KPRRKNPQEGLYNELQKDKMAEAYSEIGMKGERRR |
| | | GKGHDGLYQGLSTATKDTYDALHMQALPPRRTDGS |
| | | GATNFSLLKQAGDVEENPGPVSEAMPRGWTALCLLS |
| | | LLPSGFMSLDNNGTATPELPTQGTFSNVSTNVSYQET |
| | | TTPSTLGSTSLHPVSQHGNEATTNITETTVKFTSTSVIT |
| | | SVYGNTNSSVQSQTSVISTVFTTPANVSTPETTLKPSL |
| | | SPGNVSDLSTTSTSLATSPTKPYTSSSPILSDIKAEIKCS |
| | | GIREVKLTQGICLEQNKTSSCAEFKKDRGEGLARVLC |
| | | GEEQADADAGAQVCSLLLAQSEVRPQCLLLVLANRT |
| | | EISSKLQLMKKHQSDLKKLGILDFTEQDVASHQSYSQ |
| | | KTLIALVTSGALLAVLGITGYFLMNRRSWSPI |
| CD2-CAR-4- | SEQ ID | MALPVTALLLPLALLLHAARPDIVMTQAAPSVPVTPG |
| 1BB_CD34 | NO: 38 | ESVSISCRSSKTLLHSNGNTYLYWFLQRPGQSPQVLIY |
| | | RMSNLASGVPNRFSGSGSETTFTLRISRVEAEDVGIYY |
| | | CMQHLEYPYTFGGGTKLEIERGGGGSGGGGSGGGGS |
| | | GGGGSEVQLEESGAELVRPGTSVKLSCKASGYTFTSY |
| | | WMHWIKQRPEQGLEWIGRIDPYDSETHYNEKFKDKA |
| | | ILSVDKSSSTAYIQLSSLTSDDSAVYYCSRRDAKYDG |
| | | YALDYWGQGTSVTVSSPRASTTTPAPRPPTPAPTIAS |
| | | QPLSLRPEACRPAAGGAVHTRGLDFACDFWVLVVV |
| | | GGVLACYSLLVTVAFIIFWVKRGRKKLLYIFKQPFMR |
| | | PVQTTQEEDGCSCRFPEEEEGGCELRVKFSRSADAPA |
| | | YKQGQNQLYNELNLGRREEYDVLDKRRGRDPEMGG |
| | | KPRRKNPQEGLYNELQKDKMAEAYSEIGMKGERRR |
| | | GKGHDGLYQGLSTATKDTYDALHMQALPPRRTDGS |
| | | GATNFSLLKQAGDVEENPGPVSEAMPRGWTALCLLS |
| | | LLPSGFMSLDNNGTATPELPTQGTFSNVSTNVSYQET |
| | | TTPSTLGSTSLHPVSQHGNEATTNITETTVKFTSTSVIT |
| | | SVYGNTNSSVQSQTSVISTVFTTPANVSTPETTLKPSL |
| | | SPGNVSDLSTTSTSLATSPTKPYTSSSPILSDIKAEIKCS |
| | | GIREVKLTQGICLEQNKTSSCAEFKKDRGEGLARVLC |
| | | GEEQADADAGAQVCSLLLAQSEVRPQCLLLVLANRT |
| | | EISSKLQLMKKHQSDLKKLGILDFTEQDVASHQSYSQ |
| | | KTLIALVTSGALLAVLGITGYFLMNRRSWSPI |
| CD3-CD28- | SEQ ID | MALPVTALLLPLALLLHAARPGSQVQLQQSGAELAR |
| CD34 | NO: 39 | PGASVKMSCKASGYTFTRYTMHWVKQRPGQGLEWI |
| | | GYINPSRGYTNYNQKFKDKATLTTDKSSSTAYMQLS |
| | | SLTSEDSAVYYCARYYDDHYCLDYWGQGTTLTVSS |
| | | GGGGSGGGGSGGGGSGGGGSQIVLTQSPAIMSASPG |
| | | EKVTMTCSASSSVSYMNWYQQKSGTSPKRWIYDTSK |
| | | LASGVPAHFRGSGSGTSYSLTISGMEAEDAATYYCQ |
| | | QWSSNPFTFGSGTKLEINRPRASTTTPAPRPPTPAPTIA |
| | | SQPLSLRPEACRPAAGGAVHTRGLDFACDFWVLVVV |
| | | GGVLACYSLLVTVAFIIFWVRSKRSRLLHSDYMNMT |
| | | PRRPGPTRKHYQPYAPPRDFAAYRSRVKFSRSADAPA |
| | | YKQGQNQLYNELNLGRREEYDVLDKRRGRDPEMGG |
| | | KPRRKNPQEGLYNELQKDKMAEAYSEIGMKGERRR |
| | | GKGHDGLYQGLSTATKDTYDALHMQALPPRRTDGS |
| | | GATNFSLLKQAGDVEENPGPVSEAMPRGWTALCLLS |
| | | LLPSGFMSLDNNGTATPELPTQGTFSNVSTNVSYQET |
| | | TTPSTLGSTSLHPVSQHGNEATTNITETTVKFTSTSVIT |
| | | SVYGNTNSSVQSQTSVISTVFTTPANVSTPETTLKPSL |
| | | SPGNVSDLSTTSTSLATSPTKPYTSSSPILSDIKAEIKCS |
| | | GIREVKLTQGICLEQNKTSSCAEFKKDRGEGLARVLC |
| | | GEEQADADAGAQVCSLLLAQSEVRPQCLLLVLANRT |
| | | EISSKLQLMKKHQSDLKKLGILDFTEQDVASHQSYSQ |
| | | KTLIALVTSGALLAVLGITGYFLMNRRSWSPI |

In a similar manner, other mono-CAR-T cells may be constructed and are given below in Table 4.

TABLE 4
Mono-CARs and CAR-Ts.

| | Antigen | |
|---|---|---|
| | Target of | Antigen |
| | CAR-T | Deletion/ |
| Example | cells | Suppression |
| M1 | APRIL | — |
| M2 | APRIL | APRIL |
| M3 | APRIL | APRIL + |
| | | TRAC |
| M4 | APRIL | APRIL + |
| | | CD3ε |
| M5 | APRIL | CD3ε |
| M6 | BCMA | — |
| M7 | CD117 | — |
| M8 | CD117 | CD117 |
| M9 | CD123 | — |
| M10 | CD123 | CD123 |
| M11 | CD135 | — |
| M12 | CD135 | CD135 |
| M13 | CD138 | — |
| M14 | CD19 | — |
| M15 | CD1a | — |
| M16 | CD1a | CD3ε |
| M17 | CD1a | TRAC |
| M18 | CD1a | CD1a + |
| | | TRAC |
| M19 | CD1a | CD1a + CD3ε |
| M20 | CD2 | — |
| M21 | CD2 | CD2 |
| M22 | CD2 | CD2 + TRAC |
| M23 | CD2 | CD2 + CD3ε |
| M24 | CD20 | |
| M25 | CD21 | |
| M26 | CD22 | |
| M27 | CD23 | |
| M28 | CD3 | — |
| M29 | CD3 | CD3ε |
| M30 | CD3 | CD3ε + |
| | | TRAC |
| M31 | CD33 | — |
| M32 | CD33 | CD33 |
| M33 | CD371 | — |
| M34 | CD371 | CD371 |
| M35 | CD38 | — |
| M36 | CD38 | CD38 |
| M37 | CD4 | — |
| M38 | CD4 | CD4 |
| M39 | CD4 | CD4 + TRAC |
| M40 | CD4 | CD4 + CD3ε |
| M41 | CD5 | — |
| M42 | CD5 | CD5 |
| M43 | CD5 | CD5 + TRAC |
| M44 | CD5 | CD5 + CD3ε |
| M45 | CD56 | — |
| M46 | CD56 | CD56 |
| M47 | CD56 | CD56 + |
| | | TRAC |
| M48 | CD56 | CD56 + CD3ε |
| M49 | CD56 | CD3ε |
| M50 | CD56 | TRAC |
| M51 | CD7 | — |
| M52 | CD7 | CD7 |
| M53 | CD7 | CD7 + TRAC |
| M54 | CD7 | CD7 + CD3ε |
| M55 | CD79A | — |
| M56 | CD79B | — |
| M57 | CS1 | — |
| M58 | CS1 | CS1 |
| M59 | Tim-3 | — |
| M60 | Tim-3 | Tim-3 |
| M61 | Tim-3 | Tim-3 + |
| | | TRAC |
| M62 | Tim-3 | TRAC |
| M63 | Tim-3 | CD3ε |
| M64 | Tim-3 | Tim-3 + |
| | | CD3ε |

Dual CAR-T Cells (dCAR-T)

A dual CAR-T cell (dCAR-T) may be generated by cloning a protein encoding sequence of a first extracellular ligand-binding domain into a lentiviral vector containing one or more costimulatory domains and a signaling transducing domain and cloning a second protein encoding sequence of a second extracellular ligand-binding domain into the same lentiviral vector containing an additional one or more costimulatory domains and a signaling transducing domain resulting in a plasmid from which the two CAR constructs are expressed from the same vector.

In one embodiment, the disclosure provides an engineered T cell comprising a dual Chimeric Antigen Receptor (dCAR), i.e., protein encoding sequence of two CARs expressed from a single lentivirus construct, that specifically binds CD5 and TCR receptor alpha chain (TRAC), wherein the T cell is deficient in CD5 and TRAC (e.g., CD5*TRAC-dCARTΔCD5ΔTRAC cell). In non-limiting examples the deficiency in CD5 and the TCR receptor alpha chain (TRAC) resulted from (a) modification of CD5 and the TCR receptor alpha chain (TRAC) expressed by the T cell such that the chimeric antigen receptor no longer specifically binds the modified CD5 and the TCR receptor alpha chain (TRAC), (b) modification of the T cell such that expression of the CD5 and the TCR receptor alpha chain (TRAC) is reduced in the T cell by at least 50%, at least 60%, at least 70%, at least 80%, at least 90% or more, or (c) modification of the T cell such that CD5 and the TCR receptor alpha chain (TRAC) is not expressed (e.g., by deletion or disruption of the gene encoding CD5 and/or the TCR receptor alpha chain (TRAC). In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA and is expressed in the CD5*TRAC-CARTΔCD5ΔTRAC cells.

In a second embodiment, the disclosure provides an engineered T cell comprising a dCAR that specifically binds CD7 and TCR receptor alpha chain (TRAC), wherein the T cell is deficient in CD7 and TRAC, e.g., CD7*TRAC-dCARTΔCD7ΔTRAC cell. In non-limiting examples the deficiency in CD7 and the TCR receptor alpha chain (TRAC) resulted from (a) modification of CD5 and the TCR receptor alpha chain (TRAC) expressed by the T cell such that the chimeric antigen receptor no longer specifically binds the modified CD7 and the TCR receptor alpha chain (TRAC), (b) modification of the T cell such that expression of the CD7 and the TCR receptor alpha chain (TRAC) is reduced in the T cell by at least 50%, at least 60%, at least 70%, at least 80%, at least 90% or more, or (c) modification of the T cell such that CD7 and the TCR receptor alpha chain (TRAC) is not expressed (e.g., by deletion or disruption of the gene encoding CD7 and/or the TCR receptor alpha chain (TRAC). In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA and is expressed in the CD7*TRAC-dCARTΔCD7ΔTRAC cells.

In a third embodiment, the disclosure provides an engineered T cell comprising a dCAR that specifically binds CD2 and TCR receptor alpha chain (TRAC), wherein the T cell is deficient in CD2 and TRAC, e.g., CD2*TRAC-dCARTΔCD2ΔTRAC cell. In non-limiting examples the deficiency in CD2 and the TCR receptor alpha chain (TRAC) resulted from (a) modification of CD2 and the TCR receptor alpha chain (TRAC) expressed by the T cell such that the chimeric antigen receptor no longer specifically binds the modified CD2 and the TCR receptor alpha chain (TRAC), (b) modification of the T cell such that expression of the CD7 and the TCR receptor alpha chain (TRAC) is reduced in the T cell by at least 50%, at least 60%, at least 70%, at least 80%, at least 90% or more, or (c) modification of the T cell such that CD2 and the TCR receptor alpha chain (TRAC) is not expressed (e.g., by deletion or disruption of the gene encoding CD2 and/or the TCR receptor alpha chain (TRAC). In further embodiments, the T cell comprises a suicide gene. In non-limiting examples, a protein coding sequence of a modified Human-Herpes Simplex Virus-1-thymidine kinase (TK) gene fused is in-frame to the extracellular and transmembrane domains of the human CD34 cDNA and is expressed in CD2*TRAC-dCARTΔCD2ΔTRAC cells.

In another embodiment, the disclosure provides an engineered T cell comprising a dCAR that specifically binds CD4 and TCR receptor alpha chain (TRAC), wherein the T cell is deficient in CD4 and TRAC, e.g., CD4*TRAC-dCARTΔCD4ΔTRAC cell. In non-limiting examples the deficiency in CD4 and the TCR receptor alpha chain (TRAC) resulted from (a) modification of CD4 and the TCR receptor alpha chain (TRAC) expressed by the T cell such that the chimeric antigen receptor no longer specifically binds the modified CD4 and the TCR receptor alpha chain (TRAC), (b) modification of the T cell such that expression of the CD7 and the TCR receptor alpha chain (TRAC) is reduced in the T cell by at least 50%, at least 60%, at least 70%, at least 80%, at least 90% or more, or (c) modification of the T cell such that CD4 and the TCR receptor alpha chain (TRAC) is not expressed (e.g., by deletion or disruption of the gene encoding CD4 and/or the TCR receptor alpha chain (TRAC). In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA and is expressed in the CD4*TRAC-dCARTΔCD4ΔTRAC cells.

In another embodiment, the disclosure provides an engineered T cell comprising a dCAR that specifically binds CD3 and TCR receptor alpha chain (TRAC), wherein the T cell is deficient in CD3 and TRAC, e.g., CD3*TRAC-dCARTΔCD3TRAC cell. In non-limiting examples the deficiency in CD3 and the TCR receptor alpha chain (TRAC) resulted from (a) modification of CD3 and the TCR receptor alpha chain (TRAC) expressed by the T cell such that the chimeric antigen receptor no longer specifically binds the modified CD3 and the TCR receptor alpha chain (TRAC), (b) modification of the T cell such that expression of the CD3 and the TCR receptor alpha chain (TRAC) is reduced in the T cell by at least 50%, at least 60%, at least 70%, at least 80%, at least 90% or more, or (c) modification of the T cell such that CD3 and the TCR receptor alpha chain (TRAC) is not expressed (e.g., by deletion or disruption of the gene encoding CD3 and/or the TCR receptor alpha chain (TRAC). In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA and is expressed in the CD3*TRAC-dCARTΔCD3ΔTRAC cells.

In another embodiment, the disclosure provides an engineered T cell comprising a dCAR that specifically binds CD2 and the CD3 epsilon (ε) chain, wherein the T cell is deficient in CD2 and CD3 epsilon, e.g., CD2*CD3ε-dCARTΔCD2ΔCD3ε cell. In non-limiting examples the deficiency in CD2 and the CD3 epsilon (8) chain resulted from (a) modification of CD2 and CD3 epsilon expressed by the T cell such that the chimeric antigen receptor no longer specifically binds the modified CD2 and CD3 epsilon, (b) modification of the T cell such that expression of the CD2 and CD3 epsilon is reduced in the T cell by at least 50%, at least 60%, at least 70%, at least 80%, at least 90% or more, or (c) modification of the T cell such that CD2 and CD3 epsilon is not expressed (e.g., by deletion or disruption of the gene encoding CD2 and/or CD3 epsilon. In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA and is expressed in CD2*CD3ε-dCARTΔCD2ΔCD3ε cells.

In another embodiment, the disclosure provides an engineered T cell comprising a dCAR that specifically binds CD4 and the CD3 epsilon (8) chain, wherein the T cell is deficient in CD2 and CD3ε, e.g., CD4*CD3ε-dCARTΔCD4ΔCD3ε cell. In non-limiting examples the deficiency in CD4 and the CD3ε chain resulted from (a) modification of CD4 and CD3 epsilon expressed by the T cell such that the chimeric antigen receptor no longer specifically binds the modified CD4 and CD3ε, (b) modification of the T cell such that expression of the CD4 and CD3ε is reduced in the T cell by at least 50%, at least 60%, at least 70%, at least 80%, at least 90% or more, or (c) modification of the T cell such that CD4 and CD3ε is not expressed (e.g., by deletion or disruption of the gene encoding CD4 and/or CD3ε. In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA and is expressed in CD4*CD3ε-dCARTΔCD4ΔCD3ε cells.

In another embodiment, the disclosure provides an engineered T cell comprising a dCAR that specifically binds CD5 and the TCR beta (0) chain, wherein the T cell is deficient in CD5 and TCRβ, e.g., CD5*TCRβ-dCARTΔCD5ΔTCRβ cell. In non-limiting examples the deficiency in CD5 and the TCRβ chain resulted from (a) modification of CD5 and TCRβ expressed by the T cell such that the chimeric antigen receptor no longer specifically binds the modified CD5 and TCRβ, (b) modification of the T cell such that expression of the CD5 and TCRβ is reduced in the T cell by at least 50%, at least 60%, at least 70%, at least 80%, at least 90% or more, or (c) modification of the T cell such that CD5 and TCRβ is not expressed (e.g., by deletion or disruption of the gene encoding CD5 and/or TCR. In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA is expressed in CD5*TCRβ-dCARTΔCD5ΔTCRβ cells.

In another embodiment, the disclosure provides an engineered T cell comprising a dCAR that specifically binds CD7 and the TCR beta (0) chain, wherein the T cell is deficient in CD5 and TCR beta, e.g., CD7*TCRβ-dCARTΔCD7ΔTCRβ cell. In non-limiting examples the deficiency in CD7 and the TCRβ chain resulted from (a) modification of CD7 and TCRβ expressed by the T cell such that the chimeric antigen receptor no longer specifically binds the modified CD7 and TCRβ, (b) modification of the T cell such that expression of the CD7 and TCRβ is reduced in the T cell by at least 50%, at least 60%, at least 70%, at least 80%, at least 90% or more, or (c) modification of the T cell such that CD7 and TCRβ is not expressed (e.g., by deletion or disruption of the gene encoding CD7 and/or TCRβ. In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA is expressed in the CD7*TCRβ-dCARTΔCD7ΔTCRβ cells.

In another embodiment, the disclosure provides an engineered T cell comprising a dCAR that specifically binds CD2 and the TCR beta (0) chain, wherein the T cell is deficient in CD2 and TCR 3, e.g., CD2*TCRβ-dCARTΔCD7ΔTCRβ cell. In non-limiting examples the deficiency in CD2 and the TCRβ chain resulted from (a) modification of CD2 and TCR 3 expressed by the T cell such that the chimeric antigen receptor no longer specifically binds the modified CD2 and TCRβ, (b) modification of the T cell such that expression of the CD2 and TCRβ is reduced in the T cell by at least 50%, at least 60%, at least 70%, at least 80%, at least 90% or more, or (c) modification of the T cell such that CD2 and TCRβ is not expressed (e.g., by deletion or disruption of the gene encoding CD2 and/or TCRβ. In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA and is expressed in CD2*TCRβ-dCARTΔCD2ΔTCRβ cells.

In another embodiment, the disclosure provides an engineered T cell comprising a dCAR that specifically binds CD4 and the TCR beta ((3) chain, wherein the T cell is deficient in CD2 and TCRβ, e.g., CD4*TCRβ-dCARTΔCD4ΔTCRβ cell. In non-limiting examples the deficiency in CD4 and the TCRβ chain resulted from (a) modification of CD4 and TCRβ expressed by the T cell such that the chimeric antigen receptor no longer specifically binds the modified CD4 and TCRβ, (b) modification of the T cell such that expression of the CD4 and TCRβ is reduced in the T cell by at least 50%, at least 60%, at least 70%, at least 80%, at least 90% or more, or (c) modification of the T cell such that CD4 and TCRβ is not expressed (e.g., by deletion or disruption of the gene encoding CD4 and/or TCRβ. In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA and is expressed in CD4*TCRβ-dCARTΔCD4ΔTCRβ cells.

In another embodiment, the disclosure provides an engineered T cell comprising a dCAR that specifically binds CD7 and CD2, wherein the T cell is deficient in CD7 and CD2, e.g., CD7*CD2-dCARTΔCD7ΔCD2 cell. In non-limiting examples the deficiency in CD7 and CD2 resulted from (a) modification of CD7 and CD2 expressed by the T cell such that the chimeric antigen receptor no longer specifically binds the modified CD7 and CD2, (b) modification of the T cell such that expression of the CD7 and CD2 is reduced in the T cell by at least 50%, at least 60%, at least 70%, at least 80%, at least 90% or more, or (c) modification of the T cell such that CD7 and CD2 is not expressed (e.g., by deletion or disruption of the gene encoding CD7 and/or CD2. In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA and is expressed in CD7*CD2-dCARTΔCD7ΔCD2 cells.

In another embodiment, the disclosure provides an engineered T cell comprising a dCAR that specifically binds CD7 and CD5, wherein the T cell is deficient in CD7 and CD5, e.g., CD7*CD5-dCARTΔCD7ΔCD5 cell. In non-limiting examples the deficiency in CD7 and CD5 resulted from (a) modification of CD7 and CD5 expressed by the T cell such that the chimeric antigen receptor no longer specifically binds the modified CD7 and CD5, (b) modification of the T cell such that expression of the CD7 and CD5 is reduced in the T cell by at least 50%, at least 60%, at least 70%, at least 80%, at least 90% or more, or (c) modification of the T cell such that CD7 and CD5 is not expressed (e.g., by deletion or disruption of the gene encoding CD7 and/or CD5. In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA and is expressed in CD7*CD5-dCARTΔCD7ΔCD5 cells.

In another embodiment, the disclosure provides an engineered T cell comprising a dCAR that specifically binds CD7 and CD4, wherein the T cell is deficient in CD7 and CD4 (e.g., CD7*CD4-dCARTΔCD7ΔCD4 cell). In non-limiting examples the deficiency in CD7 and CD4 resulted from (a) modification of CD7 and CD4 expressed by the T cell such that the chimeric antigen receptor no longer specifically binds the modified CD7 and CD4, (b) modification of the T cell such that expression of the CD7 and CD4 is reduced in the T cell by at least 50%, at least 60%, at least 70%, at least 80%, at least 90% or more, or (c) modification of the T cell such that CD7 and CD4 is not expressed (e.g., by deletion or disruption of the gene encoding CD7 and/or CD4. In further embodiments, the T cell comprises a suicide gene. In non-limiting examples the suicide gene expressed in the CD7*CD4-dCARTΔCD7ΔCD4 cells encodes a modified Human-Herpes Simplex Virus-1-thymidine kinase (TK) gene is fused in-frame to the extracellular and transmembrane domains of the human CD34 cDNA and is expressed in CD7*CD4-dCARTΔCD7ΔCD4 cells.

In another embodiment, the disclosure provides an engineered T cell comprising a dCAR that specifically binds CD2 and CD5, wherein the T cell is deficient in CD2, CD5, and TRAC, e.g., CD2*CD5-dCARTΔCD2ΔCD5ΔTRAC cell. In non-limiting examples the deficiency in CD2 and CD5 resulted from (a) modification of CD2 and CD5 expressed by the T cell such that the chimeric antigen receptor no longer specifically binds the modified CD2 and CD5, (b) modification of the T cell such that expression of the CD2 and CD5 is reduced in the T cell by at least 50%, at least 60%, at least 70%, at least 80%, at least 90% or more, or (c) modification of the T cell such that CD2 and CD5 is not expressed (e.g., by deletion or disruption of the gene encoding CD2 and/or CD5. In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA and is expressed in CD2*CD5-dCARTΔCD2ΔCD5 cells.

In another embodiment, the disclosure provides an engineered T cell comprising a dCAR that specifically binds CD2 and CD4, wherein the T cell is deficient in CD2, CD4, and TRAC, e.g., CD2*CD4-dCARTΔCD2ΔCD4ΔTRAC cell. In non-limiting examples the deficiency in CD2 and CD4 resulted from (a) modification of CD2 and CD4 expressed by the T cell such that the chimeric antigen receptor no longer specifically binds the modified CD2 and CD4, (b) modification of the T cell such that expression of the CD2 and CD4 is reduced in the T cell by at least 50%, at least 60%, at least 70%, at least 80%, at least 90% or more, or (c) modification of the T cell such that CD2 and CD4 is not expressed (e.g., by deletion or disruption of the gene encoding CD2 and/or CD4. In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA and is expressed in CD2*CD4-dCARTΔCD2ΔCD4 cells.

In another embodiment, the disclosure provides an engineered T cell comprising a dCAR that specifically binds CD5 and CD4, wherein the T cell is deficient in CD5 and CD4, e.g., CD5*CD4-dCARTΔCD5ΔCD4 cell. In non-limiting examples the deficiency in CD5 and CD4 resulted from (a) modification of CD5 and CD4 expressed by the T cell such that the chimeric antigen receptor no longer specifically binds the modified CD5 and CD4, (b) modification of the T cell such that expression of the CD5 and CD4 is reduced in the T cell by at least 50%, at least 60%, at least 70%, at least 80%, at least 90% or more, or (c) modification of the T cell such that CD5 and CD4 is not expressed (e.g., by deletion or disruption of the gene encoding CD5 and/or CD4. In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA and is expressed in CD5*CD4-dCARTΔCD5ΔCD4 cells.

In one embodiment, a dual CAR-T cell comprises (i) a first chimeric antigen receptor (CAR) polypeptide comprising a first signal peptide, a first extracellular ligand-binding domain, a first hinge region, a first transmembrane domain, one or more co-stimulatory domains, and a first signaling transducing domain; and (ii) a second chimeric antigen receptor polypeptide comprising a second signaling peptide, a second extracellular ligand-binding domain, a second hinge region, a second transmembrane domain, one or more co-stimulatory domains, and a second signaling transducing domain; wherein the first extracellular ligand-binding domain and the second extracellular ligand-binding domain have affinities for different cell surface molecules; and wherein the dual CAR-T cell possesses one or more genetic disruptions resulting in reduced expression of the cell surface molecule in the dual CAR-T cell.

In a second embodiment, the first signal peptide is a CD8a signal sequence.

In a third embodiment, the first extracellular ligand-binding domain is a fusion protein of the variable regions of immunoglobulin heavy and light chains, designated V_{H}1 and V_{L}1, and connected by a short linker peptide of 5 amino acids (GGGGS). In some embodiments, this linker peptide is repeated 3 or 4 times. In some embodiments, the first antigen recognition domain can be selected from V_{H}1-(GGGGS)₃₋₄ (SEQ ID NO:449)-V_{L}1 or V_{L}1-(GGGGS)₃₋₄ (SEQ ID NO:449)-V_{H}1.

In another embodiment, the first hinge region comprises CD8a.

In another embodiment, the first transmembrane domain is CD8 or CD28.

In some embodiments, the first co-stimulatory domain comprises 4-1BB, CD28, or a combination of both, in either order, i.e., 4-1BB-CD28 or CD28-4-1BB.

In some embodiments, the first signaling domain is CD3ζ or a CD3ζ bi-peptide, i.e. CD3ζ-CD3ζ.

In some embodiments, the second signal peptide is a CD8a signal sequence of SEQ NO:1.

In some embodiments, the second extracellular ligand-binding domain is fusion protein of the variable regions of immunoglobulin heavy and light chains, designated V_{H}2 and V_{L}2, and connected by a short linker peptide of 5 amino acids (GGGGS). In some embodiments, this linker peptide is repeated 3 or 4 times. In some embodiments, the second antigen recognition domain can be selected from V_{H}2-(GGGGS)₃ (SEQ ID NO:449)-V_{L}2 or V_{L}2-(GGGGS)₃₋₄ (SEQ ID NO:449)-V_{H}2.

In another embodiment, the second hinge region comprises CD8a.

In another embodiment, the second transmembrane domain is CD8 or CD28.

In some embodiments, the second co-stimulatory domain comprises 4-1BB, CD28, or a combination of both, in either order, i.e. 4-1BB-CD28 or CD28-4-1BB.

In some embodiments, the second signaling domain is CD3ζ or a CD3ζ bi-peptide, i.e. CD3ζ-CD3ζ.

In some embodiments, the CAR polypeptide comprises a first extracellular ligand-binding domain fusion protein of V_{H}1-(GGGGS)₃₋₄ (SEQ ID NO:449)-V_{L}1 and a second extracellular ligand-binding domain fusion protein of V_{H}2-(GGGGS)₃₋₄ (SEQ ID NO:449)-V_{L}².

In some embodiments, the CAR polypeptide comprises a first extracellular ligand-binding domain fusion protein of V_{L}1-(GGGGS)₃₋₄ (SEQ ID NO:449)-V_{H}1 and a second extracellular ligand-binding domain fusion protein of V_{L}2-(GGGGS)₃₋₄-V_{H}2.

In some embodiments, the CAR polypeptide comprises a first extracellular ligand-binding domain fusion protein of V_{H}2-(GGGGS)₃₋₄ (SEQ ID NO:449)-V_{L}² and a second extracellular ligand-binding domain fusion protein of V_{H}1-(GGGGS)₃₋₄-V_{L}1.

In some embodiments, the CAR polypeptide comprises a first extracellular ligand-binding domain fusion protein of V_{L}2-(GGGGS)₃₋₄ (SEQ ID NO:449)-V_{H}2 and a second extracellular ligand-binding domain fusion protein of V_{L}1-(GGGGS)₃₋₄-V_{H}1.

In some embodiments, the CAR polypeptide comprises a first extracellular ligand-binding domain fusion protein of V_{H}1-(GGGGS)₃₋₄ (SEQ ID NO:449)-V and a second extracellular ligand-binding domain fusion protein of V_{L}2-(GGGGS)₃₋₄ (SEQ ID NO:449)-V_{H}2.

In some embodiments, the CAR polypeptide comprises a first extracellular ligand-binding domain fusion protein of V_{H}1-(GGGGS)₃₋₄ (SEQ ID NO:449)-V_{H}1 and a second extracellular ligand-binding domain fusion protein of V_{H}2-(GGGGS)₃₋₄ (SEQ ID NO:449)-V_{L}2.

In some embodiments, the CAR polypeptide comprises a first extracellular ligand-binding domain fusion protein of V_{H}2-(GGGGS)₃₋₄ (SEQ ID NO:449)-V_{L}2 and a second extracellular ligand-binding domain fusion protein of V_{L}1-(GGGGS)₃₋₄-V_{H}1.

In some embodiments, the CAR polypeptide comprises a first extracellular ligand-binding domain fusion protein of V_{L}2-(GGGGS)₃₋₄ (SEQ ID NO:449)-V_{H}2 and a second extracellular ligand-binding domain fusion protein of V_{H}1-(GGGGS)₃₋₄ (SEQ ID NO:449)-V_{H}1.

In some embodiments, the CAR polypeptide comprises at least one high efficiency cleavage site, wherein the high efficiency cleavage site is selected from P2A, T2A, E2A, and F2A.

In some embodiments, the CAR polypeptide comprises a suicide gene.

In some embodiments, the CAR polypeptide comprises a mutant cytokine receptor.

In some embodiments, the dual CAR-T cell targets two antigens selected from CD5, CD7, CD2, CD4, CD3, CD33, CD123 (IL3RA), CD371 (CLL-1; CLEC12A), CD117 (c-kit), CD135 (FLT3), BCMA, CS1, CD38, CD79A, CD79B, CD138, and CD19, APRIL, and TACI.

Additional examples of dual CARs are given below in Table 5.

TABLE 5
Dual CARs and dCAR-Ts

| | Antigen Targets of | Antigen Deletion/ |
|---|---|---|
| Example | CARs in dCAR-T cell | Suppression |
| D1 | APRIL × BCMA | — |
| D2 | APRIL × CD19 | — |
| D3 | APRIL × CD38 | — |
| D4 | APRIL × CD38 | CD38 |
| D5 | APRIL × CS1 | — |
| D6 | APRIL × CS1 | CS1 |
| D7 | BCMA × CD19 | — |
| D8 | BCMA × CD38 | — |
| D9 | BCMA × CD38 | CD38 |
| D10 | BCMA × CS1 | — |
| D11 | BCMA × CS1 | CS1 |
| D12 | CD138 × APRIL | |
| D13 | CD138 × BCMA | |
| D14 | CD138 × CD19 | |
| D15 | CD138 × CD38 | |
| D16 | CD138 × CD38 | CD38 |
| D17 | CD138 × CD79A | |
| D18 | CD138 × CD79B | |
| D19 | CD138 × CS1 | |
| D20 | CD138 × CS1 | CS1 |
| D21 | CD19 × CD38 | — |
| D22 | CD19 × CD38 | CD38 |
| D23 | CD2 × CD3ε | — |
| D24 | CD2 × CD3ε | CD2 |
| D25 | CD2 × CD3ε | CD3ε |
| D26 | CD2 × CD3ε | CD2 and CD3ε |
| D27 | CD2 × CD4 | — |
| D28 | CD2 × CD4 | CD2 |
| D29 | CD2 × CD4 | CD4 |
| D30 | CD2 × CD4 | CD2 and CD4 |
| D31 | CD2 × CD4 | CD2 and TRAC |
| D32 | CD2 × CD4 | CD4 and TRAC |
| D33 | CD2 × CD4 | CD2 and CD4 and TRAC |
| D34 | CD2 × CD5 | — |
| D35 | CD2 × CD5 | CD2 |
| D36 | CD2 × CD5 | CD5 |
| D37 | CD2 × CD5 | CD2 and CD5 |
| D38 | CD2 × CD5 | CD2 and TRAC |
| D39 | CD2 × CD5 | CD5 and TRAC |
| D40 | CD2 × CD5 | CD2 and CD5 and TRAC |
| D41 | CD2 × CD7 | — |
| D42 | CD2 × CD7 | CD2 |
| D43 | CD2 × CD7 | CD7 |
| D44 | CD2 × CD7 | CD2 and CD7 |
| D45 | CD2 × CD7 | CD2 and TRAC |
| D46 | CD2 × CD7 | CD7 and TRAC |
| D47 | CD2 × CD7 | CD2 and CD7 and TRAC |
| D48 | CD3ε × CD4 | — |
| D49 | CD3ε × CD4 | CD3ε |
| D50 | CD3ε × CD4 | CD4 |
| D51 | CD3ε × CD4 | CD3ε and CD4 |
| D52 | CD3ε × CD5 | — |
| D53 | CD3ε × CD5 | CD3ε |
| D54 | CD3ε × CD5 | CD5 |
| D55 | CD3ε × CD5 | CD3ε and CD5 |
| D56 | CD3ε × CD7 | — |
| D57 | CD3ε × CD7 | CD3ε |
| D58 | CD3ε × CD7 | CD7 |
| D59 | CD3ε × CD7 | CD3ε and CD7 |
| D60 | CD4 × CD5 | — |
| D61 | CD4 × CD5 | CD4 |
| D62 | CD4 × CD5 | CD5 |
| D63 | CD4 × CD5 | CD4 and CD5 |
| D64 | CD4 × CD5 | CD4 and TRAC |
| D65 | CD4 × CD5 | CD5 and TRAC |
| D66 | CD4 × CD5 | CD4 and CD5 and TRAC |
| D67 | CD4 × CD7 | — |
| D68 | CD4 × CD7 | CD4 |
| D69 | CD4 × CD7 | CD7 |
| D70 | CD4 × CD7 | CD4 and CD7 |
| D71 | CD4 × CD7 | CD4 and TRAC |
| D72 | CD4 × CD7 | CD7 and TRAC |
| D73 | CD4 × CD7 | CD4 and CD7 and TRAC |
| D74 | CD5 × CD7 | — |
| D75 | CD5 × CD7 | CD5 |
| D76 | CD5 × CD7 | CD7 |
| D77 | CD5 × CD7 | CD5 and CD7 |
| D78 | CD5 × CD7 | CD5 and TRAC |
| D79 | CD5 × CD7 | CD7 and TRAC |
| D80 | CD5 × CD7 | CD5 and CD7 and TRAC |
| D81 | CD79A × APRIL | |
| D82 | CD79A × BCMA | |
| D83 | CD79A × CD19 | |
| D84 | CD79A × CD38 | |
| D85 | CD79A × CD38 | CD38 |
| D86 | CD79A × CD79B | |
| D87 | CD79A × CS1 | |
| D88 | CD79A × CS1 | CS1 |
| D89 | CD79B × APRIL | |
| D90 | CD79B × BCMA | |
| D91 | CD79B × CD19 | |
| D92 | CD79B × CD38 | |
| D93 | CD79B × CD38 | CD38 |
| D94 | CD79B × CD79A | |
| D95 | CD79B × CS1 | |
| D96 | CD79B × CS1 | CS1 |
| D97 | CS1 × CD19 | — |
| D98 | CS1 × CD19 | CS1 |
| D99 | CS1 × CD38 | — |
| D100 | CS1 × CD38 | CS1 |
| D101 | CS1 × CD38 | CD38 |
| D102 | CS1 × CD38 | CS1 and CD38 |
| D103 | TCRβ × CD2 | — |
| D104 | TCRβ × CD2 | TCRβ |
| D105 | TCRβ × CD2 | CD2 |
| D106 | TCRβ × CD2 | TCRβ and CD2 |
| D107 | TCRβ × CD3ε | — |
| D108 | TCRβ × CD3ε | TCRβ |
| D109 | TCRβ × CD3ε | CD3ε |
| D110 | TCRβ × CD3ε | TCRβ and CD3ε |
| D111 | TCRβ × CD4 | — |
| D112 | TCRβ × CD4 | TCRβ |
| D113 | TCRβ × CD4 | CD4 |
| D114 | TCRβ × CD4 | TCRβ and CD4 |
| D115 | TCRβ × CD5 | — |
| D116 | TCRβ × CD5 | TCRβ |
| D117 | TCRβ × CD5 | CD5 |
| D118 | TCRβ × CD5 | TCRβ and CD5 |
| D119 | TCRβ × CD7 | — |
| D120 | TCRβ × CD7 | TCRβ |
| D121 | TCRβ × CD7 | CD7 |
| D122 | TCRβ × CD7 | TCRβ and CD7 |
| D123 | TRAC × CD2 | — |
| D124 | TRAC × CD2 | TRAC |
| D125 | TRAC × CD2 | CD2 |
| D126 | TRAC × CD2 | TRAC and CD2 |
| D127 | TRAC × CD3ε | — |
| D128 | TRAC × CD3ε | TRAC |
| D129 | TRAC × CD3ε | CD3ε |
| D130 | TRAC × CD3ε | TRAC and CD3ε |
| D131 | TRAC × CD4 | — |
| D132 | TRAC × CD4 | TRAC |
| D133 | TRAC × CD4 | CD4 |
| D134 | TRAC × CD4 | TRAC and CD4 |
| D135 | TRAC × CD5 | — |
| D136 | TRAC × CD5 | TRAC |
| D137 | TRAC × CD5 | CD5 |
| D138 | TRAC × CD5 | TRAC and CD5 |
| D139 | TRAC × CD7 | — |
| D140 | TRAC × CD7 | TRAC |
| D141 | TRAC × CD7 | CD7 |
| D142 | TRAC × CD7 | TRAC and CD7 |

Tandem CAR-T Cells (tCAR-T)

A tandem CAR-T cell (tCAR-T), is a T cell with a single chimeric antigen polypeptide comprising two distinct extracellular ligand-binding domains capable of interacting with two different cell surface molecules, wherein the extracellular ligand-binding domains are linked together by a flexible linker and share one or more costimulatory domains, wherein the binding of the first or the second extracellular ligand-binding domain will signal through one or more the costimulatory domains and a signaling transducing domain.

In one embodiment, an engineered T cell comprises a tandem Chimeric Antigen Receptor (tCAR), wherein one extracellular ligand-binding domain specifically binds CD5 and the second extracellular ligand-binding domain binds the TCR receptor alpha chain (TRAC), wherein the T cell is deficient in CD5 and TRAC, e.g., CD5*TRAC-tCARTΔCD5ΔTRAC cell. In non-limiting examples the deficiency in CD5 and the TCR receptor alpha chain (TRAC) resulted from (a) modification of CD5 and the TCR receptor alpha chain (TRAC) expressed by the T cell such that the tCAR no longer specifically binds the modified CD5 and the TCR receptor alpha chain (TRAC), (b) modification of the T cell such that expression of the CD5 and the TCR receptor alpha chain (TRAC) is reduced in the T cell by at least 50%, at least 60%, at least 70%, at least 80%, at least 90% or more, or (c) modification of the T cell such that CD5 and the TCR receptor alpha chain (TRAC) is not expressed (e.g., by deletion or disruption of the gene encoding CD5 and/or the TCR receptor alpha chain (TRAC). In further embodiments, the T cell comprises a suicide gene. In non-limiting examples, a protein-coding sequence of a modified Human-Herpes Simplex Virus-1-thymidine kinase (TK) gene is fused in-frame to the extracellular and transmembrane domains of human CD34 cDNA and is expressed in the CD5*TRAC-tCARTΔCD5ΔTRAC cells.

In a second embodiment, an engineered T cell comprises a tandem Chimeric Antigen Receptor (tCAR), wherein one extracellular ligand-binding domain specifically binds CD7 and the second extracellular ligand-binding domain binds the TCR receptor alpha chain (TRAC), wherein the T cell is deficient in CD7 and TRAC, e.g., CD7*TRAC-tCARTΔCD7ΔTRAC cell. In non-limiting examples the deficiency in CD7 and the TCR receptor alpha chain (TRAC) resulted from (a) modification of CD7 and the TCR receptor alpha chain (TRAC) expressed by the T cell such that the tCAR no longer specifically binds the modified CD7 and the TCR receptor alpha chain (TRAC), (b) modification of the T cell such that expression of the CD7 and the TCR receptor alpha chain (TRAC) is reduced in the T cell by at least 50%, at least 60%, at least 70%, at least 80%, at least 90% or more, or (c) modification of the T cell such that CD7 and the TCR receptor alpha chain (TRAC) is not expressed (e.g., by deletion or disruption of the gene encoding CD7 and/or the TCR receptor alpha chain (TRAC). In further embodiments, the T cell comprises a suicide gene. In non-limiting examples, a protein-coding sequence of a modified Human-Herpes Simplex Virus-1-thymidine kinase (TK) gene is fused in-frame to the extracellular and transmembrane domains of the human CD34 cDNA and is expressed in the CD7*TRAC-tCARTΔCD7ΔTRAC cells.

In a third embodiment, an engineered T cell comprises a tandem Chimeric Antigen Receptor (tCAR), wherein one extracellular ligand-binding domain specifically binds CD2 and the second extracellular ligand-binding domain binds the TCR receptor alpha chain (TRAC), wherein the T cell is deficient in CD2 and TRAC, e.g., CD2*TRAC-tCARTΔCD2ΔTRAC cell. In non-limiting examples the deficiency in CD2 and the TCR receptor alpha chain (TRAC) resulted from (a) modification of CD2 and the TCR receptor alpha chain (TRAC) expressed by the T cell such that the tCAR no longer specifically binds the modified CD2 and the TCR receptor alpha chain (TRAC), (b) modification of the T cell such that expression of the CD2 and the TCR receptor alpha chain (TRAC) is reduced in the T cell by at least 50%, at least 60%, at least 70%, at least 80%, at least 90% or more, or (c) modification of the T cell such that CD2 and the TCR receptor alpha chain (TRAC) is not expressed (e.g., by deletion or disruption of the gene encoding CD2 and/or the TCR receptor alpha chain (TRAC). In further embodiments, the T cell comprises a suicide gene. In non-limiting examples, a protein-coding sequence of a modified Human-Herpes Simplex Virus-1-thymidine kinase (TK) gene is fused in-frame to the extracellular and transmembrane domains of the human CD34 cDNA is expressed in the CD2*TRAC-tCARTΔCD2ΔTRAC cells.

In another embodiment, an engineered T cell comprises a tandem Chimeric Antigen Receptor (tCAR), wherein one extracellular ligand-binding domain specifically binds CD4 and the second extracellular ligand-binding domain binds the TCR receptor alpha chain (TRAC), wherein the T cell is deficient in CD4 and TRAC, e.g., CD4*TRAC-tCARTΔCD4ΔTRAC cell. In non-limiting examples the deficiency in CD4 and the TCR receptor alpha chain (TRAC) resulted from (a) modification of CD4 and the TCR receptor alpha chain (TRAC) expressed by the T cell such that the tCAR no longer specifically binds the modified CD4 and the TCR receptor alpha chain (TRAC), (b) modification of the T cell such that expression of the CD4 and the TCR receptor alpha chain (TRAC) is reduced in the T cell by at least 50%, at least 60%, at least 70%, at least 80%, at least 90% or more, or (c) modification of the T cell such that CD4 and the TCR receptor alpha chain (TRAC) is not expressed (e.g., by deletion or disruption of the gene encoding CD4 and/or the TCR receptor alpha chain (TRAC). In further embodiments, the T cell comprises a suicide gene. In non-limiting examples, a protein-coding sequence of a modified Human-Herpes Simplex Virus-1-thymidine kinase (TK) gene is fused in-frame to the extracellular and transmembrane domains of the human CD34 cDNA and is expressed in the CD4*TRAC-tCARTΔCD4ΔTRAC cells.

In another embodiment, an engineered T cell comprises a tandem Chimeric Antigen Receptor (tCAR), wherein one extracellular ligand-binding domain specifically binds CD3 epsilon (Chimeric) chain and the second extracellular ligand-binding domain binds the TCR receptor alpha chain (TRAC), wherein the T cell is deficient in CD3ε and TRAC, e.g., a CD3ε*TRAC-tCARTΔCD3εΔTRAC cell. In non-limiting examples the deficiency in CD3ε and the TCR receptor alpha chain (TRAC) resulted from (a) modification of CD3ε and the TCR receptor alpha chain (TRAC) expressed by the T cell such that the tCAR no longer specifically binds the modified CD3ε and the TCR receptor alpha chain (TRAC), (b) modification of the T cell such that expression of the CD3ε and the TCR receptor alpha chain (TRAC) is reduced in the T cell by at least 50%, at least 60%, at least 70%, at least 80%, at least 90% or more, or (c) modification of the T cell such that CD3ε and the TCR receptor alpha chain (TRAC) is not expressed (e.g., by deletion or disruption of the gene encoding CD3ε and/or the TCR receptor alpha chain (TRAC). In further embodiments, the T cell comprises a suicide gene. In non-limiting examples, a protein-coding sequence of a modified Human-Herpes Simplex Virus-1-thymidine kinase (TK) gene is fused in-frame to the extracellular and transmembrane domains of the human CD34 cDNA and is expressed in the CD3ε*TRAC-tCARTΔCD3εΔTRAC cells.

In another embodiment, an engineered T cell comprises a tandem Chimeric Antigen Receptor (tCAR), wherein one extracellular ligand-binding domain specifically binds CD2 and the second extracellular ligand-binding domain binds the CD3 epsilon (8) chain, wherein the T cell is deficient in CD2 and CD3ε, e.g., CD2*CD3ε-tCARTΔCD2ΔCD3ε cell. In non-limiting examples the deficiency in CD2 and the CD3ε resulted from (a) modification of CD2 and CD3ε expressed by the T cell such that the tCAR no longer specifically binds the modified CD2 and CD3ε, (b) modification of the T cell such that expression of the CD2 and CD3ε is reduced in the T cell by at least 50%, at least 60%, at least 70%, at least 80%, at least 90% or more, or (c) modification of the T cell such that CD2 and CD3ε is not expressed (e.g., by deletion or disruption of the gene encoding CD2 and/or CD3ε. In further embodiments, the T cell comprises a suicide gene. In non-limiting examples, a protein-coding sequence of a modified Human-Herpes Simplex Virus-1-thymidine kinase (TK) gene is fused in-frame to the extracellular and transmembrane domains of the human CD34 cDNA and is expressed in the CD2*CD3ε-tCARTΔCD2ΔCD3ε cells.

In another embodiment, an engineered T cell comprises a tandem Chimeric Antigen Receptor (tCAR), wherein one extracellular ligand-binding domain specifically binds CD4 and the second extracellular ligand-binding domain binds the CD3 epsilon (8) chain, wherein the T cell is deficient in CD4 and CD3ε, e.g., CD4*CD3ε-tCARTΔCD4ΔCD3ε cell. In non-limiting examples the deficiency in CD4 and the CD3ε resulted from (a) modification of CD4 and CD3ε expressed by the T cell such that the tCAR no longer specifically binds the modified CD4 and CD3ε, (b) modification of the T cell such that expression of the CD4 and CD3ε is reduced in the T cell by at least 50%, at least 60%, at least 70%, at least 80%, at least 90% or more, or (c) modification of the T cell such that CD4 and CD3ε is not expressed (e.g., by deletion or disruption of the gene encoding CD4 and/or CD3ε. In further embodiments, the T cell comprises a suicide gene. In non-limiting examples, a protein-coding sequence of a modified Human-Herpes Simplex Virus-1-thymidine kinase (TK) gene is fused in-frame to the extracellular and transmembrane domains of the human CD34 cDNA and is expressed in the CD4*CD3ε-tCARTΔCD4ΔCD3ε cells.

In another embodiment, an engineered T cell comprises a tandem Chimeric Antigen Receptor (tCAR), wherein one extracellular ligand-binding domain specifically binds CD5 and the second extracellular ligand-binding domain binds the TCRβ chain, wherein the T cell is deficient in CD5 and TCRβ chain, e.g., a CD5*TCRβ-tCARTΔCD5ΔTCRβ cell. In non-limiting examples the deficiency in CD5 and the TCRβ chain resulted from (a) modification of CD5 and TCRβ expressed by the T cell such that the tCAR no longer specifically binds the modified CD5 and TCRβ, (b) modification of the T cell such that expression of the CD5 and TCRβ is reduced in the T cell by at least 50%, at least 60%, at least 70%, at least 80%, at least 90% or more, or (c) modification of the T cell such that CD5 and TCRβ is not expressed (e.g., by deletion or disruption of the gene encoding CD5 and/or TCRβ. In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA and is expressed in the CD5*TCRβ-tCARTΔCD5ΔTCRβ cells.

In another embodiment, an engineered T cell comprises a tandem Chimeric Antigen Receptor (tCAR), wherein one extracellular ligand-binding domain specifically binds CD7 and the second extracellular ligand-binding domain binds the TCRβ chain, wherein the T cell is deficient in CD7 and TCRβ chain, e.g., a CD7*TCRβ-tCARTΔCD7ΔTCRβ cell. In non-limiting examples the deficiency in CD7 and the TCRβ chain resulted from (a) modification of CD7 and TCRβ expressed by the T cell such that the tCAR no longer specifically binds the modified CD7 and TCRβ, (b) modification of the T cell such that expression of the CD7 and TCRβ is reduced in the T cell by at least 50%, at least 60%, at least 70%, at least 80%, at least 90% or more, or (c) modification of the T cell such that CD7 and TCRβ is not expressed (e.g., by deletion or disruption of the gene encoding CD7 and/or TCRβ. In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA and is expressed in the CD7*TCRβ-tCARTΔCD7ΔTCRβ cells.

In another embodiment, an engineered T cell comprises a tandem Chimeric Antigen Receptor (tCAR), wherein one extracellular ligand-binding domain specifically binds CD2 and the second extracellular ligand-binding domain binds the TCRβ chain, wherein the T cell is deficient in CD2 and TCRβ chain, e.g., a CD2*TCRβ-tCARTΔCD7ΔTCRβ cell. In non-limiting examples the deficiency in CD2 and the TCRβ chain resulted from (a) modification of CD2 and TCRβ expressed by the T cell such that the tCAR no longer specifically binds the modified CD2 and TCRβ, (b) modification of the T cell such that expression of the CD2 and TCRβ is reduced in the T cell by at least 50%, at least 60%, at least 70%, at least 80%, at least 90% or more, or (c) modification of the T cell such that CD2 and TCRβ is not expressed (e.g., by deletion or disruption of the gene encoding CD2 and/or TCRβ. In further embodiments, the T cell comprises a suicide gene. In non-limiting examples, a protein-coding sequence of a modified Human-Herpes Simplex Virus-1-thymidine kinase (TK) gene is fused in-frame to the extracellular and transmembrane domains of the human CD34 cDNA and is expressed in the CD2*TCRβ-tCARTΔCD2ΔTCRβ cells.

In another embodiment, an engineered T cell comprises a tandem Chimeric Antigen Receptor (tCAR), wherein one extracellular ligand-binding domain specifically binds CD4 and the second extracellular ligand-binding domain binds the TCRβ chain, wherein the T cell is deficient in CD4 and TCRβ chain, e.g., a CD4*TCRβ-tCARTΔCD4ΔTCRβ cell. In non-limiting examples the deficiency in CD4 and the TCRβ chain resulted from (a) modification of CD4 and TCRβ expressed by the T cell such that the tCAR no longer specifically binds the modified CD4 and TCRβ, (b) modification of the T cell such that expression of the CD4 and TCRβ is reduced in the T cell by at least 50%, at least 60%, at least 70%, at least 80%, at least 90% or more, or (c) modification of the T cell such that CD4 and TCRβ is not expressed (e.g., by deletion or disruption of the gene encoding CD4 and/or TCRβ. In further embodiments, the T cell comprises a suicide gene. In non-limiting examples, a protein-coding sequence of a modified Human-Herpes Simplex Virus-1-thymidine kinase (TK) gene is fused in-frame to the extracellular and transmembrane domains of the human CD34 cDNA and is expressed in the CD4*TCRβ-tCARTΔCD4ΔTCRβ cells.

In another embodiment, an engineered T cell comprises a tandem Chimeric Antigen Receptor (tCAR), wherein one extracellular ligand-binding domain specifically binds CD7 and the second extracellular ligand-binding domain binds CD2, wherein the T cell is deficient in CD7 and CD2, e.g., CD7*CD2-tCARTΔCD7ΔCD2 cell. In non-limiting examples the deficiency in CD7 and CD2 resulted from (a) modification of CD7 and CD2 expressed by the T cell such that the tCAR no longer specifically binds the modified CD7 and CD2, (b) modification of the T cell such that expression of the CD7 and CD2 is reduced in the T cell by at least 50%, at least 60%, at least 70%, at least 80%, at least 90% or more, or (c) modification of the T cell such that CD7 and CD2 is not expressed (e.g., by deletion or disruption of the gene encoding CD7 and/or CD2. In further embodiments, the T cell comprises a suicide gene. In non-limiting examples, a protein-coding sequence of a modified Human-Herpes Simplex Virus-1-thymidine kinase (TK) gene is fused in-frame to the extracellular and transmembrane domains of the human CD34 cDNA and is expressed in the CD7*CD2-tCARTΔCD7ΔCD2 cells.

In another embodiment, an engineered T cell comprises a tandem Chimeric Antigen Receptor (tCAR), wherein one extracellular ligand-binding domain specifically binds CD7 and the second extracellular ligand-binding domain binds CD5, wherein the T cell is deficient in CD7 and CD5, e.g., CD7*CD5-tCARTΔCD7ΔCD5 cell. In non-limiting examples the deficiency in CD7 and CD5 resulted from (a) modification of CD7 and CD5 expressed by the T cell such that the tCAR no longer specifically binds the modified CD7 and CD5, (b) modification of the T cell such that expression of the CD7 and CD5 is reduced in the T cell by at least 50%, at least 60%, at least 70%, at least 80%, at least 90% or more, or (c) modification of the T cell such that CD7 and CD5 is not expressed (e.g., by deletion or disruption of the gene encoding CD7 and/or CD5. In further embodiments, the T cell comprises a suicide gene. In non-limiting examples, a protein coding sequence of a modified Human-Herpes Simplex Virus-1-thymidine kinase (TK) gene is fused in-frame to the extracellular and transmembrane domains of the human CD34 cDNA and is expressed in the CD7*CD5-tCARTΔCD7ΔCD5 cells.

In another embodiment, an engineered T cell comprises a tandem Chimeric Antigen Receptor (tCAR), wherein one extracellular ligand-binding domain specifically binds CD7 and the second extracellular ligand-binding domain binds CD4, wherein the T cell is deficient in CD7 and CD4, e.g., CD7*CD4-tCARTΔCD7ΔCD4 cell. In non-limiting examples the deficiency in CD7 and CD4 resulted from (a) modification of CD7 and CD4 expressed by the T cell such that the tCAR no longer specifically binds the modified CD7 and CD4, (b) modification of the T cell such that expression of the CD7 and CD4 is reduced in the T cell by at least 50%, at least 60%, at least 70%, at least 80%, at least 90% or more, or (c) modification of the T cell such that CD7 and CD4 is not expressed (e.g., by deletion or disruption of the gene encoding CD7 and/or CD4. In further embodiments, the T cell comprises a suicide gene. In non-limiting examples, a protein-coding sequence of a modified Human-Herpes Simplex Virus-1-thymidine kinase (TK) gene is fused in-frame to the extracellular and transmembrane domains of the human CD34 cDNA and is expressed in the CD7*CD4-tCARTΔCD7ΔCD4 cells.

In another embodiment, an engineered T cell comprises a tandem Chimeric Antigen Receptor (tCAR), wherein one extracellular ligand-binding domain specifically binds CD2 and the second extracellular ligand-binding domain binds CD5, wherein the T cell is deficient in CD2 and CD5, e.g., CD2*CD5-tCARTΔCD2ΔCD5 cell. In non-limiting examples the deficiency in CD2 and CD5 resulted from (a) modification of CD2 and CD5 expressed by the T cell such that the tCAR no longer specifically binds the modified CD2 and CD5, (b) modification of the T cell such that expression of the CD2 and CD5 is reduced in the T cell by at least 50%, at least 60%, at least 70%, at least 80%, at least 90% or more, or (c) modification of the T cell such that CD2 and CD5 is not expressed (e.g., by deletion or disruption of the gene encoding CD2 and/or CD5. In further embodiments, the T cell comprises a suicide gene. In non-limiting examples, a protein-coding sequence of a modified Human-Herpes Simplex Virus-1-thymidine kinase (TK) gene is fused in-frame to the extracellular and transmembrane domains of the human CD34 cDNA and is expressed in the CD2*CD5-tCARTΔCD2ΔCD5 cells.

In another embodiment, an engineered T cell comprises a tandem Chimeric Antigen Receptor (tCAR), wherein one extracellular ligand-binding domain specifically binds CD2 and the second extracellular ligand-binding domain binds CD4, wherein the T cell is deficient in CD2 and CD4, e.g., CD2*CD4-tCARTΔCD2ΔCD4 cell. In non-limiting examples the deficiency in CD2 and CD4 resulted from (a) modification of CD2 and CD4 expressed by the T cell such that the tCAR no longer specifically binds the modified CD2 and CD4, (b) modification of the T cell such that expression of the CD2 and CD4 is reduced in the T cell by at least 50%, at least 60%, at least 70%, at least 80%, at least 90% or more, or (c) modification of the T cell such that CD2 and CD4 is not expressed (e.g., by deletion or disruption of the gene encoding CD2 and/or CD4. In further embodiments, the T cell comprises a suicide gene. In non-limiting examples, a protein-coding sequence of a modified Human-Herpes Simplex Virus-1-thymidine kinase (TK) gene is fused in-frame to the extracellular and transmembrane domains of the human CD34 cDNA and is expressed in the CD2*CD4-tCARTΔCD2ΔCD4 cells.

In another embodiment, an engineered T cell comprises a tandem Chimeric Antigen Receptor (tCAR), wherein one extracellular ligand-binding domain specifically binds CD5 and the second extracellular ligand-binding domain binds CD4, wherein the T cell is deficient in CD5 and CD4, e.g., CD5*CD4-tCARTΔCD5ΔCD4 cell. In non-limiting examples the deficiency in CD5 and CD4 resulted from (a) modification of CD5 and CD4 expressed by the T cell such that the chimeric antigen receptor no longer specifically binds the modified CD5 and CD4, (b) modification of the T cell such that expression of the CD5 and CD4 is reduced in the T cell by at least 50%, at least 60%, at least 70%, at least 80%, at least 90% or more, or (c) modification of the T cell such that CD5 and CD4 is not expressed (e.g., by deletion or disruption of the gene encoding CD5 and/or CD4. In further embodiments, the T cell comprises a suicide gene. In non-limiting examples, a protein-coding sequence of a modified Human-Herpes Simplex Virus-1-thymidine kinase (TK) gene fused in-frame to the extracellular and transmembrane domains of the human CD34 cDNA and is expressed in the CD5*CD4-tCARTΔCD5ΔCD4 cells.

In another embodiment, a linear tandem CAR-T cell comprises a chimeric antigen receptor (CAR) polypeptide comprising a first signal peptide, a first extracellular ligand-binding domain, a second extracellular ligand-binding domain, a hinge region, a transmembrane domain, one or more co-stimulatory domains, and a signaling transducing domain, wherein the first extracellular ligand-binding antigen recognition domain and the second extracellular ligand-binding antigen recognition domain have affinities for different cell surface molecules, i.e., antigens on a cancer cell, for example, a malignant T cell, malignant B cell, or malignant plasma cell; and wherein the linear tandem CAR-T cell possesses one or more genetic modifications, deletions, or disruptions resulting in reduced expression of the cell surface molecules in the linear tandem CAR-T cell.

In another embodiment, the signal peptide is the signal peptide from human CD8c (SEQ ID NO:1).

In a third embodiment, the first extracellular ligand-binding domain comprises a single chain antibody fragment (scFv), comprising the light (V_{L}) and the heavy (V_{H}) variable fragment, designated V_{H}1 and V_{L}1 and joined by a linker (e.g., GGGGS). In some embodiments, this linker peptide is repeated 2, 3, 4, 5 or 6 times. In some embodiments, the first antigen recognition domain can be selected from: 1) V_{H}1-(GGGGS)₃ (SEQ ID NO:449)-V_{L}1 or 2) V_{L}1-(GGGGS)₃ (SEQ ID NO:449)-V_{H}1.

In some embodiments, the second extracellular ligand-binding domain comprises a single chain antibody fragment (scFv), comprising the light (V_{L}) and the heavy (V_{H}) variable fragment, designated V_{H}2 and V_{L}2 and joined by a linker (e.g., GGGGS). In some embodiments, this linker peptide is repeated 2, 3, 4, 5 or 6 times. In some embodiments, the first antigen recognition domain can be selected from: 1) V_{H}2-(GGGGS)₃₋₄ (SEQ ID NO:449)-V_{L}2 or 2) V_{L}2-(GGGGS)₃₋₄ (SEQ ID NO:449)-V_{H}2.

In further embodiments, the first antigen recognition domain and second antigen recognition domain are connected by a short linker peptide of 5 amino acids (GGGGS). In some embodiments, this linker peptide is repeated 2, 3, 4, 5 or 6 times.

### Linear Tandem CAR Constructs

In one embodiment of a linear tandem CAR construct, the first extracellular ligand-binding domain comprises a single chain antibody fragment (scFv), comprising the heavy (V_{H}) and the light (V_{L}) variable fragment, designated V_{H}1 and V_{L}1, and joined by a linker (e.g., GGGGS)₂₋₆ (SEQ ID NO:447). The second extracellular ligand-binding domain antigen recognition comprises a single chain antibody fragment (scFv), comprising the light (V_{L}) and the heavy (V_{H}) variable fragment, designated V_{L}2 and V_{H}2, and joined by a linker (e.g., GGGGS)₂₋₆ (SEQ ID NO:447).

In a second embodiment of a linear tandem CAR construct, the first extracellular ligand-binding domain comprises a single chain antibody fragment (scFv), comprising the heavy (V_{H}) and the light (V_{L}) variable fragment, designated V_{H}2 and V_{L}2, and joined by a linker (e.g., GGGGS)₂₋₆ (SEQ ID NO:447). The second extracellular ligand-binding domain antigen recognition comprises a single chain antibody fragment (scFv), comprising the light (V_{L}) and the heavy (V_{H}) variable fragment, designated V_{L}1 and V_{H}1, and joined by a linker (e.g., GGGGS)₂₋₆ (SEQ ID NO:447).

In a third embodiment of a linear tandem CAR construct, the first extracellular ligand-binding domain comprises a single chain antibody fragment (scFv), comprising the heavy (V_{L}) and the light (V_{H}) variable fragment, designated V_{L}1 and V_{H}1, and joined by a linker (e.g., GGGGS)₂₋₆ (SEQ ID NO:447). The second extracellular ligand-binding domain antigen recognition comprises a single chain antibody fragment (scFv), comprising the light (V_{H}) and the heavy (V_{L}) variable fragment, designated V_{H}2 and V_{L}2, and joined by a linker (e.g., GGGGS)₂₋₆ (SEQ ID NO:447).

In a fourth embodiment of a linear tandem CAR construct, the first extracellular ligand-binding domain comprises a single chain antibody fragment (scFv), comprising the heavy (V_{L}) and the light (V_{H}) variable fragment, designated V_{L}2 and V_{H}2, and joined by a linker (e.g., GGGGS)₂₋₆ (SEQ ID NO:447). The second extracellular ligand-binding domain antigen recognition comprises a single chain antibody fragment (scFv), comprising the light (V_{H}) and the heavy (V_{L}) variable fragment, designated V_{H}1 and V_{L}1, and joined by a linker (e.g., GGGGS)₂₋₆ (SEQ ID NO:447).

For each of the linear tandem CAR construct embodiments, the first and second extracellular ligand-binding domains targets a surface molecule, i.e., an antigen expressed on a malignant T cell is selected from, but not limited to, BCMA, CS1, CD38, CD138, CD19, CD33, CD123, CD371, CD117, CD135, Tim-3, CD5, CD7, CD2, CD4, CD3, CD79A, CD79B, APRIL, CD56, and CD1a.

Further examples of linear tandem CARs are given below in Table 6.

TABLE 6
Tandem CARs and CAR-Ts (Linear or Hairpin).

| | Antigen Target | Antigen Deletion/ |
|---|---|---|
| Example | CAR-T cell | Suppression |
| T1 | APRIL × BCMA | — |
| T2 | APRIL × CD19 | — |
| T3 | APRIL × CD38 | — |
| T4 | APRIL × CD38 | CD38 |
| T5 | APRIL × CS1 | — |
| T6 | APRIL × CS1 | CS1 |
| T7 | BCMA × CD19 | — |
| T8 | BCMA × CD38 | — |
| T9 | BCMA × CD38 | CD38 |
| T10 | BCMA × CS1 | — |
| T11 | BCMA × CS1 | CS1 |
| T12 | CD138 × APRIL | |
| T13 | CD138 × BCMA | |
| T14 | CD138 × CD19 | |
| T15 | CD138 × CD38 | |
| T16 | CD138 × CD38 | CD38 |
| T17 | CD138 × CD79A | |
| T18 | CD138 × CD79B | |
| T19 | CD138 × CS1 | |
| T20 | CD138 × CS1 | CS1 |
| T21 | CD19 × CD38 | — |
| T22 | CD19 × CD38 | CD38 |
| T23 | CD2 × CD3ε | — |
| T24 | CD2 × CD3ε | CD2 |
| T25 | CD2 × CD3ε | CD3ε |
| T26 | CD2 × CD3ε | CD2 and CD3ε |
| T27 | CD2 × CD4 | — |
| T28 | CD2 × CD4 | CD2 |
| T29 | CD2 × CD4 | CD4 |
| T30 | CD2 × CD4 | CD2 and CD4 |
| T31 | CD2 × CD4 | CD2 and TRAC |
| T32 | CD2 × CD4 | CD4 and TRAC |
| T33 | CD2 × CD4 | CD2 and CD4 and TRAC |
| T34 | CD2 × CD5 | — |
| T35 | CD2 × CD5 | CD2 |
| T36 | CD2 × CD5 | CD5 |
| T37 | CD2 × CD5 | CD2 and CD5 |
| T38 | CD2 × CD5 | CD2 and TRAC |
| T39 | CD2 × CD5 | CD5 and TRAC |
| T40 | CD2 × CD5 | CD2 and CD5 and TRAC |
| T41 | CD2 × CD7 | — |
| T42 | CD2 × CD7 | CD2 |
| T43 | CD2 × CD7 | CD7 |
| T44 | CD2 × CD7 | CD2 and CD7 |
| T45 | CD2 × CD7 | CD2 and TRAC |
| T46 | CD2 × CD7 | CD7 and TRAC |
| T47 | CD2 × CD7 | CD2 and CD7 and TRAC |
| T48 | CD3ε × CD4 | — |
| T49 | CD3ε × CD4 | CD3ε |
| T50 | CD3ε × CD4 | CD4 |
| T51 | CD3ε × CD4 | CD3ε and CD4 |
| T52 | CD3ε × CD5 | — |
| T53 | CD3ε × CD5 | CD3ε |
| T54 | CD3ε × CD5 | CD5 |
| T55 | CD3ε × CD5 | CD3ε and CD5 |
| T56 | CD3ε × CD7 | — |
| T57 | CD3ε × CD7 | CD3ε |
| T58 | CD3ε × CD7 | CD7 |
| T59 | CD3ε × CD7 | CD3ε and CD7 |
| T60 | CD4 × CD5 | — |
| T61 | CD4 × CD5 | CD4 |
| T62 | CD4 × CD5 | CD5 |
| T63 | CD4 × CD5 | CD4 and CD5 |
| T64 | CD4 × CD5 | CD4 and TRAC |
| T65 | CD4 × CD5 | CD5 and TRAC |
| T66 | CD4 × CD5 | CD4 and CD5 and TRAC |
| T67 | CD4 × CD7 | — |
| T68 | CD4 × CD7 | CD4 |
| T69 | CD4 × CD7 | CD7 |
| T70 | CD4 × CD7 | CD4 and CD7 |
| T71 | CD4 × CD7 | CD4 and TRAC |
| T72 | CD4 × CD7 | CD4 and TRAC |
| T73 | CD4 × CD7 | CD4 and CD7 and TRAC |
| T74 | CD5 × CD7 | — |
| T75 | CD5 × CD7 | CD5 |
| T76 | CD5 × CD7 | CD7 |
| T77 | CD5 × CD7 | CD5 and CD7 |
| T78 | CD5 × CD7 | CD5 and TRAC |
| T79 | CD5 × CD7 | CD7 and TRAC |
| T80 | CD5 × CD7 | CD5 and CD7 and TRAC |
| T81 | CD79A × APRIL | |
| T82 | CD79A × BCMA | |
| T83 | CD79A × CD19 | |
| T84 | CD79A × CD38 | |
| T85 | CD79A × CD38 | CD38 |
| T86 | CD79A × CD79B | |
| T87 | CD79A × CS1 | |
| T88 | CD79A × CS1 | CS1 |
| T89 | CD79B × APRIL | |
| T90 | CD79B × BCMA | |
| T91 | CD79B × CD19 | |
| T92 | CD79B × CD38 | |
| T93 | CD79B × CD38 | CD38 |
| T94 | CD79B × CD79A | |
| T95 | CD79B × CS1 | |
| T96 | CD79B × CS1 | CS1 |
| T97 | CS1 × CD19 | — |
| T98 | CS1 × CD19 | CS1 |
| T99 | CS1 × CD38 | — |
| T100 | CS1 × CD38 | CS1 |
| T101 | CS1 × CD38 | CD38 |
| T102 | CS1 × CD38 | CS1 and CD38 |
| T103 | TCRβ × CD2 | — |
| T104 | TCRβ × CD2 | TCRβ |
| T105 | TCRβ × CD2 | CD2 |
| T106 | TCRβ × CD2 | TCRβ and CD2 |
| T107 | TCRβ × CD3ε | — |
| T108 | TCRβ × CD3ε | TCRβ |
| T109 | TCRβ × CD3ε | CD3ε |
| T110 | TCRβ × CD3ε | TCRβ and CD3ε |
| T111 | TCRβ × CD4 | — |
| T112 | TCRβ × CD4 | TCRβ |
| T113 | TCRβ × CD4 | CD4 |
| T114 | TCRβ × CD4 | TCRβ and CD4 |
| T115 | TCRβ × CD5 | — |
| T116 | TCRβ × CD5 | TCRβ |
| T117 | TCRβ × CD5 | CD5 |
| T118 | TCRβ × CD5 | TCRβ and CD5 |
| T119 | TCRβ × CD7 | — |
| T120 | TCRβ × CD7 | TCRβ |
| T121 | TCRβ × CD7 | CD7 |
| T122 | TCRβ × CD7 | TCRβ and CD7 |
| T123 | TRAC × CD2 | — |
| T124 | TRAC × CD2 | TRAC |
| T125 | TRAC × CD2 | CD2 |
| T126 | TRAC × CD2 | TRAC and CD2 |
| T127 | TRAC × CD3ε | — |
| T128 | TRAC × CD3ε | TRAC |
| T129 | TRAC × CD3ε | CD3ε |
| T130 | TRAC × CD3ε | TRAC and CD3ε |
| T131 | TRAC × CD4 | — |
| T132 | TRAC × CD4 | TRAC |
| T133 | TRAC × CD4 | CD4 |
| T134 | TRAC × CD4 | TRAC and CD4 |
| T135 | TRAC × CD5 | — |
| T136 | TRAC × CD5 | TRAC |
| T137 | TRAC × CD5 | CD5 |
| T138 | TRAC × CD5 | TRAC and CD5 |
| T139 | TRAC × CD7 | — |
| T140 | TRAC × CD7 | TRAC |
| T141 | TRAC × CD7 | CD7 |
| T142 | TRAC × CD7 | TRAC and CD7 |

For example, provided herein are linear tandem CAR constructs which may incorporate the V_{H} and V_{L} domains of scFvs targeting any of the antigen pairs provided in Table 6 above.

TABLE 7
Linear Tandem CAR Constructs.

| 7-I | 7-II | 7-III | 7-IV | 7-V | 7-VI | 7-VII | 7-VIII |
|---|---|---|---|---|---|---|---|
| CD8a | CD8a | CD8a | CD8a | CD8a | CD8a | CD8a | CD8a |
| V_{H}1 | V_{H}1 | V_{H}1 | V_{H}1 | V_{H}1 | V_{H}1 | V_{H}1 | V_{H}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{L}1 | V_{L}1 | V_{L}1 | V_{L}1 | V_{L}1 | V_{L}1 | V_{L}1 | V_{L}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{L}2 | V_{L}2 | V_{L}2 | V_{L}2 | V_{L}2 | V_{L}2 | V_{L}2 | V_{L}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ ID | (SEQ ID | (SEQ ID | (SEQ ID | (SEQ ID | (SEQ ID | (SEQ ID | (SEQ ID |
| NO: 449) | NO: 449) | NO: 449) | NO: 449) | NO: 449) | NO: 449) | NO: 449) | NO: 449) |
| V_{H}2 | V_{H}2 | V_{H}2 | V_{H}2 | V_{H}2 | V_{H}2 | V_{H}2 | V_{H}2 |
| CD8 Tm | CD8 Tm | CD8 Tm | CD8 Tm | CD28 Tm | CD28 Tm | CD28 Tm | CD28 Tm |
| 41BB | CD28 | 41BB-CD28 | CD28-41BB | 41BB | CD28 | 41BB-CD28 | CD28-41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |
| 7-IX | 7-X | 7-XI | 7-XII | 7-XIII | 7-XIV | 7-XV | 7-XVI |
| CD8a | CD8a | CD8a | CD8a | CD8a | CD8a | CD8a | CD8a |
| V_{H}2 | V_{H}2 | V_{H}2 | V_{H}2 | V_{H}2 | V_{H}2 | V_{H}2 | V_{H}2 |
| GGGGS₍₂₋₆₎ | GGGGS₍₂₋₆₎ | GGGGSS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{L}2 | V_{L}2 | V_{L}2 | V_{L}2 | V_{L}2 | V_{L}2 | V_{L}2 | V_{L}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| V_{L}1 | V_{L}1 | V_{L}1 | V_{L}1 | V_{L}1 | V_{L}1 | V_{L}1 | V_{L}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ ID | (SEQ ID | (SEQ ID | (SEQ ID | (SEQ ID | (SEQ ID | (SEQ ID | (SEQ ID |
| NO: 449) | NO: 449) | NO: 449) | NO: 449) | NO: 449) | NO: 449) | NO: 449) | NO: 449) |
| V_{H}1 | V_{H}1 | V_{H}1 | V_{H}1 | V_{H}1 | V_{H}1 | V_{H}1 | V_{H}1 |
| CD8 Tm | CD8 Tm | CD8 Tm | CD8 Tm | CD28 Tm | CD28 Tm | CD28 Tm | CD28 Tm |
| 41BB | CD28 | 41BB-CD28 | CD28-41BB | 41BB | CD28 | 41BB-CD28 | CD28-41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |
| 7-XVII | 7-XVIII | 7-XIX | 7-XX | 7-XXI | 7-XXII | 7-XXIII | 7-XIV |
| CD8a | CD8a | CD8a | CD8a | CD8a | CD8a | CD8a | CD8a |
| V_{L}1 | V_{L}1 | V_{L}1 | V_{L}1 | V_{L}1 | V_{L}1 | V_{L}1 | V_{L}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{H}1 | V_{H}1 | V_{H}1 | V_{H}1 | V_{H}1 | V_{H}1 | V_{H}1 | V_{H}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{H}2 | V_{H}2 | V_{H}2 | V_{H}2 | V_{H}2 | V_{H}2 | V_{H}2 | V_{H}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ ID | (SEQ ID | (SEQ ID | (SEQ ID | (SEQ ID | (SEQ ID | (SEQ ID | (SEQ ID |
| NO: 449) | NO: 449) | NO: 449) | NO: 449) | NO: 449) | NO: 449) | NO: 449) | NO: 449) |
| V_{L}2 | V_{L}2 | V_{L}2 | V_{L}2 | V_{L}2 | V_{L}2 | V_{L}2 | V_{L}2 |
| CD8 Tm | CD8 Tm | CD8 Tm | CD8 Tm | CD28 Tm | CD28 Tm | CD28 Tm | CD28 Tm |
| 41BB | CD28 | 41BB-CD28 | CD28-41BB | 41BB | CD28 | 41BB-CD28 | CD28-41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |
| 7-XXV | 7-XXVI | 7-XXVII | 7-XXVIII | 7-XIX | 7-XXX | 7-XXXI | 7-XXXII |
| CD8a | CD8a | CD8a | CD8a | CD8a | CD8a | CD8a | CD8a |
| V_{L}2 | V_{L}2 | V_{L}2 | V_{L}2 | V_{L}2 | V_{L}2 | V_{L}2 | V_{L}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{H}2 | V_{H}2 | V_{H}2 | V_{H}2 | V_{H}2 | V_{H}2 | V_{H}2 | V_{H}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{H}1 | V_{H}1 | V_{H}1 | V_{H}1 | V_{H}1 | V_{H}1 | V_{H}1 | V_{H}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ ID | (SEQ ID | (SEQ ID | (SEQ ID | (SEQ ID | (SEQ ID | (SEQ ID | (SEQ ID |
| NO: 449) | NO: 449) | NO: 449) | NO: 449) | NO: 449) | NO: 449) | NO: 449) | NO: 449) |
| V_{L}1 | V_{L}1 | V_{L}1 | V_{L}1 | V_{L}1 | V_{L}1 | V_{L}1 | V_{L}1 |
| CD8 Tm | CD8 Tm | CD8 Tm | CD8 Tm | CD28 Tm | CD28 Tm | CD28 Tm | CD28 Tm |
| 41BB | CD28 | 41BB-CD28 | CD28-41BB | 41BB | CD28 | 41BB-CD28 | CD28-41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |

### Hairpin Tandem CAR Constructs

In one embodiment of a hairpin tandem CAR construct, the first extracellular ligand-binding domain comprises a single chain antibody fragment (scFv), comprising two heavy chain variable fragments, designated V_{H}1 and V_{H}2, and joined by a linker (e.g., GGGGS)₂₋₆ (SEQ ID NO:447). The second extracellular ligand-binding domain antigen recognition comprises a single chain antibody fragment (scFv), comprising two light chain variable fragments, designated V_{L}2 and V_{L}1, and joined by a linker (e.g., GGGGS)₂₋₆ (SEQ ID NO:447).

In a second embodiment of a hairpin tandem CAR construct, the first extracellular ligand-binding domain comprises a single chain antibody fragment (scFv), comprising two heavy chain variable fragments, designated V_{H}2 and V_{H}1, and joined by a linker (e.g., GGGGS)₂₋₆ (SEQ ID NO:447). The second extracellular ligand-binding domain antigen recognition comprises a single chain antibody fragment (scFv), comprising two light chain variable fragments, designated V_{L}1 and V_{L}2, and joined by a linker (e.g., GGGGS)₂₋₆ (SEQ ID NO:447).

In a third embodiment of a hairpin tandem CAR construct, the first extracellular ligand-binding domain comprises a single chain antibody fragment (scFv), comprising two light chain variable fragments, designated V_{L}1 and V_{L}2, and joined by a linker (e.g., GGGGS)₂₋₆ (SEQ ID NO:447). The second extracellular ligand-binding domain antigen recognition comprises a single chain antibody fragment (scFv), comprising two heavy chain variable fragments, designated V_{H}2 and V_{H}1, and joined by a linker (e.g., GGGGS)₂₋₆ (SEQ ID NO:447).

In a fourth embodiment of a hairpin tandem CAR construct, the first extracellular ligand-binding domain comprises a single chain antibody fragment (scFv), comprising two light chain variable fragments, designated V_{L}2 and V_{L}1, and joined by a linker (e.g., GGGGS)₂₋₆ (SEQ ID NO:447). The second extracellular ligand-binding domain antigen recognition comprises a single chain antibody fragment (scFv), comprising two heavy chain variable fragments, designated V_{H}1 and V_{H}2, and joined by a linker (e.g., GGGGS)₂₋₆ (SEQ ID NO:447).

For each of the hairpin tandem CAR construct embodiments, the first and second extracellular ligand-binding domains targets a surface molecule, i.e., an antigen expressed on a malignant T cell is selected from, but not limited to, BCMA, CS1, CD38, CD138, CD19, CD33, CD123, CD371, CD117, CD135, Tim-3, CD5, CD7, CD2, CD4, CD3, CD79A, CD79B, APRIL, CD56, and CD1a.

Additional examples of hairpin tandem CARs are given above in Table 6.

Furthermore, provided herein are CAR constructs and CAR-T cells which may incorporate the V_{H} and V_{L} domains of scFvs targeting (1) CD2 and CD3; and (2) CD2 and CD7 and are provided below in Table 8.

TABLE 8
Amino Acid Sequences of Hairpin Tandem Chimeric Antigen
Receptors (CARs).

| Hairpin | | | |
|---|---|---|---|
| Tandem CAR | Designation | SEQ ID | |
| Constructs | in Examples | NO: | Amino acid sequence |
| OKT3 V_{L}- | WC5 | SEQ ID | MALPVTALLLPLALLLHAARPQIVLTQSPAIM |
| CD2 V_{L}- | | NO: 41 | SASPGEKVTMTCSASSSVSYMNWYQQKSGTSP |
| CD2 V_{H}- | | | KRWIYDTSKLASGVPAHFRGSGSGTSYSLTIS |
| OKT3 V_{H} | | | GMEAEDAATYYCQQWSSNPFTFGSGTKLEINR |
| | | | GGGGSGGGGSGGGGSGGGGSDIKNITQSPSSM |
| | | | YVSLGERVTITCKASQDINSFLSWFQQKPGKS |
| | | | PKTLIYRANRLVDGVPSRFSGSGSGQDYSLTI |
| | | | SSLEYEDMEIYYCLQYDEFPYTFGGGTKLEMK |
| | | | RGGGGSGGGGSGGGGSGGGGSGGGGSGGGGSG |
| | | | GGGSGGGGSGGGGSGGGGSEVKLEESGAELVK |
| | | | PGASVKLSCRTSGFNIKDTIHWVKQRPEQGLK |
| | | | WIGRIDPANGNTKYDPKFQDKATVTADTSSNT |
| | | | AYLQLSSLTSEDTAVYYCVTYAYDGNWYFDVW |
| | | | GAGTAVTVSSGGGGSGGGGSGGGGSGGGGSGS |
| | | | QVQLQQSGAELARPGASVKMSCKASGYTFTRY |
| | | | TMHWVKQRPGQGLEWIGYINPSRGYTNYNQKF |
| | | | KDKATLTTDKSSSTAYMQLSSLTSEDSAVYYC |
| | | | ARYYDDHYCLDYWGQGTTLTVSSPRASTTTPA |
| | | | PRPPTPAPTIASQPLSLRPEACRPAAGGAVHT |
| | | | RGLDFACDFWVLVVVGGVLACYSLLVTVAFII |
| | | | FWVRSKRSRLLHSDYMNMTPRRPGPTRKHYQP |
| | | | YAPPRDFAAYRSRVKFSRSADAPAYKQGQNQL |
| | | | YNELNLGRREEYDVLDKRRGRDPEMGGKPRRK |
| | | | NPQEGLYNELQKDKMAEAYSEIGMKGERRRGK |
| | | | GHDGLYQGLSTATKDTYDALHMQALPPRRTDG |
| | | | SGATNFSLLKQAGDVEENPGPVSEAMPRGWTA |
| | | | LCLLSLLPSGFMSLDNNGTATPELPTQGTFSN |
| | | | VSTNVSYQETTTPSTLGSTSLHPVSQHGNEAT |
| | | | TNITETTVKFTSTSVITSVYGNTNSSVQSQTS |
| | | | VISTVFTTPANVSTPETTLKPSLSPGNVSDLS |
| | | | TTSTSLATSPTKPYTSSSPILSDIKAEIKCSG |
| | | | IREVKLTQGICLEQNKTSSCAEFKKDRGEGLA |
| | | | RVLCGEEQADADAGAQVCSLLLAQSEVRPQCL |
| | | | LLVLANRTEISSKLQLMKKHQSDLKKLGILDF |
| | | | TEQDVASHQSYSQKTLIALVTSGALLAVLGIT |
| | | | GYFLMNRRSWSPI |
| CD3 V_{L}- | WC7 | SEQ ID | MALPVTALLLPLALLLHAARPDIQMTQSPSSL |
| CD2 V_{L}- | | NO: 42 | SASVGDRVTITCRASQDIRNYLNWYQQKPGKA |
| CD2-V_{H}- | | | PKLLIYYTSRLESGVPSRFSGSGSGTDYTLTI |
| CD3 V_{H} | | | SSLQPEDFATYYCQQGNTLPWTFGCGTKVEIK |
| | | | GGGGSGGGGSGGGGSGGGGSDIKNITQSPSSM |
| | | | YVSLGERVTITCKASQDINSFLSWFQQKPGKS |
| | | | PKTLIYRANRLVDGVPSRFSGSGSGQDYSLTI |
| | | | SSLEYEDMEIYYCLQYDEFPYTFGGGTKLEMK |
| | | | RGGGGSGGGGSGGGGSGGGGSGGGGSGGGGSG |
| | | | GGGSGGGGSGGGGSGGGGSEVKLEESGAELVK |
| | | | PGASVKLSCRTSGFNIKDTIHWVKQRPEQGLK |
| | | | WIGRIDPANGNTKYDPKFQDKATVTADTSSNT |
| | | | AYLQLSSLTSEDTAVYYCVTYAYDGNWYFDVW |
| | | | GAGTAVTVSSGGGGSGGGGSGGGGSGGGGSEV |
| | | | QLVESGGGLVQPGGSLRLSCAASGYSFTGYTM |
| | | | NWVRQAPGKCLEWVALINPYKGVSTYNQKFKD |
| | | | RFTISVDKSKNTAYLQMNSLRAEDTAVYYCAR |
| | | | SGYYGDSDWYFDVWGQGTLVTVSSPRASTTTP |
| | | | APRPPTPAPTIASQPLSLRPEACRPAAGGAVH |
| | | | TRGLDFACDFWVLVVVGGVLACYSLLVTVAFI |
| | | | IFWVRSKRSRLLHSDYMNMTPRRPGPTRKHYQ |
| | | | PYAPPRDFAAYRSRVKFSRSADAPAYKQGQNQ |
| | | | LYNELNLGRREEYDVLDKRRGRDPEMGGKPRR |
| | | | KNPQEGLYNELQKDKMAEAYSEIGMKGERRRG |
| | | | KGHDGLYQGLSTATKDTYDALHMQALPPRRTD |
| | | | GSGATNFSLLKQAGDVEENPGPVSEAMPRGWT |
| | | | ALCLLSLLPSGFMSLDNNGTATPELPTQGTFS |
| | | | NVSTNVSYQETTTPSTLGSTSLHPVSQHGNEA |
| | | | TTNITETTVKFTSTSVITSVYGNTNSSVQSQT |
| | | | SVISTVFTTPANVSTPETTLKPSLSPGNVSDL |
| | | | STTSTSLATSPTKPYTSSSPILSDIKAEIKCS |
| | | | GIREVKLTQGICLEQNKTSSCAEFKKDRGEGL |
| | | | ARVLCGEEQADADAGAQVCSLLLAQSEVRPQC |
| | | | LLLVLANRTEISSKLQLMKKHQSDLKKLGILD |
| | | | FTEQDVASHQSYSQKTLIALVTSGALLAVLGI |
| | | | TGYFLMNRRSWSPI |
| CD2 V_{L}- | WC15 | SEQ ID | MALPVTALLLPLALLLHAARPDIKNITQSPSS |
| CD3 V_{L}- | | NO: 43 | MYVSLGERVTITCKASQDINSFLSWFQQKPGK |
| CD3 V_{H}- | | | SPKTLIYRANRLVDGVPSRFSGSGSGQDYSLT |
| CD2-V_{H} | | | ISSLEYEDMEIYYCLQYDEFPYTFGGGTKLEM |
| | | | KRGGGGSGGGGSGGGGSGGGGSDIQMTQSPSS |
| | | | LSASVGDRVTITCRASQDIRNYLNWYQQKPGK |
| | | | APKLLIYYTSRLESGVPSRFSGSGSGTDYTLT |
| | | | ISSLQPEDFATYYCQQGNTLPWTFGCGTKVEI |
| | | | KGGGGSGGGGSGGGGSGGGGSGGGGSGGGGSG |
| | | | GGGSGGGGSGGGGSGGGGSEVQLVESGGGLVQ |
| | | | PGGSLRLSCAASGYSFTGYTMNWVRQAPGKCL |
| | | | EWVALINPYKGVSTYNQKFKDRFTISVDKSKN |
| | | | TAYLQMNSLRAEDTAVYYCARSGYYGDSDWYF |
| | | | DVWGQGTLVTVSSGGGGSGGGGSGGGGSGGGG |
| | | | SEVKLEESGAELVKPGASVKLSCRTSGFNIKD |
| | | | TIHWVKQRPEQGLKWIGRIDPANGNTKYDPKF |
| | | | QDKATVTADTSSNTAYLQLSSLTSEDTAVYYC |
| | | | VTYAYDGNWYFDVWGAGTAVTVSSPRASTTTP |
| | | | APRPPTPAPTIASQPLSLRPEACRPAAGGAVH |
| | | | TRGLDFACDFWVLVVVGGVLACYSLLVTVAFI |
| | | | IFWVRSKRSRLLHSDYMNMTPRRPGPTRKHYQ |
| | | | PYAPPRDFAAYRSRVKFSRSADAPAYKQGQNQ |
| | | | LYNELNLGRREEYDVLDKRRGRDPEMGGKPRR |
| | | | KNPQEGLYNELQKDKMAEAYSEIGMKGERRRG |
| | | | KGHDGLYQGLSTATKDTYDALHMQALPPRRTD |
| | | | GSGATNFSLLKQAGDVEENPGPVSEAMPRGWT |
| | | | ALCLLSLLPSGFMSLDNNGTATPELPTQGTFS |
| | | | NVSTNVSYQETTTPSTLGSTSLHPVSQHGNEA |
| | | | TTNITETTVKFTSTSVITSVYGNTNSSVQSQT |
| | | | SVISTVFTTPANVSTPETTLKPSLSPGNVSDL |
| | | | STTSTSLATSPTKPYTSSSPILSDIKAEIKCS |
| | | | GIREVKLTQGICLEQNKTSSCAEFKKDRGEGL |
| | | | ARVLCGEEQADADAGAQVCSLLLAQSEVRPQC |
| | | | LLLVLANRTEISSKLQLMKKHQSDLKKLGILD |
| | | | FTEQDVASHQSYSQKTLIALVTSGALLAVLGI |
| | | | TGYFLMNRRSWSPI |
| CD2 V_{L}- | WC13 | SEQ ID | MALPVTALLLPLALLLHAARPDIKNITQSPSS |
| OKT3 V_{L}- | | NO: 44 | MYVSLGERVTITCKASQDINSFLSWFQQKPGK |
| OKT3 V_{H}- | | | SPKTLIYRANRLVDGVPSRFSGSGSGQDYSLT |
| CD2 V_{H} | | | ISSLEYEDMEIYYCLQYDEFPYTFGGGTKLEM |
| | | | KRGGGGSGGGGSGGGGSGGGGSQIVLTQSPAI |
| | | | MSASPGEKVTMTCSASSSVSYMNWYQQKSGTS |
| | | | PKRWIYDTSKLASGVPAHFRGSGSGTSYSLTI |
| | | | SGMEAEDAATYYCQQWSSNPFTFGSGTKLEIN |
| | | | RGGGGSGGGGSGGGGSGGGGSGGGGSGGGGSG |
| | | | GGGSGGGGSGGGGSGGGGSGSQVQLQQSGAEL |
| | | | ARPGASVKMSCKASGYTFTRYTMHWVKQRPGQ |
| | | | GLEWIGYINPSRGYTNYNQKFKDKATLTTDKS |
| | | | SSTAYMQLSSLTSEDSAVYYCARYYDDHYCLD |
| | | | YWGQGTTLTVSSGGGGSGGGGSGGGGSGGGGS |
| | | | EVKLEESGAELV |
| | | | KPGASVKLSCRTSGFNIKDTIHWVKQRPEQGL |
| | | | KWIGRIDPANGNTKYDPKFQDKATVTADTSSN |
| | | | TAYLQLSSLTSEDTAVYYCVTYAYDGNWYFDV |
| | | | WGAGTAVTVSSPRASTTTPAPRPPTPAPTIAS |
| | | | QPLSLRPEACRPAAGGAVHTRGLDFACDFWVL |
| | | | VVVGGVLACYSLLVTVAFIIFWVRSKRSRLLH |
| | | | SDYMNMTPRRPGPTRKHYQPYAPPRDFAAYRS |
| | | | RVKFSRSADAPAYKQGQNQLYNELNLGRREEY |
| | | | DVLDKRRGRDPEMGGKPRRKNPQEGLYNELQK |
| | | | DKMAEAYSEIGMKGERRRGKGHDGLYQGLSTA |
| | | | TKDTYDALHMQALPPRRTDGSGATNFSLLKQA |
| | | | GDVEENPGPVSEAMPRGWTALCLLSLLPSGFM |
| | | | SLDNNGTATPELPTQGTFSNVSTNVSYQETTT |
| | | | PSTLGSTSLHPVSQHGNEATTNITETTVKFTS |
| | | | TSVITSVYGNTNSSVQSQTSVISTVFTTPANV |
| | | | STPETTLKPSLSPGNVSDLSTTSTSLATSPTK |
| | | | PYTSSSPILSDIKAEIKCSGIREVKLTQGICL |
| | | | EQNKTSSCAEFKKDRGEGLARVLCGEEQADAD |
| | | | AGAQVCSLLLAQSEVRPQCLLLVLANRTEISS |
| | | | KLQLMKKHQSDLKKLGILDFTEQDVASHQSYS |
| | | | QKTLIALVTSGALLAVLGITGYFLMNRRSWSP |
| | | | I |
| CD7 V_{L}- | | SEQ ID | MALPVTALLLPLALLLHAARPDIQMTQTTSSL |
| CD2 V_{L}- | | NO: 45 | SASLGDRVTISCSASQGISNYLNWYQQKPDGT |
| CD2 V_{H}- | | | VKLLIYYTSSLHSGVPSRFSGSGSGTDYSLTI |
| CD7 V_{H} | | | SNLEPEDIATYYCQQYSKLPYTFGGGTKLEIK |
| | | | RGGGGSGGGGSGGGGSGGGGSDIKNITQSPSS |
| | | | MYVSLGERVTITCKASQDINSFLSWFQQKPGK |
| | | | SPKTLIYRANRLVDGVPSRFSGSGSGQDYSLT |
| | | | ISSLEYEDMEIYYCLQYDEFPYTFGGGTKLEM |
| | | | KRGGGGSGGGGSGGGGSGGGGSGGGGSGGGGS |
| | | | GGGGSGGGGSGGGGSGGGGSEVKLEESGAELV |
| | | | KPGASVKLSCRTSGFNIKDTIHWVKQRPEQGL |
| | | | KWIGRIDPANGNTKYDPKFQDKATVTADTSSN |
| | | | TAYLQLSSLTSEDTAVYYCVTYAYDGNWYFDV |
| | | | WGAGTAVTVSSGGGGSGGGGSGGGGSGGGGSE |
| | | | VQLVESGGGLVKPGGSLKLSCAASGLTFSSYA |
| | | | MSWVRQTPEKRLEWVASISSGGFTYYPDSVKG |
| | | | RFTISRDNARNILYLQMSSLRSEDTAMYYCAR |
| | | | DEVRGYLDVWGAGTTVTVSPRASTTTPAPRPP |
| | | | TPAPTIASQPLSLRPEACRPAAGGAVHTRGLD |
| | | | FACDFWVLVVVGGVLACYSLLVTVAFIIFWVR |
| | | | SKRSRLLHSDYMNMTPRRPGPTRKHYQPYAPP |
| | | | RDFAAYRSRVKFSRSADAPAYKQGQNQLYNEL |
| | | | NLGRREEYDVLDKRRGRDPEMGGKPRRKNPQE |
| | | | GLYNELQKDKMAEAYSEIGMKGERRRGKGHDG |
| | | | LYQGLSTATKDTYDALHMQALPPRRTDGSGAT |
| | | | NFSLLKQAGDVEENPGPVSEAMPRGWTALCLL |
| | | | SLLPSGFMSLDNNGTATPELPTQGTFSNVSTN |
| | | | VSYQETTTPSTLGSTSLHPVSQHGNEATTNIT |
| | | | TSTSVITSVYGNTNSSVQSQTSVISTVFTTPA |
| | | | NVSTPETTLKPSLSPGNVSDLSTTSTSLATSP |
| | | | TKPYTSSSPILSDIKAEIKCSGIREVKLTQGI |
| | | | CLEQNKTSSCAEFKKDRGEGLARVLCGEEQAD |
| | | | ADAGAQVCSLLLAQSEVRPQCLLLVLANRTEI |
| | | | SSKLQLMKKHQSDLKKLGILDFTEQDVASHQS |
| | | | YSQKTLIALVTSGALLAVLGITGYFLMNRRSW |
| | | | SPI |
| CD2 V_{L}- | | SEQ ID | MALPVTALLLPLALLLHAARPDIKNITQSPSS |
| CD7 V_{L}- | | NO: 46 | MYVSLGERVTITCKASQDINSFLSWFQQKPGK |
| CD7 V_{H}- | | | SPKTLIYRANRLVDGVPSRFSGSGSGQDYSLT |
| CD2 V_{H} | | | ISSLEYEDMEIYYCLQYDEFPYTFGGGTKLEM |
| | | | KRGGGGSGGGGSGGGGSGGGGSDIQMTQTTSS |
| | | | LSASLGDRVTISCSASQGISNYLNWYQQKPDG |
| | | | TVKLLIYYTSSLHSGVPSRFSGSGSGTDYSLT |
| | | | ISNLEPEDIATYYCQQYSKLPYTFGGGTKLEI |
| | | | KRGGGGSGGGGSGGGGSGGGGSGGGGSGGGGS |
| | | | GGGGSGGGGSGGGGSGGGGSEVQLVESGGGLV |
| | | | KPGGSLKLSCAASGLTFSSYAMSWVRQTPEKR |
| | | | LEWVASISSGGFTYYPDSVKGRFTISRDNARN |
| | | | ILYLQMSSLRSEDTAMYYCARDEVRGYLDVWG |
| | | | AGTTVTVSGGGGSGGGGSGGGGSGGGGSEVKL |
| | | | EESGAELVKPGASVKLSCRTSGFNIKDTIHWV |
| | | | KQRPEQGLKWIGRIDPANGNTKYDPKFQDKAT |
| | | | VTADTSSNTAYLQLSSLTSEDTAVYYCVTYAY |
| | | | DGNWYFDVWGAGTAVTVSSPRASTTTPAPRPP |
| | | | TPAPTIASQPLSLRPEACRPAAGGAVHTRGLD |
| | | | FACDFWVLVVVGGVLACYSLLVTVAFIIFWVR |
| | | | SKRSRLLHSDYMNMTPRRPGPTRKHYQPYAPP |
| | | | RDFAAYRSRVKFSRSADAPAYKQGQNQLYNEL |
| | | | NLGRREEYDVLDKRRGRDPEMGGKPRRKNPQE |
| | | | GLYNELQKDKMAEAYSEIGMKGERRRGKGHDG |
| | | | LYQGLSTATKDTYDALHMQALPPRRTDGSGAT |
| | | | NFSLLKQAGDVEENPGPVSEAMPRGWTALCLL |
| | | | SLLPSGFMSLDNNGTATPELPTQGTFSNVSTN |
| | | | VSYQETTTPSTLGSTSLHPVSQHGNEATTNIT |
| | | | ETTVKFTSTSVITSVYGNTNSSVQSQTSVIST |
| | | | VFTTPANVSTPETTLKPSLSPGNVSDLSTTST |
| | | | SLATSPTKPYTSSSPILSDIKAEIKCSGIREV |
| | | | KLTQGICLEQNKTSSCAEFKKDRGEGLARVLC |
| | | | GEEQADADAGAQVCSLLLAQSEVRPQCLLLVL |
| | | | ANRTEISSKLQLMKKHQSDLKKLGILDFTEQD |
| | | | VASHQSYSQKTLIALVTSGALLAVLGITGYFL |
| | | | MNRRSWSPI |

Additionally, provided herein are hairpin tandem CAR constructs which may incorporate the V_{H} and V_{L} domains of scFvs targeting any of the antigen pairs provided in Table 6.

TABLE 9
Hairpin Tandem CAR Constructs.

| 9-I | 9-II | 9-III | 9-IV | 9-V | 9-VI | 9-VII | 9-VIII |
|---|---|---|---|---|---|---|---|
| CD8a | CD8a | CD8a | CD8a | CD8a | CD8a | CD8a | CD8a |
| V_{H}1 | V_{H}1 | V_{H}1 | V_{H}1 | V_{H}1 | V_{H}1 | V_{H}1 | V_{H}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{H}2 | V_{H}2 | V_{H}2 | V_{H}2 | V_{H}2 | V_{H}2 | V_{H}2 | V_{H}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{L}2 | V_{L}2 | V_{L}2 | V_{L}2 | V_{L}2 | V_{L}2 | V_{L}2 | V_{L}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ ID | (SEQ ID | (SEQ ID | (SEQ ID | (SEQ ID | (SEQ ID | (SEQ ID | (SEQ ID |
| NO: 449) | NO: 449) | NO: 449) | NO: 449) | NO: 449) | NO: 449) | NO: 449) | NO: 449) |
| V_{L}1 | V_{L}1 | V_{L}1 | V_{L}1 | V_{L}1 | V_{L}1 | V_{L}1 | V_{L}1 |
| CD8 Tm | CD8 Tm | CD8 Tm | CD8 Tm | CD28 Tm | CD28 Tm | CD28 Tm | CD28 Tm |
| 41BB | CD28 | 41BB - | CD28 - | 41BB | CD28 | 41BB - | CD28 - |
| | | CD28 | 41BB | | | CD28 | 41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |
| 9-IX | 9-X | 9-XI | 9-XII | 9-XIII | 9-XIV | 9-XV | 9-XVI |
| CD8a | CD8a | CD8a | CD8a | CD8a | CD8a | CD8a | CD8a |
| V_{H}2 | V_{H}2 | V_{H}2 | V_{H}2 | V_{H}2 | V_{H}2 | V_{H}2 | V_{H}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{H}1 | V_{H}1 | V_{H}1 | V_{H}1 | V_{H}1 | V_{H}1 | V_{H}1 | V_{H}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{L}1 | V_{L}1 | V_{L}1 | V_{L}1 | V_{L}1 | V_{L}1 | V_{L}1 | V_{L}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ ID | (SEQ ID | (SEQ ID | (SEQ ID | (SEQ ID | (SEQ ID | (SEQ ID | (SEQ ID |
| NO: 449) | NO: 449) | NO: 449) | NO: 449) | NO: 449) | NO: 449) | NO: 449) | NO: 449) |
| V_{L}2 | V_{L}2 | V_{L}2 | V_{L}2 | V_{L}2 | V_{L}2 | V_{L}2 | V_{L}2 |
| CD8 Tm | CD8 Tm | CD8 Tm | CD8 Tm | CD28 Tm | CD28 Tm | CD28 Tm | CD28 Tm |
| 41BB | CD28 | 41BB - | CD28 - | 41BB | CD28 | 41BB - | CD28 - |
| | | CD28 | 41BB | | | CD28 | 41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |
| 9-XVII | 9-XVIII | 9-XIX | 9-XX | 9-XXI | 9-XXII | 9-XXIII | 9-XIV |
| CD8a | CD8a | CD8a | CD8a | CD8a | CD8a | CD8a | CD8a |
| V_{L}1 | V_{L}1 | V_{L}1 | V_{L}1 | V_{L}1 | V_{L}1 | V_{L}1 | V_{L}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{L}2 | V_{L}2 | V_{L}2 | V_{L}2 | V_{L}2 | V_{L}2 | V_{L}2 | V_{L}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{H}2 | V_{H}2 | V_{H}2 | V_{H}2 | V_{H}2 | V_{H}2 | V_{H}2 | V_{H}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ ID | (SEQ ID | (SEQ ID | (SEQ ID | (SEQ ID | (SEQ ID | (SEQ ID | (SEQ ID |
| NO: 449) | NO: 449) | NO: 449) | NO: 449) | NO: 449) | NO: 449) | NO: 449) | NO: 449) |
| V_{H}1 | V_{H}1 | V_{H}1 | V_{H}1 | V_{H}1 | V_{H}1 | V_{H}1 | V_{H}1 |
| CD8 Tm | CD8 Tm | CD8 Tm | CD8 Tm | CD28 Tm | CD28 Tm | CD28 Tm | CD28 Tm |
| 41BB | CD28 | 41BB - | CD28 - | 41BB | CD28 | 41BB - | CD28 - |
| | | CD28 | 41BB | | | CD28 | 41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |
| 9-XXV | 9-XXVI | 9-XX VII | 9-XXVIII | 9-XIX | 9-XXX | 9-XXXI | 9-XXXII |
| CD8a | CD8a | CD8a | CD8a | CD8a | CD8a | CD8a | CD8a |
| V_{L}2 | V_{L}2 | V_{L}2 | V_{L}2 | V_{L}2 | V_{L}2 | V_{L}2 | V_{L}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{L}1 | V_{L}1 | V_{L}1 | V_{L}1 | V_{L}1 | V_{L}1 | V_{L}1 | V_{L}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{H}1 | V_{H}1 | V_{H}1 | V_{H}1 | V_{H}1 | V_{H}1 | V_{H}1 | V_{H}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ ID | (SEQ ID | (SEQ ID | (SEQ ID | (SEQ ID | (SEQ ID | (SEQ ID | (SEQ ID |
| NO: 449) | NO: 449) | NO: 449) | NO: 449) | NO: 449) | NO: 449) | NO: 449) | NO: 449) |
| V_{H}2 | V_{H}2 | V_{H}2 | V_{H}2 | V_{H}2 | V_{H}2 | V_{H}2 | V_{H}2 |
| CD8 Tm | CD8 Tm | CD8 Tm | CD8 Tm | CD28 Tm | CD28 Tm | CD28 Tm | CD28 Tm |
| 41BB | CD28 | 41BB - | CD28 - | 41BB | CD28 | 41BB - | CD28 - |
| | | CD28 | 41BB | | | CD28 | 41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |

For example, provided herein in Table 10 are hairpin tandem CAR constructs which incorporate the V_{H} and V_{L} domains of CD2 and CD3 scFvs.

TABLE 10
Hairpin Tandem CAR Constructs Targeting CD2 and CD3.

| Clone 5 | Clone 6 | Clone 7 | Clone 8 | Clone 13 | Clone 14 | Clone 15 | Clone 16 |
|---|---|---|---|---|---|---|---|
| CD8a | CD8a | CD8a | CD8a | CD8a | CD8a | CD8a | CD8a |
| CD3-V_{L} | CD3-V_{L} | CD3-V_{L} | CD3-V_{L} | CD2-V_{L} | CD2-V_{L} | CD3-V_{L} | CD3-V_{L} |
| GGGGS₄ | GGGGS₄ | GGGGS₄ | GGGGS₄ | GGGGS₄ | GGGGS₄ | GGGGS₄ | GGGGS₄ |
| (SEQ ID | (SEQ ID | (SEQ ID | (SEQ ID | (SEQ ID | (SEQ ID | (SEQ ID | (SEQ ID |
| NO: 450) | NO: 450) | NO: 450) | NO: 450) | NO: 450) | NO: 450) | NO: 450) | NO: 450) |
| CD2-V_{L} | CD2-V_{L} | CD2-V_{L} | CD2-V_{L} | CD3-V_{L} | CD3-V_{L} | CD2-V_{L} | CD2-V_{L} |
| (GGGGS)₁₀ | (GGGGS)₄ | (GGGGS)₁₀ | (GGGGS)₄ | (GGGGS)₁₀ | (GGGGS)₄ | (GGGGS)₁₀ | (GGGGS)₄ |
| (SEQ | GGGGP | (SEQ | GGGGP | (SEQ | GGGGP | (SEQ | GGGGP |
| ID | (GGGGS) | ID | (GGGGS) | ID | (GGGGS) | ID | (GGGGS)₄ |
| NO: 451) | (SEQ ID | NO: 451) | (SEQ ID | NO: 451) | (SEQ ID | NO: 451) | (SEQ ID |
| | NO: 452) | | NO: 452) | | NO: 452) | | NO: 452) |
| CD2-V_{H} | CD2-V_{H} | CD2-V_{H} | CD2-V_{H} | CD3-V_{H} | CD3-V_{H} | CD2-V_{H} | CD2-V_{H} |
| GGGGS₄ | GGGGS₄ | GGGGS₄ | GGGGS₄ | GGGGS₄ | GGGGS₄ | GGGGS₄ | GGGGS₄ |
| (SEQ ID | (SEQ ID | (SEQ ID | (SEQ ID | (SEQ ID | (SEQ ID | (SEQ ID | (SEQ ID |
| NO: 450) | NO: 450) | NO: 450) | NO: 450) | NO: 450) | NO: 450) | NO: 450) | NO: 450) |
| CD3-V_{H} | CD3-V_{H} | CD3-V_{H} | CD3-V_{H} | CD2-V_{H} | CD2-V_{H} | CD3-V_{H} | CD3-V_{H} |
| CD28 Tm | CD28 Tm | CD28 Tm | CD28 Tm | CD28 Tm | CD28 Tm | CD28 Tm | CD28 Tm |
| CD28 | CD28 | CD28 | CD28 | CD28 | CD28 | CD28 | CD28 |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |
| P2A | P2A | P2A | P2A | P2A | P2A | P2A | P2A |
| CD3₄ | CD3₄ | CD3₄ | CD3₄ | CD3₄ | CD3₄ | CD3₄ | CD3₄ |

Also provided herein in Table 11 are hairpin tandem CAR constructs with a (Cys=Cys) double-stranded bond (DSB) which may incorporate the V_{H} and V_{L} domains of scFvs targeting any of the antigen pairs provided in Table 6.

TABLE 11
Hairpin Tandem DSB CAR Constructs with a (Cys = Cys) Double-Stranded Bond
(DSB).

| 11-I | 11-II | 11-III | 11-IV | 11-V | 11-VI | 11-VII | 11-VIII |
|---|---|---|---|---|---|---|---|
| CD8a | CD8a | CD8a | CD8a | CD8a | CD8a | CD8a | CD8a |
| V_{H}1 | V_{H}1 | V_{H}1 | V_{H}1 | V_{H}1 | V_{H}1 | V_{H}1 | V_{H}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{H}2 | V_{H}2 | V_{H}2 | V_{H}2 | V_{H}2 | V_{H}2 | V_{H}2 | V_{H}2 |
| GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGGS₍₁₋₂₎ | GGGGS₍₁₋₂₎ | GGGGS₍₁₋₂₎ | GGGGS₍₁₋₂₎ | GGGGS₍₁₋₂₎ | GGGGS₍₁₋₂₎ | GGGGS₍₁₋₂₎ | GGGGS₍₁₋₂₎ |
| GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ |
| GGGGS₍₂₋₃₎ | GGGGS₍₂₋₃₎ | GGGGS₍₂₋₃₎ | GGGGS₍₂₋₃₎ | GGGGS₍₂₋₃₎ | GGGGS₍₂₋₃₎ | GGGGS₍₂₋₃₎ | GGGGS₍₂₋₃₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ |
| (SEQ ID | (SEQ ID | (SEQ ID | (SEQ ID | (SEQ ID | (SEQ ID | (SEQ ID | (SEQ ID |
| NO: 448) | NO: 448) | NO: 448) | NO: 448) | NO: 448) | NO: 448) | NO: 448) | NO: 448) |
| V_{L}2 | V_{L}2 | V_{L}2 | V_{L}2 | V_{L}2 | V_{L}2 | V_{L}2 | V_{L}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ ID | (SEQ ID | (SEQ ID | (SEQ ID | (SEQ ID | (SEQ ID | (SEQ ID | (SEQ ID |
| NO: 449) | NO: 449) | NO: 449) | NO: 449) | NO: 449) | NO: 449) | NO: 449) | NO: 449) |
| V_{L}1 | V_{L}1 | V_{L}1 | V_{L}1 | V_{L}1 | V_{L}1 | V_{L}1 | V_{L}1 |
| CD8 Tm | CD8 Tm | CD8 Tm | CD8 Tm | CD28 Tm | CD28 Tm | CD28 Tm | CD28 Tm |
| 41BB | CD28 | 41BB - | CD28 - | 41BB | CD28 | 41BB - | CD28 - |
| | | CD28 | 41BB | | | CD28 | 41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ |
| 11-IX | 11-X | 11-XI | 11-XII | 11-XIII | 11-XIV | 11-XV | 11-XVI |
| CD8a | CD8a | CD8a | CD8a | CD8a | CD8a | CD8a | CD8a |
| V_{H}2 | V_{H}2 | V_{H}2 | V_{H}2 | V_{H}2 | V_{H}2 | V_{H}2 | V_{H}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{H}1 | V_{H}1 | V_{H}1 | V_{H}1 | V_{H}1 | V_{H}1 | V_{H}1 | V_{H}1 |
| GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGGS₍₁₋₂₎ | GGGGS₍₁₋₂₎ | GGGGS₍₁₋₂₎ | GGGGS₍₁₋₂₎ | GGGGS₍₁₋₂₎ | GGGGS₍₁₋₂₎ | GGGGS₍₁₋₂₎ | GGGGS₍₁₋₂₎ |
| GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ |
| GGGGS₍₂₋₃₎ | GGGGS₍₂₋₃₎ | GGGGS₍₂₋₃₎ | GGGGS₍₂₋₃₎ | GGGGS₍₂₋₃₎ | GGGGS₍₂₋₃₎ | GGGGS₍₂₋₃₎ | GGGGS₍₂₋₃₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ |
| (SEQ ID | (SEQ ID | (SEQ ID | (SEQ ID | (SEQ ID | (SEQ ID | (SEQ ID | (SEQ ID |
| NO: 448) | NO: 448) | NO: 448) | NO: 448) | NO: 448) | NO: 448) | NO: 448) | NO: 448) |
| V_{L}1 | V_{L}1 | V_{L}1 | V_{L}1 | V_{L}1 | V_{L}1 | V_{L}1 | V_{L}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ ID | (SEQ ID | (SEQ ID | (SEQ ID | (SEQ ID | (SEQ ID | (SEQ ID | (SEQ ID |
| NO: 449) | NO: 449) | NO: 449) | NO: 449) | NO: 449) | NO: 449) | NO: 449) | NO: 449) |
| V_{L}2 | V_{L}2 | V_{L}2 | V_{L}2 | V_{L}2 | V_{L}2 | V_{L}2 | V_{L}2 |
| CD8 Tm | CD8 Tm | CD8 Tm | CD8 Tm | CD28 Tm | CD28 Tm | CD28 Tm | CD28 Tm |
| 41BB | CD28 | 41BB - | CD28 - | 41BB | CD28 | 41BB - | CD28 - |
| | | CD28 | 41BB | | | CD28 | 41BB |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |
| 11-XVII | 11-XVIII | 11-XIX | 11-XX | 11-XXI | 11-XXII | 11-XXIII | 11-XXIV |
| CD8a | CD8a | CD8a | CD8a | CD8a | CD8a | CD8a | CD8a |
| V_{L}1 | V_{L}1 | V_{L}1 | V_{L}1 | V_{L}1 | V_{L}1 | V_{L}1 | V_{L}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{L}2 | V_{L}2 | V_{L}2 | V_{L}2 | V_{L}2 | V_{L}2 | V_{L}2 | V_{L}2 |
| GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGGS₍₁₋₂₎ | GGGGS₍₁₋₂₎ | GGGGS₍₁₋₂₎ | GGGGS₍₁₋₂₎ | GGGGS₍₁₋₂₎ | GGGGS₍₁₋₂₎ | GGGGS₍₁₋₂₎ | GGGGS₍₁₋₂₎ |
| GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ |
| GGGGS₍₂₋₃₎ | GGGGS₍₂₋₃₎ | GGGGS₍₂₋₃₎ | GGGGS₍₂₋₃₎ | GGGGS₍₂₋₃₎ | GGGGS₍₂₋₃₎ | GGGGS₍₂₋₃₎ | GGGGS₍₂₋₃₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ |
| (SEQ ID | (SEQ ID | (SEQ ID | (SEQ ID | (SEQ ID | (SEQ ID | (SEQ ID | (SEQ ID |
| NO: 448) | NO: 448) | NO: 448) | NO: 448) | NO: 448) | NO: 448) | NO: 448) | NO: 448) |
| V_{H}2 | V_{H}2 | V_{H}2 | V_{H}2 | V_{H}2 | V_{H}2 | V_{H}2 | V_{H}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ ID | (SEQ ID | (SEQ ID | (SEQ ID | (SEQ ID | (SEQ ID | (SEQ ID | (SEQ ID |
| NO: 449) | NO: 449) | NO: 449) | NO: 449) | NO: 449) | NO: 449) | NO: 449) | NO: 449) |
| V_{H}1 | V_{H}1 | V_{H}1 | V_{H}1 | V_{H}1 | V_{H}1 | V_{H}1 | V_{H}1 |
| CD8 Tm | CD8 Tm | CD8 Tm | CD8 Tm | CD28 Tm | CD28 Tm | CD28 Tm | CD28 Tm |
| 41BB | CD28 | 41BB - | CD28 - | 41BB | CD28 | 41BB - | CD28 - |
| | | CD28 | 41BB | | | CD28 | 41BB |
| CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3ζ₍₁₋₂₎ | CD3(12)) |
| 11-XXV | 11-XXVI | 11-XXVII | 11-XXVIII | 11-XXIX | 11-XXX | 11-XXXI | 11-XXXII |
| CD8a | CD8a | CD8a | CD8a | CD8a | CD8a | CD8a | CD8a |
| V_{L}2 | V_{L}2 | V_{L}2 | V_{L}2 | V_{L}2 | V_{L}2 | V_{L}2 | V_{L}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ ID | (SEQ ID | (SEQ ID | (SEQ ID | (SEQ ID | (SEQ ID | (SEQ ID | (SEQ ID |
| NO: 447) | NO: 447) | NO: 447) | NO: 447) | NO: 447) | NO: 447) | NO: 447) | NO: 447) |
| V_{L}1 | V_{L}1 | V_{L}1 | V_{L}1 | V_{L}1 | V_{L}1 | V_{L}1 | V_{L}1 |
| GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGGS₍₁₋₂₎ | GGGGS₍₁₋₂₎ | GGGGS₍₁₋₂₎ | GGGGS₍₁₋₂₎ | GGGGS₍₁₋₂₎ | GGGGS₍₁₋₂₎ | GGGGS₍₁₋₂₎ | GGGGS₍₁₋₂₎ |
| GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ |
| GGGGS₍₂₋₃₎ | GGGGS₍₂₋₃₎ | GGGGS₍₂₋₃₎ | GGGGS₍₂₋₃₎ | GGGGS₍₂₋₃₎ | GGGGS₍₂₋₃₎ | GGGGS₍₂₋₃₎ | GGGGS₍₂₋₃₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ |
| (SEQ | (SEQ | (SEQ | (SEQ | (SEQ | (SEQ | (SEQ | (SEQ |
| ID | ID | ID | ID | ID | ID | ID | ID |
| NO: 448) | NO: 448) | NO: 448) | NO: 448) | NO: 448) | NO: 448) | NO: 448) | NO: 448) |
| V_{H}1 | V_{H}1 | V_{H}1 | V_{H}1 | V_{H}1 | V_{H}1 | V_{H}1 | V_{H}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 449) | NO: 449) | NO: 449) | NO: 449) | NO: 449) | NO: 449) | NO: 449) | NO: 449) |
| V_{H}2 | V_{H}2 | V_{H}2 | V_{H}2 | V_{H}2 | V_{H}2 | V_{H}2 | V_{H}2 |
| CD8 Tm | CD8 Tm | CD8 Tm | CD8 Tm | CD28 Tm | CD28 Tm | CD28 Tm | CD28 Tm |
| 41BB | CD28 | 41BB - | CD28 - | 41BB | CD28 | 41BB - | CD28 - |
| | | CD28 | 41BB | | | CD28 | 41BB |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |

Methods for Engineering CARs in a Dual or Tandem Construction with Gene Editing

In a further aspect, a CAR-T cell control may be created. For example, the control CAR-T cell may include an extracellular domain that binds to an antigen not expressed on a malignant T-cell. For example, if the therapeutic CAR-T cell targets a T-cell antigen such as CD7, or multiple T cell antigens, such as CD2 and CD3, the antigen the control CAR-T cell binds to may be CD19. CD19 is an antigen expressed on B cells but not on T cells, so a CAR-T cell with an extracellular domain adapted to bind to CD 19 will not bind to T cells. These CAR-T cells may be used as controls to analyze the binding efficiencies and non-specific binding of CAR-T cells targeted to the cancer of interest and/or recognizing the antigen of interest.

CARs may be further designed as disclosed in WO2018027036A1, optionally employing variations which will be known to those of skill in the art. Lentiviral vectors and cell lines can be obtained, and guide RNAs designed, validated, and synthesized, as disclosed therein as well as by methods known in the art and from commercial sources.

Engineered CARs may be introduced into T cells using retroviruses, which efficiently and stably integrate a nucleic acid sequence encoding the chimeric antigen receptor into the target cell genome. Other methods known in the art include, but are not limited to, lentiviral transduction, transposon-based systems, direct RNA transfection, and CRISPR/Cas systems (e.g., type I, type II, or type III systems using a suitable Cas protein such Cas3, Cas4, Cas5, Cas5e (or CasD), Cas6, Cas6e, Cas6f, Cas7, Cas8a1, Cas8a2, Cas8b, Cas8c, Cas9, Cas10, Casl Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966, etc.). Zinc finger nucleases (ZFNs) and transcription activator-like effector nucleases (TALENs) may also be used. See, e.g., Shearer RF and Saunders D N, “Experimental design for stable genetic manipulation in mammalian cell lines: lentivirus and alternatives,” Genes Cells 2015 January; 20(1):1-10.

Manipulation of PI3K signaling can be used to prevent altered CAR-T cell differentiation due to constitutive CAR self-signaling and foster long-lived memory T cell development. pharmacologic blockade of PI3K during CAR-T manufacture and ex vivo expansion can abrogate preferential effector T cell development and restore CAR-T effector/memory ratio to that observed in empty vector transduced T cells, which can improve in vivo T cell persistence and therapeutic activity. Inhibition of p110δ PI3K can enhance efficacy and memory in tumor-specific therapeutic CD8 T cells, while inhibition of p110α PI3K can increase cytokine production and antitumor response.

This is proposed to be because the presence of a CAR on a T cell's surface can alter its activation and differentiation, even in the absence of ligand. Constitutive self-signaling through CAR, related to both the scFv framework and the signaling domains, can lead to aberrant T cell behavior, including altered differentiation and decreased survival. This is significant as the effectiveness of CAR-T cells in patients is directly associated with their in vivo longevity. The presence of the CD28 costimulatory domain increased CAR-T cell exhaustion induced by persistent CAR self-signaling; the 4-1BB costimulatory domain had a lesser effect. Furthermore, CD3-zeta significantly enhances the constitutive activation of the PI3K, AKT, mTOR, and glycolysis pathways, and fostered formation of short-lived effector cells over central/stem memory cells. See, e.g., Zhang W. et al., “Modulation of PI3K signaling to improve CAR T cell function,” Oncotarget, 2018 Nov. 9; 9(88): 35807-35808.

### Cytokine Gene Deletion or Suppression

In addition to gene-editing the TCR and cell surface proteins and antigens, genes for secretable proteins such as cytokines and chemokines may be edited. Such editing would be done, e.g., to reduce or prevent the development or maintenance of cytokine release syndrome (CRS). CRS is caused by a large, rapid release of cytokines from immune cells in response to immunotherapy (or other immunological stimulus). Modifying, disrupting, or deleting one or more cytokine or chemokine genes can be accomplished using the methods known in the art, such as genetic ablation (gene silencing) in which gene expression is abolished through the alteration or deletion of genetic sequence information. This can be accomplished using known genetic engineering tools in the art, such as Transcription Activator-like Effector Nucleases (TALENs), Zinc Finger Nucleases (ZFNs), CRISPR, by transduction of small hairpin RNAs (shRNAs), by targeted transduction of a CAR into the gene sequence of the cytokine, and the like.

Cytokines or chemokines that can be deleted from immune effector cells as disclosed herein, e.g., using Cas9-CRISPR or by targeted transduction of a CAR into the gene sequence of the cytokine, include without limitation the following: XCL1, XCL2, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, CX3CL1, IL-1a, IL-1β, IL-1RA, IL-18, IL-2, IL-4, IL-7, IL-9, IL-13, IL-15, IL-3, IL-5, GM-CSF, IL-6, IL-11, G-CSF, IL-12, LIF, OSM, IL-10, IL-20, IL-14, IL-16, IL-17, IFN-α, IFN-β, IFN-γ, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β1, TGF-β2, TGF-β3, Epo, Tpo, Flt-3L, SCF, M-CSF, MSP, A2M, ACKR1, ACKR2, ACKR3, ACVR1, ACVR2B, ACVRL1, ADIPOQ, AGER, AGRN, AIMP1, AREG, BMP1, BMP10, BMP15, BMP2, BMP3, BMP4, BMP5, BMP6, BMP7, BMP8A, BMP8B, BMPR2, C10orf99, C1QTNF4, C5, CCL28, CCR1, CCR2, CCR3, CCR5, CCR6, CCR7, CD109, CD36, CD4, CD40LG, CD74, CER1, CHRD, CKLF, CLCF1, CMTM1, CMTM2, CMTM3, CMTM4, CMTM5, CMTM6, CMTM7, CMTM8, CNTF, CNTFR, COPS5, CRLF1, CSF1, CSF1R, CSF2, CSF3, CSF3R, CTF1, CX3CR1, CXCL16, CXCL17, CXCR1, CXCR2, CXCR3, CXCR4, CXCR6, EBI3, EDN1, ELANE, ENG, FAM3B, FAM3C, FAM3D, FAS, FASLG, FGF2, FLT3LG, FZD4, GBP1, GDF1, GDF10, GDF11, GDF15, GDF2, GDF3, GDF5, GDF6, GDF7, GDF9, GPI, GREM1, GREM2, GRN, HAX1, HFE2, HMGB1, HYAL2, IFNA10, IFNA14, IFNA16, IFNA2, IFNA5, IFNA6, IFNA8, IFNAR1, IFNAR2, IFNB1, IFNE, IFNG, IFNGR1, IFNK, IFNL1, IFNL3, IFNW1, IL10RA, IL11RA, IL12A, IL12B, IL12RB1, IL17A, IL17B, IL17C, IL17D, IL17F, IL18BP, IL-19, IL1F10, IL1R1, IL1R2, IL1RAPL1, IL1RL1, IL1RN, IL20RA, IL20RB, IL21, IL22, IL22RA1, IL22RA2, IL23A, IL23R, IL24, IL25, IL26, IL27, IL2RA, IL2RB, IL2RG, IL31, IL31RA, IL32, IL33, IL34, IL36A, IL36B, IL36G, IL36RN, IL37, IL6R, IL6ST, INHA, INHBA, INHBB, INHBC, INHBE, ITGA4, ITGAV, ITGB1, ITGB3, KIT, KITLG, KLHL20, LEFTY1, LEFTY2, LIFR, LTA, LTB, LTBP1, LTBP3, LTBP4, MIF, MINOS1-, MSTN, NAMPT, NBL1, NDP, NLRP7, NODAL, NOG, NRG1, NRP1, NRP2, OSMR, PARK7, PDPN, PF4, PF4V1, PGLYRP1, PLP2, PPBP, PXDN, SCG2, SCGB3A1, SECTM1, SLURP1, SOSTDC1, SP100, SPP1, TCAP, TGFBR1, TGFBR2, TGFBR3, THBS1, THNSL2, THPO, TIMP1, TNF, TNFRSF11, TNFRSF1A, TNFRSF9, TNFRSF10, TNFSF11, TNFSF12, TNFSF12-, TNFSF13, TNFSF13B, TNFSF14, TNFSF15, TNFSF18, TNFSF4, TNFSF8, TNFSF9, TRIM16, TSLP, TWSG1, TXLNA, VASN, VEGFA, VSTM1, WFIKKN1, WFIKKN2, WNT1, WNT2, WNT5A, WNT7A, and ZFP36.

The sequences of these genes are known and available in the art.

### Indications and Standards of Care in ACT (CAR-T) Therapy

In some embodiment, the genome-edited immune effector cells disclosed herein, and/or generated using the methods disclosed herein, express one or more chimeric antigen receptors (CARs) and can be used as a medicament, i.e., for the treatment of disease. In many embodiments, the cells are CAR-T cells.

Cells disclosed herein, and/or generated using the methods disclosed herein, may be used in immunotherapy and adoptive cell transfer, for the treatment, or the manufacture of a medicament for treatment, of cancers, autoimmune diseases, infectious diseases, and other conditions.

The cancer may be a hematologic malignancy or solid tumor. Hematologic malignancies include leukemias, lymphomas, multiple myeloma, and subtypes thereof. Lymphomas can be classified various ways, often based on the underlying type of malignant cell, including Hodgkin's lymphoma (often cancers of Reed-Sternberg cells, but also sometimes originating in B cells; all other lymphomas are non-Hodgkin's lymphomas), B-cell lymphomas, T-cell lymphomas, mantle cell lymphomas, Burkitt's lymphoma, follicular lymphoma, and others as defined herein and known in the art.

B-cell lymphomas include, but are not limited to, diffuse large B-cell lymphoma (DLBCL), chronic lymphocytic leukemia (CLL)/small lymphocytic lymphoma (SLL), and others as defined herein and known in the art.

T-cell lymphomas include T-cell acute lymphoblastic leukemia/lymphoma (T-ALL), peripheral T-cell lymphoma (PTCL), T-cell chronic lymphocytic leukemia (T-CLL) Sezary syndrome, and others as defined herein and known in the art.

Leukemias include Acute myeloid (or myelogenous) leukemia (AML), chronic myeloid (or myelogenous) leukemia (CML), acute lymphocytic (or lymphoblastic) leukemia (ALL), chronic lymphocytic leukemia (CLL) hairy cell leukemia (sometimes classified as a lymphoma), and others as defined herein and known in the art.

Plasma cell cell malignancies include lymphoplasmacytic lymphoma, plasmacytoma, and multiple myeloma.

In some embodiments, the medicament can be used for treating cancer in a patient, particularly for the treatment of solid tumors such as melanomas, neuroblastomas, gliomas or carcinomas such as tumors of the brain, head and neck, breast, lung (e.g., non-small cell lung cancer, NSCLC), reproductive tract (e.g., ovary), upper digestive tract, pancreas, liver, renal system (e.g., kidneys), bladder, prostate and colorectum.

In another embodiment, the medicament can be used for treating cancer in a patient, particularly for the treatment of hematologic malignancies selected from multiple myeloma and acute myeloid leukemia (AML) and for T-cell malignancies selected from T-cell acute lymphoblastic leukemia (T-ALL), non-Hodgkin's lymphoma, and T-cell chronic lymphocytic leukemia (T-CLL).

In some embodiments, the cells may be used in the treatment of autoimmune diseases such as lupus, autoimmune (rheumatoid) arthritis, multiple sclerosis, transplant rejection, Crohn's disease, ulcerative colitis, dermatitis, and the like. In some embodiments, the cells are chimeric autoantibody receptor T-cells, or CAR-Ts displaying antigens or fragments thereof, instead of antibody fragments; in this version of adoptive cell transfer, the B cells that cause autoimmune diseases will attempt to attack the engineered T cells, which will respond by killing them.

In some embodiments, the cells may be used in the treatment of infectious diseases such as HIV and tuberculosis.

In another embodiment, the CAR-T cells of the present disclosure can undergo robust in vivo T cell expansion and can persist for an extended amount of time.

In some embodiments, the treatment of a patient with CAR-T cells of the present disclosure can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic, is meant that the cells or population of cells used for treating patients are not originating from the patient but from a donor.

The treatment of cancer with CAR-T cells of the present disclosure may be in combination with one or more therapies selected from antibody therapy, chemotherapy, cytokine therapy, dendritic cell therapy, gene therapy, hormone therapy, radiotherapy, laser light therapy, and radiation therapy.

The administration of CAR-T cells or a population of CAR-T cells of the present disclosure of the present disclosure be carried out by aerosol inhalation, injection, ingestion, transfusion, implantation or transplantation. The CAR-T cells compositions described herein, i.e., mono CAR, dual CAR, tandem CARs, may be administered to a patient subcutaneously, intradermally, intratumorally, intranodally, intramedullary, intramuscularly, by intravenous or intralymphatic injection, or intraperitoneally. In one embodiment, the cell compositions of the present disclosure are preferably administered by intravenous injection.

The administration of CAR-T cells or a population of CAR-T cells can consist of the administration of 10⁴-10⁹ cells per kg body weight, preferably 10⁵ to 10⁶ cells/kg body weight including all integer values of cell numbers within those ranges. The CAR-T cells or a population of CAR-T cells can be administrated in one or more doses. In another embodiment, the effective amount of CAR-T cells or a population of CAR-T cells are administrated as a single dose. In another embodiment, the effective amount of cells are administered as more than one dose over a period time. Timing of administration is within the judgment of a health care provider and depends on the clinical condition of the patient. The CAR-T cells or a population of CAR-T cells may be obtained from any source, such as a blood bank or a donor. While the needs of a patient vary, determination of optimal ranges of effective amounts of a given CAR-T cell population(s) for a particular disease or conditions are within the skill of the art. An effective amount means an amount which provides a therapeutic or prophylactic benefit. The dosage administered will be dependent upon the age, health and weight of the patient recipient, type of concurrent treatment, if any, frequency of treatment, and the nature of the effect desired.

In another embodiment, the effective amount of CAR-T cells or a population of CAR-T cells or composition comprising those CAR-T cells are administered parenterally. The administration can be an intravenous administration. The administration of CAR-T cells or a population of CAR-T cells or composition comprising those CAR-T cells can be directly done by injection within a tumor.

In one embodiment of the present disclosure, the CAR-T cells or a population of the CAR-T cells are administered to a patient in conjunction with, e.g., before, simultaneously or following, any number of relevant treatment modalities, including but not limited to, treatment with cytokines, or expression of cytokines from within the CAR-T, that enhance T-cell proliferation and persistence and, include but not limited to, IL-2, IL-7, and IL-15 or analogues thereof.

In some embodiments, the CAR-T cells or a population of CAR-T cells of the present disclosure may be used in combination with agents that inhibit immunosuppressive pathways, including but not limited to, inhibitors of TGFβ, interleukin 10 (IL-10), adenosine, VEGF, indoleamine 2,3 dioxygenase 1 (IDO1), indoleamine 2,3-dioxygenase 2 (IDO2), tryptophan 2-3-dioxygenase (TDO), lactate, hypoxia, arginase, and prostaglandin E2.

In another embodiment, the CAR-T cells or a population of CAR-T cells of the present disclosure may be used in combination with T-cell checkpoint inhibitors, including but not limited to, anti-CTLA4 (Ipilimumab) anti-PD1 (Pembrolizumab, Nivolumab, Cemiplimab), anti-PDL1 (Atezolizumab, Avelumab, Durvalumab), anti-PDL2, anti-BTLA, anti-LAG3, anti-TIM3, anti-VISTA, anti-TIGIT, and anti-KIR.

In another embodiment, the CAR-T cells or a population of CAR-T cells of the present disclosure may be used in combination with T cell agonists, including but not limited to, antibodies that stimulate CD28, ICOS, OX-40, CD27, 4-1BB, CD137, GITR, and HVEM.

In another embodiment, the CAR-T cells or a population of CAR-T cells of the present disclosure may be used in combination with therapeutic oncolytic viruses, including but not limited to, retroviruses, picornaviruses, rhabdoviruses, paramyxoviruses, reoviruses, parvoviruses, adenoviruses, herpesviruses, and poxviruses.

In another embodiment, the CAR-T cells or a population of CAR-T cells of the present disclosure may be used in combination with immunostimulatory therapies, such as toll-like receptors agonists, including but not limited to, TLR3, TLR4, TLR7 and TLR9 agonists.

In another embodiment, the CAR-T cells or a population of CAR-T cells of the present disclosure may be used in combination with stimulator of interferon gene (STING) agonists, such as cyclic GMP-AMP synthase (cGAS).

Immune effector cell aplasia, particularly T cell aplasia is also a concern after adoptive cell transfer therapy. When the malignancy treated is a T-cell malignancy, and CAR-T cells target a T cell antigen, normal T cells and their precursors expressing the antigen will become depleted, and the immune system will be compromised. Accordingly, methods for managing these side effects are attendant to therapy. Such methods include selecting and retaining non-malignant T cells or precursors, either autologous or allogeneic (optionally engineered not to cause rejection or be rejected), for later expansion and re-infusion into the patient, after CAR-T cells are exhausted or deactivated. Alternatively, CAR-T cells which recognize and kill subsets of TCR-bearing cells, such as normal and malignant TRBC1⁺, but not TRBC2⁺ cells, or alternatively, TRBC2⁺, but not TRBC1⁺ cells, may be used to eradicate a T cell malignancy while preserving sufficient normal T cells to maintain normal immune system function.

### Definitions

Unless specifically defined herein, all technical and scientific terms used have the same meaning as commonly understood by a skilled artisan in the fields of gene therapy, biochemistry, genetics, and molecular biology. All disclosed compositions and methods similar or equivalent to those described herein can be used in the practice or testing of the present disclosure.

As used herein, the terms below have the meanings indicated. When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “activation” (and other conjugations thereof) in reference to cells is generally understood to be synonymous with “stimulating” and as used herein refers to treatment of cells that results in expansion of cell populations. In T cells, activation is often accomplished by exposure to CD2 and CD28 (and sometimes CD2 as well) agonists, typically antibodies, optionally coated onto magnetic beads or conjugated to a colloidal polymeric matrix.

The term “antigen” as used herein is a cell surface protein recognized by (i.e., that is the target of) T cell receptor or chimeric antigen receptor. In the classical sense antigens are substances, typically proteins, that are recognized by antibodies, but the definitions overlap insofar as the CAR comprises antibody-derived domains such as light (V_{L}) and heavy (V_{H}) chains recognizing one or more antigen(s).

The term “cancer” refers to a malignancy or abnormal growth of cells in the body. Many different cancers can be characterized or identified by particular cell surface proteins or molecules. Thus, in general terms, cancer in accordance with the present disclosure may refer to any malignancy that may be treated with an immune effector cell, such as a CAR-T cell as described herein, in which the immune effector cell recognizes and binds to the cell surface protein on the cancer cell. As used herein, cancer may refer to a hematologic malignancy, such as multiple myeloma, a T-cell malignancy, or a B cell malignancy. T cell malignancies may include, but are not limited to, T-cell acute lymphoblastic leukemia (T-ALL) or non-Hodgkin's lymphoma. A cancer may also refer to a solid tumor, such as including, but not limited to, cervical cancer, pancreatic cancer, ovarian cancer, mesothelioma, and lung cancer.

A “cell surface protein” as used herein is a protein (or protein complex) expressed by a cell at least in part on the surface of the cell. Examples of cell surface proteins include the TCR (and subunits thereof) and CD7.

A “chimeric antigen receptor” or “CAR” as used herein and generally used in the art, refers to a recombinant fusion protein that has an extracellular ligand-binding domain, a transmembrane domain, and a signaling transducing domain that directs the cell to perform a specialized function upon binding of the extracellular ligand-binding domain to a component present on the target cell. For example, a CAR can have an antibody-based specificity for a desired antigen (e.g., tumor antigen) with a T cell receptor-activating intracellular domain to generate a chimeric protein that exhibits specific anti-target cellular immune activity. First-generation CARs include an extracellular ligand-binding domain and signaling transducing domain, commonly CD3ζ or FcεRIγ. Second generation CARs are built upon first generation CAR constructs by including an intracellular costimulatory domain, commonly 4-1BB or CD28. These costimulatory domains help enhance CAR-T cell cytotoxicity and proliferation compared to first generation CARs. The third generation CARs include multiple costimulatory domains, primarily to increase CAR-T cell proliferation and persistence. Chimeric antigen receptors are distinguished from other antigen binding agents by their ability both to bind MHC-independent antigens and transduce activation signals via their intracellular domain.

A “CAR-bearing immune effector cell” is an immune effector cell which has been transduced with at least one CAR. A “CAR-T cell” is a T cell which has been transduced with at least one CAR; CAR-T cells can be mono, dual, or tandem CAR-T cells. CAR-T cells can be autologous, meaning that they are engineered from a subject's own cells, or allogeneic, meaning that the cells are sourced from a healthy donor, and in many cases, engineered so as not to provoke a host-vs-graft or graft-vs-host reaction. Donor cells may also be sourced from cord blood or generated from induced pluripotent stem cells.

A dual CAR-T cell (dCAR-T), can be defined as a T cell with two distinct chimeric antigen receptor polypeptides with affinity to different target antigen expressed within the same effector cell, wherein each CAR functions independently. The CAR may be expressed from single or multiple polynucleotide sequences.

A tandem CAR-T cell (tCAR-T), can be defined as a T cell with a single chimeric antigen polypeptide containing two distinct extracellular ligand-binding domains with affinity to different targets wherein the extracellular ligand-binding domains are linked through a peptide linker and share one or more common costimulatory domains, wherein binding of either extracellular ligand-binding domain will signal though one or more common costimulatory domains and signal transducing domain.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “composition” as used herein refers to an immunotherapeutic cell population combination with one or more therapeutically acceptable carriers.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “fratricide” as used herein means a process which occurs when a CAR-T cell becomes the target of, and is killed by, another CAR-T cell comprising the same chimeric antigen receptor as the target of CAR-T cell, because the targeted cell expresses the antigen specifically recognized by the chimeric antigen receptor on both cells. CAR-T cell comprising a chimeric antigen receptor which are deficient in an antigen to which the chimeric antigen receptor specifically binds will be “fratricide-resistant.”

The term “genome-edited” as used herein means having a gene added, deleted, or modified to be non-functional. Thus, in certain embodiments, a “gene-edited CAR-T cell” is an CAR-T cell that has had a gene such as a CAR recognizing at least one antigen added; and/or has had a gene such as the gene(s) to the antigen(s) that are recognized by the CAR deleted; and/or has had a gene such as the TCR, or a subunit thereof (e.g., the α or β chain) deleted or modified to be non-functional, or a subunit of the associated CD3 signal transduction complex, or a subunit thereof (e.g. the γ, δ, ε, or (chains) deleted or modified to be non-functional.

As used herein, “suicide gene” refers to a nucleic acid sequence introduced to a CAR-T cell by standard methods known in the art, that when activated result in the death of the CAR-T cell. If required suicide genes may facilitate the tracking and elimination, i.e., killing, of CAR-T cells in vivo. Facilitated killing of CAR-T cells by activating a suicide gene can be accomplished by standard methods known in the art. Suicide gene systems known in the art include, but are not limited to, include (a) herpes simplex virus (HSV)-tk which turns the nontoxic prodrug ganciclovir (GCV) into GCV-triphosphate, leading to cell death by halting DNA replication, (b) iCasp9 can bind to the small molecule AP1903 and result in dimerization, which activates the intrinsic apoptotic pathway, and (c) Targetable surface antigen expressed in the transduced T cells (e.g., CD20 and truncated EGFR), allowing eliminating the modified cells efficiently through complement/antibody-dependent cellular cytotoxicity (CDC/ADCC) after administration of the associated monoclonal antibody.

A “cancer cell”, for example, is a malignant T cell, malignant B cell, or malignant plasma cell.

A “malignant B cell” is a B cell derived from a B-cell malignancy. B cell malignancies include, without limitation, (DLBCL), chronic lymphocytic leukemia (CLL)/small lymphocytic lymphoma (SLL), and B cell-precursor acute lymphoblastic leukemia (ALL).

A “malignant T cell” is a T cell derived from a T-cell malignancy.

The term “T-cell malignancy” refers to a broad, highly heterogeneous grouping of malignancies derived from T-cell precursors, mature T cells, or natural killer cells. Non-limiting examples of T-cell malignancies include T-cell acute lymphoblastic leukemia/lymphoma (T-ALL), human T-cell leukemia virus type 1-positive (HTLV-1+) adult T-cell leukemia/lymphoma (ATL), T-cell prolymphocytic leukemia (T-PLL), Adult T-cell lymphoma/leukemia (HTLV-1 associated), Aggressive NK-cell leukemia, Anaplastic large-cell lymphoma (ALCL), ALK positive, Anaplastic large-cell lymphoma (ALCL), ALK negative, Angioimmunoblastic T-cell lymphoma (AITL), Breast implant-associated anaplastic large-cell lymphoma, Chronic lymphoproliferative disorder of NK cells, Extra nodal NK/T-cell lymphoma, nasal type, Enteropathy-type T-cell lymphoma, Follicular T-cell lymphoma, Hepatosplenic T-cell lymphoma, Indolent T-cell lymphoproliferative disorder of the GI tract, Monomorphic epitheliotrophic intestinal T-cell lymphoma, Mycosis fungoides, Nodal peripheral T-cell lymphoma with TFH phenotype, Peripheral T-cell lymphoma (PTCL), NOS, Primary cutaneous γδ T-cell lymphoma, Primary cutaneous CD8+ aggressive epidermotropic cytotoxic T-cell lymphoma, Primary cutaneous acral CD8+ T-cell lymphoma, Primary cutaneous CD4+ small/medium T-cell lymphoproliferative disorders [Primary cutaneous anaplastic large-cell lymphoma (C-ALCL), lymphoid papulosis], Sezary syndrome, Subcutaneous, panniculitis-like T-cell lymphoma, Systemic EBV+ T-cell lymphoma of childhood, and T-cell large granular lymphocytic leukemia (LGL).

A “healthy donor,” as used herein, is one who does not have a hematologic malignancy (e.g. a T-cell malignancy).

The term “therapeutically acceptable” refers to substances which are suitable for use in contact with the tissues of patients without undue toxicity, irritation, and allergic response, are commensurate with a reasonable benefit/risk ratio, and/or are effective for their intended use.

The term “therapeutically effective” is intended to qualify the amount of active ingredients used in the treatment of a disease or disorder or on the effecting of a clinical endpoint.

As used herein, a “secretable protein” is s protein secreted by a cell which has an effect on other cells. By way of example, secretable proteins include ctyokines, chemokines, and transcription factors.

The term “donor template” refers to the reference genomic material that the cell uses as a template to repair the a double-stranded break through the homology-directed repair (HDR) DNA repair pathway. The donor template contains the piece of DNA to be inserted into the genome (containing the gene to be expressed, CAR, or marker) with two homology arms flanking the site of the double-stranded break. In some embodiments, a donor template may be an adeno-associated virus, a single-stranded DNA, or a double-stranded DNA.

The term “exposing to,” as used herein, in the context of bringing compositions of matter (such as antibodies) into intimate contact with other compositions of matter (such as cells), is intended to be synonymous with “incubated with,” and no lengthier period of time in contact is intended by the use of one term instead of the other.

The term “patient” is generally synonymous with the term “subject” and includes all mammals including humans.

The invention is further illustrated by the following examples.

### EXAMPLES

### Example 1—Method of Making and Testing a Genome-Edited CAR-T Cells by Insertion of CAR into CD3e Loci

The following steps may be taken to provide a genome-edited CAR-T cell in which the car is expressed from the gene edited loci (CAR-T) disclosed herein. This example describes the making of a CD7CART ΔCD7 ΔCD3ε cell. As those of skill in the art will recognize, certain of the steps may be conducted sequentially or out of the order listed below, though perhaps leading to different efficiency.

TABLE 12
Guide RNA sequences for use in removing surface antigens on immune effector
cells.

| Target | |
|---|---|
| gene | gRNA sequence |
| CD7 | 5′_2′OMe(A(ps)U(ps)C(ps))ACGGAGGUCAAUGUCUAGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps)U_3′ (SEQ ID NO: 47) |
| CD7g10 | 5′_2′OMe(G(ps)U(ps)A(ps))GACAUUGACCUCCGUGAGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps)U_3′ (SEQ ID NO: 48) |
| CD7g4 | 5′_2′OMe(A(ps)U(ps)C(ps))ACGGAGGUCAAUGUCUAGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps)U_3′(SEQ ID NO: 49) |
| TRACg | 5′_2′OMe(G(ps)A(ps)G(ps))AAUCAAAAUCGGUGAAUGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps)U_3′ (SEQ ID NO: 50) |
| CS1 | 5′_2′OMe(G(ps)A(ps)C(ps))CAAUCUGACAUGCUGCAGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps)U_3′ (SEQ ID NO: 51) |
| CD2 | 5′_2′OMe(A(ps)C(ps)A(ps))GCUGACAGGCUCGACACGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps) U_3′ (SEQ ID NO: 52) |
| CD2g | 5′_2′OMe(G(ps)A(ps)G(ps))AAUCAAAAUCGGUGAAUGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps) U 3′ (SEQ ID NO: 53) |
| CD3εg | 5′_2′OMe(A(ps)G(ps)G(ps))GCAUGUCAAUAUUACUGGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps) U 3′ (SEQ ID NO: 54) |
| CD5 | 5′_2′OMe(C(ps)G(ps)U(ps))UCCAACUCGAAGUGCCAGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps))U3′ (SEQ ID NO: 55) |
| CD5g | 5′_2′OMe(C(ps)G(ps)U(ps))uCCAACUCGAAGUGCCAGUUUUAGAGCUAGA |
| | AAUAGCAAGUUAAAAUAAGGCUAGUCCGUUAUCAACUUGAAAAAGUG |
| | GCACCGAGUCGGUGC2′OMe(U(ps)U(ps)U(ps)U_3′ (SEQ ID NO: 56) |

RNA; (ps) indicate phosphorothioate. Underlined bases denote target sequence.

### Step 1—T Cell Activation (Day 0).

Purify T cells from leukapheresis chamber using Miltenyi human PanT isolation kit. Resuspend in media. Count cells. Determine number of human T cell activation CD3/CD28 beads required to obtain 3:1 bead:cell ratio. Wash beads 2× with T cell media. Dilute cells at 1.256 cells/mL in hXcyte media. Add human T cell activation CD3/CD28 beads. Aliquot 4 mL/well of 1.256 cell/mL solution into 6 well plate. Incubate cells at 37 C.

### Step 2—CRISPR (Day 2).

The target gene is genetically deleted and the CAR inserted into the gene edited loci. The DNA double strand break can be repaired using homolopgy directed repair using a donor template to repair the break and insert the desired sequence into the edited loci. Target deletion may be accomplished by electroporating with Cas9 mRNA and gRNA against the target(s). The donor template may be, a DNA plasmid, or double stranded linear DNA containing homology to the DNA surrounding the double strand breaks electroporated with the Cas9/gRNA. Additionally, a viral vector such as AAV may be used as the source of the donor template. Other techniques, however, could be used to induce DNA double strand breaks. These include other genome editing techniques such as TALENs and mega-nucleases.

TABLE 13
CRISPR Protocol

| Sample | | | | Nuecleofection |
|---|---|---|---|---|
| ID | gRNA#1 | gRNA#2 | Cas9 | Buffer P3 |
| UCART7 | 20 ug gCD7 | 20 ug gCD3ε | 15 ug Cas9 | 100 ul |
| | | | mRNA | |

Protocol—Nucleofection using nucleofector 4D—4×10⁶ cells per reaction. Program EO-115-100 ul transfection volume. The entire supplement needs to be added to the Nucleofector™ Solution P3. Prepare cell culture plates by filling appropriate number of wells with desired volume of recommended culture media (2 ml in 6 well plate) and pre-incubate/equilibrate plates in a humidified 37° C./5% CO₂ incubator. Magnetically Remove beads (do this twice to ensure complete removal). Count cells and determine cell density. Centrifuge the required number of cells at 90×g for 10 minutes at room temperature. Remove supernatant completely. Resuspend in PBS (1 ml) and transfer to a microcentrifuge tube and centrifuge the required number of cells at 90×g for 10 minutes at room temperature. Remove supernatant completely. Resuspend the cell pellet carefully in complete room temperature 4DNucleofector™ Solution P3 4×10⁶ per 100 ul). Add 20 ug of each gRNA (gCD7 and gCD3ε) to each tube of 15 ug cas9. Add 100 ul of cells to each tube of Cas9/gRNA, gently mix and transfer everything into the Nucleocuvette™. Gently tap to remove bubbles. Electroporate using program (Human T cell stim EO-115). After run completion, carefully remove the Nucleocuvette™ Vessel from the retainer using special. Resuspend cells with pre-warmed medium. Take up media from destination well, add to cuvette and gently pipetting up and down two to three times. Transfer to well. Repeat with media from same well. Incubate at 37° C.

Step 3—Transduction of T Cells with AAV Vector Containing HDR Repair Construct.

Recombinant AAV6 donor vector is added to the cell culture 2-4 hrs after electroporation with a MOI between 1e4 and 1e6.

### Step 4—Assessment of CRISPR Activity and Td Efficiency (Day 10).

Take 5×10⁵ cells from each sample and analyze by flow cytometry. Wash samples with RB. Add 3 ul of anti-CD34 PE antibody (This detects the CAR as our construct contains human truncated CD34). Add 5 ul of CD3 APC and 2 ul of CD2 BV421. Wash. Perform Flow cytometry. Cells should be CD3ε negative, CD7 negative. Harvest T cells (Day 11).

Purification of CAR-T cells. CD34+ (CAR+) and TCR negative cells can be purified in a single step using a positive selection of CD34+ cells on the Miltenyi Automacs. This enriches the CAR+ cells and removes and TCR+ cells (as CAR insertion disrupts TCR signaling)

### Step 5—Assessment of CAR-T Activity In Vivo

Inject tumor in NSG mice (5e5 MOLT3 or HH: containing Luciferase) if performing in vivo imaging experiment. (Day 7)

Image tumor burden in mouse using bioluminescent imaging. Inject 2×10⁶ CD34+ CAR-T per mouse I.V. via tail vein or perform a 4 hr chromium release assay against targets cell (MOLT3 or HH) (Day 11). Those of skill in the art will appreciate that some flexibility is possible in the time frames specified in Example 1.

### Example 2—Method of Making a Genome-Edited Tandem tCAR-T Cells

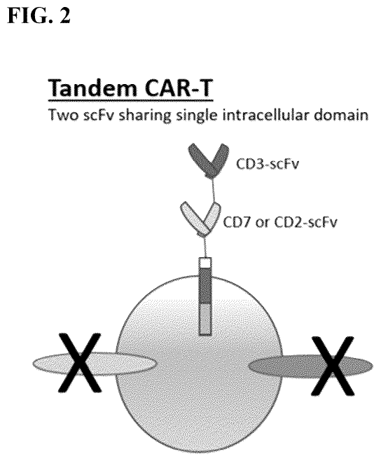

In a variation of the protocol in Example 1, a tandem CAR-T cell recognizing two antigens can be made. In Step 2, the two antigens can be deleted from the cell surface, or suppressed as described above, by electroporating with gRNA for each of the two targets and Cas9 mRNA. In Step 3, This CAR-T cell is then transduced with a CAR that recognizes two targets. The variations of a tandem CAR-T cell shown in the schematic in FIG. 2. Additional examples of tCAR-T cells are shown in Table 6.

### Example 3—Genome-Edited Dual CAR-T Cells or Genome-Edited Tandem CAR-T Cells

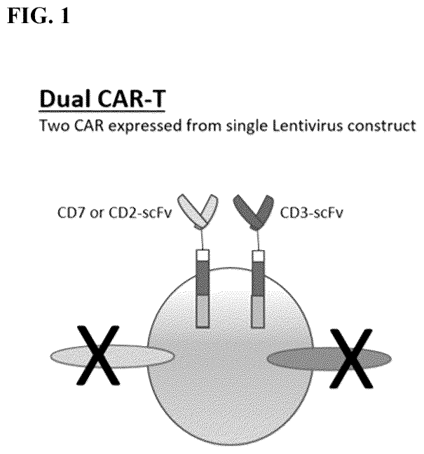

Several types of genome-edited dual or tandem CAR-T cells may be made using the methods above. FIG. 1 and FIG. 2 show the examples of tandem and dual CAR-T cells. The figures state the antigens to be targeted and does not indicate order of scFv expression in tandem CAR construct. Further examples are provided below in Tables 6-11.

Additional examples of tandem and dual CAR-T cells are provided herein with deletion, without deletion, or suppression of one or more surface proteins that are target antigens of the CARs and expressed on CAR-T cells. In general, examples with deletion or suppression of more than one antigen will more likely have the benefit of greater fratricide-resistance for these CAR-T cells. It should be further noted that the order in which the scFvs are oriented in the tandem CARs are set forth in Tables 6-11 and is not limiting. For example, the CD2*CD3ε encompasses a tCAR with the orientation CD2*CD3ε or the orientation CD3ε*CD2.

Additional examples of mono, tandem, and dual CAR-T cells targeting antigens expresses on multiple myeloma cells are provided herein, without deletion, with deletion, or suppression of one or more surface proteins that are target antigens of the CARs and expressed on CAR-T cells. In general, examples with deletion or suppression of more than one antigen will more likely have the benefit of greater fratricide-resistance for these CAR-T cells.

### Example 4—Treatment of Patient(s) with Genome-Edited Dual or Tandem CAR-T Cells

Patients may be treated using cells made by the methods above, as shown in FIG. 1 and FIG. 2. For example, an expanded population of dual or tandem CAR-T cells may be infused into a patient

Dual or Tandem CAR-T cells target cancer cells without inducing alloreactivity. For example, CD2*CD3249-dCARTΔCD2ΔCD3ε cells would target cancer cells (and other non-cancer cells) bearing the CD2 and CDε surface antigens.

### Example 5—Testing Efficacy of CD2*CD3Δ-dCARTΔCD2ΔCD3ε in a Xenogeneic Model of T-ALL

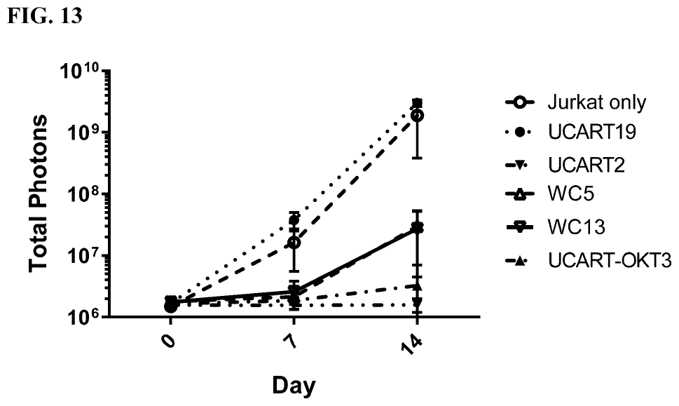

Testing efficacy of CD2*CD3Δ-dCARTΔCD2ΔCD3ε in a xenogeneic model of T-ALL: 5×10⁵ Click Beetle Red luciferase (CBR) labeled Jurkat (T-ALL—99% CD2÷, 99% % CD3% by FACS) cells were injected I.V. into NSG recipients prior to infusion of CD2*CD3ε-dCARTΔCD2ΔCD3ε(WC5 or WC13), CD3CARTΔCD2ΔCD3ε(UCART3), CD2CARTΔCD2ΔCD3ε (UCART2) or non-targeting CD19-CARΔCD2ΔCD3ε(UCART19) control cells i.v. on day +4. In contrast to mice receiving CD19-CARΔCD2ΔCD3ε or mice injected with tumor only, mice receiving CD2*CD3ε-dCARTΔCD2ΔCD3ε demonstrate significantly prolonged survival and reduced tumor burden as determined by bioluminescent imaging shown in FIG. 13. In future models, CD2*CD3ε-dCARTΔCD2ΔCD3ε would provide a survival advantage over CD3CARTΔCD2ΔCD3ε, CD2CARTΔCD2ΔCD3ε, and reduce tumor burden in a version of this model in which the target cell is missing either CD2 (CD2CARTΔCD2ΔCD3ε) or CD3 (CD3CARTΔCD2ΔCD3ε).

### Example 6—Genome-Edited Mono CAR-T Cells

Examples of genome-edited mono CAR-T cells targeting antigens expresses on hematologic malignancies are provided below, without deletion, with deletion, or suppression of one or more surface proteins that are target antigens of the CARs and expressed on CAR-T cells. In general, examples with deletion or suppression of more than one antigen will more likely have the benefit of greater fratricide-resistance for these CAR-T cells.

### Example 7—Genome-Edited UCART2/3 Cells Made by Editing Before Activation

On Day 0, cells were thawed in a thaw buffer. Thereafter, cells were resuspended in media and allowed to rest for two hours. Cells were harvested and counted. The required number of cells were centrifuged at 100×g for 10 minutes at room temperature. Supernatant was removed completely, cells resuspended in PBS (1 ml) and transfer to a microcentrifuge tube, and centrifuged at 100×g for 10 minutes at room temperature. Supernatant was removed completely, and cells then resuspended in a buffer P3, counted, and the count adjusted to 5×10⁷ per mL. A cell pool volume of 100 μL was added to a tube containing Cas9/gRNA, gently mixed, and everything transferred into the Nucleocuvette™, which was gently tapped to remove bubbles. Electroporation was thereafter commenced using program (Human T cell stim EO-115). After this procedure, the activated cells were transferred to pre-warmed media and distributed in 2 mL aliquots in a 12-well plate. Aliquoted samples were rested for 24 hours.

On day 1, cells were activated with T Cell TransAct™ as shown in Table 14.

TABLE 14
T Cell TransAct ™ Activation.

| | Name | Media | Stimulation | Cas9 p | gRNA | Virus |
|---|---|---|---|---|---|---|
| 1 | WT | TexMacs | T Cell TransAct ™ | — | — | |
| | | | (50 μl) | | | |
| 2 | WC5 | TexMacs | T Cell TransAct ™ | 2 ul | 20 ug iDT CD2 + | WC5 |
| | | | (50 μl) | | CD3ε | |
| 3 | WC6 | TexMacs | T Cell TransAct ™ | 2 ul | 20ug iDT CD2 + | WC6 |
| | | | (50 μl) | | CD3ε | |
| 4 | WC7 | TexMacs | T Cell TransAct ™ | 2 ul | 20 ug iDT CD2 + | WC7 |
| | | | (50 μl) | | CD3ε | |
| 5 | WC8 | TexMacs | T Cell TransAct ™ | 2 ul | 20 ug iDT CD2 + | WC8 |
| | | | (50 μl) | | CD3ε | |
| 6 | WC13 | TexMacs | T Cell TransAct ™ | 2 ul | 20 ug iDT CD2 + | WC13 |
| | | | (50 μl) | | CD3ε | |
| 7 | WC14 | TexMacs | T Cell TransAct ™ | 2 ul | 20 ug iDT CD2 + | WC14 |
| | | | (50 μl) | | CD3ε | |
| 8 | WC15 | TexMacs | T Cell TransAct ™ | 2 ul | 20 ug iDT CD2 + | WC15 |
| | | | (50 μl) | | CD3ε | |
| 9 | WC16 | TexMACS | T Cell TransAct ™ | 2 ul | 20 ug iDT CD2 + | WC16 |
| | | | (50 μl) | | CD3ε | |

On day 2, 1 μl of polybrene was added for each ml media (8 mg/ml stock). The required amount of virus was added to give required M.O.I (Multiplicity of Infection). Cells and virus were mixed and placed back in incubator at 37° C.

On day 3, activated cells were washed to remove stimulation.

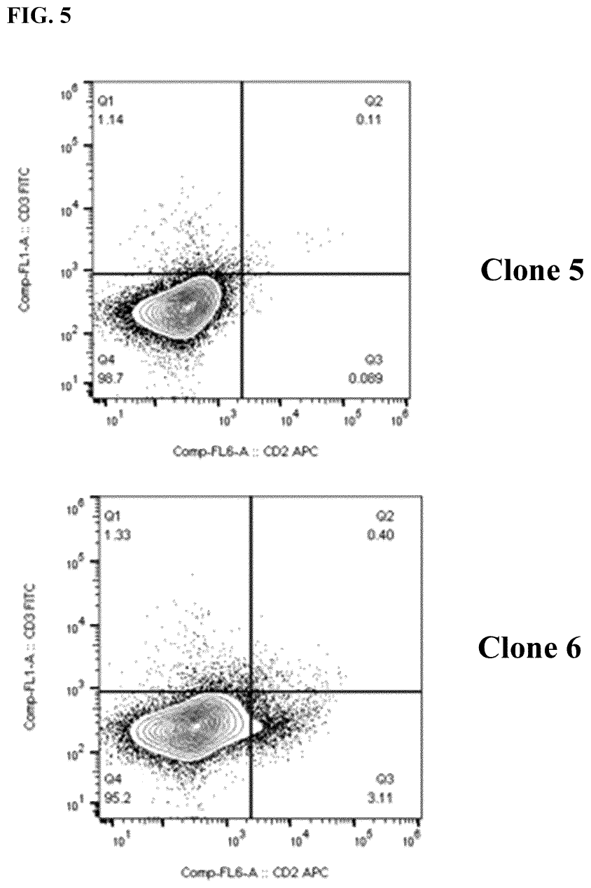

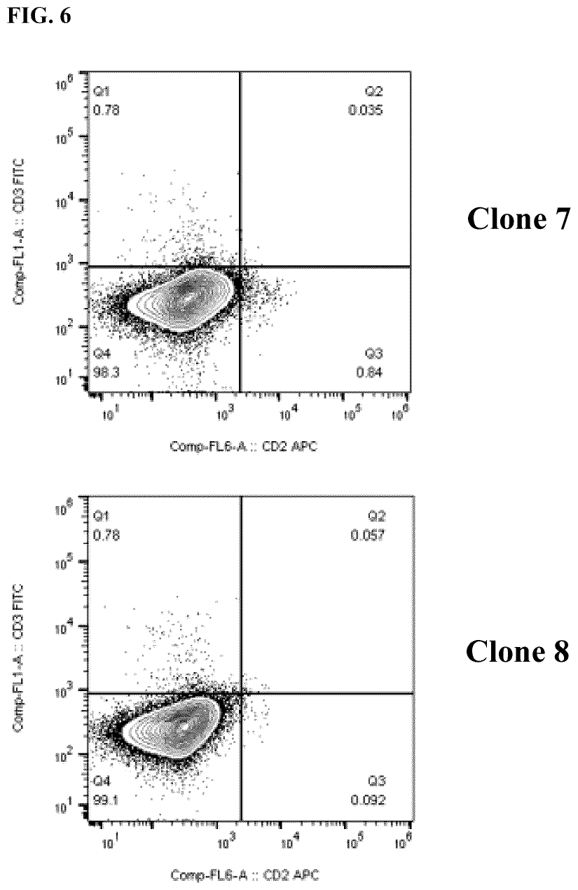

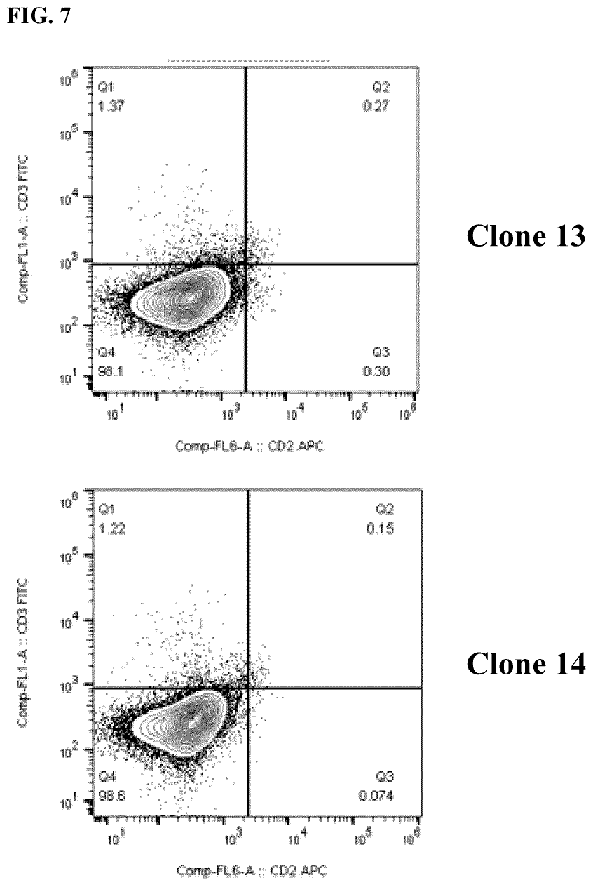

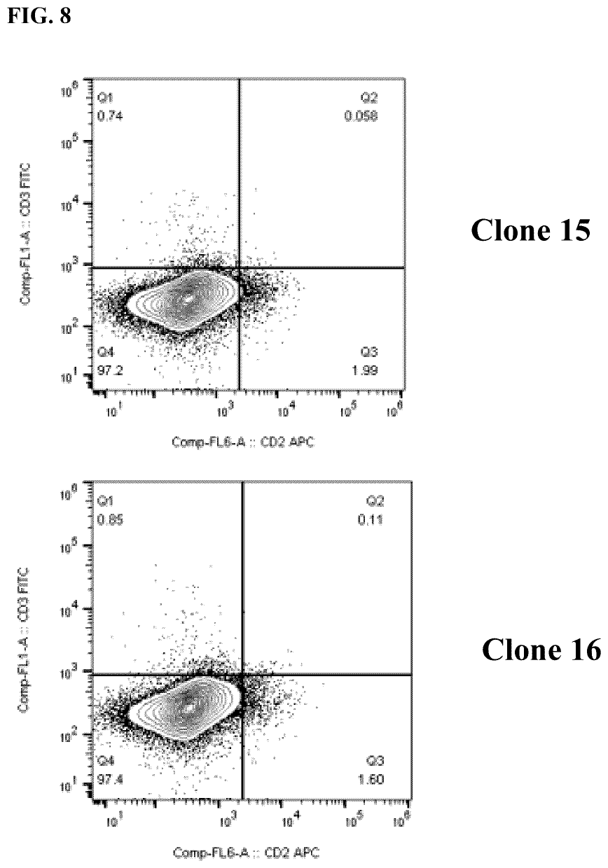

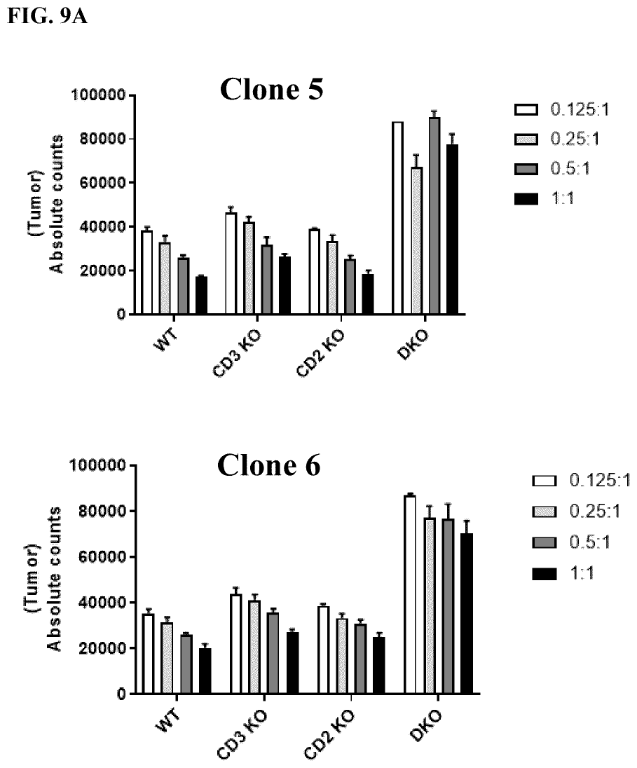

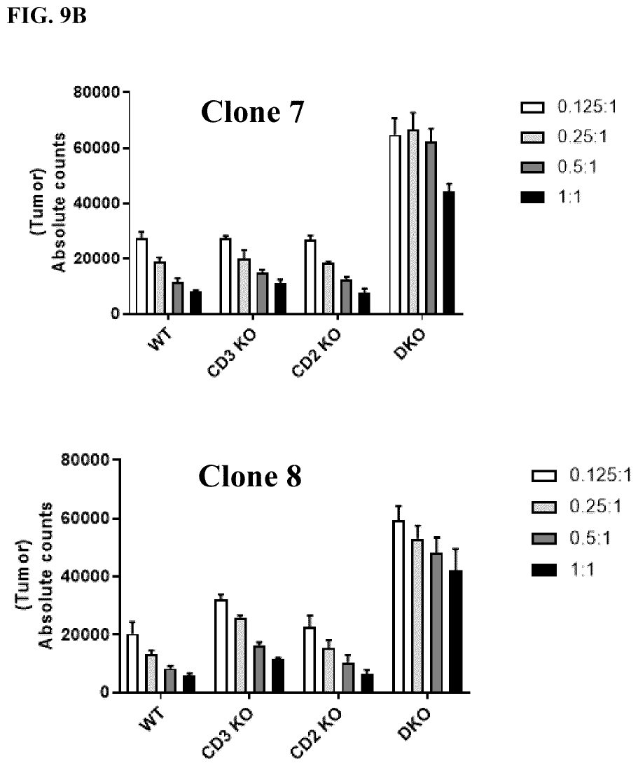

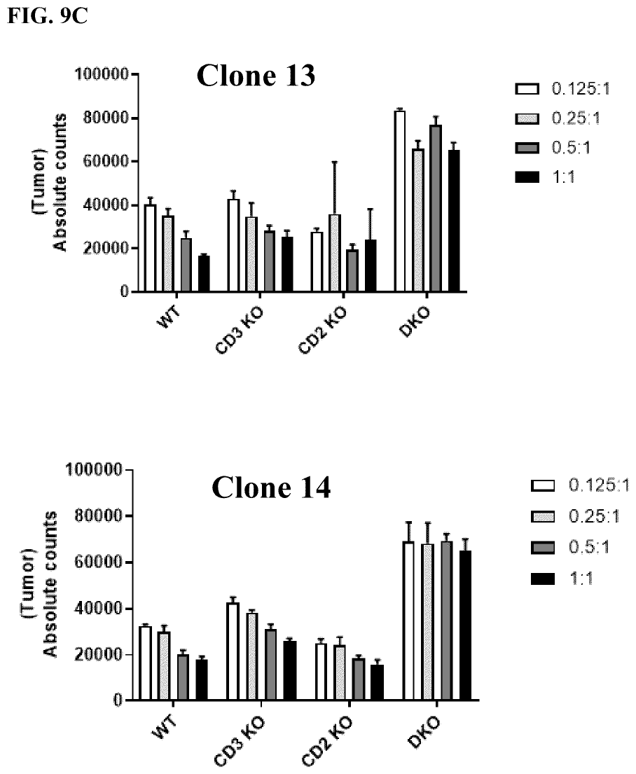

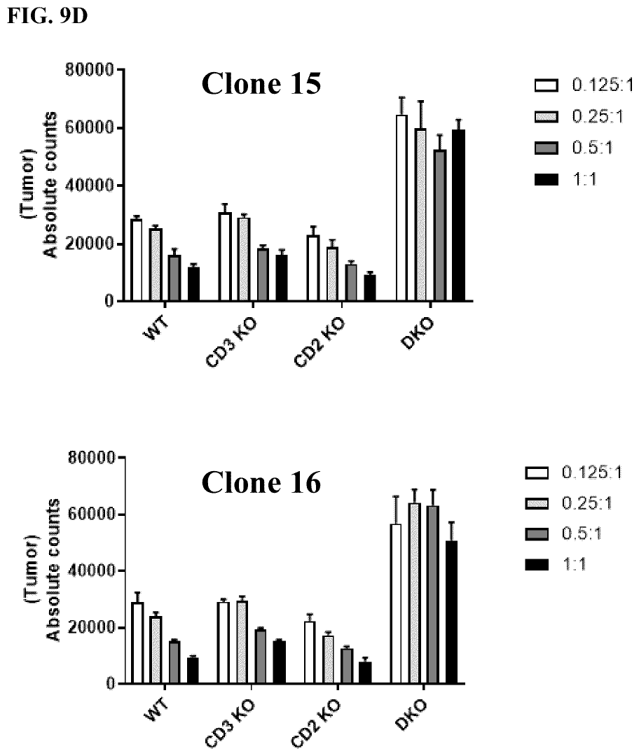

On Day 12, FACS analysis showed the high purity of CD3⁻CD2⁻/CAR-T cells; see FIG. 5 (clone 5 and clone 6), FIG. 6 (clone 7 and clone 8), FIG. 7 (clone 13 and clone 14), and FIG. 8 (clone 15 and clone 16). Standard four-hour chromium release (⁵¹Cr) assays were performed using (⁵¹Cr) labeled genome-edited Jurkat cells (ΔCD2, ΔCD3, and ΔCD2ΔCD3. These experiments showed a functional tumor killing response to CD2 and CD3 targets independent of one another (see FIG. 9A, FIG. 9B3, FIG. 9C, and FIG. 9D)).

### Example 8—Tumor Cell Killing of BCMA-CAR-T Cells

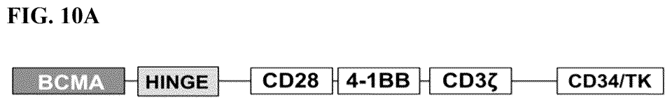

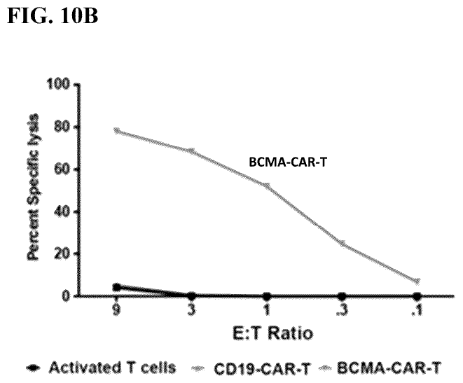

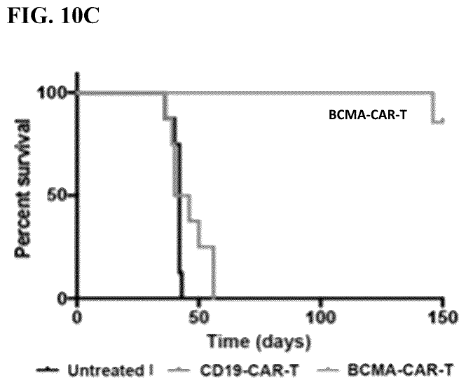

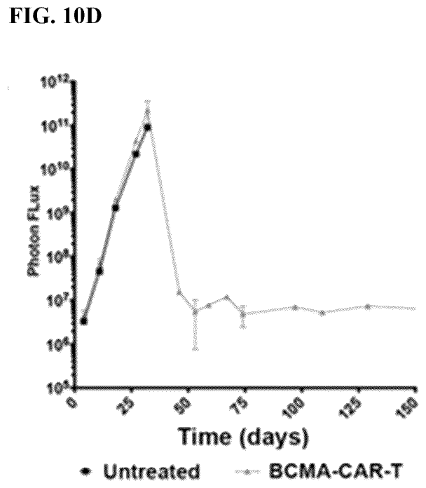

BCMA CAR-Ts were first tested in vitro for efficacy using a standard four-hour chromium release (51Cr) assays using 51Cr labeled MM.1S target cells. To enable in vivo tracking, the human myeloma cell line (BCMA+/CD19−), was modified to express click beetle red luciferase fused to GFP (MM.1S-CG). The CAR-T cells were incubated with 51Cr-labeled MM.1S-CG cells for four hours at a range of effector (CAR-T) to target (MM.1S-CG) ratios and released 51Cr was measured as a marker of MM.1S-CG cell death (FIG. 10B). Efficient killing was observed at multiple Effector to Target (E:T) ratios. Non-transduced activated T cells and CD19-CAR-Ts were used as negative controls and did not induce killing of MM.1S-CG cells. Next, in vivo efficacy was tested by engrafting NSG mice with 500,000 MM.1S-CG human myeloma cells (i.v.). Twenty-eight days later, when tumor burden was high, mice were left untreated or were treated with 2×106 CD19-CAR-Ts or BCMA CAR-Ts. All seven mice treated with BCMA CAR-Ts lived to almost 150 days or more compared to controls which died around day 50 (FIG. 10C). The cause of death of the one mouse that died in the BCMA CAR-T cohort is unknown. Flow cytometry analysis revealed no GFP+ tumor cells in that mouse. Serial bioluminescent imaging (BLI) revealed a robust reduction of signal to background levels that never increased throughout the duration of the experiment (FIG. 10D).

### Example 9—Tumor Cell Killing of CS1-CAR-T Cells

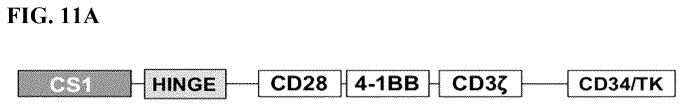

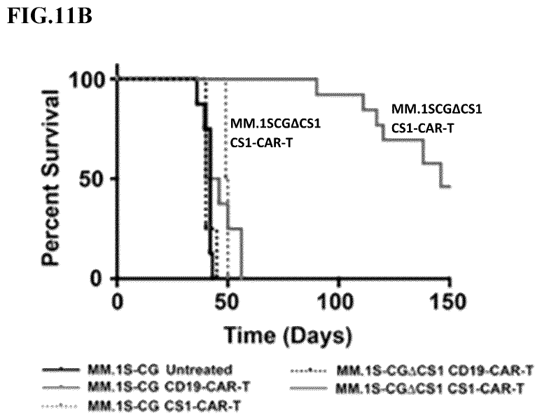

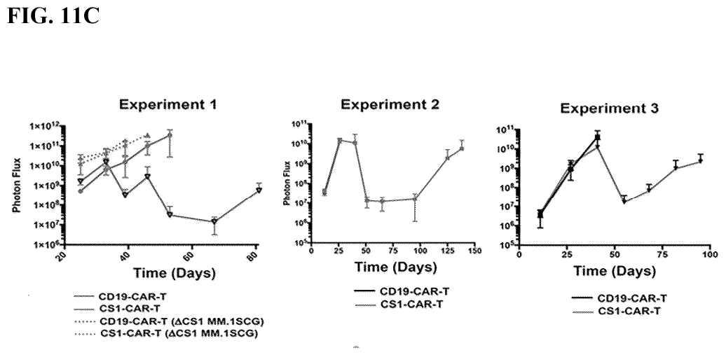

In vivo efficacy of CS1-CAR-T cells by injecting 5×10⁵ MM.1S-CG into NSG mice and 28 days later when tumor burden was high (BLI signal 1010 photon flux), injected 2×10⁶ CS1-CAR-T cells or negative control CD19-CAR-T cells. Mice were also engrafted with MM.1S-CG cells lacking CS1 (using CAS9/CRISPR technology; MM.1S-CGACS1) as a method to test the specificity of CS1-CAR-T cells. All mice treated with CS1-CAR-T cells (n=10) lived >90 days (FIG. 11B) while median survival of CD19-control mice (n=8) was 43 days. We treated mice engrafted with MM.1S-CGACS1 with CS1-CAR-T or CD19 CAR-T, as above. Survival of those mice was similar to control mice (49 days), demonstrating in vivo specificity. Serial Bioluminescent imaging (BLI) showed CS1-CAR-Ts treated mice had a three log decrease in photon flux and clearance of marrow tumor (FIG. 11C). A subset of CS1-CAR-T mice developed extramedullary tumors that retained expression of CS1, suggesting antigen escape did not occur.

### Example 10—Efficacy and Cell Killing of a Tandem (tCAR) which Targets BCMA and CS1

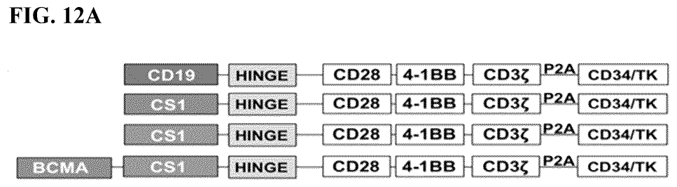

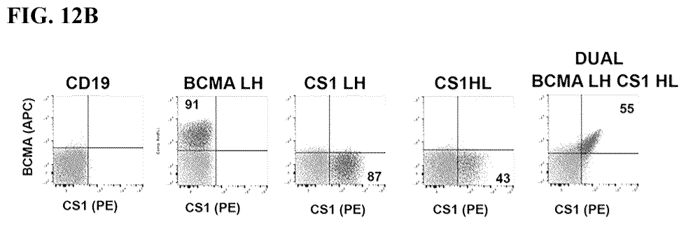

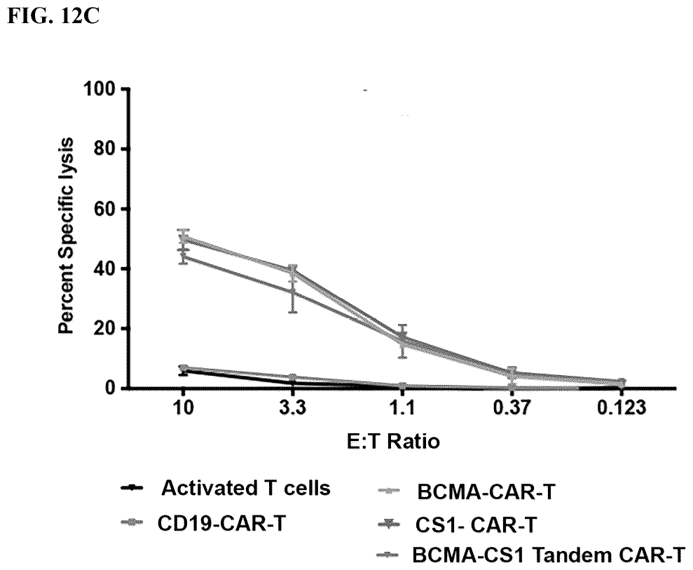

Bi-targeted CAR-T that express two scFvs in a tandem (tCAR) that target BCMA and CS1 were designed in an attempt to improve efficacy and killing of myeloma CAR-T cells. For a control, the tandem CAR was tested side by side with single-targeted BCMA-CAR-T cells and single-targeted CS1-CAR-T cells. CD19-CAR-T cells were used as a negative control. First, each scFv was confirmed to be expressed in the tCAR. To accomplish this, Jurkat cells were infected with lentivirus expressing each CAR construct as described in FIG. 12A. The CAR-T cells were incubated with human recombinant BCMA and CS1 proteins each labeled with separate fluorescent fluorophores. Negative control CAR-T cells were gated (blue color) and the experimental CAR-T cells were overlayed (red color). As expected, Jurkat cells expressing CD19 CAR did not bind to either BCMA or CS1 protein (lower left quadrant, FIG. 12B). Jurkat cells expressing BCMA CAR protein bound BCMA protein (upper left quadrant, FIG. 12B). Jurkat cells expressing CS1 CAR protein bound CS1 protein (lower right quadrant, FIG. 12B). Jurkat cells expressing the tandem BCMA-CS1 CAR protein bound to both recombinant proteins (upper right quadrant, FIG. 12B), suggesting expression of both scFvs.

Single and tandem CAR-T cells were tested for in vitro efficacy with standard four-hour chromium release (⁵¹Cr) assays. For these experiments, CAR-T cells were incubated with a range of effector to target cells (E:T ratio). BCMA-CS1 tCAR T cells killed MM.1S-CG cells with similar efficacy of both single targeted CAR-T cells. Additional experiments will optimize bi-targeted BCMA-CS1 CAR-T cells for in vivo efficacy.

### Example 11—Off Target Analysis for gRNA Selection

Guide RNA were designed and validated for activity by Washington University Genome Engineering & iPSC. Guide RNA were designed and validated for activity by Washington University Genome Engineering & iPSC. Sequences complementary to a given gRNA may exist throughout the genome, including but not limited to the target locus. A short sequence is likelier to hybridize off-target. Similarly, some long sequences within the gRNA may have exact matches (long_0) or near matches (long_1, long_2, representing, respectively, a single or two nucleotide difference) throughout the genome. These may also hybridize off-target, in effect leading to editing of the wrong gene and diminishing editing efficiency.

Off target analysis of selected gRNA was performed for 2 exons of hCD2 (CF58 and CF59) to determine the number of sites in human genome which are an exact match or contains up to 1 or 2 mismatches, which may include the target site. The results are listed in Table 15 for Exon CF58 and Table 16 for Exon CF59.

TABLE 15
Guide RNA (gRNA) Off Target Analysis for hCD2 (Exon CF58)

| | | long_ | long_ | long_ | short_ | |
|---|---|---|---|---|---|---|
| Name | gRNA | 0 | 1 | 2 | 0 | SNP |
| CF58.CD2.g1 | CAAAGAGATTACGAATGCCTN | 1 | 1 | 1 | 3 | NA |
| | GG (SEQ ID NO: 57) | | | | | |
| CF58.CD2.g23 | CAAGGCATTCGTAATCTCTTN | 1 | 1 | 1 | 5 | NA |
| | GG (SEQ ID NO: 58) | | | | | |
| CF58.CD2.g18 | CTTGTAGATATCCTGATCATNG | 1 | 1 | 1 | 13 | NA |
| | G (SEQ ID NO: 59) | | | | | |
| CF58.CD2.g8 | CTTGGGTCAGGACATCAACTNG | 1 | 1 | 1 | 14 | NA |
| | G (SEQ ID NO: 60) | | | | | |
| CF58.CD2.g14 | CGATGATCAGGATATCTACANG | 1 | 1 | 1 | 17 | NA |
| | G (SEQ ID NO: 61) | | | | | |
| CF58.CD2.g2 | TTACGAATGCCTTGGAAACCNG | 1 | 1 | 1 | 27 | NA |
| | G (SEQ ID NO: 62) | | | | | |
| CF58.CD2.g3 | TACGAATGCCTTGGAAACCTNG | 1 | 1 | 1 | 34 | NA |
| | G (SEQ ID NO: 63) | | | | | |
| CF58.CD2.g4 | ACGAATGCCTTGGAAACCTGNG | 1 | 1 | 1 | 40 | NA |
| | G (SEQ ID NO: 64) | | | | | |
| CF58.CD2.g10 | TGATATTGACGATATAAAATNG | 1 | 1 | 2 | 3 | NA |
| | G (SEQ ID NO: 65) | | | | | |
| CF58.CD2.g9 | ATGATATTGACGATATAAAANG | 1 | 1 | 2 | 4 | NA |
| | G (SEQ ID NO: 66) | | | | | |
| CF58.CD2.g13 | GCATCTGAAGACCGATGATCNG | 1 | 1 | 2 | 4 | NA |
| | G (SEQ ID NO: 67) | | | | | |
| CF58.CD2.g7 | AACCTGGGGTGCCTTGGGTCNG | 1 | 1 | 2 | 22 | NA |
| | G (SEQ ID NO: 68) | | | | | |
| CF58.CD2.g6 | TTGGAAACCTGGGGTGCCTTNG | 1 | 1 | 2 | 33 | NA |
| | G (SEQ ID NO: 69) | | | | | |
| CF58.CD2.g15 | GTATCAATATATGATACAAANG | 1 | 1 | 2 | 35 | NA |
| | G (SEQ ID NO: 70) | | | | | |
| CF58.CD2.g22 | CAAGGCACCCCAGGTTTCCANG | 1 | 1 | 2 | 45 | NA |
| | G (SEQ ID NO: 71) | | | | | |
| CF58.CD2.g5 | CTTGGAAACCTGGGGTGCCTNG | 1 | 1 | 2 | 62 | NA |
| | G (SEQ ID NO: 72) | | | | | |
| CF58.CD2.g19 | TCATCACTCATTTGAAAACTNG | 1 | 1 | 3 | 56 | NA |
| | G (SEQ ID NO: 73) | | | | | |
| CF58.CD2.g20 | CAAGTTGATGTCCTGACCCANG | 1 | 1 | 4 | 27 | NA |
| | G (SEQ ID NO: 74) | | | | | |
| CF58.CD2.g21 | GTCCTGACCCAAGGCACCCCNG | 1 | 1 | 4 | 33 | NA |
| | G (SEQ ID NO: 75) | | | | | |
| CF58.CD2.g17 | ATATTTGATTTGAAGATTCANG | 1 | 1 | 6 | 35 | NA |
| | G (SEQ ID NO: 76) | | | | | |
| CF58.CD2.g16 | TACAAAAGGAAAAAATGTGTN | 1 | 1 | 7 | 64 | NA |
| | GG (SEQ ID NO: 77) | | | | | |
| CF58.CD2.g12 | ACATATAAGCTATTTAAAAANG | 1 | 1 | 8 | 58 | NA |
| | G (SEQ ID NO: 78) | | | | | |
| CF58.CD2.g11 | AAAAGAGAAAGAGACTTTCAN | 1 | 1 | 15 | 42 | NA |
| | GG (SEQ ID NO: 79) | | | | | |

TABLE 16
Guide RNA (gRNA) Off Target Analysis for hCD2 (CF59)

| | | long_ | long_ | long_ | short_ | |
|---|---|---|---|---|---|---|
| Name | gRNA | 0 | 1 | 2 | 0 | SNP |
| CF59.CD2.g20 | CTTGATACAGGTTTAATTCG | 1 | 1 | 1 | 2 | NA |
| | NGG (SEQ ID NO: 80) | | | | | |
| CF59.CD2.g13 | ACAGCTGACAGGCTCGACAC | 1 | 1 | 1 | 4 | NA |
| | NGG (SEQ ID NO: 81) | | | | | |
| CF59.CD2.g17 | GATGTTTCCCATCTTGATAC | 1 | 1 | 1 | 8 | NA |
| | NGG (SEQ ID NO: 82) | | | | | |
| CF59.CD2.g12 | GTCGAGCCTGTCAGCTGTCCN | 1 | 1 | 1 | 24 | NA |
| | GG (SEQ ID NO: 83) | | | | | |
| CF59.CD2.g10 | CAAAATTCAAGTGCACAGCAN | 1 | 1 | 1 | 33 | NA |
| | GG (SEQ ID NO: 84) | | | | | |
| CF59.CD2.g16 | GAATTTTGCACTCAGGCTGGN | 1 | 1 | 1 | 245 | NA |
| | GG (SEQ ID NO: 85) | | | | | |
| CF59.CD2.g4 | GAATTAAACCTGTATCAAGAN | 1 | 1 | 2 | 7 | NA |
| | GG (SEQ ID NO: 86) | | | | | |
| CF59.CD2.g5 | AATTAAACCTGTATCAAGATN | 1 | 1 | 2 | 7 | NA |
| | GG (SEQ ID NO: 87) | | | | | |
| CF59.CD2.g21 | AGTTCCATTCATTACCTCACNG | 1 | 1 | 2 | 14 | NA |
| | G (SEQ ID NO: 88) | | | | | |
| CF59.CD2.g8 | AGAGGGTCATCACACACAAGN | 1 | 1 | 2 | 20 | NA |
| | GG (SEQ ID NO: 89) | | | | | |
| CF59.CD2.g25 | ATACAAGTCCAGGAGATCTTN | 1 | 1 | 2 | 21 | NA |
| | GG (SEQ ID NO: 90) | | | | | |
| CF59.CD2.g19 | TCTTGATACAGGTTTAATTCNG | 1 | 1 | 2 | 25 | NA |
| | G (SEQ ID NO: 91) | | | | | |
| CF59.CD2.g3 | CTGACCTGTGAGGTAATGAAN | 1 | 1 | 2 | 29 | NA |
| | GG (SEQ ID NO: 92) | | | | | |
| CF59.CD2.g7 | ACATCTAAAACTTTCTCAGAN | 1 | 1 | 2 | 41 | NA |
| | GG (SEQ ID NO: 93) | | | | | |
| CF59.CD2.g9 | GCAAAATTCAAGTGCACAGCN | 1 | 1 | 2 | 46 | NA |
| | GG (SEQ ID NO: 94) | | | | | |
| CF59.CD2.g24 | GGTTGTGTTGATACAAGTCCN | 1 | 1 | 3 | 8 | NA |
| | GG (SEQ ID NO: 95) | | | | | |
| CF59.CD2.g18 | ATCTTGATACAGGTTTAATTNG | 1 | 1 | 3 | 24 | NA |
| | G (SEQ ID NO: 96) | | | | | |
| CF59.CD2.g23 | ATTCATTACCTCACAGGTCAN | 1 | 1 | 3 | 35 | NA |
| | GG (SEQ ID NO: 97) | | | | | |
| CF59.CD2.g6 | AACATCTAAAACTTTCTCAGN | 1 | 1 | 3 | 43 | NA |
| | GG (SEQ ID NO: 98) | | | | | |
| CF59.CD2.g11 | AGCAGGGAACAAAGTCAGCAN | 1 | 1 | 3 | 45 | NA |
| | GG (SEQ ID NO: 99) | | | | | |
| CF59.CD2.g2 | CAACACAACCCTGACCTGTGN | 1 | 1 | 3 | 47 | NA |
| | GG (SEQ ID NO: 100) | | | | | |
| CF59.CD2.g15 | CTTGAATTTTGCACTCAGGCNG | 1 | 1 | 4 | 21 | NA |
| | G (SEQ ID NO: 101) | | | | | |
| CF59.CD2.g22 | CATTCATTACCTCACAGGTCNG | 1 | 1 | 10 | 29 | NA |
| | G (SEQ ID NO: 102) | | | | | |
| CF59.CD2.g14 | TGCACTTGAATTTTGCACTCNG | 1 | 2 | 3 | 26 | NA |
| | G (SEQ ID NO: 103) | | | | | |
| CF59.CD2.g1 | TCTCAAAACCAAAGATCTCCN | 1 | 2 | 5 | 19 | NA |
| | GG (SEQ ID NO: 104) | | | | | |

The gRNA sequences in Table 15 and Table 16 were normalized (% Normalization to NHEJ) for gRNA activity via next generation sequencing (NGS). GFP was used as a control. Following sequencing analysis, the following gRNAs were recommended based on off-target profile: CF58.CD2.g1 (41.2%), CF58.CD2.g23 (13.2%), CF59.CD2.g20 (26.6%), CF59.CD2.g13 (66.2%), CF59.CD2.g17 (17.5%). Guide RNA (gRNA) with normalized NHEJ frequencies equal to or greater than 15% are good candidates for cell line and animal model creation projects.

Off target analysis of selected gRNA was performed for hCD3E to determine the number of sites in human genome which are an exact match or contains up to 1 or 2 mismatches, which may include the target site. The results are listed in Table 17 for hCD3E.

TABLE 17
Guide RNA (gRNA) Off Target Analysis for hCD3E

| | | long_ | long_ | long_ | long_ | short_ | |
|---|---|---|---|---|---|---|---|
| Name | gRNA | 0 | 1 | 2 | 3 | 0 | SNP |
| MS1044.CD3E. | TTGACATGCCCTCAGTATC | 1 | 1 | 1 | 21 | 73 | NA |
| sp2 | CNGG (SEQ ID NO: 105) | | | | | | |
| MS1044.CD3E. | CTGGATTACCTCTTGCCCT | 1 | 1 | 1 | 24 | 114 | NA |
| sp17 | CNGG (SEQ ID NO: 106) | | | | | | |
| MS1044.CD3E. | GAGATGGAGACTTTATA | 1 | 1 | 1 | 30 | 44 | NA |
| sp28 | TGCNGG (SEQ ID NO: 107) | | | | | | |
| MS1044.CD3E. | AGATGGAGACTTTATATG | 1 | 1 | 1 | 33 | 55 | NA |
| sp29 | CTNGG (SEQ ID NO: 108) | | | | | | |
| MS1044.CD3E. | AGGGCATGTCAATATTAC | 1 | 1 | 1 | 23 | 60 | NA |
| sp26 | TGNGG (SEQ ID NO: 109) | | | | | | |
| MS1044.CD3E. | GATGGAGACTTTATATGC | 1 | 1 | 2 | 26 | 64 | NA |
| sp30 | TGNGG (SEQ ID NO: 110) | | | | | | |
| MS1044.CD3E. | TATTATGTCTGCTACCCC | 1 | 1 | 2 | 20 | 61 | NA |
| sp12 | AGNGG (SEQ ID NO: 111) | | | | | | |
| MS1044.CD3E. | TGCCATAGTATTTCAGATC | 1 | 1 | 2 | 21 | 55 | NA |
| sp23 | CNGG (SEQ ID NO: 112) | | | | | | |
| MS1044.CD3E. | AGATAAAAGTTCGCATCT | 1 | 1 | 2 | 33 | 6 | NA |
| sp18 | TCNGG (SEQ ID NO: 113) | | | | | | |
| MS1044.CD3E. | CTGAAAATTCCTTCAGTG | 1 | 1 | 2 | 44 | 60 | NA |
| sp22 | ACNGG (SEQ ID NO: 114) | | | | | | |
| MS1044.CD3E. | CTGAGGGCAAGAGGTAAT | 1 | 1 | 3 | 30 | 41 | NA |
| sp16 | CCNGG (SEQ ID NO: 115) | | | | | | |
| MS1044.CD3E. | TTTCAGATCCAGGATACT | 1 | 1 | 3 | 38 | 63 | NA |
| sp25 | GANGG (SEQ ID NO: 116) | | | | | | |
| MS1044.CD3E. | TATCTCTACCTGAGGGCA | 1 | 1 | 3 | 22 | 134 | NA |
| sp15 | AGNGG (SEQ ID NO: 117) | | | | | | |
| MS1044.CD3E. | TGAGGATCACCTGTCACT | 1 | 1 | 3 | 44 | 54 | NA |
| sp9 | GANGG (SEQ ID NO: 118) | | | | | | |

The gRNA sequences in Table 17 were normalized (% Normalization to NHEJ) for gRNA activity via next generation sequencing (NGS). GFP was used as a control. Following sequencing analysis, the following gRNAs were recommended based on off-target profile: MS1044.CD3E.sp28 (>15%) and MS1044.CD3E.sp12 (>15%). Guide RNA (gRNA) with normalized NHEJ frequencies equal to or greater than 15% are good candidates for cell line and animal model creation projects.

Off target analysis of selected gRNA was performed for 3 exons of hCD5 (Exon 3, Exon 4, and Exon 5) to determine the number of sites in human genome which are an exact match or contains up to 1 or 2 mismatches, which may include the target site. The results are listed in Table 18 for Exon 3, Table 19 for Exon 4, and Table 20 for Exon 5.

TABLE 18
Guide RNA (gRNA) Off Target Analysis for hCD5 (Exon 3)

| | | long_ | long_ | long_ | short_ | |
|---|---|---|---|---|---|---|
| Name | gRNA | 0 | 1 | 2 | 0 | SNP |
| SP597.CD5. | AATCATCTGCTACGGACAAC | 1 | 1 | 1 | 1 | NA |
| g22 | NGG (SEQ ID NO: 119) | | | | | |
| SP597.CD5. | GCAGACTTTTGACGCTTGAC | 1 | 1 | 1 | 1 | NA |
| g39 | NGG (SEQ ID NO: 120) | | | | | |
| SP597.CD5. | CCGTTCCAACTCGAAGTGCCN | 1 | 1 | 1 | 2 | NA |
| g1 | GG (SEQ ID NO: 121) | | | | | |
| SP597.CD5. | CGTTCCAACTCGAAGTGCCA | 1 | 1 | 1 | 2 | NA |
| g2 | NGG (SEQ ID NO: 122) | | | | | |
| SP597.CD5. | CTGGCACTTCGAGTTGGAACN | 1 | 1 | 1 | 2 | NA |
| g50 | GG (SEQ ID NO: 123) | | | | | |
| SP597.CD5. | GTCTGCCAGCGGCTGAACTGN | 1 | 1 | 1 | 3 | NA |
| g17 | GG (SEQ ID NO: 124) | | | | | |
| SP597.CD5. | ATCATCTGCTACGGACAACTN | 1 | 1 | 1 | 3 | NA |
| g23 | GG (SEQ ID NO: 125) | | | | | |
| SP597.CD5. | AGACTTTTGACGCTTGACTGN | 1 | 1 | 1 | 3 | NA |
| g41 | GG (SEQ ID NO: 126) | | | | | |
| SP597.CD5. | CAGACTTTTGACGCTTGACTN | 1 | 1 | 1 | 5 | NA |
| g40 | GG (SEQ ID NO: 127) | | | | | |
| SP597.CD5. | CCTGGCACTTCGAGTTGGAAN | 1 | 1 | 1 | 5 | NA |
| g49 | GG (SEQ ID NO: 128) | | | | | |
| SP597.CD5. | GCACCCCACAGTTCAGCCGCN | 1 | 1 | 1 | 8 | NA |
| g38 | GG (SEQ ID NO: 129) | | | | | |
| SP597.CD5. | CCTTGAGGTAGACCTCCAG | 1 | 1 | 1 | 9 | NA |
| g46 | CNGG (SEQ ID NO: 130) | | | | | |
| SP597.CD5. | AGGTCTACCTCAAGGACGGA | 1 | 1 | 1 | 11 | NA |
| g7 | NGG (SEQ ID NO: 131) | | | | | |
| SP597.CD5. | TGGAACGGGTGAGCCTTGCCN | 1 | 1 | 1 | 13 | NA |
| g51 | GG (SEQ ID NO: 132) | | | | | |
| SP597.CD5. | TGTGGGGTGCCCTTAAGCCTN | 1 | 1 | 1 | 19 | NA |
| g20 | GG (SEQ ID NO: 133) | | | | | |
| SP597.CD5. | AAGCGTCAAAAGTCTGCCAG | 1 | 1 | 1 | 20 | NA |
| g16 | NGG (SEQ ID NO: 134) | | | | | |
| SP597.CD5. | TAGCAGATGATTGAGCTCTGN | 1 | 1 | 1 | 25 | NA |
| g29 | GG (SEQ ID NO: 135) | | | | | |
| SP597.CD5. | GATTGAGCTCTGAGGTGTGTN | 1 | 1 | 1 | 33 | NA |
| g30 | GG (SEQ ID NO: 136) | | | | | |
| SP597.CD5. | GGGGCCGGAGCTCCAAGCAG | 1 | 1 | 1 | 42 | NA |
| g13 | NGG (SEQ ID NO: 137) | | | | | |
| SP597.CD5. | GGTGTGTAGGTGACAAGGAA | 1 | 1 | 1 | 48 | NA |
| g33 | NGG (SEQ ID NO: 138) | | | | | |
| SP597.CD5. | CCGGAGCTCCAAGCAGTGGG | 1 | 1 | 1 | 58 | NA |
| g15 | NGG (SEQ ID NO: 139) | | | | | |
| SP597.CD5. | GGTAGACCTCCAGCTGGCCCN | 1 | 1 | 1 | 78 | NA |
| g47 | GG (SEQ ID NO: 140) | | | | | |
| SP597.CD5. | CTCGAAGTGCCAGGGCCAGC | 1 | 1 | 1 | 121 | NA |
| g3 | NGG (SEQ ID NO: 141) | | | | | |
| SP597.CD5. | CTGGCCCTGGCACTTCGAGTN | 1 | 1 | 2 | 1 | NA |
| g48 | GG (SEQ ID NO: 142) | | | | | |
| SP597.CD5. | TCTGCCAGCGGCTGAACTGTN | 1 | 1 | 2 | 5 | NA |
| g18 | GG (SEQ ID NO: 143) | | | | | |
| SP597.CD5. | CCATGTGCCATCCGTCCTTGN | 1 | 1 | 2 | 5 | NA |
| g45 | GG (SEQ ID NO: 144) | | | | | |
| SP597.CD5. | CCAGCTGGAGGTCTACCTCAN | 1 | 1 | 2 | 14 | NA |
| g5 | GG (SEQ ID NO: 145) | | | | | |
| SP597.CD5. | TCTGAGGTGTGTAGGTGACAN | 1 | 1 | 2 | 18 | NA |
| g31 | GG (SEQ ID NO: 146) | | | | | |
| SP597.CD5. | AGGAAGGGGCCAAGGCTTAA | 1 | 1 | 2 | 18 | NA |
| g37 | NGG (SEQ ID NO: 147) | | | | | |
| SP597.CD5. | CAGAGCTCAATCATCTGCTAN | 1 | 1 | 2 | 19 | NA |
| g21 | GG (SEQ ID NO: 148) | | | | | |
| SP597.CD5. | GGGCCGGAGCTCCAAGCAGT | 1 | 1 | 2 | 23 | NA |
| g14 | NGG (SEQ ID NO: 149) | | | | | |
| SP597.CD5. | CCTCCCACTGCTTGGAGCTCN | 1 | 1 | 2 | 30 | NA |
| g43 | GG (SEQ ID NO: 150) | | | | | |
| SP597.CD5. | TGGAGCTCCGGCCCCAGCTCN | 1 | 1 | 2 | 38 | NA |
| g44 | GG (SEQ ID NO: 151) | | | | | |
| SP597.CD5. | GTGTGTAGGTGACAAGGAAG | 1 | 1 | 2 | 48 | NA |
| g34 | NGG (SEQ ID NO: 152) | | | | | |
| SP597.CD5. | ATGGTTTGCAGCCAGAGCTGN | 1 | 1 | 2 | 108 | NA |
| g11 | GG (SEQ ID NO: 153) | | | | | |
| SP597.CD5. | CTGGAGGTCTACCTCAAGGAN | 1 | 1 | 3 | 16 | NA |
| g6 | GG (SEQ ID NO: 154) | | | | | |
| SP597.CD5. | CTGCCAGCGGCTGAACTGTGN | 1 | 1 | 3 | 25 | NA |
| g19 | GG (SEQ ID NO: 155) | | | | | |
| SP597.CD5. | AATGACATGTGTCACTCTCTN | 1 | 1 | 3 | 25 | NA |
| g25 | GG (SEQ ID NO: 156) | | | | | |
| SP597.CD5. | ACATGGTTTGCAGCCAGAGCN | 1 | 1 | 3 | 30 | NA |
| g9 | GG (SEQ ID NO: 157) | | | | | |
| SP597.CD5. | CATGGTTTGCAGCCAGAGCTN | 1 | 1 | 3 | 52 | NA |
| g10 | GG (SEQ ID NO: 158) | | | | | |
| SP597.CD5. | GACACATGTCATTTCTGCTGN | 1 | 1 | 3 | 53 | NA |
| g26 | GG (SEQ ID NO: 159) | | | | | |
| SP597.CD5. | ACTGGGGTCCTCCCACTGCTN | 1 | 1 | 3 | 91 | NA |
| g42 | GG (SEQ ID NO: 160) | | | | | |
| SP597.CD5. | CCTCAAGGACGGATGGCACA | 1 | 1 | 4 | 5 | NA |
| g8 | NGG (SEQ ID NO: 161) | | | | | |
| SP597.CD5. | AGGTGTGTAGGTGACAAGGA | 1 | 1 | 4 | 49 | NA |
| g32 | NGG (SEQ ID NO: 162) | | | | | |
| SP597.CD5. | AAGGAAGGGGCCAAGGCTTA | 1 | 1 | 5 | 16 | NA |
| g36 | NGG (SEQ ID NO: 163) | | | | | |
| SP597.CD5. | GAAGTGCCAGGGCCAGCTGG | 1 | 1 | 5 | 93 | NA |
| g4 | NGG (SEQ ID NO: 164) | | | | | |
| SP597.CD5. | TTTGCAGCCAGAGCTGGGGCN | 1 | 1 | 8 | 257 | NA |
| g12 | GG (SEQ ID NO: 165) | | | | | |
| SP597.CD5. | AAATGACATGTGTCACTCTCN | 1 | 1 | 10 | 33 | NA |
| g24 | GG (SEQ ID NO: 166) | | | | | |
| SP597.CD5. | AGGTGACAAGGAAGGGGCCA | 1 | 1 | 10 | 202 | NA |
| g35 | NGG (SEQ ID NO: 167) | | | | | |
| SP597.CD5. | ATTTCTGCTGTGGCTGCAGTN | 1 | 2 | 4 | 70 | NA |
| g27 | GG (SEQ ID NO: 168) | | | | | |
| SP597.CD5. | GCTGTGGCTGCAGTTGGAGAN | 1 | 2 | 19 | 49 | NA |
| g28 | GG (SEQ ID NO: 169) | | | | | |

TABLE 19
Guide RNA (gRNA) Off Target Analysis for hCD5 (Exon 4)

| | | long_ | long_ | long_ | short_ | |
|---|---|---|---|---|---|---|
| Name | gRNA | 0 | 1 | 2 | 0 | SNP |
| SP598.CD5. | GGCGGGGGCCTTGTCGTTG | 1 | 1 | 1 | 1 | NA |
| g10 | GNGG (SEQ ID NO: 170) | | | | | |
| SP598.CD5. | CTCTGGAGTTGTGGTGGGC | 1 | 1 | 1 | 16 | NA |
| g7 | GNGG (SEQ ID NO: 171) | | | | | |
| SP598.CD5. | TCTGGAGTTGTGGTGGGCGGN | 1 | 1 | 1 | 40 | NA |
| g8 | GG (SEQ ID NO: 172) | | | | | |
| SP598.CD5. | CGTTGGAGGTGTTGTCTTCTN | 1 | 1 | 1 | 46 | NA |
| g12 | GG (SEQ ID NO: 173) | | | | | |
| SP598.CD5. | AGACAACACCTCCAACGACAN | 1 | 1 | 2 | 2 | NA |
| g1 | GG (SEQ ID NO: 174) | | | | | |
| SP598.CD5. | GTGGGCGGGGGCCTTGTCGTN | 1 | 1 | 2 | 5 | NA |
| g9 | GG (SEQ ID NO: 175) | | | | | |
| SP598.CD5. | TCGTTGGAGGTGTTGTCTTCN | 1 | 1 | 2 | 13 | NA |
| g11 | GG (SEQ ID NO: 176) | | | | | |
| SP598.CD5. | ACCACAACTCCAGAGCCCACN | 1 | 1 | 2 | 60 | NA |
| g2 | GG (SEQ ID NO: 177) | | | | | |
| SP598.CD5. | GCTCTGGAGTTGTGGTGGGCN | 1 | 1 | 4 | 74 | NA |
| g6 | GG (SEQ ID NO: 178) | | | | | |
| SP598.CD5. | GTGGGCTCTGGAGTTGTGGTN | 1 | 1 | 6 | 35 | NA |
| g4 | GG (SEQ ID NO: 179) | | | | | |
| SP598.CD5. | TGTGGGCTCTGGAGTTGTGGN | 1 | 1 | 8 | 54 | NA |
| g3 | GG (SEQ ID NO: 180) | | | | | |
| SP598.CD5. | GTTGGAGGTGTTGTCTTCTGN | 1 | 2 | 2 | 48 | NA |
| g13 | GG (SEQ ID NO: 181) | | | | | |
| SP598.CD5. | GGCTCTGGAGTTGTGGTGGGN | 1 | 3 | 9 | 51 | NA |
| g5 | GG (SEQ ID NO: 182) | | | | | |

TABLE 20
Guide RNA (gRNA) Off Target Analysis for hCD5 (Exon 5)

| | | long_ | long_ | long_ | short_ | |
|---|---|---|---|---|---|---|
| Name | gRNA | 0 | 1 | 2 | 0 | SNP |
| SP599.CD5. | CATAGCTGATGGTACCCC | 1 | 1 | 1 | 1 | NA |
| g58 | CCNGG (SEQ ID NO: 183) | | | | | |
| SP599.CD5. | CGGCCAGCACTGTGCCGG | 1 | 1 | 1 | 2 | NA |
| g5 | CGNGG (SEQ ID NO: 184) | | | | | |
| SP599.CD5. | CAAGAACTCGGCCACTTT | 1 | 1 | 1 | 6 | NA |
| g30 | TCNGG (SEQ ID NO: 185) | | | | | |
| SP599.CD5. | GGTGTTCCCGTGGCTCCCCT | 1 | 1 | 1 | 11 | rs2241002: |
| g44 | NGG (SEQ ID NO: 186) | | | | | 0.158 |
| SP599.CD5. | CCAGCACTGTGCCGGCGTG | 1 | 1 | 1 | 13 | NA |
| g6 | GNGG (SEQ ID NO: 187) | | | | | |
| SP599.CD5. | GGCAAGGGCTGGTGTTCC | 1 | 1 | 1 | 13 | NA |
| g42 | CGNGG (SEQ ID NO: 188) | | | | | |
| SP599.CD5. | GGCGTGGTGGAGTTCTACA | 1 | 1 | 1 | 14 | NA |
| g7 | GNGG (SEQ ID NO: 189) | | | | | |
| SP599.CD5. | CCACCACGCCGGCACAGTG | 1 | 1 | 1 | 15 | NA |
| g60 | CNGG (SEQ ID NO: 190) | | | | | |
| SP599.CD5. | GGAGTTCTACAGCGGCAGC | 1 | 1 | 1 | 17 | NA |
| g8 | CNGG (SEQ ID NO: 191) | | | | | |
| SP599.CD5. | GTTCTACAGCGGCAGCCTG | 1 | 1 | 1 | 18 | NA |
| g11 | GNGG (SEQ ID NO: 192) | | | | | |
| SP599.CD5. | ACCAGCCCTTGCCAATCCA | 1 | 1 | 1 | 20 | NA |
| g25 | ANGG (SEQ ID NO: 193) | | | | | |
| SP599.CD5. | AGTTCTACAGCGGCAGCCT | 1 | 1 | 1 | 24 | NA |
| g10 | GNGG (SEQ ID NO: 194) | | | | | |
| SP599.CD5. | CCAGGTCCTGGGTCTTGTC | 1 | 1 | 1 | 25 | NA |
| g55 | CNGG (SEQ ID NO: 195) | | | | | |
| SP599.CD5. | TGGTGTTCCCGTGGCTCCCC | 1 | 1 | 1 | 25 | rs2241002: |
| g43 | NGG (SEQ ID NO: 196) | | | | | 0.158 |
| SP599.CD5. | GAGTTCTACAGCGGCAGCC | 1 | 1 | 1 | 26 | NA |
| g9 | TNGG (SEQ ID NO: 197) | | | | | |
| SP599.CD5. | GAACTCAAGCTGTACCTCC | 1 | 1 | 1 | 29 | NA |
| g26 | CNGG (SEQ ID NO: 198) | | | | | |
| SP599.CD5. | AAGAACTCGGCCACTTTTC | 1 | 1 | 1 | 29 | NA |
| g31 | TNGG (SEQ ID NO: 199) | | | | | |
| SP599.CD5. | TCCATTGGATTGGCAAGGG | 1 | 1 | 1 | 32 | NA |
| g41 | CNGG (SEQ ID NO: 200) | | | | | |
| SP599.CD5. | TTCTACAGCGGCAGCCTGG | 1 | 1 | 1 | 33 | NA |
| g12 | GNGG (SEQ ID NO: 201) | | | | | |
| SP599.CD5. | AGAACTCGGCCACTTTTCT | 1 | 1 | 1 | 37 | NA |
| g32 | GNGG (SEQ ID NO: 202) | | | | | |
| SP599.CD5. | GCTTCAAGAAGGAGCCACA | 1 | 1 | 1 | 48 | NA |
| g49 | CNGG (SEQ ID NO: 203) | | | | | |
| SP599.CD5. | GATCTTCCATTGGATTGGC | 1 | 1 | 2 | 7 | NA |
| g39 | ANGG (SEQ ID NO: 204) | | | | | |
| SP599.CD5. | GCTGTAGAACTCCACCACG | 1 | 1 | 2 | 11 | NA |
| g59 | CNGG (SEQ ID NO: 205) | | | | | |
| SP599.CD5. | GTCCTGGGCCTCATAGCTG | 1 | 1 | 2 | 13 | NA |
| g57 | ANGG (SEQ ID NO: 206) | | | | | |
| SP599.CD5. | TACCATCAGCTATGAGGCC | 1 | 1 | 2 | 14 | NA |
| g14 | CNGG (SEQ ID NO: 207) | | | | | |
| SP599.CD5. | GGGGGGTACCATCAGCTAT | 1 | 1 | 2 | 16 | NA |
| g13 | GNGG (SEQ ID NO: 208) | | | | | |
| SP599.CD5. | CCTGAAGCAATGCTCCAGG | 1 | 1 | 2 | 18 | NA |
| g35 | GNGG (SEQ ID NO: 209) | | | | | |
| SP599.CD5. | TTTTCCTGAAGCAATGCTCC | 1 | 1 | 2 | 24 | NA |
| g33 | NGG (SEQ ID NO: 210) | | | | | |
| SP599.CD5. | CTCTGGCAGATGCTTCAAG | 1 | 1 | 2 | 25 | NA |
| g48 | ANGG (SEQ ID NO: 211) | | | | | |
| SP599.CD5. | AGAGGAAGTTCTCCAGGTC | 1 | 1 | 2 | 53 | NA |
| g53 | CNGG (SEQ ID NO: 212) | | | | | |
| SP599.CD5. | TCTGGCGGCCAGCACTGTG | 1 | 1 | 2 | 166 | NA |
| g4 | CNGG (SEQ ID NO: 213) | | | | | |
| SP599.CD5. | TTGAGTTCTGGATCTTCCAT | 1 | 1 | 3 | 9 | NA |
| g37 | NGG (SEQ ID NO: 214) | | | | | |
| SP599.CD5. | TTCTGGATCTTCCATTGGAT | 1 | 1 | 3 | 13 | NA |
| g38 | NGG (SEQ ID NO: 215) | | | | | |
| SP599.CD5. | ATCTTCCATTGGATTGGCA | 1 | 1 | 3 | 18 | NA |
| g40 | ANGG (SEQ ID NO: 216) | | | | | |
| SP599.CD5. | TCAAGAAGGAGCCACACTG | 1 | 1 | 3 | 31 | NA |
| g50 | GNGG (SEQ ID NO: 217) | | | | | |
| SP599.CD5. | GGGAGGTACAGCTTGAGTT | 1 | 1 | 3 | 37 | NA |
| g36 | CNGG (SEQ ID NO: 218) | | | | | |
| SP599.CD5. | CCCGTGGCTCCCCTGGGTC | 1 | 1 | 3 | 43 | rs2241002: |
| g45 | TNGG (SEQ ID NO: 219) | | | | | 0.158 |
| SP599.CD5. | CCAGGACAAGACCCAGGAC | 1 | 1 | 3 | 57 | NA |
| g16 | CNGG (SEQ ID NO: 220) | | | | | |
| SP599.CD5. | CTCTGCAACAACCTCCAGT | 1 | 1 | 3 | 67 | NA |
| g17 | GNGG (SEQ ID NO: 221) | | | | | |
| SP599.CD5. | TGTTGCAGAGGAAGTTCTC | 1 | 1 | 3 | 236 | NA |
| g52 | CNGG (SEQ ID NO: 222) | | | | | |
| SP599.CD5. | CAGGTCCTGGGTCTTGTCCT | 1 | 1 | 4 | 24 | NA |
| g56 | NGG (SEQ ID NO: 223) | | | | | |
| SP599.CD5. | TGAGGCCCAGGACAAGACC | 1 | 1 | 4 | 30 | NA |
| g15 | CNGG (SEQ ID NO: 224) | | | | | |
| SP599.CD5. | CTGTGCCACCAGCTGCAGC | 1 | 1 | 4 | 133 | NA |
| g61 | CNGG (SEQ ID NO: 225) | | | | | |
| SP599.CD5. | TGTGCCACCAGCTGCAGCC | 1 | 1 | 4 | 139 | NA |
| g62 | TNGG (SEQ ID NO: 226) | | | | | |
| SP599.CD5. | CATCTGCCAGAGACTGAGG | 1 | 1 | 4 | 1253 | NA |
| g19 | CNGG (SEQ ID NO: 227) | | | | | |
| SP599.CD5. | CTGCAGCTGGTGGCACAGT | 1 | 1 | 5 | 17 | NA |
| g2 | CNGG (SEQ ID NO: 228) | | | | | |
| SP599.CD5. | CACACTGGAGGTTGTTGCA | 1 | 1 | 5 | 28 | NA |
| g51 | GNGG (SEQ ID NO: 229) | | | | | |
| SP599.CD5. | CAGCTGGTGGCACAGTCTG | 1 | 1 | 5 | 31 | NA |
| g3 | GNGG (SEQ ID NO: 230) | | | | | |
| SP599.CD5. | AGCAAAGGAGGGCAAGAA | 1 | 1 | 6 | 53 | NA |
| g29 | CTNGG (SEQ ID NO: 231) | | | | | |
| SP599.CD5. | GAGGAAGTTCTCCAGGTCC | 1 | 1 | 6 | 53 | NA |
| g54 | TNGG (SEQ ID NO: 232) | | | | | |
| SP599.CD5. | GCCACCAGCTGCAGCCTGG | 1 | 1 | 6 | 287 | NA |
| g63 | GNGG (SEQ ID NO: 233) | | | | | |
| SP599.CD5. | GCAGGCAGAGCCCAAGACC | 1 | 1 | 7 | 40 | rs2241002: |
| g20 | CNGG (SEQ ID NO: 234) | | | | | 0.158 |
| SP599.CD5. | CAGGCAGAGCCCAAGACCC | 1 | 1 | 8 | 45 | rs2241002: |
| g21 | ANGG (SEQ ID NO: 235) | | | | | 0.158 |
| SP599.CD5. | TCCTCCCAGGCTGCAGCTG | 1 | 1 | 8 | 140 | NA |
| g1 | GNGG (SEQ ID NO: 236) | | | | | |
| SP599.CD5. | GCTCTGCCTGCCTCAGTCTC | 1 | 1 | 26 | 412 | NA |
| g47 | NGG (SEQ ID NO: 237) | | | | | |
| SP599.CD5. | CCTCCCTGGAGCATTGCTTC | 1 | 2 | 3 | 22 | NA |
| g27 | NGG (SEQ ID NO: 238) | | | | | |
| SP599.CD5. | TTTCCTGAAGCAATGCTCC | 1 | 2 | 4 | 32 | NA |
| g34 | ANGG (SEQ ID NO: 239) | | | | | |
| SP599.CD5. | CCGTGGCTCCCCTGGGTCTT | 1 | 2 | 5 | 37 | rs2241002: |
| g46 | NGG (SEQ ID NO: 240) | | | | | 0.158 |
| SP599.CD5. | AAAATCAAGCCCCAGAAAA | 1 | 2 | 5 | 60 | NA |
| g28 | GNGG (SEQ ID NO: 241) | | | | | |
| SP599.CD5. | GAAGCATCTGCCAGAGACT | 1 | 2 | 7 | 98 | NA |
| g18 | GNGG (SEQ ID NO: 242) | | | | | |
| SP599.CD5. | CCAAGACCCAGGGGAGCCA | 1 | 2 | 8 | 56 | rs2241002: |
| g24 | CNGG (SEQ ID NO: 243) | | | | | 0.158 |
| SP599.CD5. | AGGCAGAGCCCAAGACCCA | 1 | 2 | 10 | 41 | rs2241002: |
| g22 | GNGG (SEQ ID NO: 244) | | | | | 0.158 |
| SP599.CD5. | CCCAAGACCCAGGGGAGCC | 1 | 2 | 10 | 99 | rs2241002: |
| g23 | ANGG (SEQ ID NO: 245) | | | | | 0.158 |

The gRNA sequences in Table 18, Table 19, and Table 20 were normalized (% Normalization to NHEJ) for gRNA activity via next generation sequencing (NGS). GFP was used as a control. Following sequencing analysis, the following gRNAs were recommended based on off-target profile: Exon 3: SP597.hCD5.g2 (76.5%), SP597.hCD5.g22 (36.3%), SP597.hCD5.g39 (16.0%), SP597.hCD5.g46. Exon4: SP598.hCD5.g7, SP598.hCD5.g10 (58.5%). Exon5: SP599.hCD5.g5 (51.0%), SP599.hCD5.g30, SP599.hCD5.g42, SP599.hCD5.g58 (41.0%)

Off target analysis of selected gRNA was performed for hCSF2 to determine the number of sites in human genome which are an exact match or contains up to 1 or 2 mismatches, which may include the target site. The results are listed in Table 21 for hCSF2.

TABLE 21
Guide RNA (gRNA) Off Target Analysis for hCSF2

| | | long_ | long_ | long_ | long_ | short_ | |
|---|---|---|---|---|---|---|---|
| Name | gRNA | 0 | 1 | 2 | 3 | 0 | SNP |
| MS1086.CSF2. | TACTCAGGTTCAGGAGA | 1 | 1 | 1 | 10 | 11 | NA |
| sp8 | CGCNGG (SEQ ID NO: 246) | | | | | | |
| MS1086.CSF2. | TCAGGAGACGCCGGGC | 1 | 1 | 1 | 20 | 38 | NA |
| sp10 | CTCCNGG (SEQ ID | | | | | | |
| | NO: 247) | | | | | | |
| MS1086.CSF2. | ACTCAGGTTCAGGAGACG | 1 | 1 | 1 | 20 | 16 | NA |
| sp9 | CCNGG (SEQ ID NO: 248) | | | | | | |
| MS1086.CSF2. | CAGTGTCTCTACTCAGGT | 1 | 1 | 2 | 22 | 29 | NA |
| sp7 | TCNGG (SEQ ID NO: 249) | | | | | | |
| MS1086.CSF2. | ATGCTCCCAGGGCTGCGT | 1 | 1 | 2 | 42 | 34 | rs2069622 |
| sp14 | GCNGG (SEQ ID NO: 250) | | | | | | |
| MS1086.CSF2. | GAGACGCCGGGCCTCCTG | 1 | 1 | 2 | 26 | 146 | NA |
| sp11 | GANGG (SEQ ID NO: 251) | | | | | | |
| MS1086.CSF2. | CAGCAGCAGTGTCTCTAC | 1 | 1 | 3 | 39 | 24 | NA |
| sp6 | TCNGG (SEQ ID NO: 252) | | | | | | |
| MS1086.CSF2. | GATGGCATTCACATGCTC | 1 | 1 | 3 | 28 | 59 | NA |
| sp12 | CCNGG (SEQ ID NO: 253) | | | | | | |
| MS1086.CSF2. | GGAGCATGTGAATGCCAT | 1 | 1 | 3 | 26 | 48 | NA |
| sp2 | CCNGG (SEQ ID NO: 254) | | | | | | |
| MS1086.CSF2. | TAGAGACACTGCTGCTGA | 1 | 1 | 3 | 56 | 168 | NA |
| sp5 | GANGG (SEQ ID NO: 255) | | | | | | |
| MS1086.CSF2. | GCATGTGAATGCCATCCA | 1 | 1 | 3 | 41 | 56 | NA |
| sp3 | GGNGG (SEQ ID NO: 256) | | | | | | |
| MS1086.CSF2. | ATGGCATTCACATGCTCC | 1 | 1 | 4 | 30 | 80 | NA |
| sp13 | CANGG (SEQ ID NO: 257) | | | | | | |
| MS1086.CSF2. | TGAATGCCATCCAGGAGG | 1 | 1 | 5 | 65 | 180 | NA |
| sp4 | CCNGG (SEQ ID NO: 258) | | | | | | |
| MS1086.CSF2. | TGCTCCCAGGGCTGCGTG | 1 | 1 | 6 | 57 | 29 | rs2069622 |
| sp15 | CTNGG (SEQ ID NO: 259) | | | | | | |
| MS1086.CSF2. | CAGCCCCAGCACGCAGCC | 1 | 1 | 15 | 146 | 41 | rs2069622 |
| sp1 | CTNGG (SEQ ID NO: 260) | | | | | | |
| MS1086.CSF2. | GCTCCCAGGGCTGCGTGC | 1 | 2 | 9 | 85 | 37 | rs2069622 |
| sp16 | TGNGG (SEQ ID NO: 261) | | | | | | |

The gRNA sequences in Table 21 were normalized (% Normalization to NHEJ) for gRNA activity via next generation sequencing (NGS). GFP was used as a control. Following sequencing analysis, the following gRNAs were recommended based on off-target profile: MS1086.CSF2.sp8 (>15%) and MS1086.CSF2.sp10 (>15%).

Off target analysis of selected gRNA was performed for 2 exons of hCTLA4 (Exon 1 and Exon 2) to determine the number of sites in human genome which are an exact match or contains up to 1 or 2 mismatches, which may include the target site. The results are listed in Table 22 for Exon 1 and Table 23 for Exon 2 for hCTLA4.

TABLE 22
Guide RNA (gRNA) Off Target Analysis for hCTLA4 (Exon 1)

| | | long_ | long_ | long_ | short_ | |
|---|---|---|---|---|---|---|
| Name | gRNA | 0 | 1 | 2 | 0 | SNP |
| SP621.CTLA4. | CCTTGGATTTCAGCGGC | 1 | 1 | 1 | 5 | NA |
| g2 | ACANGG (SEQ ID NO: 262) | | | | | |
| SP621.CTLA4. | CCTTGTGCCGCTGAAATC | 1 | 1 | 1 | 5 | NA |
| g12 | CANGG (SEQ ID NO: 263) | | | | | |
| SP621.CTLA4. | TGAACCTGGCTACCAGGA | 1 | 1 | 1 | 11 | rs231775: |
| g5 | CCNGG (SEQ ID NO: 264) | | | | | 0.452 |
| SP621.CTLA4. | AGGGCCAGGTCCTGGTAG | 1 | 1 | 3 | 16 | rs231775: |
| g11 | CCNGG (SEQ ID NO: 265) | | | | | 0.452 |
| SP621.CTLA4. | CTCAGCTGAACCTGGCTAC | 1 | 1 | 3 | 17 | rs231775: |
| g4 | CNGG (SEQ ID NO: 266) | | | | | 0.452 |
| SP621.CTLA4. | AGAAAAAACAGGAGAGTG | 1 | 1 | 3 | 39 | NA |
| g8 | CANGG (SEQ ID NO: 267) | | | | | |
| SP621.CTLA4. | GCACAAGGCTCAGCTGAA | 1 | 1 | 4 | 29 | NA |
| g3 | CCNGG (SEQ ID NO: 268) | | | | | |
| SP621.CTLA4. | TGGCTTGCCTTGGATTTCA | 1 | 1 | 6 | 33 | NA |
| g1 | GNGG (SEQ ID NO: 269) | | | | | |
| SP621.CTLA4. | AAACAGGAGAGTGCAGGG | 1 | 1 | 6 | 69 | NA |
| g9 | CCNGG (SEQ ID NO: 270) | | | | | |
| SP621.CTLA4. | GAGAGTGCAGGGCCAGGT | 1 | 1 | 7 | 50 | NA |
| g10 | CCNGG (SEQ ID NO: 271) | | | | | |
| SP621.CTLA4. | GGATGAAGAGAAGAAAAA | 1 | 1 | 8 | 173 | NA |
| g6 | ACNGG (SEQ ID NO: 272) | | | | | |
| SP621.CTLA4. | AAGAAAAAACAGGAGAGT | 1 | 2 | 8 | 33 | NA |
| g7 | GCNGG (SEQ ID NO: 273) | | | | | |

TABLE 23
Guide RNA (gRNA) Off Target Analysis for hCTLA4 (Exon 2)

| | | long_ | long_ | long_ | short_ | |
|---|---|---|---|---|---|---|
| Name | gRNA | 0 | 1 | 2 | 0 | SNP |
| SP622.CTLA4. | CCGGGTGACAGTGCTTCGG | 1 | 1 | 1 | 2 | NA |
| g9 | CNGG (SEQ ID NO: 274) | | | | | |
| SP622.CTLA4. | ACACAAAGCTGGCGATGCC | 1 | 1 | 1 | 4 | NA |
| g33 | TNGG (SEQ ID NO: 275) | | | | | |
| SP622.CTLA4. | CCCTCAGTCCTTGGATAGTG | 1 | 1 | 1 | 8 | NA |
| g21 | NGG (SEQ ID NO: 276) | | | | | |
| SP622.CTLA4. | GTGCGGCAACCTACATGATG | 1 | 1 | 1 | 9 | NA |
| g14 | NGG (SEQ ID NO: 277) | | | | | |
| SP622.CTLA4. | CTGTGCGGCAACCTACATGA | 1 | 1 | 1 | 13 | NA |
| g12 | NGG (SEQ ID NO: 278) | | | | | |
| SP622.CTLA4. | GGCCCAGCCTGCTGTGGTA | 1 | 1 | 1 | 17 | NA |
| g2 | CNGG (SEQ ID NO: 279) | | | | | |
| SP622.CTLA4. | GTTCACTTGATTTCCACTGGN | 1 | 1 | 1 | 17 | NA |
| g23 | GG (SEQ ID NO: 280) | | | | | |
| SP622.CTLA4. | CAACTCATTCCCCATCATGTN | 1 | 1 | 1 | 18 | NA |
| g27 | GG (SEQ ID NO: 281) | | | | | |
| SP622.CTLA4. | CCGCACAGACTTCAGTCACC | 1 | 1 | 1 | 20 | NA |
| g28 | NGG (SEQ ID NO: 282) | | | | | |
| SP622.CTLA4. | TGTGCGGCAACCTACATGAT | 1 | 1 | 1 | 30 | NA |
| g13 | NGG (SEQ ID NO: 283) | | | | | |
| SP622.CTLA4. | CCTCACTATCCAAGGACTGA | 1 | 1 | 1 | 30 | NA |
| g20 | NGG (SEQ ID NO: 284) | | | | | |
| SP622.CTLA4. | CGGACCTCAGTGGCTTTGCC | 1 | 1 | 1 | 34 | NA |
| g31 | NGG (SEQ ID NO: 285) | | | | | |
| SP622.CTLA4. | GAGGTTCACTTGATTTCCAC | 1 | 1 | 1 | 40 | NA |
| g22 | NGG (SEQ ID NO: 286) | | | | | |
| SP622.CTLA4. | CCAGGTGACTGAAGTCTGTG | 1 | 1 | 1 | 45 | NA |
| g11 | NGG (SEQ ID NO: 287) | | | | | |
| SP622.CTLA4. | ACTGGAGGTGCCCGTGCAGA | 1 | 1 | 2 | 15 | NA |
| g24 | NGG (SEQ ID NO: 288) | | | | | |
| SP622.CTLA4. | CAAGTGAACCTCACTATCCA | 1 | 1 | 2 | 16 | NA |
| g18 | NGG (SEQ ID NO: 289) | | | | | |
| SP622.CTLA4. | GTGGTACTGGCCAGCAGCCG | 1 | 1 | 2 | 29 | NA |
| g3 | NGG (SEQ ID NO: 290) | | | | | |
| SP622.CTLA4. | AGGTCCGGGTGACAGTGCTT | 1 | 1 | 2 | 29 | NA |
| g8 | NGG (SEQ ID NO: 291) | | | | | |
| SP622.CTLA4. | ATCTGCACGGGCACCTCCAG | 1 | 1 | 2 | 29 | NA |
| g17 | NGG (SEQ ID NO: 292) | | | | | |
| SP622.CTLA4. | CCGTGCAGATGGAATCATCT | 1 | 1 | 2 | 36 | NA |
| g25 | NGG (SEQ ID NO: 293) | | | | | |
| SP622.CTLA4. | CTAGATGATTCCATCTGCAC | 1 | 1 | 2 | 39 | NA |
| g16 | NGG (SEQ ID NO: 294) | | | | | |
| SP622.CTLA4. | ACCTCACTATCCAAGGACTG | 1 | 1 | 2 | 40 | NA |
| g19 | NGG (SEQ ID NO: 295) | | | | | |
| SP622.CTLA4. | CCTGCCGAAGCACTGTCACC | 1 | 1 | 2 | 47 | NA |
| g29 | NGG (SEQ ID NO: 296) | | | | | |
| SP622.CTLA4. | TGGCCAGTACCACAGCAGGC | 1 | 1 | 2 | 74 | NA |
| g36 | NGG (SEQ ID NO: 297) | | | | | |
| SP622.CTLA4. | ATCTCCAGGCAAAGCCACTG | 1 | 1 | 2 | 80 | NA |
| g5 | NGG (SEQ ID NO: 298) | | | | | |
| SP622.CTLA4. | GCACGTGGCCCAGCCTGCTG | 1 | 1 | 2 | 121 | NA |
| g1 | NGG (SEQ ID NO: 299) | | | | | |
| SP622.CTLA4. | GTGTGTGAGTATGCATCTCC | 1 | 1 | 3 | 8 | NA |
| g4 | NGG (SEQ ID NO: 300) | | | | | |
| SP622.CTLA4. | CACTGTCACCCGGACCTCAG | 1 | 1 | 3 | 9 | NA |
| g30 | NGG (SEQ ID NO: 301) | | | | | |
| SP622.CTLA4. | GCTGGCGATGCCTCGGCTGC | 1 | 1 | 3 | 17 | NA |
| g34 | NGG (SEQ ID NO: 302) | | | | | |
| SP622.CTLA4. | CTGCTGGCCAGTACCACAGC | 1 | 1 | 3 | 22 | NA |
| g35 | NGG (SEQ ID NO: 303) | | | | | |
| SP622.CTLA4. | AGGCAAAGCCACTGAGGTCC | 1 | 1 | 3 | 40 | NA |
| g7 | NGG (SEQ ID NO: 304) | | | | | |
| SP622.CTLA4. | GCAGATGGAATCATCTAGGA | 1 | 1 | 4 | 20 | NA |
| g26 | NGG (SEQ ID NO: 305) | | | | | |
| SP622.CTLA4. | CCTAGATGATTCCATCTGCA | 1 | 1 | 4 | 40 | NA |
| g15 | NGG (SEQ ID NO: 306) | | | | | |
| SP622.CTLA4. | GGCCAGTACCACAGCAGGCT | 1 | 1 | 4 | 65 | NA |
| g37 | NGG (SEQ ID NO: 307) | | | | | |
| SP622.CTLA4. | TGCATACTCACACACAAAGC | 1 | 1 | 7 | 71 | NA |
| g32 | NGG (SEQ ID NO: 308) | | | | | |
| SP622.CTLA4. | GCTTCGGCAGGCTGACAGCC | 1 | 1 | 8 | 58 | NA |
| g10 | NGG (SEQ ID NO: 309) | | | | | |
| SP622.CTLA4. | CAGGCAAAGCCACTGAGGTC | 1 | 1 | 11 | 30 | NA |
| g6 | NGG (SEQ ID NO: 310) | | | | | |

The gRNA sequences in Table 22 and Table 23 were normalized (% Normalization to NHEJ) for gRNA activity via next generation sequencing (NGS). GFP was used as a control. Following sequencing analysis, the following gRNAs were recommended based on off-target profile: Exon 1: SP621.hCTLA4.g2 (>15%) and SP621.hCTLA4.g12 (>15%). Exon 2: SP622.hCTLA4.g2 (>15%), SP622.hCTLA4.g9 (>15%), and SP622.hCTLA4.g33 (>15%).

Off target analysis of selected gRNA was performed for 2 exons of hPDCD1 (CF60 and CF61) to determine the number of sites in human genome which are an exact match or contains up to 1 or 2 mismatches, which may include the target site. The results are listed in Table 24 for Exon CF60 and Table 25 for Exon CF61.

TABLE 24
Guide RNA (gRNA) Off Target Analysis for hPDCD1 (Exon CF60)

| | | long_ | long_ | long_ | short_ | |
|---|---|---|---|---|---|---|
| Name | gRNA | 0 | 1 | 2 | 0 | SNP |
| CF60.PDCD1. | TGTAGCACCGCCCAGACGA | 1 | 1 | 1 | 1 | NA |
| g12 | CNGG (SEQ ID NO: 311) | | | | | |
| CF60.PDCD1. | GGCGCCCTGGCCAGTCGTC | 1 | 1 | 1 | 3 | NA |
| g3 | TNGG (SEQ ID NO: 312) | | | | | |
| CF60.PDCD1. | CGTCTGGGCGGTGCTACAAC | 1 | 1 | 1 | 3 | NA |
| g5 | NGG (SEQ ID NO: 313) | | | | | |
| CF60.PDCD1. | AGGCGCCCTGGCCAGTCGTC | 1 | 1 | 1 | 5 | NA |
| g2 | NGG (SEQ ID NO: 314) | | | | | |
| CF60.PDCD1. | CACCGCCCAGACGACTGGCC | 1 | 1 | 1 | 5 | NA |
| g13 | NGG (SEQ ID NO: 315) | | | | | |
| CF60.PDCD1. | ACCGCCCAGACGACTGGCCA | 1 | 1 | 1 | 5 | NA |
| g14 | NGG (SEQ ID NO: 316) | | | | | |
| CF60.PDCD1. | GGGCGGTGCTACAACTGGGC | 1 | 1 | 1 | 7 | NA |
| g7 | NGG (SEQ ID NO: 317) | | | | | |
| CF60.PDCD1. | GTCTGGGCGGTGCTACAACT | 1 | 1 | 1 | 9 | NA |
| g6 | NGG (SEQ ID NO: 318) | | | | | |
| CF60.PDCD1. | CGACTGGCCAGGGCGCCTGT | 1 | 1 | 1 | 15 | NA |
| g16 | NGG (SEQ ID NO: 319) | | | | | |
| CF60.PDCD1. | CGGTGCTACAACTGGGCTGG | 1 | 1 | 1 | 33 | NA |
| g8 | NGG (SEQ ID NO: 320) | | | | | |
| CF60.PDCD1. | TGGCGGCCAGGATGGTTCTT | 1 | 1 | 1 | 33 | NA |
| g11 | NGG (SEQ ID NO: 321) | | | | | |
| CF60.PDCD1. | ACGACTGGCCAGGGCGCCTG | 1 | 1 | 1 | 45 | NA |
| g15 | NGG (SEQ ID NO: 322) | | | | | |
| CF60.PDCD1. | CTACAACTGGGCTGGCGGCC | 1 | 1 | 1 | 57 | NA |
| g9 | NGG (SEQ ID NO: 323) | | | | | |
| CF60.PDCD1. | GCCCTGGCCAGTCGTCTGGG | 1 | 1 | 2 | 2 | NA |
| g4 | NGG (SEQ ID NO: 324) | | | | | |
| CF60.PDCD1. | TGCAGATCCCACAGGCGCCC | 1 | 1 | 2 | 23 | NA |
| g1 | NGG (SEQ ID NO: 325) | | | | | |
| CF60.PDCD1. | AACTGGGCTGGCGGCCAGGA | 1 | 1 | 3 | 17 | NA |
| g10 | NGG (SEQ ID NO: 326) | | | | | |

TABLE 25
Guide RNA (gRNA) Off Target Analysis for hPDCD1 (CF61)

| | | long_ | long_ | long_ | short_ | |
|---|---|---|---|---|---|---|
| Name | gRNA | 0 | 1 | 2 | 0 | SNP |
| CF61.PDCD1. | CGGAGAGCTTCGTGCTAAA | 1 | 1 | 1 | 1 | NA |
| g6 | CNGG (SEQ ID NO: 327) | | | | | |
| CF61.PDCD1. | GCGTGACTTCCACATGAGCG | 1 | 1 | 1 | 2 | NA |
| g14 | NGG (SEQ ID NO: 328) | | | | | |
| CF61.PDCD1. | ATGTGGAAGTCACGCCCGTT | 1 | 1 | 1 | 2 | NA |
| g17 | NGG (SEQ ID NO: 329) | | | | | |
| CF61.PDCD1. | GCCCTGCTCGTGGTGACCG | 1 | 1 | 1 | 3 | NA |
| g2 | ANGG (SEQ ID NO: 330) | | | | | |
| CF61.PDCD1. | CACGAAGCTCTCCGATGTG | 1 | 1 | 1 | 3 | NA |
| g35 | TNGG (SEQ ID NO: 331) | | | | | |
| CF61.PDCD1. | CCTGCTCGTGGTGACCGAAG | 1 | 1 | 1 | 4 | NA |
| g4 | NGG (SEQ ID NO: 332) | | | | | |
| CF61.PDCD1. | TGACACGGAAGCGGCAGTCC | 1 | 1 | 1 | 5 | NA |
| g20 | NGG (SEQ ID NO: 333) | | | | | |
| CF61.PDCD1. | CCCCTTCGGTCACCACGAGC | 1 | 1 | 1 | 5 | NA |
| g40 | NGG (SEQ ID NO: 334) | | | | | |
| CF61.PDCD1. | CAGCAACCAGACGGACAAGC | 1 | 1 | 1 | 6 | NA |
| g8 | NGG (SEQ ID NO: 335) | | | | | |
| CF61.PDCD1. | GCAGTTGTGTGACACGGAAG | 1 | 1 | 1 | 6 | NA |
| g19 | NGG (SEQ ID NO: 336) | | | | | |
| CF61.PDCD1. | CCCTTCGGTCACCACGAGCA | 1 | 1 | 1 | 6 | NA |
| g41 | NGG (SEQ ID NO: 337) | | | | | |
| CF61.PDCD1. | CCGGGCTGGCTGCGGTCCTC | 1 | 1 | 1 | 8 | NA |
| g26 | NGG (SEQ ID NO: 338) | | | | | |
| CF61.PDCD1. | AGGCGGCCAGCTTGTCCGTC | 1 | 1 | 1 | 8 | NA |
| g30 | NGG (SEQ ID NO: 339) | | | | | |
| CF61.PDCD1. | CAGCTTGTCCGTCTGGTTGCN | 1 | 1 | 1 | 8 | NA |
| g31 | GG (SEQ ID NO: 340) | | | | | |
| CF61.PDCD1. | CGGTCACCACGAGCAGGGCT | 1 | 1 | 1 | 10 | NA |
| g43 | NGG (SEQ ID NO: 341) | | | | | |
| CF61.PDCD1. | GTGTCACACAACTGCCCAAC | 1 | 1 | 1 | 13 | NA |
| g13 | NGG (SEQ ID NO: 342) | | | | | |
| CF61.PDCD1. | CTGCAGCTTCTCCAACACATN | 1 | 1 | 1 | 23 | NA |
| g5 | GG (SEQ ID NO: 343) | | | | | |
| CF61.PDCD1. | CAAGCTGGCCGCCTTCCCCG | 1 | 1 | 1 | 23 | NA |
| g9 | NGG (SEQ ID NO: 344) | | | | | |
| CF61.PDCD1. | CGTGTCACACAACTGCCCAA | 1 | 1 | 1 | 28 | NA |
| g12 | NGG (SEQ ID NO: 345) | | | | | |
| CF61.PDCD1. | CGTTGGGCAGTTGTGTGACA | 1 | 1 | 1 | 32 | NA |
| g18 | NGG (SEQ ID NO: 346) | | | | | |
| CF61.PDCD1. | GCTTGTCCGTCTGGTTGCTGN | 1 | 1 | 1 | 41 | NA |
| g33 | GG (SEQ ID NO: 347) | | | | | |
| CF61.PDCD1. | CGGAAGCGGCAGTCCTGGCC | 1 | 1 | 1 | 61 | NA |
| g22 | NGG (SEQ ID NO: 348) | | | | | |
| CF61.PDCD1. | CGATGTGTTGGAGAAGCTGC | 1 | 1 | 1 | 135 | NA |
| g36 | NGG (SEQ ID NO: 349) | | | | | |
| CF61.PDCD1. | CATGTGGAAGTCACGCCCGT | 1 | 1 | 2 | 2 | NA |
| g16 | NGG (SEQ ID NO: 350) | | | | | |
| CF61.PDCD1. | CCCTGCTCGTGGTGACCGAA | 1 | 1 | 2 | 3 | NA |
| g3 | NGG (SEQ ID NO: 351) | | | | | |
| CF61.PDCD1. | CGGGCTGGCTGCGGTCCTCG | 1 | 1 | 2 | 3 | NA |
| g27 | NGG (SEQ ID NO: 352) | | | | | |
| CF61.PDCD1. | AGCTTGTCCGTCTGGTTGCTN | 1 | 1 | 2 | 4 | NA |
| g32 | GG (SEQ ID NO: 353) | | | | | |
| CF61.PDCD1. | GAAGGTGGCGTTGTCCCCTT | 1 | 1 | 2 | 4 | NA |
| g39 | NGG (SEQ ID NO: 354) | | | | | |
| CF61.PDCD1. | ACTTCCACATGAGCGTGGTC | 1 | 1 | 2 | 6 | NA |
| g15 | NGG (SEQ ID NO: 355) | | | | | |
| CF61.PDCD1. | GCCGGGCTGGCTGCGGTCCT | 1 | 1 | 2 | 17 | NA |
| g25 | NGG (SEQ ID NO: 356) | | | | | |
| CF61.PDCD1. | TCGGTCACCACGAGCAGGGC | 1 | 1 | 2 | 23 | NA |
| g42 | NGG (SEQ ID NO: 357) | | | | | |
| CF61.PDCD1. | TCTGGTTGCTGGGGCTCATGN | 1 | 1 | 2 | 31 | NA |
| g34 | GG (SEQ ID NO: 358) | | | | | |
| CF61.PDCD1. | ACGGAAGCGGCAGTCCTGGC | 1 | 1 | 2 | 41 | NA |
| g21 | NGG (SEQ ID NO: 359) | | | | | |
| CF61.PDCD1. | CCCGAGGACCGCAGCCAGCC | 1 | 1 | 2 | 46 | NA |
| g10 | NGG (SEQ ID NO: 360) | | | | | |
| CF61.PDCD1. | CTGGCTGCGGTCCTCGGGGA | 1 | 1 | 3 | 16 | NA |
| g28 | NGG (SEQ ID NO: 361) | | | | | |
| CF61.PDCD1. | CATGAGCCCCAGCAACCAGA | 1 | 1 | 3 | 33 | NA |
| g7 | NGG (SEQ ID NO: 362) | | | | | |
| CF61.PDCD1. | AGTCCTGGCCGGGCTGGCTG | 1 | 1 | 3 | 42 | NA |
| g24 | NGG (SEQ ID NO: 363) | | | | | |
| CF61.PDCD1. | GGGGGTTCCAGGGCCTGTCT | 1 | 1 | 3 | 126 | NA |
| g55 | NGG (SEQ ID NO: 364) | | | | | |
| CF61.PDCD1. | GGTCACCACGAGCAGGGCTG | 1 | 1 | 4 | 26 | NA |
| g44 | NGG (SEQ ID NO: 365) | | | | | |
| CF61.PDCD1. | GCTGCGGTCCTCGGGGAAGG | 1 | 1 | 4 | 35 | NA |
| g29 | NGG (SEQ ID NO: 366) | | | | | |
| CF61.PDCD1. | GGACCGCAGCCAGCCCGGCC | 1 | 1 | 4 | 47 | NA |
| g11 | NGG (SEQ ID NO: 367) | | | | | |
| CF61.PDCD1. | GAGAAGGTGGGGGGGTTCCA | 1 | 1 | 5 | 8 | NA |
| g53 | NGG (SEQ ID NO: 368) | | | | | |
| CF61.PDCD1. | GGAGAAGGTGGGGGGGTTCC | 1 | 1 | 5 | 15 | NA |
| g52 | NGG (SEQ ID NO: 369) | | | | | |
| CF61.PDCD1. | AGCGGCAGTCCTGGCCGGGC | 1 | 1 | 5 | 39 | NA |
| g23 | NGG (SEQ ID NO: 370) | | | | | |
| CF61.PDCD1. | GGGGTTCCAGGGCCTGTCTG | 1 | 1 | 5 | 97 | NA |
| g56 | NGG (SEQ ID NO: 371) | | | | | |
| CF61.PDCD1. | CTTCTCCCCAGCCCTGCTCGN | 1 | 1 | 6 | 22 | NA |
| g1 | GG (SEQ ID NO: 372) | | | | | |
| CF61.PDCD1. | GTTGGAGAAGCTGCAGGTGA | 1 | 1 | 6 | 88 | NA |
| g37 | NGG (SEQ ID NO: 373) | | | | | |
| CF61.PDCD1. | GGGGGGTTCCAGGGCCTGTC | 1 | 1 | 6 | 1286 | NA |
| g54 | NGG (SEQ ID NO: 374) | | | | | |
| CF61.PDCD1. | GGAGAAGCTGCAGGTGAAGG | 1 | 1 | 9 | 66 | NA |
| g38 | NGG (SEQ ID NO: 375) | | | | | |
| CF61.PDCD1. | CACGAGCAGGGCTGGGGAGA | 1 | 1 | 10 | 448 | NA |
| g45 | NGG (SEQ ID NO: 376) | | | | | |
| CF61.PDCD1. | GCAGGGCTGGGGAGAAGGTG | 1 | 1 | 21 | 125 | NA |
| g48 | NGG (SEQ ID NO: 377) | | | | | |
| CF61.PDCD1. | CAGGGCTGGGGAGAAGGTGG | 1 | 1 | 29 | 214 | NA |
| g49 | NGG (SEQ ID NO: 378) | | | | | |
| CF61.PDCD1. | GAGCAGGGCTGGGGAGAAG | 1 | 1 | 30 | 202 | NA |
| g46 | GNGG (SEQ ID NO: 379) | | | | | |
| CF61.PDCD1. | AGCAGGGCTGGGGAGAAGGT | 1 | 2 | 11 | 136 | NA |
| g47 | NGG (SEQ ID NO: 380) | | | | | |
| CF61.PDCD1. | AGGGCTGGGGAGAAGGTGGG | 1 | 2 | 31 | 179 | NA |
| g50 | NGG (SEQ ID NO: 381) | | | | | |
| CF61.PDCD1. | GGGCTGGGGAGAAGGTGGGG | 1 | 2 | 49 | 130 | NA |
| g51 | NGG (SEQ ID NO: 382) | | | | | |

The gRNA sequences in Table 24 and Table 25 were normalized (% Normalization to NHEJ) for gRNA activity via next generation sequencing (NGS). GFP was used as a control. Following sequencing analysis, the following gRNAs were recommended based on off-target profile: CF60.PDCD1.g12 (65.6%), CF60.PDCD1.g3 (69.2%), CF61.PDCD1.g6, CF61.PDCD1.g2 (72.7%), and CF61.PDCD1.g35 (24.0%).

Off target analysis of selected gRNA was performed for 2 exons of hTIM3 (Exon 2 and Exon 3) to determine the number of sites in human genome which are an exact match or contains up to 1 or 2 mismatches, which may include the target site. The results are listed in Table 26 for Exon 2 and Table 27 for Exon 3.

TABLE 26
Guide RNA (gRNA) Off Target Analysis for hTIM3 (Exon 2)

| | | long_ | long_ | long_ | short_ | |
|---|---|---|---|---|---|---|
| Name | gRNA | 0 | 1 | 2 | 0 | SNP |
| SP619.TIM3. | AGAAGTGGAATACAGAGCGG | 1 | 1 | 1 | 2 | NA |
| g2 | NGG (SEQ ID NO: 383) | | | | | |
| SP619.TIM3. | AATGTGGCAACGTGGTGCT | 1 | 1 | 1 | 3 | NA |
| g12 | CNGG (SEQ ID NO: 384) | | | | | |
| SP619.TIM3. | CTAAATGGGGATTTCCGCAA | 1 | 1 | 1 | 4 | NA |
| g20 | NGG (SEQ ID NO: 385) | | | | | |
| SP619.TIM3. | CATCCAGATACTGGCTAAATN | 1 | 1 | 1 | 8 | NA |
| g18 | GG (SEQ ID NO: 386) | | | | | |
| SP619.TIM3. | CAGACGGGCACGAGGTTCCC | 1 | 1 | 1 | 8 | NA |
| g41 | NGG (SEQ ID NO: 387) | | | | | |
| SP619.TIM3. | GCGGCTGGGGTGTAGAAGC | 1 | 1 | 1 | 8 | NA |
| g49 | ANGG (SEQ ID NO: 388) | | | | | |
| SP619.TIM3. | GAACCTCGTGCCCGTCTGCTN | 1 | 1 | 1 | 10 | NA |
| g7 | GG (SEQ ID NO: 389) | | | | | |
| SP619.TIM3. | GACGGGCACGAGGTTCCCTG | 1 | 1 | 1 | 10 | NA |
| g43 | NGG (SEQ ID NO: 390) | | | | | |
| SP619.TIM3. | ATCCCCATTTAGCCAGTATCN | 1 | 1 | 1 | 11 | NA |
| g35 | GG (SEQ ID NO: 391) | | | | | |
| SP619.TIM3. | GTGGAATACAGAGCGGAGGT | 1 | 1 | 1 | 12 | NA |
| g3 | NGG (SEQ ID NO: 392) | | | | | |
| SP619.TIM3. | AGACGGGCACGAGGTTCCCT | 1 | 1 | 1 | 12 | NA |
| g42 | NGG (SEQ ID NO: 393) | | | | | |
| SP619.TIM3. | GGAACCTCGTGCCCGTCTGCN | 1 | 1 | 1 | 13 | NA |
| g6 | GG (SEQ ID NO: 394) | | | | | |
| SP619.TIM3. | GAGTCACATTCTCTATGGTCN | 1 | 1 | 1 | 14 | NA |
| g32 | GG (SEQ ID NO: 395) | | | | | |
| SP619.TIM3. | ATGTGACTCTAGCAGACAGTN | 1 | 1 | 1 | 16 | NA |
| g22 | GG (SEQ ID NO: 396) | | | | | |
| SP619.TIM3. | TTTTCATCATTCATTATGCCN | 1 | 1 | 1 | 16 | NA |
| g27 | GG (SEQ ID NO: 397) | | | | | |
| SP619.TIM3. | AATGTGACTCTAGCAGACAG | 1 | 1 | 1 | 17 | NA |
| g21 | NGG (SEQ ID NO: 398) | | | | | |
| SP619.TIM3. | ATCCAGATACTGGCTAAATGN | 1 | 1 | 1 | 18 | NA |
| g19 | GG (SEQ ID NO: 399) | | | | | |
| SP619.TIM3. | TGCTGCCGGATCCAAATCCCN | 1 | 1 | 1 | 22 | NA |
| g24 | GG (SEQ ID NO: 400) | | | | | |
| SP619.TIM3. | TCTACACCCCAGCCGCCCCAN | 1 | 1 | 1 | 30 | NA |
| g5 | GG (SEQ ID NO: 401) | | | | | |
| SP619.TIM3. | TTATGCCTGGGATTTGGATCN | 1 | 1 | 1 | 35 | NA |
| g30 | GG (SEQ ID NO: 402) | | | | | |
| SP619.TIM3. | CGCTCTGTATTCCACTTCTGN | 1 | 1 | 1 | 83 | NA |
| g51 | GG (SEQ ID NO: 403) | | | | | |
| SP619.TIM3. | GAGGTTCCCTGGGGCGGCTGN | 1 | 1 | 1 | 85 | NA |
| g47 | GG (SEQ ID NO: 404) | | | | | |
| SP619.TIM3. | TGCCCCAGCAGACGGGCACG | 1 | 1 | 2 | 5 | NA |
| g40 | NGG (SEQ ID NO: 405) | | | | | |
| SP619.TIM3. | ACAGTGGGATCTACTGCTGCN | 1 | 1 | 2 | 8 | NA |
| g23 | GG (SEQ ID NO: 406) | | | | | |
| SP619.TIM3. | TGTGTTTGAATGTGGCAACGN | 1 | 1 | 2 | 9 | NA |
| g11 | GG (SEQ ID NO: 407) | | | | | |
| SP619.TIM3. | TGAAAAATTTAACCTGAAGTN | 1 | 1 | 2 | 16 | NA |
| g25 | GG (SEQ ID NO: 408) | | | | | |
| SP619.TIM3. | ACATCCAGATACTGGCTAAAN | 1 | 1 | 2 | 19 | NA |
| g17 | GG (SEQ ID NO: 409) | | | | | |
| SP619.TIM3. | ATGAAAGGGATGTGAATTAT | 1 | 1 | 2 | 22 | NA |
| g15 | NGG (SEQ ID NO: 410) | | | | | |
| SP619.TIM3. | TGGTGCTCAGGACTGATGAAN | 1 | 1 | 2 | 25 | NA |
| g13 | GG (SEQ ID NO: 411) | | | | | |
| SP619.TIM3. | GGTGTAGAAGCAGGGCAGAT | 1 | 1 | 2 | 36 | NA |
| g50 | NGG (SEQ ID NO: 412) | | | | | |
| SP619.TIM3. | ACGTTGCCACATTCAAACACN | 1 | 1 | 2 | 37 | NA |
| g36 | GG (SEQ ID NO: 413) | | | | | |
| SP619.TIM3. | ACGAGGTTCCCTGGGGCGGC | 1 | 1 | 2 | 40 | NA |
| g45 | NGG (SEQ ID NO: 414) | | | | | |
| SP619.TIM3. | GCCTGTCCTGTGTTTGAATGN | 1 | 1 | 2 | 47 | NA |
| g10 | GG (SEQ ID NO: 415) | | | | | |
| SP619.TIM3. | GTGCCCGTCTGCTGGGGCAAN | 1 | 1 | 2 | 58 | NA |
| g9 | GG (SEQ ID NO: 416) | | | | | |
| SP619.TIM3. | AACCTCGTGCCCGTCTGCTGN | 1 | 1 | 3 | 15 | NA |
| g8 | GG (SEQ ID NO: 417) | | | | | |
| SP619.TIM3. | GGCGGCTGGGGTGTAGAAGC | 1 | 1 | 3 | 15 | NA |
| g48 | NGG (SEQ ID NO: 418) | | | | | |
| SP619.TIM3. | AGTCACATTCTCTATGGTCAN | 1 | 1 | 3 | 19 | NA |
| g33 | GG (SEQ ID NO: 419) | | | | | |
| SP619.TIM3. | CTGGTTTGATGACCAACTTCN | 1 | 1 | 3 | 21 | NA |
| g26 | GG (SEQ ID NO: 420) | | | | | |
| SP619.TIM3. | CATTCATTATGCCTGGGATTN | 1 | 1 | 3 | 24 | NA |
| g29 | GG (SEQ ID NO: 421) | | | | | |
| SP619.TIM3. | TGCTAGAGTCACATTCTCTAN | 1 | 1 | 3 | 49 | NA |
| g31 | GG (SEQ ID NO: 422) | | | | | |
| SP619.TIM3. | GGGCACGAGGTTCCCTGGGG | 1 | 1 | 3 | 53 | NA |
| g44 | NGG (SEQ ID NO: 423) | | | | | |
| SP619.TIM3. | GGCTCCTTTGCCCCAGCAGAN | 1 | 1 | 3 | 58 | NA |
| g38 | GG (SEQ ID NO: 424) | | | | | |
| SP619.TIM3. | ATTATTGGACATCCAGATACN | 1 | 1 | 3 | 106 | NA |
| g16 | GG (SEQ ID NO: 425) | | | | | |
| SP619.TIM3. | TTTCATCATTCATTATGCCTN | 1 | 1 | 4 | 23 | NA |
| g28 | GG (SEQ ID NO: 426) | | | | | |
| SP619.TIM3. | TTCTACACCCCAGCCGCCCCN | 1 | 1 | 4 | 29 | NA |
| g4 | GG (SEQ ID NO: 427) | | | | | |
| SP619.TIM3. | TCAGGGACACATCTCCTTTGN | 1 | 1 | 4 | 41 | NA |
| g34 | GG (SEQ ID NO: 428) | | | | | |
| SP619.TIM3. | GCTCCTTTGCCCCAGCAGACN | 1 | 1 | 4 | 42 | NA |
| g39 | GG (SEQ ID NO: 429) | | | | | |
| SP619.TIM3. | CTCAGAAGTGGAATACAGAG | 1 | 1 | 5 | 35 | NA |
| g1 | NGG (SEQ ID NO: 430) | | | | | |
| SP619.TIM3. | CGAGGTTCCCTGGGGCGGCTN | 1 | 2 | 2 | 18 | NA |
| g46 | GG (SEQ ID NO: 431) | | | | | |
| SP619.TIM3. | GCCACATTCAAACACAGGAC | 1 | 2 | 2 | 25 | NA |
| g37 | NGG (SEQ ID NO: 432) | | | | | |
| SP619.TIM3. | GGTGCTCAGGACTGATGAAA | 1 | 2 | 3 | 28 | NA |
| g14 | NGG (SEQ ID NO: 433) | | | | | |

TABLE 27
Guide RNA (gRNA) Off Target Analysis for hTIM3 (Exon 3)

| | | long_ | long_ | long_ | short_ | |
|---|---|---|---|---|---|---|
| Name | gRNA | 0 | 1 | 2 | 0 | SNP |
| SP620.TIM3. | AGGTCACCCCTGCACCGAC | 1 | 1 | 1 | 4 | rs1036199: |
| g1 | TNGG (SEQ ID NO: 434) | | | | | 0.13 |
| SP620.TIM3. | CTCTCTGCCGAGTCGGTGC | 1 | 1 | 1 | 4 | rs1036199: |
| g11 | ANGG (SEQ ID NO: 435) | | | | | 0.13 |
| SP620.TIM3. | TCTCTCTGCCGAGTCGGTG | 1 | 1 | 1 | 6 | rs1036199: |
| g10 | CNGG (SEQ ID NO: 436) | | | | | 0.13 |
| SP620.TIM3. | CCAAGGATGCTTACCACC | 1 | 1 | 1 | 8 | NA |
| g5 | AGNGG (SEQ ID NO: 437) | | | | | |
| SP620.TIM3. | TCTCTGCCGAGTCGGTGCA | 1 | 1 | 1 | 9 | rs1036199: |
| g12 | GNGG (SEQ ID NO: 438) | | | | | 0.13 |
| SP620.TIM3. | CCCCTGGTGGTAAGCATC | 1 | 1 | 1 | 10 | NA |
| g7 | CTNGG (SEQ ID NO: 439) | | | | | |
| SP620.TIM3. | TCCAAGGATGCTTACCACC | 1 | 1 | 1 | 16 | NA |
| g4 | ANGG (SEQ ID NO: 440) | | | | | |
| SP620.TIM3. | GGTGGTAAGCATCCTTGGA | 1 | 1 | 1 | 20 | NA |
| g8 | ANGG (SEQ ID NO: 441) | | | | | |
| SP620.TIM3. | GTGAAGTCTCTCTGCCGAG | 1 | 1 | 2 | 6 | rs1036199: |
| g9 | TNGG (SEQ ID NO: 442) | | | | | 0.13 |
| SP620.TIM3. | ATGCTTACCACCAGGGGAC | 1 | 1 | 2 | 34 | NA |
| g6 | ANGG (SEQ ID NO: 443) | | | | | |
| SP620.TIM3. | TTCCAAGGATGCTTACCAC | 1 | 1 | 2 | 36 | NA |
| g3 | CNGG (SEQ ID NO: 444) | | | | | |
| SP620.TIM3. | AGTCGGTGCAGGGGTGACC | 1 | 1 | 2 | 45 | NA |
| g13 | TNGG (SEQ ID NO: 445) | | | | | |
| SP620.TIM3. | ACTTCACTGCAGCCTTTCC | 1 | 1 | 4 | 38 | NA |
| g2 | ANGG (SEQ ID NO: 446) | | | | | |

The gRNA sequences in Table 26 and Table 27 were normalized (% Normalization to NHEJ) for gRNA activity via next generation sequencing (NGS). GFP was used as a control. Following sequencing analysis, the following gRNAs were recommended based on off-target profile: Exon 2: SP619.hTIM3.g12 (45.0%), SP619.hTIM3.g20 (60.9%), and SP619.hTIM3.g49 (45.4%). Exon 3: SP620.hTIM3.g5 (58.0%) and SP620.hTIM3.g7 (2.9%).

The methods disclosed above can be varied appropriately by those skilled in the art to make and confirm activity of other mono, dual, and tandem CAR-T cells disclosed herein.

Although the present invention has been described with reference to specific details of certain embodiments thereof in the above examples, it will be understood that modification and variation are encompassed within the spirit and scope of the invention.

1-104. (canceled)

105. A T cell, comprising a chimeric antigen receptor (CAR) that specifically binds CD7, wherein the CAR comprises residues 1-495 of SEQ ID NO:32, and wherein the T cell comprises a disruption of the gene encoding CD7 and a disruption of the gene encoding TCR receptor alpha chain (TRAC).

106. A therapeutic composition comprising a population of immune effector cells, the composition comprising:
a. one or more of a T cell according to claim 105, and
b. at least one therapeutically acceptable carrier and/or adjuvant.

107. The composition of claim 106, wherein the population comprises between about 10⁴-10⁹ immune effector cells per kg of body weight of a subject to whom the therapeutic composition is administered.

108. A method for treating a hematologic malignancy in a human subject, the method comprising administering at least one infusion of between about 10⁴-10⁹ immune effector cells per kg of body weight of the subject, wherein:
a. the cells are modified to express a chimeric antigen receptor (CAR) that specifically binds CD7;
b. the CAR comprises amino acid residues 1-495 of SEQ ID NO:32; and
c. the cells comprise a disruption of the gene encoding CD7 and a disruption of the gene encoding TCR receptor alpha chain (TRAC).

109. The method of claim 108, wherein the genes encoding CD7 and TRAC are disrupted using a CRISPR system.

110. The method of claim 109, wherein endogenous T cell receptor mediated signaling is blocked in the immune effector cells.

111. The method of claim 110, wherein the immune effector cells are human T cells.

112. The method of claim 111, wherein the human T cells are sourced from a healthy donor, from cord blood, or generated from induced pluripotent stem cells.

113. The method of claim 112, wherein the T cells comprise CD4+ T cells, CD8+ T cells, or a combination thereof.

114. The method of claim 113, wherein cells of the hematologic malignancy from the human subject overexpress cell surface CD7.

115. The method of claim 114, wherein the hematologic malignancy is T-cell acute lymphoblastic leukemia (T-ALL), T-cell lymphoblastic lymphoma (T-LBL), non-Hodgkin's Lymphoma (NHL) or acute myeloid leukemia (AML).

116. The method of claim 115, wherein the infusion is administered to the subject once and comprises an effective amount of the immune effector cells.

117. The method of claim 116, wherein the cells demonstrate reduced T cell fratricide compared to CAR-bearing immune effector cells that are not genetically engineered to be deficient in CD7.

118. The method of claim 116, wherein the immune effector cells demonstrate improved in vivo persistence compared to CAR-bearing immune effector cells that are not genetically engineered to be deficient in one or more of TRAC and CD7.

119. The method of claim 116, wherein the immune effector cells demonstrate improved in vivo expansion compared to CAR-bearing immune effector cells that are not genetically engineered to be deficient in one or more of TRAC and CD7.

120. The method of claim 116, wherein the immune effector cells demonstrate improved therapeutic activity compared to CAR-bearing immune effector cells that are not genetically engineered to be deficient in one or more of TRAC and CD7.

121. The method of claim 120, wherein the immune effector cells preserve sufficient normal T cells in the human subject to maintain normal immune system function compared to CAR-bearing immune effector cells that are not engineered to be deficient in one or more of TRAC and CD7.

122. The method of claim 121, wherein the immune effector cells demonstrate fewer side effects in the human subject, compared to CAR-bearing immune effector cells that are not engineered to be deficient in one or more of TRAC and CD7, wherein the side effects comprise cytokine release syndrome (CRS), alloreactivity or graft-versus-host disease (GvHD), and/or T cell aplasia.

123. The method of claim 122, wherein the cells are administered following another treatment modality.

124. The method of claim 123, wherein the other treatment modality is one or more of chemotherapy and radiation therapy.


SEQUENCE LISTING (452 sequences)

SEQ ID NO: 1 - protein, 21 aa, synthetic construct
  REGION 1..21: note = Description of Artificial Sequence: Synthetic peptide
MALPVTALLLPLALLLHAARP

SEQ ID NO: 2 - protein, 45 aa, synthetic construct
  REGION 1..45: note = Description of Artificial Sequence: Synthetic polypeptide
TTTPAPRPPTPAPTIASQPLSLRPEACRPAAGGAVHTRGLDFACD

SEQ ID NO: 3 - protein, 27 aa, synthetic construct
  REGION 1..27: note = Description of Artificial Sequence: Synthetic peptide
FWVLVVVGGVLACYSLLVTVAFIIFWV

SEQ ID NO: 4 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 5 - protein, 42 aa, synthetic construct
  REGION 1..42: note = Description of Artificial Sequence: Synthetic polypeptide
KRGRKKLLYIFKQPFMRPVQTTQEEDGCSCRFPEEEEGGCEL

SEQ ID NO: 6 - protein, 41 aa, synthetic construct
  REGION 1..41: note = Description of Artificial Sequence: Synthetic polypeptide
RSKRSRLLHSDYMNMTPRRPGPTRKHYQPYAPPRDFAAYRS

SEQ ID NO: 7 - protein, 112 aa, synthetic construct
  REGION 1..112: note = Description of Artificial Sequence: Synthetic polypeptide
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 22 aa, synthetic construct
  REGION 1..22: note = Description of Artificial Sequence: Synthetic peptide
GSGATNFSLLKQAGDVEENPGP

SEQ ID NO: 9 - protein, 20 aa, synthetic construct
  REGION 1..20: note = Description of Artificial Sequence: Synthetic peptide
GGGGSGGGGSGGGGSGGGGS

SEQ ID NO: 10 - protein, 308 aa, synthetic construct
  REGION 1..308: note = Description of Artificial Sequence: Synthetic polypeptide
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 11 - protein, 710 aa, synthetic construct
  REGION 1..710: note = Description of Artificial Sequence: Synthetic polypeptide
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGEGGGGGDLGGVKLPHLFGKRLVEARMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLLLDRHPIAVMLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 12 - protein, 119 aa, synthetic construct
  REGION 1..119: note = Description of Artificial Sequence: Synthetic polypeptide
EVKLEESGAELVKPGASVKLSCRTSGFNLKDTIHWVKQRPEQGLKWIGRIDPANGNTKYDPKFQDKATVTADTSSNTAYLQLSSLTSEDTAVYYCVTYAYDGNWYFDVWGAGTAVTVSS

SEQ ID NO: 13 - protein, 109 aa, synthetic construct
  REGION 1..109: note = Description of Artificial Sequence: Synthetic polypeptide
DIKNITQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKR

SEQ ID NO: 14 - protein, 121 aa, synthetic construct
  REGION 1..121: note = Description of Artificial Sequence: Synthetic polypeptide
EVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSS

SEQ ID NO: 15 - protein, 113 aa, synthetic construct
  REGION 1..113: note = Description of Artificial Sequence: Synthetic polypeptide
DIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIER

SEQ ID NO: 16 - protein, 121 aa, synthetic construct
  REGION 1..121: note = Description of Artificial Sequence: Synthetic polypeptide
GSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 17 - protein, 107 aa, synthetic construct
  REGION 1..107: note = Description of Artificial Sequence: Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 18 - protein, 122 aa, synthetic construct
  REGION 1..122: note = Description of Artificial Sequence: Synthetic polypeptide
EVQLVESGGGLVQPGGSLRLSCAASGYSFTGYTMNWVRQAPGKCLEWVALINPYKGVSTYNQKFKDRFTISVDKSKNTAYLQMNSLRAEDTAVYYCARSGYYGDSDWYFDVWGQGTLVTVSS

SEQ ID NO: 19 - protein, 107 aa, synthetic construct
  REGION 1..107: note = Description of Artificial Sequence: Synthetic polypeptide
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIK

SEQ ID NO: 20 - protein, 116 aa, synthetic construct
  REGION 1..116: note = Description of Artificial Sequence: Synthetic polypeptide
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVS

SEQ ID NO: 21 - protein, 108 aa, synthetic construct
  REGION 1..108: note = Description of Artificial Sequence: Synthetic polypeptide
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKR

SEQ ID NO: 22 - protein, 119 aa, synthetic construct
  REGION 1..119: note = Description of Artificial Sequence: Synthetic polypeptide
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGVGAHDAFDIWGQGTTVTVSS

SEQ ID NO: 23 - protein, 112 aa, synthetic construct
  REGION 1..112: note = Description of Artificial Sequence: Synthetic polypeptide
DVVMTQSPLSLPVTPGEPASISCRSSQSLLHSNGNNYLDWYLQKPGQSPQLLIYLGSNRASGVPDRFSGSGSDTDFTLQISRVEAEDVGVYYCMQGTHPAISFGQGTRLEIK

SEQ ID NO: 24 - protein, 122 aa, synthetic construct
  REGION 1..122: note = Description of Artificial Sequence: Synthetic polypeptide
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSS

SEQ ID NO: 25 - protein, 107 aa, synthetic construct
  REGION 1..107: note = Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIK

SEQ ID NO: 26 - protein, 118 aa, synthetic construct
  REGION 1..118: note = Description of Artificial Sequence: Synthetic polypeptide
EVQLVQSGAEVKKPGASVKVSCKASGYTFTSYYMHWARQAPGQGLEWMGIINPSGGSTSYAQKFQGRVTMTRDTSTSTVYMELSSLRSEDTAVYYCARVVAAAVADYWGQGTLVTVSS

SEQ ID NO: 27 - protein, 112 aa, synthetic construct
  REGION 1..112: note = Description of Artificial Sequence: Synthetic polypeptide
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQSLQTPFTFGPGTKVDIK

SEQ ID NO: 28 - protein, 120 aa, synthetic construct
  REGION 1..120: note = Description of Artificial Sequence: Synthetic polypeptide
QVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSS

SEQ ID NO: 29 - protein, 107 aa, synthetic construct
  REGION 1..107: note = Description of Artificial Sequence: Synthetic polypeptide
DIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 30 - protein, 120 aa, synthetic construct
  REGION 1..120: note = Description of Artificial Sequence: Synthetic polypeptide
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSSG

SEQ ID NO: 31 - protein, 115 aa, synthetic construct
  REGION 1..115: note = Description of Artificial Sequence: Synthetic polypeptide
GSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 32 - protein, 832 aa, synthetic construct
  REGION 1..832: note = Description of Artificial Sequence: Synthetic polypeptide
MALPVTALLLPLALLLHAARPDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSPRASTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 33 - protein, 1234 aa, synthetic construct
  REGION 1..1234: note = Description of Artificial Sequence: Synthetic polypeptide
MALPVTALLLPLALLLHAARPDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSPRASTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGEGGGGGDLGGVKLPHLFGKRLVEARMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLLLDRHPIAVMLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 34 - protein, 831 aa, synthetic construct
  REGION 1..831: note = Description of Artificial Sequence: Synthetic polypeptide
MALPVTALLLPLALLLHAARPDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 35 - protein, 1233 aa, synthetic construct
  REGION 1..1233: note = Description of Artificial Sequence: Synthetic polypeptide
MALPVTALLLPLALLLHAARPDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGEGGGGGDLGGVKLPHLFGKRLVEARMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLLLDRHPIAVMLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 36 - protein, 1248 aa, synthetic construct
  REGION 1..1248: note = Description of Artificial Sequence: Synthetic polypeptide
MALPVTALLLPLALLLHAARPGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRGGGGSGGGGSGGGGSGGGGSGGGGSEVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGEGGGGGFKRDLGGVKLPHLFGKRLVEARMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLLLDRHPIAVMLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 37 - protein, 841 aa, synthetic construct
  REGION 1..841: note = Description of Artificial Sequence: Synthetic polypeptide
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 38 - protein, 842 aa, synthetic construct
  REGION 1..842: note = Description of Artificial Sequence: Synthetic polypeptide
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSPRASTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 39 - protein, 835 aa, synthetic construct
  REGION 1..835: note = Description of Artificial Sequence: Synthetic polypeptide
MALPVTALLLPLALLLHAARPGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 40 - protein, 835 aa, synthetic construct; identical to SEQ ID NO: 39
  REGION 1..835: note = Description of Artificial Sequence: Synthetic polypeptide

SEQ ID NO: 41 - protein, 1133 aa, synthetic construct
  REGION 1..1133: note = Description of Artificial Sequence: Synthetic polypeptide
MALPVTALLLPLALLLHAARPQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSGGGGSGGGGSDIKNITQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSGGGGSGGGGSGGGGSGGGGSGGGGSGGGGSGGGGSEVKLEESGAELVKPGASVKLSCRTSGFNLKDTIHWVKQRPEQGLKWIGRIDPANGNTKYDPKFQDKATVTADTSSNTAYLQLSSLTSEDTAVYYCVTYAYDGNWYFDVWGAGTAVTVSSGGGGSGGGGSGGGGSGGGGSGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 42 - protein, 1134 aa, synthetic construct
  REGION 1..1134: note = Description of Artificial Sequence: Synthetic polypeptide
MALPVTALLLPLALLLHAARPDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIKGGGGSGGGGSGGGGSGGGGSDIKNITQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSGGGGSGGGGSGGGGSGGGGSGGGGSGGGGSGGGGSEVKLEESGAELVKPGASVKLSCRTSGFNLKDTIHWVKQRPEQGLKWIGRIDPANGNTKYDPKFQDKATVTADTSSNTAYLQLSSLTSEDTAVYYCVTYAYDGNWYFDVWGAGTAVTVSSGGGGSGGGGSGGGGSGGGGSEVQLVESGGGLVQPGGSLRLSCAASGYSFTGYTMNWVRQAPGKCLEWVALINPYKGVSTYNQKFKDRFTISVDKSKNTAYLQMNSLRAEDTAVYYCARSGYYGDSDWYFDVWGQGTLVTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 43 - protein, 1134 aa, synthetic construct
  REGION 1..1134: note = Description of Artificial Sequence: Synthetic polypeptide
MALPVTALLLPLALLLHAARPDIKNITQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIKGGGGSGGGGSGGGGSGGGGSGGGGSGGGGSGGGGSGGGGSGGGGSGGGGSEVQLVESGGGLVQPGGSLRLSCAASGYSFTGYTMNWVRQAPGKCLEWVALINPYKGVSTYNQKFKDRFTISVDKSKNTAYLQMNSLRAEDTAVYYCARSGYYGDSDWYFDVWGQGTLVTVSSGGGGSGGGGSGGGGSGGGGSEVKLEESGAELVKPGASVKLSCRTSGFNLKDTIHWVKQRPEQGLKWIGRIDPANGNTKYDPKFQDKATVTADTSSNTAYLQLSSLTSEDTAVYYCVTYAYDGNWYFDVWGAGTAVTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 44 - protein, 1133 aa, synthetic construct
  REGION 1..1133: note = Description of Artificial Sequence: Synthetic polypeptide
MALPVTALLLPLALLLHAARPDIKNITQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSGGGGSGGGGSGGGGSGGGGSGGGGSGGGGSGGGGSGGGGSGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSEVKLEESGAELVKPGASVKLSCRTSGFNLKDTIHWVKQRPEQGLKWIGRIDPANGNTKYDPKFQDKATVTADTSSNTAYLQLSSLTSEDTAVYYCVTYAYDGNWYFDVWGAGTAVTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 45 - protein, 1129 aa, synthetic construct
  REGION 1..1129: note = Description of Artificial Sequence: Synthetic polypeptide
MALPVTALLLPLALLLHAARPDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSDIKNITQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSGGGGSGGGGSGGGGSGGGGSGGGGSGGGGSGGGGSEVKLEESGAELVKPGASVKLSCRTSGFNLKDTIHWVKQRPEQGLKWIGRIDPANGNTKYDPKFQDKATVTADTSSNTAYLQLSSLTSEDTAVYYCVTYAYDGNWYFDVWGAGTAVTVSSGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 46 - protein, 1129 aa, synthetic construct
  REGION 1..1129: note = Description of Artificial Sequence: Synthetic polypeptide
MALPVTALLLPLALLLHAARPDIKNITQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSGGGGSDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSGGGGSGGGGSGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSGGGGSGGGGSGGGGSGGGGSEVKLEESGAELVKPGASVKLSCRTSGFNLKDTIHWVKQRPEQGLKWIGRIDPANGNTKYDPKFQDKATVTADTSSNTAYLQLSSLTSEDTAVYYCVTYAYDGNWYFDVWGAGTAVTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 47 - RNA, 100 nt, synthetic construct
  misc_feature 1..100: note = Description of Artificial Sequence: Synthetic polynucleotide
  source 1..100: mol_type = other RNA
atcacggaggtcaatgtctagttttagagctagaaatagcaagttaaaataaggctagtccgttatcaacttgaaaaagtggcaccgagtcggtgctttt

SEQ ID NO: 48 - RNA, 100 nt, synthetic construct
  misc_feature 1..100: note = Description of Artificial Sequence: Synthetic polynucleotide
  source 1..100: mol_type = other RNA
gtagacattgacctccgtgagttttagagctagaaatagcaagttaaaataaggctagtccgttatcaacttgaaaaagtggcaccgagtcggtgctttt

SEQ ID NO: 49 - RNA, 100 nt, synthetic construct; identical to SEQ ID NO: 47
  misc_feature 1..100: note = Description of Artificial Sequence: Synthetic polynucleotide
  source 1..100: mol_type = other RNA

SEQ ID NO: 50 - RNA, 100 nt, synthetic construct
  misc_feature 1..100: note = Description of Artificial Sequence: Synthetic polynucleotide
  source 1..100: mol_type = other RNA
gagaatcaaaatcggtgaatgttttagagctagaaatagcaagttaaaataaggctagtccgttatcaacttgaaaaagtggcaccgagtcggtgctttt

SEQ ID NO: 51 - RNA, 100 nt, synthetic construct
  misc_feature 1..100: note = Description of Artificial Sequence: Synthetic polynucleotide
  source 1..100: mol_type = other RNA
gaccaatctgacatgctgcagttttagagctagaaatagcaagttaaaataaggctagtccgttatcaacttgaaaaagtggcaccgagtcggtgctttt

SEQ ID NO: 52 - RNA, 100 nt, synthetic construct
  misc_feature 1..100: note = Description of Artificial Sequence: Synthetic polynucleotide
  source 1..100: mol_type = other RNA
acagctgacaggctcgacacgttttagagctagaaatagcaagttaaaataaggctagtccgttatcaacttgaaaaagtggcaccgagtcggtgctttt

SEQ ID NO: 53 - RNA, 100 nt, synthetic construct; identical to SEQ ID NO: 50
  misc_feature 1..100: note = Description of Artificial Sequence: Synthetic polynucleotide
  source 1..100: mol_type = other RNA

SEQ ID NO: 54 - RNA, 100 nt, synthetic construct
  misc_feature 1..100: note = Description of Artificial Sequence: Synthetic polynucleotide
  source 1..100: mol_type = other RNA
agggcatgtcaatattactggttttagagctagaaatagcaagttaaaataaggctagtccgttatcaacttgaaaaagtggcaccgagtcggtgctttt

SEQ ID NO: 55 - RNA, 100 nt, synthetic construct
  misc_feature 1..100: note = Description of Artificial Sequence: Synthetic polynucleotide
  source 1..100: mol_type = other RNA
cgttccaactcgaagtgccagttttagagctagaaatagcaagttaaaataaggctagtccgttatcaacttgaaaaagtggcaccgagtcggtgctttt

SEQ ID NO: 56 - RNA, 100 nt, synthetic construct; identical to SEQ ID NO: 55
  misc_feature 1..100: note = Description of Artificial Sequence: Synthetic polynucleotide
  source 1..100: mol_type = other RNA

SEQ ID NO: 57 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
caaagagattacgaatgcctngg

SEQ ID NO: 58 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
caaggcattcgtaatctcttngg

SEQ ID NO: 59 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cttgtagatatcctgatcatngg

SEQ ID NO: 60 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cttgggtcaggacatcaactngg

SEQ ID NO: 61 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cgatgatcaggatatctacangg

SEQ ID NO: 62 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ttacgaatgccttggaaaccngg

SEQ ID NO: 63 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tacgaatgccttggaaacctngg

SEQ ID NO: 64 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
acgaatgccttggaaacctgngg

SEQ ID NO: 65 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgatattgacgatataaaatngg

SEQ ID NO: 66 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atgatattgacgatataaaangg

SEQ ID NO: 67 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcatctgaagaccgatgatcngg

SEQ ID NO: 68 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aacctggggtgccttgggtcngg

SEQ ID NO: 69 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ttggaaacctggggtgccttngg

SEQ ID NO: 70 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gtatcaatatatgatacaaangg

SEQ ID NO: 71 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
caaggcaccccaggtttccangg

SEQ ID NO: 72 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cttggaaacctggggtgcctngg

SEQ ID NO: 73 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tcatcactcatttgaaaactngg

SEQ ID NO: 74 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
caagttgatgtcctgacccangg

SEQ ID NO: 75 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gtcctgacccaaggcaccccngg

SEQ ID NO: 76 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atatttgatttgaagattcangg

SEQ ID NO: 77 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tacaaaaggaaaaaatgtgtngg

SEQ ID NO: 78 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
acatataagctatttaaaaangg

SEQ ID NO: 79 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aaaagagaaagagactttcangg

SEQ ID NO: 80 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cttgatacaggtttaattcgngg

SEQ ID NO: 81 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
acagctgacaggctcgacacngg

SEQ ID NO: 82 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gatgtttcccatcttgatacngg

SEQ ID NO: 83 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gtcgagcctgtcagctgtccngg

SEQ ID NO: 84 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
caaaattcaagtgcacagcangg

SEQ ID NO: 85 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gaattttgcactcaggctggngg

SEQ ID NO: 86 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gaattaaacctgtatcaagangg

SEQ ID NO: 87 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aattaaacctgtatcaagatngg

SEQ ID NO: 88 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agttccattcattacctcacngg

SEQ ID NO: 89 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agagggtcatcacacacaagngg

SEQ ID NO: 90 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atacaagtccaggagatcttngg

SEQ ID NO: 91 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tcttgatacaggtttaattcngg

SEQ ID NO: 92 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctgacctgtgaggtaatgaangg

SEQ ID NO: 93 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
acatctaaaactttctcagangg

SEQ ID NO: 94 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcaaaattcaagtgcacagcngg

SEQ ID NO: 95 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggttgtgttgatacaagtccngg

SEQ ID NO: 96 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atcttgatacaggtttaattngg

SEQ ID NO: 97 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
attcattacctcacaggtcangg

SEQ ID NO: 98 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aacatctaaaactttctcagngg

SEQ ID NO: 99 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agcagggaacaaagtcagcangg

SEQ ID NO: 100 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
caacacaaccctgacctgtgngg

SEQ ID NO: 101 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cttgaattttgcactcaggcngg

SEQ ID NO: 102 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cattcattacctcacaggtcngg

SEQ ID NO: 103 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgcacttgaattttgcactcngg

SEQ ID NO: 104 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tctcaaaaccaaagatctccngg

SEQ ID NO: 105 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ttgacatgccctcagtatccngg

SEQ ID NO: 106 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctggattacctcttgccctcngg

SEQ ID NO: 107 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gagatggagactttatatgcngg

SEQ ID NO: 108 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agatggagactttatatgctngg

SEQ ID NO: 109 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agggcatgtcaatattactgngg

SEQ ID NO: 110 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gatggagactttatatgctgngg

SEQ ID NO: 111 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tattatgtctgctaccccagngg

SEQ ID NO: 112 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgccatagtatttcagatccngg

SEQ ID NO: 113 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agataaaagttcgcatcttcngg

SEQ ID NO: 114 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctgaaaattccttcagtgacngg

SEQ ID NO: 115 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctgagggcaagaggtaatccngg

SEQ ID NO: 116 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tttcagatccaggatactgangg

SEQ ID NO: 117 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tatctctacctgagggcaagngg

SEQ ID NO: 118 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgaggatcacctgtcactgangg

SEQ ID NO: 119 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aatcatctgctacggacaacngg

SEQ ID NO: 120 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcagacttttgacgcttgacngg

SEQ ID NO: 121 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccgttccaactcgaagtgccngg

SEQ ID NO: 122 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cgttccaactcgaagtgccangg

SEQ ID NO: 123 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctggcacttcgagttggaacngg

SEQ ID NO: 124 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gtctgccagcggctgaactgngg

SEQ ID NO: 125 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atcatctgctacggacaactngg

SEQ ID NO: 126 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agacttttgacgcttgactgngg

SEQ ID NO: 127 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cagacttttgacgcttgactngg

SEQ ID NO: 128 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cctggcacttcgagttggaangg

SEQ ID NO: 129 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcaccccacagttcagccgcngg

SEQ ID NO: 130 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccttgaggtagacctccagcngg

SEQ ID NO: 131 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aggtctacctcaaggacggangg

SEQ ID NO: 132 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tggaacgggtgagccttgccngg

SEQ ID NO: 133 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgtggggtgcccttaagcctngg

SEQ ID NO: 134 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aagcgtcaaaagtctgccagngg

SEQ ID NO: 135 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tagcagatgattgagctctgngg

SEQ ID NO: 136 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gattgagctctgaggtgtgtngg

SEQ ID NO: 137 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggggccggagctccaagcagngg

SEQ ID NO: 138 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggtgtgtaggtgacaaggaangg

SEQ ID NO: 139 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccggagctccaagcagtgggngg

SEQ ID NO: 140 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggtagacctccagctggcccngg

SEQ ID NO: 141 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctcgaagtgccagggccagcngg

SEQ ID NO: 142 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctggccctggcacttcgagtngg

SEQ ID NO: 143 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tctgccagcggctgaactgtngg

SEQ ID NO: 144 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccatgtgccatccgtccttgngg

SEQ ID NO: 145 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccagctggaggtctacctcangg

SEQ ID NO: 146 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tctgaggtgtgtaggtgacangg

SEQ ID NO: 147 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aggaaggggccaaggcttaangg

SEQ ID NO: 148 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cagagctcaatcatctgctangg

SEQ ID NO: 149 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gggccggagctccaagcagtngg

SEQ ID NO: 150 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cctcccactgcttggagctcngg

SEQ ID NO: 151 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tggagctccggccccagctcngg

SEQ ID NO: 152 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gtgtgtaggtgacaaggaagngg

SEQ ID NO: 153 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atggtttgcagccagagctgngg

SEQ ID NO: 154 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctggaggtctacctcaaggangg

SEQ ID NO: 155 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctgccagcggctgaactgtgngg

SEQ ID NO: 156 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aatgacatgtgtcactctctngg

SEQ ID NO: 157 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
acatggtttgcagccagagcngg

SEQ ID NO: 158 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
catggtttgcagccagagctngg

SEQ ID NO: 159 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gacacatgtcatttctgctgngg

SEQ ID NO: 160 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
actggggtcctcccactgctngg

SEQ ID NO: 161 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cctcaaggacggatggcacangg

SEQ ID NO: 162 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aggtgtgtaggtgacaaggangg

SEQ ID NO: 163 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aaggaaggggccaaggcttangg

SEQ ID NO: 164 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gaagtgccagggccagctggngg

SEQ ID NO: 165 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tttgcagccagagctggggcngg

SEQ ID NO: 166 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aaatgacatgtgtcactctcngg

SEQ ID NO: 167 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aggtgacaaggaaggggccangg

SEQ ID NO: 168 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atttctgctgtggctgcagtngg

SEQ ID NO: 169 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gctgtggctgcagttggagangg

SEQ ID NO: 170 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggcgggggccttgtcgttggngg

SEQ ID NO: 171 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctctggagttgtggtgggcgngg

SEQ ID NO: 172 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tctggagttgtggtgggcggngg

SEQ ID NO: 173 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cgttggaggtgttgtcttctngg

SEQ ID NO: 174 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agacaacacctccaacgacangg

SEQ ID NO: 175 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gtgggcgggggccttgtcgtngg

SEQ ID NO: 176 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tcgttggaggtgttgtcttcngg

SEQ ID NO: 177 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
accacaactccagagcccacngg

SEQ ID NO: 178 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gctctggagttgtggtgggcngg

SEQ ID NO: 179 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gtgggctctggagttgtggtngg

SEQ ID NO: 180 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgtgggctctggagttgtggngg

SEQ ID NO: 181 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gttggaggtgttgtcttctgngg

SEQ ID NO: 182 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggctctggagttgtggtgggngg

SEQ ID NO: 183 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
catagctgatggtaccccccngg

SEQ ID NO: 184 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cggccagcactgtgccggcgngg

SEQ ID NO: 185 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
caagaactcggccacttttcngg

SEQ ID NO: 186 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggtgttcccgtggctcccctngg

SEQ ID NO: 187 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccagcactgtgccggcgtggngg

SEQ ID NO: 188 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggcaagggctggtgttcccgngg

SEQ ID NO: 189 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggcgtggtggagttctacagngg

SEQ ID NO: 190 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccaccacgccggcacagtgcngg

SEQ ID NO: 191 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggagttctacagcggcagccngg

SEQ ID NO: 192 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gttctacagcggcagcctggngg

SEQ ID NO: 193 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
accagcccttgccaatccaangg

SEQ ID NO: 194 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agttctacagcggcagcctgngg

SEQ ID NO: 195 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccaggtcctgggtcttgtccngg

SEQ ID NO: 196 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tggtgttcccgtggctccccngg

SEQ ID NO: 197 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gagttctacagcggcagcctngg

SEQ ID NO: 198 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gaactcaagctgtacctcccngg

SEQ ID NO: 199 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aagaactcggccacttttctngg

SEQ ID NO: 200 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tccattggattggcaagggcngg

SEQ ID NO: 201 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ttctacagcggcagcctgggngg

SEQ ID NO: 202 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agaactcggccacttttctgngg

SEQ ID NO: 203 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcttcaagaaggagccacacngg

SEQ ID NO: 204 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gatcttccattggattggcangg

SEQ ID NO: 205 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gctgtagaactccaccacgcngg

SEQ ID NO: 206 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gtcctgggcctcatagctgangg

SEQ ID NO: 207 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
taccatcagctatgaggcccngg

SEQ ID NO: 208 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggggggtaccatcagctatgngg

SEQ ID NO: 209 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cctgaagcaatgctccagggngg

SEQ ID NO: 210 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ttttcctgaagcaatgctccngg

SEQ ID NO: 211 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctctggcagatgcttcaagangg

SEQ ID NO: 212 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agaggaagttctccaggtccngg

SEQ ID NO: 213 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tctggcggccagcactgtgcngg

SEQ ID NO: 214 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ttgagttctggatcttccatngg

SEQ ID NO: 215 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ttctggatcttccattggatngg

SEQ ID NO: 216 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atcttccattggattggcaangg

SEQ ID NO: 217 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tcaagaaggagccacactggngg

SEQ ID NO: 218 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gggaggtacagcttgagttcngg

SEQ ID NO: 219 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cccgtggctcccctgggtctngg

SEQ ID NO: 220 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccaggacaagacccaggaccngg

SEQ ID NO: 221 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctctgcaacaacctccagtgngg

SEQ ID NO: 222 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgttgcagaggaagttctccngg

SEQ ID NO: 223 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
caggtcctgggtcttgtcctngg

SEQ ID NO: 224 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgaggcccaggacaagacccngg

SEQ ID NO: 225 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctgtgccaccagctgcagccngg

SEQ ID NO: 226 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgtgccaccagctgcagcctngg

SEQ ID NO: 227 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
catctgccagagactgaggcngg

SEQ ID NO: 228 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctgcagctggtggcacagtcngg

SEQ ID NO: 229 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cacactggaggttgttgcagngg

SEQ ID NO: 230 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cagctggtggcacagtctggngg

SEQ ID NO: 231 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agcaaaggagggcaagaactngg

SEQ ID NO: 232 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gaggaagttctccaggtcctngg

SEQ ID NO: 233 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gccaccagctgcagcctgggngg

SEQ ID NO: 234 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcaggcagagcccaagacccngg

SEQ ID NO: 235 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
caggcagagcccaagacccangg

SEQ ID NO: 236 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tcctcccaggctgcagctggngg

SEQ ID NO: 237 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gctctgcctgcctcagtctcngg

SEQ ID NO: 238 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cctccctggagcattgcttcngg

SEQ ID NO: 239 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tttcctgaagcaatgctccangg

SEQ ID NO: 240 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccgtggctcccctgggtcttngg

SEQ ID NO: 241 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aaaatcaagccccagaaaagngg

SEQ ID NO: 242 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gaagcatctgccagagactgngg

SEQ ID NO: 243 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gaagcatctgccagagactgngg

SEQ ID NO: 244 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aggcagagcccaagacccagngg

SEQ ID NO: 245 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cccaagacccaggggagccangg

SEQ ID NO: 246 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tactcaggttcaggagacgcngg

SEQ ID NO: 247 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tcaggagacgccgggcctccngg

SEQ ID NO: 248 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
actcaggttcaggagacgccngg

SEQ ID NO: 249 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cagtgtctctactcaggttcngg

SEQ ID NO: 250 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atgctcccagggctgcgtgcngg

SEQ ID NO: 251 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gagacgccgggcctcctggangg

SEQ ID NO: 252 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cagcagcagtgtctctactcngg

SEQ ID NO: 253 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gatggcattcacatgctcccngg

SEQ ID NO: 254 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggagcatgtgaatgccatccngg

SEQ ID NO: 255 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tagagacactgctgctgagangg

SEQ ID NO: 256 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcatgtgaatgccatccaggngg

SEQ ID NO: 257 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atggcattcacatgctcccangg

SEQ ID NO: 258 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgaatgccatccaggaggccngg

SEQ ID NO: 259 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgctcccagggctgcgtgctngg

SEQ ID NO: 260 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cagccccagcacgcagccctngg

SEQ ID NO: 261 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gctcccagggctgcgtgctgngg

SEQ ID NO: 262 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccttggatttcagcggcacangg

SEQ ID NO: 263 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccttgtgccgctgaaatccangg

SEQ ID NO: 264 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgaacctggctaccaggaccngg

SEQ ID NO: 265 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agggccaggtcctggtagccngg

SEQ ID NO: 266 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctcagctgaacctggctaccngg

SEQ ID NO: 267 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agaaaaaacaggagagtgcangg

SEQ ID NO: 268 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcacaaggctcagctgaaccngg

SEQ ID NO: 269 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tggcttgccttggatttcagngg

SEQ ID NO: 270 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aaacaggagagtgcagggccngg

SEQ ID NO: 271 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gagagtgcagggccaggtccngg

SEQ ID NO: 272 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggatgaagagaagaaaaaacngg

SEQ ID NO: 273 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aagaaaaaacaggagagtgcngg

SEQ ID NO: 274 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccgggtgacagtgcttcggcngg

SEQ ID NO: 275 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
acacaaagctggcgatgcctngg

SEQ ID NO: 276 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccctcagtccttggatagtgngg

SEQ ID NO: 277 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gtgcggcaacctacatgatgngg

SEQ ID NO: 278 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctgtgcggcaacctacatgangg

SEQ ID NO: 279 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggcccagcctgctgtggtacngg

SEQ ID NO: 280 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gttcacttgatttccactggngg

SEQ ID NO: 281 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
caactcattccccatcatgtngg

SEQ ID NO: 282 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccgcacagacttcagtcaccngg

SEQ ID NO: 283 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgtgcggcaacctacatgatngg

SEQ ID NO: 284 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cctcactatccaaggactgangg

SEQ ID NO: 285 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cggacctcagtggctttgccngg

SEQ ID NO: 286 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gaggttcacttgatttccacngg

SEQ ID NO: 287 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccaggtgactgaagtctgtgngg

SEQ ID NO: 288 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
actggaggtgcccgtgcagangg

SEQ ID NO: 289 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
caagtgaacctcactatccangg

SEQ ID NO: 290 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gtggtactggccagcagccgngg

SEQ ID NO: 291 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aggtccgggtgacagtgcttngg

SEQ ID NO: 292 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atctgcacgggcacctccagngg

SEQ ID NO: 293 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccgtgcagatggaatcatctngg

SEQ ID NO: 294 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctagatgattccatctgcacngg

SEQ ID NO: 295 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
acctcactatccaaggactgngg

SEQ ID NO: 296 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cctgccgaagcactgtcaccngg

SEQ ID NO: 297 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tggccagtaccacagcaggcngg

SEQ ID NO: 298 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atctccaggcaaagccactgngg

SEQ ID NO: 299 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcacgtggcccagcctgctgngg

SEQ ID NO: 300 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gtgtgtgagtatgcatctccngg

SEQ ID NO: 301 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cactgtcacccggacctcagngg

SEQ ID NO: 302 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gctggcgatgcctcggctgcngg

SEQ ID NO: 303 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctgctggccagtaccacagcngg

SEQ ID NO: 304 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aggcaaagccactgaggtccngg

SEQ ID NO: 305 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcagatggaatcatctaggangg

SEQ ID NO: 306 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cctagatgattccatctgcangg

SEQ ID NO: 307 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggccagtaccacagcaggctngg

SEQ ID NO: 308 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgcatactcacacacaaagcngg

SEQ ID NO: 309 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcttcggcaggctgacagccngg

SEQ ID NO: 310 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
caggcaaagccactgaggtcngg

SEQ ID NO: 311 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgtagcaccgcccagacgacngg

SEQ ID NO: 312 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggcgccctggccagtcgtctngg

SEQ ID NO: 313 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cgtctgggcggtgctacaacngg

SEQ ID NO: 314 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aggcgccctggccagtcgtcngg

SEQ ID NO: 315 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
caccgcccagacgactggccngg

SEQ ID NO: 316 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
accgcccagacgactggccangg

SEQ ID NO: 317 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gggcggtgctacaactgggcngg

SEQ ID NO: 318 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gtctgggcggtgctacaactngg

SEQ ID NO: 319 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cgactggccagggcgcctgtngg

SEQ ID NO: 320 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cggtgctacaactgggctggngg

SEQ ID NO: 321 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tggcggccaggatggttcttngg

SEQ ID NO: 322 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
acgactggccagggcgcctgngg

SEQ ID NO: 323 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctacaactgggctggcggccngg

SEQ ID NO: 324 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gccctggccagtcgtctgggngg

SEQ ID NO: 325 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgcagatcccacaggcgcccngg

SEQ ID NO: 326 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aactgggctggcggccaggangg

SEQ ID NO: 327 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cggagagcttcgtgctaaacngg

SEQ ID NO: 328 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcgtgacttccacatgagcgngg

SEQ ID NO: 329 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atgtggaagtcacgcccgttngg

SEQ ID NO: 330 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gccctgctcgtggtgaccgangg

SEQ ID NO: 331 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cacgaagctctccgatgtgtngg

SEQ ID NO: 332 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cctgctcgtggtgaccgaagngg

SEQ ID NO: 333 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgacacggaagcggcagtccngg

SEQ ID NO: 334 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccccttcggtcaccacgagcngg

SEQ ID NO: 335 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cagcaaccagacggacaagcngg

SEQ ID NO: 336 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcagttgtgtgacacggaagngg

SEQ ID NO: 337 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cccttcggtcaccacgagcangg

SEQ ID NO: 338 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccgggctggctgcggtcctcngg

SEQ ID NO: 339 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aggcggccagcttgtccgtcngg

SEQ ID NO: 340 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cagcttgtccgtctggttgcngg

SEQ ID NO: 341 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cggtcaccacgagcagggctngg

SEQ ID NO: 342 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gtgtcacacaactgcccaacngg

SEQ ID NO: 343 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctgcagcttctccaacacatngg

SEQ ID NO: 344 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
caagctggccgccttccccgngg

SEQ ID NO: 345 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cgtgtcacacaactgcccaangg

SEQ ID NO: 346 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cgttgggcagttgtgtgacangg

SEQ ID NO: 347 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcttgtccgtctggttgctgngg

SEQ ID NO: 348 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cggaagcggcagtcctggccngg

SEQ ID NO: 349 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cgatgtgttggagaagctgcngg

SEQ ID NO: 350 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
catgtggaagtcacgcccgtngg

SEQ ID NO: 351 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccctgctcgtggtgaccgaangg

SEQ ID NO: 352 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cgggctggctgcggtcctcgngg

SEQ ID NO: 353 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agcttgtccgtctggttgctngg

SEQ ID NO: 354 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gaaggtggcgttgtccccttngg

SEQ ID NO: 355 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
acttccacatgagcgtggtcngg

SEQ ID NO: 356 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gccgggctggctgcggtcctngg

SEQ ID NO: 357 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tcggtcaccacgagcagggcngg

SEQ ID NO: 358 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tctggttgctggggctcatgngg

SEQ ID NO: 359 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
acggaagcggcagtcctggcngg

SEQ ID NO: 360 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cccgaggaccgcagccagccngg

SEQ ID NO: 361 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctggctgcggtcctcggggangg

SEQ ID NO: 362 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
catgagccccagcaaccagangg

SEQ ID NO: 363 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agtcctggccgggctggctgngg

SEQ ID NO: 364 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gggggttccagggcctgtctngg

SEQ ID NO: 365 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggtcaccacgagcagggctgngg

SEQ ID NO: 366 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gctgcggtcctcggggaaggngg

SEQ ID NO: 367 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggaccgcagccagcccggccngg

SEQ ID NO: 368 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gagaaggtgggggggttccangg

SEQ ID NO: 369 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggagaaggtgggggggttccngg

SEQ ID NO: 370 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agcggcagtcctggccgggcngg

SEQ ID NO: 371 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggggttccagggcctgtctgngg

SEQ ID NO: 372 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cttctccccagccctgctcgngg

SEQ ID NO: 373 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gttggagaagctgcaggtgangg

SEQ ID NO: 374 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggggggttccagggcctgtcngg

SEQ ID NO: 375 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggagaagctgcaggtgaaggngg

SEQ ID NO: 376 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cacgagcagggctggggagangg

SEQ ID NO: 377 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcagggctggggagaaggtgngg

SEQ ID NO: 378 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cagggctggggagaaggtggngg

SEQ ID NO: 379 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gagcagggctggggagaaggngg

SEQ ID NO: 380 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agcagggctggggagaaggtngg

SEQ ID NO: 381 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agggctggggagaaggtgggngg

SEQ ID NO: 382 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gggctggggagaaggtggggngg

SEQ ID NO: 383 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agaagtggaatacagagcggngg

SEQ ID NO: 384 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aatgtggcaacgtggtgctcngg

SEQ ID NO: 385 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctaaatggggatttccgcaangg

SEQ ID NO: 386 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
catccagatactggctaaatngg

SEQ ID NO: 387 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cagacgggcacgaggttcccngg

SEQ ID NO: 388 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcggctggggtgtagaagcangg

SEQ ID NO: 389 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gaacctcgtgcccgtctgctngg

SEQ ID NO: 390 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gacgggcacgaggttccctgngg

SEQ ID NO: 391 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atccccatttagccagtatcngg

SEQ ID NO: 392 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gtggaatacagagcggaggtngg

SEQ ID NO: 393 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agacgggcacgaggttccctngg

SEQ ID NO: 394 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggaacctcgtgcccgtctgcngg

SEQ ID NO: 395 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gagtcacattctctatggtcngg

SEQ ID NO: 396 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atgtgactctagcagacagtngg

SEQ ID NO: 397 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ttttcatcattcattatgccngg

SEQ ID NO: 398 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aatgtgactctagcagacagngg

SEQ ID NO: 399 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atccagatactggctaaatgngg

SEQ ID NO: 400 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgctgccggatccaaatcccngg

SEQ ID NO: 401 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tctacaccccagccgccccangg

SEQ ID NO: 402 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ttatgcctgggatttggatcngg

SEQ ID NO: 403 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cgctctgtattccacttctgngg

SEQ ID NO: 404 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gaggttccctggggcggctgngg

SEQ ID NO: 405 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgccccagcagacgggcacgngg

SEQ ID NO: 406 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
acagtgggatctactgctgcngg

SEQ ID NO: 407 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgtgtttgaatgtggcaacgngg

SEQ ID NO: 408 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgaaaaatttaacctgaagtngg

SEQ ID NO: 409 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
acatccagatactggctaaangg

SEQ ID NO: 410 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atgaaagggatgtgaattatngg

SEQ ID NO: 411 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tggtgctcaggactgatgaangg

SEQ ID NO: 412 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggtgtagaagcagggcagatngg

SEQ ID NO: 413 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
acgttgccacattcaaacacngg

SEQ ID NO: 414 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
acgaggttccctggggcggcngg

SEQ ID NO: 415 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gcctgtcctgtgtttgaatgngg

SEQ ID NO: 416 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gtgcccgtctgctggggcaangg

SEQ ID NO: 417 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aacctcgtgcccgtctgctgngg

SEQ ID NO: 418 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggcggctggggtgtagaagcngg

SEQ ID NO: 419 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agtcacattctctatggtcangg

SEQ ID NO: 420 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctggtttgatgaccaacttcngg

SEQ ID NO: 421 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cattcattatgcctgggattngg

SEQ ID NO: 422 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tgctagagtcacattctctangg

SEQ ID NO: 423 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gggcacgaggttccctggggngg

SEQ ID NO: 424 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggctcctttgccccagcagangg

SEQ ID NO: 425 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
attattggacatccagatacngg

SEQ ID NO: 426 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tttcatcattcattatgcctngg

SEQ ID NO: 427 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ttctacaccccagccgccccngg

SEQ ID NO: 428 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tcagggacacatctcctttgngg

SEQ ID NO: 429 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gctcctttgccccagcagacngg

SEQ ID NO: 430 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctcagaagtggaatacagagngg

SEQ ID NO: 431 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cgaggttccctggggcggctngg

SEQ ID NO: 432 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gccacattcaaacacaggacngg

SEQ ID NO: 433 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggtgctcaggactgatgaaangg

SEQ ID NO: 434 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
aggtcacccctgcaccgactngg

SEQ ID NO: 435 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ctctctgccgagtcggtgcangg

SEQ ID NO: 436 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tctctctgccgagtcggtgcngg

SEQ ID NO: 437 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ccaaggatgcttaccaccagngg

SEQ ID NO: 438 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tctctgccgagtcggtgcagngg

SEQ ID NO: 439 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
cccctggtggtaagcatcctngg

SEQ ID NO: 440 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
tccaaggatgcttaccaccangg

SEQ ID NO: 441 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ggtggtaagcatccttggaangg

SEQ ID NO: 442 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
gtgaagtctctctgccgagtngg

SEQ ID NO: 443 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
atgcttaccaccaggggacangg

SEQ ID NO: 444 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
ttccaaggatgcttaccaccngg

SEQ ID NO: 445 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
agtcggtgcaggggtgacctngg

SEQ ID NO: 446 - DNA, 23 bp, synthetic construct
  misc_feature 1..23: note = Description of Artificial Sequence: Synthetic polynucleotide
  misc_feature 21: note = n is a, c, g, or t
  source 1..23: mol_type = other DNA
acttcactgcagcctttccangg

SEQ ID NO: 447 - protein, 30 aa, synthetic construct
  REGION 1..30: note = Description of Artificial Sequence: Synthetic polypeptide
  REGION 1..30: note = MISC_FEATURE - This sequence may encompass 2-6 "Gly Gly Gly Gly Ser" repeating units
GGGGSGGGGSGGGGSGGGGSGGGGSGGGGS

SEQ ID NO: 448 - protein, 50 aa, synthetic construct
  REGION 1..50: note = Description of Artificial Sequence: Synthetic polypeptide
  REGION 1..50: note = MISC_FEATURE - This sequence may encompass 1-3 "Gly Gly Gly Gly Ser" repeating units, 1 "Gly Gly Gly Gly Pro" repeating unit, and 1 "Gly Gly Gly Gly Cys" repeating unit.
  REGION 1..50: note = MISC_FEATURE - This sequence may encompass 1-3 "Gly Gly Gly Gly Ser" repeating units, 1 "Gly Gly Gly Gly Pro" unit, and 1 "Gly Gly Gly Gly Cys" unit.
GGGGSGGGGCGGGGSGGGGSGGGGPGGGGSGGGGSGGGGSGGGGCGGGGS

SEQ ID NO: 449 - protein, 20 aa, synthetic construct
  REGION 1..20: note = Description of Artificial Sequence: Synthetic polypeptide
  REGION 1..20: note = MISC_FEATURE - This sequence may encompass 3-4 "Gly Gly Gly Gly Ser" repeating units
GGGGSGGGGSGGGGSGGGGS

SEQ ID NO: 450 - protein, 20 aa, synthetic construct
  REGION 1..20: note = Description of Artificial Sequence: Synthetic polypeptide
GGGGSGGGGSGGGGSGGGGS

SEQ ID NO: 451 - protein, 50 aa, synthetic construct
  REGION 1..50: note = Description of Artificial Sequence: Synthetic polypeptide
GGGGSGGGGSGGGGSGGGGSGGGGSGGGGSGGGGSGGGGSGGGGSGGGGS

SEQ ID NO: 452 - protein, 45 aa, synthetic construct
  REGION 1..45: note = Description of Artificial Sequence: Synthetic polypeptide
GGGGSGGGGSGGGGSGGGGSGGGGPGGGGSGGGGSGGGGSGGGGS